(19) United States
(12) Patent Application Publication
(10) Pub. No.: US 2026/0356860 A1
(43) Pub. Date: Nov. 12, 2026

(54) 2-SUBSTITUTED 3,4A,5,7,8,8A-HEXAHYDRO-4H-THIOPYRANO[4,3-DJPYRIMIDIN-4-ONES FOR WOUND TREATMENT

(71) Applicant: ELUCIDERM INC., San Diego, CA (US)

(72) Inventor: Daniel D. Holsworth

(21) Appl. No.: 19/122,155

(22) PCT Filed: Oct. 18, 2023
(86) PCT No.: PCT/US2023/035442

§ 371 Date: Apr. 17, 2025

Related U.S. Application Data
(60) Provisional application No. 63/417,257, filed on Oct. 18, 2022.
(60) Provisional application No. 63/418,947, filed on Oct. 24, 2022.
(60) Provisional application No. 63/418,956, filed on Oct. 24, 2022.

(51) Int. Cl. C07F 5/02; A61K 31/519; A61K 31/69; A61P 17/02; C07D 495/04

(52) U.S. Cl. CPC C07F 5/025; A61K 31/519; A61K 31/69; A61P 17/02; C07D 495/04

(57) ABSTRACT

Provided herein are compounds, pharmaceutical compositions comprising the compounds, methods of preparing the compounds, and methods of using the compounds and compositions.

### CROSS-REFERENCE TO RELATED APPLICATIONS

This application is a PCT International Application which claims the benefit of U.S. Provisional Application No. 63/417,257, filed on Oct. 18, 2022; U.S. Provisional Application No. 63/418,947, filed on Oct. 24, 2022; and U.S. Provisional Application No. 63/418,956, filed on Oct. 24, 2022; the contents of each application are hereby incorporated by reference in their entireties.

### FIELD

Provided herein are compounds, pharmaceutical compositions comprising the compounds, methods of preparing the compounds, and methods of using the compounds and compositions in treating wounds, and in particular, the enhanced tissue regeneration following treatment of a wound. Also provided herein are methods of treating conditions associated with Wnt transcription products or Wnt signaling pathway activity in a mammal comprising administering a therapeutically effective amount of a compound or composition to a mammal.

### BACKGROUND

The Wnt pathway has been shown to play a key role in dermal fibrosis and scarring. The Wnt pathway is an evolutionary conserved pathway that regulates crucial aspects of cell fate determination, cell polarity, cell migration, neural patterning, and organogenesis during embryonic development. This pathway is instrumental in ensuring proper tissue development in embryos and tissue maintenance in adults. Wnt signaling is involved at the beginning stages of skin development, where following gastrulation, embryonic cells of the ectoderm and the mesoderm differentiate to form the epidermis and dermis, respectively.

Although there are at least three distinct Wnt signaling pathways involved in the signal transduction process, the canonical (or β-catenin dependent) Wnt pathway is the most understood. β-Catenin is the key effector molecule resulting from the signaling of the canonical Wnt pathway, and its protein levels are regulated through a “destruction complex.” In the absence of a Wnt signal, the transcriptional activator β-catenin is actively degraded in the cell by the actions of a protein complex, designated the “destruction complex.” Within this complex, Axin-1 and -2 with adenomatous polypsis coli form a scaffold that facilitates β-catenin phosphorylation by casein-kinase 19a and glycogen synthase kinase 30. Phosphorylated β-catenin is recognized and ubiquitinylated, resulting in its proteosomal degradation. Tankryase I and II (TK1 and TK2) are poly(ADP-ribose) polymerases (PARPs) that function to parsylate and destabilize Axin-1 and -2 proteins, thus destabilizing the β-catenin destruction complex. Once the destruction complex is destabilized, this allows β-catenin to be dephosphorylated, and subsequently stabilized and allowed to accumulate in the cytoplasm and enter the cell nucleus, where it interacts with members of the Tcf/Lef family. β-catenin converts the Tcf proteins into potent transcriptional activators by recruiting co-activator proteins, thus ensuring efficient activation of Wnt target genes. The Wnt pathway, once activated by the Wnt family of natural ligands, upregulates TNK1 and 2 to help destabilize the destruction complex. Studies have shown that TNK1 and 2 are critical regulators of canonical Wnt signaling.

XAV939 is a small molecule that selectively inhibits Wnt/β-catenin-mediated transcription through TK 1 and TK2 inhibition with an IC₅₀ of 11 nM/4 nM in cell-free assays, regulates axin levels, and does not affect CRE, F-κB, or TGF-β. Recently, topical application of XAV939 in a mouse ear punch assay demonstrated that XAV939 significantly increased rate of wound closure with reduced fibrosis (scarring) (Bastakoty, D. et al. FASEB J., 2015, 29(12): 4881-4892). However, XAV939 was dissolved in dimethyl sulfoxide (DMSO) and used only as a “research tool” compound due to its very low aqueous solubility (<1 μg/mL). The problem with this approach is that humans cannot tolerate the use of DMSO.

Utility has been shown for a matrix component comprising graphene oxide (GO) and hyaluronic acid (HA) as providing a supportive matrix for XAV939 and allowing the use of XAV939 as a therapeutic for wound healing in humans and animals, see for example US20210000959, where XAV939 in a GO-HA matrix provides substantial improvement to the quality of wound healing; specifically causing the tissues to limit scarring following a fibrotic wound healing pathway. Further, increased cartilage regeneration and healing following acute injury has been observed, for example by way of a 2 mm biopsy punch wound made in the center of the cartilaginous region of a C57B1/CJ mouse, treated with XAV939 (Bastakoty, D. et al. FASEB J., 2015, 29(12): 4881-4892 and WO2023/039298).

While the therapeutic utility of XAV939 has been demonstrated, XAV939 has poor solubility in water-based solutions. Therefore, there is a need for Wnt inhibitors with improved/different characteristics, such as increased solubility in water, increased Wnt inhibition, increased bioavailability, and/or modified biological stability.

### SUMMARY

In the first aspect, provided herein is a compound of Formula (I), or any prodrug, pharmaceutically acceptable salt, metabolite, polymorph, solvate, hydrate, stereoisomer, and/or tautomer thereof:

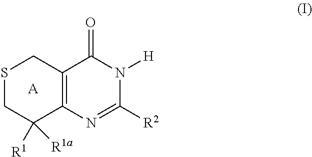

(I)
wherein
R¹ is hydrogen, deuterium, C₁-C₃alkyl, —OH, —O—C₁-C₃alkyl, —CH₂OH, or —B(OH)₂;
R^{1a} is hydrogen, deuterium, or C₁-C₃alkyl;
R² is
   (a) phenyl substituted with R³ and additionally optionally substituted with 1, 2, or 3 R^{3a} groups;
   (b) phenyl substituted with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups,
   (c) phenyl optionally substituted with 1, 2, or 3 R^{3a} groups and additionally substituted with -phenyl-R³ where the phenyl in -phenyl-R³ is optionally substituted with 1, 2, or 3 R^{3a} groups;
   (d) 5- or 6-membered heteroaryl substituted with R³ and additionally optionally substituted with 1, 2, or 3 R^{3a} groups;
   (e) 5- or 6-membered heteroaryl optionally substituted with 1, 2, or 3 R^{3a} groups and additionally substituted with -phenyl-R³ where the phenyl is additionally optionally substituted with 1, 2, or 3 R^{3a} groups;
   (f) 5- or 6-membered heteroaryl optionally substituted with 1, 2, or 3 R^{3a} groups and additionally substituted with -(5- or 6-membered heteroaryl)-R³ where the 5- or 6-membered heteroaryl in -(5- or 6-membered heteroaryl)-R³ is optionally substituted with 1, 2, or 3 R^{3a} groups;
   (g) C₃-C₆cycloalkyl substituted with R³ and additionally optionally substituted with 1 or 2 R^{3a} groups;
   (h) C₃-C₆cycloalkyl substituted with 5- or 6-membered heteroaryl where the 5- or 6-membered heteroaryl is substituted with R³ and additionally optionally substituted with 1, 2, or 3 R^{3a} groups, and where the cycloalkyl is optionally substituted with 1 or 2 R^{3a} groups;
   (i) C₃-C₆cycloalkyl substituted with phenyl where the phenyl is substituted with R³ and additionally optionally substituted with 1, 2, or 3 R^{3a} groups, and where the cycloalkyl is optionally substituted with 1 or 2 R^{3a} groups;
   (j) 3- to 8-membered heterocycloalkyl substituted with phenyl or substituted with 5- or 6-membered heteroaryl, where the phenyl and the 5- to 6-membered heteroaryl are substituted with R³ and additionally optionally substituted with 1, 2, or 3 R^{3a} groups;
   (k) —CH═CH—R⁵ where R⁵ is phenyl or 5- or 6-membered heteroaryl, where the phenyl and the 5- or 6-membered heteroaryl are substituted with R³ and additionally optionally substituted with 1, 2, or 3 R^{3a} groups;
R³ is independently selected from —B(OH)₂, cyano, halo, halo-C₁-C₆alkyl, —(C₀-C₆alkylene)-O—R⁴, or 5- to 10-membered heterocyclic wherein the 5- to 10-membered heterocyclic is optionally substituted with cyano; or when R² is (a), then R³ and one R^{3a}, when on adjacent carbons, together with the carbons to which they are attached form

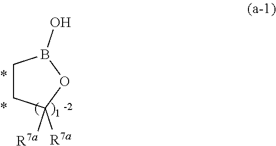

where the * indicate the carbons shared with the phenyl ring and where the remaining optional R^{3a} on the phenyl portion are as defined below, and each R^{7a} is independently hydrogen or C₁-C₆alkyl;
each R^{3a} is independently selected from cyano, halo, —OH, C₁-C₆alkyl, halo-C₁-C₆alkyl, and C₁-C₆alkoxy;
R⁴ is hydroxy-C₁-C₆alkyl, C₁-C₆alkoxy-C₁-C₆alkyl, or C₁-C₆alkoxycarbonyl-NH—C₁-C₆alkyl; and
provided that the compound is not
   2-(4-(trifluoromethyl)phenyl)-3,5,7,8-tetrahydro-4H-thiopyrano[4,3-d]pyrimidin-4-one or a single stereoisomer or mixture of stereoisomers thereof, a single tautomer or mixture of tautomers thereof; and/or a pharmaceutically acceptable salt thereof;
   2-(4-(4-methoxyphenyl)piperazin-1-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
   2-(4-(3-methoxyphenyl)piperazin-1-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
   2-(4-(2-methoxyphenyl)piperazin-1-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
   2-(4-chlorophenyl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof,
   2-(5-(trifluoromethyl)pyridin-2-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
   2-(3-(trifluoromethyl)phenyl)-3,5,7,8-tetrahydro-4H-thiopyrano[4,3-d]pyrimidin-4-one or a single stereoisomer or mixture of stereoisomers thereof, a single tautomer or mixture of tautomers thereof; and/or a pharmaceutically acceptable salt thereof;
   2-(3-(trifluoromethyl)phenyl)-3,5,7,8-tetrahydro-4H-thiopyrano[4,3-d]pyrimidin-4-one or a single stereoisomer or mixture of stereoisomers thereof, a single tautomer or mixture of tautomers thereof; and/or a pharmaceutically acceptable salt thereof; or
   2-(5-chlorothiophen-3-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
      In some embodiments, provided is a compound of Formula (I) (or any embodiments thereof) or a single stereoisomer or mixture of stereoisomers thereof; a single tautomer or mixture of tautomers thereof, and/or a pharmaceutically acceptable salt thereof.

In a second aspect, provided herein is a pharmaceutical composition comprising a compound of Formula (I) (or any embodiments thereof), or any prodrug, pharmaceutically acceptable salt, metabolite, polymorph, solvate, hydrate, stereoisomer, and/or tautomer thereof; and a pharmaceutically acceptable carrier. In one or more embodiments, provided is a compound of Formula (I) (or any embodiments thereof) or a single stereoisomer or mixture of stereoisomers thereof, a single tautomer or mixture of tautomers thereof, and/or a pharmaceutically acceptable salt thereof; and a pharmaceutically acceptable carrier.

In a third aspect, provided is a method for inhibiting Wnt transcription products or Wnt signaling pathway activity in a subject comprising contacting an effective amount of a compound of Formula (I) (or any embodiments thereof), or any prodrug, pharmaceutically acceptable salt, metabolite, polymorph, solvate, hydrate, stereoisomer, and/or tautomer thereof. In some embodiments, the compound of Formula (I) (or any embodiments thereof) is provided as a single stereoisomer or mixture of stereoisomers thereof, a single tautomer or mixture of tautomers thereof, and/or a pharmaceutically acceptable salt thereof.

In a fourth aspect, provided herein is a method of treating a disease, disorder, or condition associated with Wnt transcription products or Wnt signaling pathway activity in a mammal in need thereof, comprising administering a compound of Formula (I) (or any embodiments thereof) or administering a pharmaceutical composition comprising a compound of Formula (I), or any prodrug, pharmaceutically acceptable salt, metabolite, polymorph, solvate, hydrate, stereoisomer, and/or tautomer thereof. In some embodiments, the compound of Formula (I) (or any embodiments thereof) is provided as a single stereoisomer or mixture of stereoisomers thereof, a single tautomer or mixture of tautomers thereof, and/or a pharmaceutically acceptable salt thereof. In some embodiments, the method is for stimulating regeneration of tissue at a wound in the mammal in need thereof.

In a fifth aspect, provided is a method of inducing bacteriostasis associated with Wnt transcription products or Wnt signaling pathway activity in a mammal in need thereof, comprising administering XAV939 or tautomer thereof and/or pharmaceutically acceptable salt thereof, optionally as a pharmaceutical composition thereof, administering a compound of Formula (I) (or any embodiments thereof), or a single stereoisomer or mixture of stereoisomers thereof, a single tautomer or mixture of tautomers thereof, and/or a pharmaceutically acceptable salt thereof; or administering a pharmaceutical composition of the second aspect (or any embodiments thereof) to a mammal in need thereof.

In a sixth aspect, provided is a compound, or a salt thereof, and/or a stereoisomer or mixture of stereoisomers according to any one of the following formulas:

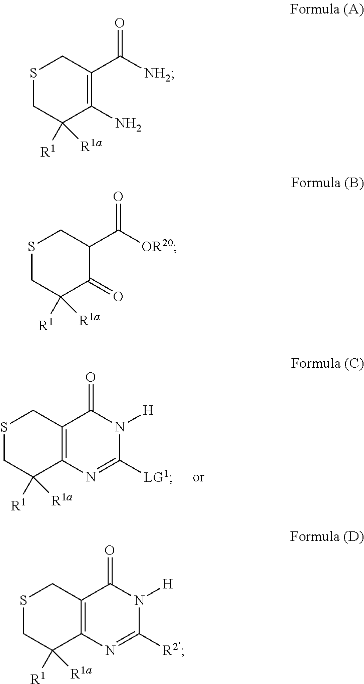

wherein
LG¹ is a leaving group, such as fluoro, chloro, bromo, iodo, triflate, mesylate, a triazole, a pyrazole, boronic acid, boronic ester, or aryl trifluoroborate;
R¹ is hydrogen, deuterium, C₁-C₃alkyl, —OH, —O—C₁-C₃alkyl, —CH₂OH, or —B(OH)₂;
R^{1a} is hydrogen, deuterium, or C₁-C₃alkyl;
R²⁰ is alkyl, preferably methyl or ethyl, or CD₃;
R^{2′} is
   (b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups,
   (c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups;
   (e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups;
   (f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups;
   (h1) C₃-C₆cycloalkyl substituted with NH₂ or OH and additionally optionally substituted with 1 or 2 R^{3a} groups; or
   (i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and
provided that the compound is not:
methyl 4-oxotetrahydro-2H-thiopyran-3-carboxylate

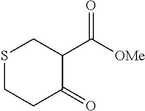 [SMILES: COC(=O)C1CSCCC1=O]

or a salt thereof and/or a stereoisomer or mixture of stereoisomers thereof.

In a seventh aspect, provided is a method of preparing a compound of Formula (I), comprising:
a) contacting a compound of Formula (A):

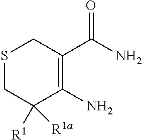

with R²—C(O)H; or
b) contacting a compound of Formula (B):

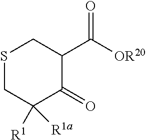

with R²—C(NH)NH₂, wherein R²⁰ is Me or CD₃; or
c) contacting a compound of Formula (C):

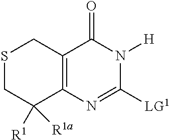

with R²—H, wherein LG¹ is fluoro, chloro, bromo, iodo, triflate, mesylate, a triazole, a pyrazole, boronic acid, boronic ester, or aryl trifluoroborate;
and optionally isolating the compound of Formula (I). In one or more embodiments, the contacting is under basic conditions (in presence of a base).

### BRIEF DESCRIPTION OF FIGURES

FIG. 1 shows inhibition of the Wnt transcription signaling pathway activity by a compound of Formula (I) (Compound 1) compared to a compound of Formula (I) and GO-HA (GO-HA+Compound 1) (Biological Example 3).

FIG. 2 shows inhibition of the Wnt transcription signaling pathway activity by a compound of Formula (I) (Compound 7) compared to a compound of Formula (I) and GO-HA (GO-HA+Compound 7) (Biological Example 3).

FIG. 3 shows inhibition of the Wnt transcription signaling pathway activity by a compound of Formula (I) (Compound 8) compared to a compound of Formula (I) and GO-HA (GO-HA+Compound 8) (Biological Example 3).

FIG. 4 illustrates the results of a rabbit ear study (Biological Example 5), comparing eight injury sites (L1, L2, L3, L4, R5, R6, R7, and R8) on three specimens at day 0 (top) and at day 23 (bottom). The specimens were treated with saline as a control, and a compound of Formula (I) (Compound 8 or Compound 18). Saline was applied via a spray, Compound 8 (1 mg/mL) was applied via a phospholipid spray, and Compound 18 (1 mg/mL) was applied via a phospholipid spray (Biological Example 5).

FIG. 5A shows rate of ear closure from days 1-21 (test 1). Saline control is compared to compounds of Formula (I) (Compound 8 and Compound 18) (Biological Example 5).

FIG. 5B shows rate of ear closure from days 14-39 (test 1). Saline control is compared to compounds of Formula (I) (Compound 8 and Compound 18) (Biological Example 5).

FIG. 6 shows rate of ear closure from days 14-39 (test 2). Saline control compared to compounds of Formula (I) (Compound 8 and Compound 18).

FIG. 7 (top) shows the location of an embed cut across the center point of a healed wound for purposes of processing tissue after cartilage regeneration tests (rabbit ear study, see Biological Example 5). FIG. 7 (bottom) shows a slide with the cross section of sectioned tissue at the center of the wound. Resultant tissue sections placed on the slide are shown in images in FIGS. 9-11.

FIG. 8 shows the average distance between opposing cartilage endplates (mm) after 45 days in cartilage regeneration tests (rabbit ear studies). The measurements were taken from Safranin O stained cross sections of healed ear punch wounds 45 days after treating with saline (control) or a compound of Formula (I) (Compound 8 or Compound 18).

FIG. 9 shows a representative sample of a cross section from the cartilage regeneration test (Biological Example 5) after treatment with saline (control), collected at day 45. Analysis is shown 9-10 mm from the wound margin. Gray lines represent wound margins, top image is at 0.3× magnification, middle image is at 2.5× magnification, and bottom image is at 10× magnification. The gray square at the middle image represents the image shown in the bottom image. Gray arrows in the bottom image show an area with regenerating cartilage.

FIG. 10 shows a representative sample of a cross section from the cartilage regeneration test (Biological Example 5) after treatment with a compound of Formula (I) (Compound 8), collected at day 45. Analysis is shown 9-10 mm from the wound margin. Gray lines represent wound margins, top image is at 0.3× magnification, middle image is at 2.5× magnification, and bottom image is at 10× magnification. The gray square at the middle image represents the image shown in the bottom image. Gray arrows in the bottom image show an area with regenerating cartilage.

FIG. 11 shows a representative sample of a cross section from the cartilage regeneration test (see Biological Example 5) after treatment with a compound of Formula (I) (Compound 18), collected at day 45. Analysis is shown 9-10 mm from the wound margin. Gray lines represent wound margins, top image is at 0.3× magnification, middle image is at 2.5× magnification, and bottom image is at 10× magnification. The gray square at the middle image represents the image shown in the bottom image. Gray arrows in the bottom image show an area with regenerating cartilage.

FIG. 12 shows a schematic diagram of wound site locations on a mammal (swine) in a full thickness excisional wound healing study.

FIG. 13 (top) illustrates suture formation with a simple interrupted closure used in the full thickness excisional wound healing study (see Biological Example 6), with the first throw 3401, the second throw 3403, and a portion of the wound closed by an interrupted suture 3405 shown. FIG. 13 (bottom) shows a cross section view of the portion of the wound closed by the interrupted suture 3405.

FIG. 14A shows results of the full thickness open excisional wound healing, rete ridge formation, with a compound of Formula (I) and GO-HA (Compound 8+GO-HA), a saline control, and uninjured skin.

FIG. 14B shows results of the full thickness open excisional wound healing rete ridge formation (highest outliers removed), with compounds of Formula (I) (Compound 7, Compound 8), and compounds of Formula (I) and GO-HA (Compound 7+GO-HA, Compound 8+GO-HA), a saline control, and GO-HA alone (p*>0.05; p**>0.01; and p***>0.001).

FIG. 15 depicts results of the 3^{rd} degree burn wound healing study (Biological Example 6) comparing a compound of Formula (I) (Compound 8) to a saline control. Wound area (cm²) was measured at 2-6 day intervals. Results from day 16 to day 22 are shown.

FIG. 16 depicts results of the 3^{rd} degree burn wound healing study (Biological Example 6) comparing a compound of Formula (I) (Compound 7) to a saline control. Wound area (cm²) was measured at 2-6 day intervals. Results from day 16 to day 22 are shown.

FIG. 17 depicts results of the 3^{rd} degree burn wound healing study (Biological Example 6) comparing a compound of Formula (I) and GO-HA (Compound 8+GO-HA) to a saline control. Wound area (cm²) was measured at 2-6 day intervals. Results from day 16 to day 22 are shown.

FIG. 18 depicts results of the 3^{rd} degree burn wound healing study (Biological Example 6) comparing a compound of Formula (I) and GO-HA (Compound 7+GO-HA) to a saline control. Wound area (cm²) was measured at 2-6 day intervals. Results from day 16 to day 22 are shown.

FIG. 19 shows results of the closed excisional wound study (Biological Example 6): saline control, serum control (aka serum formulation control), and GO-HA control, from day 1 to day 21. The sutures were removed at day 13.

FIG. 20 shows results of the closed excisional wound study (Biological Example 6): compounds of Formula (I) (Compound 1, Compound 8, and Compound 10), from day 1 to day 21. The sutures were removed at day 13.

FIG. 21 shows results of the closed excisional wound study (Biological Example 6): compounds of Formula (I) with GO-HA (Compound 1+GO-HA, Compound 8+GO-HA, and Compound 10+GO-HA), from day 1 to day 21. The sutures were removed at day 13.

FIG. 22 shows results of the closed excisional wound study (Biological Example 6): cross sectional tissue samples stained with Trichrome Blue. Wound sites treated with saline control, serum formulation control, GO-HA control, compounds of Formula (I) (Compound 1, Compound 8, Compound 10), and compounds of Formula (I) with GO-HA (Compound 1+GO-HA, Compound 8+GO-HA, and Compound 10+GO-HA) are compared.

FIG. 23 shows results of the closed excisional wound study (see Biological Example 6): cross sectional tissue samples under polarized microscopy. Wound sites treated with saline control, serum formulation control, GO-HA control, compounds of Formula (I) (Compound 1, Compound 8, Compound 10), and compounds of Formula (I) with GO-HA (Compound 1+GO-HA, Compound 8+GO-HA, and Compound 10+GO-HA) are compared.

FIG. 24 shows results of closed excisional wound polarized image collagen infiltration (see Biological Example 6). The number of wounds indicating collagen infiltration in the scar are shown for A-saline (aka saline control); B-serum (aka serum formulation control); C-GO-HA (aka GO-HA control); D-Compound 1; E-Compound 1+GO-HA; F-Compound 8; G-Compound 8+GO-HA; H-Compound 10; and J-Compound 10+GO-HA.

FIG. 25 shows results of the 3^{rd} degree burn wound healing study (Biological Example 6) comparing a compound of Formula (I) (Compound 7, Compound 7+GO-HA, Compound 8, Compound 8+GO-HA) to a saline control and GO-HA Control. Histology demonstrated that the compound promoted regeneration of tissue (improved organized reticular collagen and rete ridge formation) and reduced scar formation as compared to saline Control and GO-HA Control.

### DETAILED DESCRIPTION

The present disclosure provides for novel compounds, compositions, and methods of administration thereof to: induce healing of a wound, healing of a burn (including first, second, and third degree burns), or healing of a lesion (including lesions caused by HPV and/or a virus selected from the Poxviridae family of viruses); treat an inflammatory dermatitis disease, a cartilage disease, a bone disease, organ fibrosis, or cancer; induce tissue regeneration (including but not limited to regeneration of damaged elastic cartilage); induce bacteriostasis; induce bacterial growth inhibition; maintain bacteriostasis; induce antifungal activity; induce neovascularization (in tissues in need thereof); induce reinnervation (of a de-nerved body part); inhibit osteoclast differentiation; enhance osteoblast differentiation; and/or inhibit bone destruction associate with breast cancer.

The wound may include but is not limited to one or more selected from the group consisting of a chronic wound, an acute wound, and alkali-burned corneal wound. The inflammatory dermatitis disease may include but is not limited to one or more selected from the group consisting of acne, psoriasis, rosacea, and scleroderma. The cartilage disease may include but is not limited to one or more selected from the group consisting of osteoarthritis, rheumatoid arthritis, internal derangement of the joints, and degenerative cartilage disease. The bone disease may include but is not limited to a bone disease with impaired bone formation, e.g., osteoporosis.

The organ fibrosis may include but is not limited to one or more selected from the group consisting of lung fibrosis, heart fibrosis, liver fibrosis, and kidney fibrosis.

The cancer may include but is not limited to melanoma, breast cancer, and/or prostate cancer.

Further provided herein are compounds, pharmaceutical compositions comprising the compounds, methods of preparing the compounds, and methods of using the compounds and compositions in treating wounds, in particular, the enhanced tissue regeneration following treatment of a wound. Also provided herein are methods of treating conditions associated with Wnt transcription products or Wnt signaling pathway activity in a mammal comprising administering a therapeutically effective amount of a compound or composition to a mammal. In one or more embodiments, the mammal is a human.

### DEFINITIONS

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise. Unless specified otherwise, where a term is defined as being substituted, the groups in the list of substituents are themselves unsubstituted. For example, a substituted alkyl group can be substituted, for example, with a cycloalkyl group, and the cycloalkyl group is not further substituted unless specified otherwise.

Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” or “an,” as used in herein means one or more, unless context clearly dictates otherwise. For example, “pharmaceutically acceptable carrier” includes one or more ingredients as provided herein.

As used herein for GO-HA linker “alkyl” refers to straight or branched hydrocarbon. An alkyl may be linear, branched, cyclic, or a combination thereof, and may contain, for example, from one to sixty carbon atoms, in some embodiments, 2-25 carbons. Examples of alkyl groups include but are not limited to ethyl, ethyl, propyl, isopropyl, cyclopropyl, butyl isomers (e.g. n-butyl, iso-butyl, tert-butyl, etc.) cyclobutyl isomers (e.g. cyclobutyl, methylcyclopropyl, etc.), pentyl isomers, cyclopentyl isomers, hexyl isomers, cyclohexyl isomers, and the like.

The term “alkyl,” as used herein for a compound of Formula (I), unless otherwise specified, refers to a saturated straight or branched, monovalent hydrocarbon. In one or more embodiments, the alkyl group is a primary, secondary, or tertiary hydrocarbon. An alkyl may be linear or branched, and may contain, for example, from one to eight carbon atoms. In one or more embodiments, the alkyl group has one to six carbon atoms, i.e., C₁ to C₆ alkyl. In one or more embodiments, the alkyl is a C₁₋₃alkyl. In one or more embodiments, the alkyl group is selected from the group consisting of methyl, ethyl, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. Examples of alkyl groups include but are not limited to methyl, ethyl, propyl, isopropyl, cyclopropyl, butyl isomers (e.g. n-butyl, iso-butyl, tert-butyl, etc.) pentyl isomers, hexyl isomers, and the like.

As used herein for the GO-HA linker, the term “linear alkyl” refers to a chain of carbon and hydrogen atoms (e.g., ethane, propane, butane, pentane, hexane, etc.).

As used herein for the GO-HA linker, the term “branched alkyl” refers to a chain of carbon and hydrogen atoms, without double or triple bonds that contains a fork, branch, and/or split in the chain. “Branching” refers to the divergence of a carbon chain, whereas “substitution” refers to the presence of non-carbon/non-hydrogen atoms in a moiety.

The term “alkylene,” as used herein, unless otherwise specified, refers to a divalent alkyl group, as defined herein for either the GO-HA linker or as defined for a compound of formula (I), as applicable.

As used herein for the GO-HA linker, the term “cycloalkyl” refers to a completely saturated mono- or multi-cyclic hydrocarbon ring system. When composed of two or more rings, the rings may be joined together in a fused, bridged or spiro-connected fashion. A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl (but not substituted alkyl) or selected from those indicated above with regard to substitution of an alkyl group unless otherwise indicated. Unless specified otherwise (e.g., substituted cycloalkyl group, heterocyclyl, cycloalkoxy group, halocycloalkyl, cycloalkylamine, thiocycloalkyl, etc.), an alkyl group contains carbon and hydrogen atoms only. In some or any embodiments, the cycloalkyl group includes three to six carbon atoms, i.e., C₃ to C₆ cycloalkyl. In some or any embodiments, the cycloalkyl has 3, 4, 5, or 6 (C₃₋₆) 3, 4, or 5 (C₃₋₅); 3 or 4 (C₃₋₄); 3 (C₃); 4 (C₄); or 5 (C₅) carbon atoms. In some or any embodiments, the cycloalkyl group is cyclopropyl, cyclobutyl, cyclopentyl, or cyclohexyl. In some or any embodiments, the cycloalkyl group is cyclopropyl, cyclobutyl, or cyclopentyl. In one or more embodiments, the cycloalkyl group is cyclobutyl.

The term “C₃-C₁₀-cycloalkyl,” as used herein for the Compound of Formula (I), refers to a monovalent, saturated, monocyclic hydrocarbon or bicyclic (fused, bridged, or spirocyclic) ring. In some or any embodiments, the terms “fused cycloalkyl” and “spirocycloalkyl” are embodiments of the cycloalkyl group. In some or any embodiments, the cycloalkyl group includes three to six carbon atoms, i.e., C₃ to C₆ cycloalkyl. In some or any embodiments, the cycloalkyl has 3, 4, or 5 (C₃₋₅); 3 or 4 (C₃₋₄); 3 (C₃); 4 (C₄); or 5 (C₅) carbon atoms. In some or any embodiments, the cycloalkyl group is cyclopropyl, cyclobutyl, cyclopentyl, or cyclohexyl. In some or any embodiments, the cycloalkyl group is cyclopropyl, cyclobutyl, or cyclopentyl. In some or any embodiments, the cycloalkyl group is cyclopropyl. In some or any embodiments, the cycloalkyl group is cyclobutyl. In some or any embodiments, the cycloalkyl group is cyclopentyl. In some or any embodiments, the cycloalkyl group is bicyclo[3.2.1]octyl, bicyclo[2.2.2]octyl, bicyclo[3.3.1]nonyl, bicyclo[3.3.2.]decyl, bicyclo[2.2.2]octyl, or adamantyl. In one or more embodiments, the cycloalkyl group is cyclobutyl.

The term “alkoxy” and “alkyloxy,” as used herein, and unless otherwise specified, refer to the group —OR′ where R′ is alkyl. In one or more embodiments, alkoxy is C₁₋₆alkoxy. In one or more embodiments, alkoxy is C₁-C₃ alkoxy. Alkoxy and alkyloxy groups include, in one or more embodiments, methoxy, ethoxy, n-propoxy, isopropoxy, n-butoxy, tert-butoxy, sec-butoxy, n-pentoxy, n-hexyloxy, 1,2-dimethylbutoxy, and the like.

The term “alkoxyalkyl,” as used herein for a compound of Formula (I), and unless otherwise specified, refers to an alkyl group, as defined herein, substituted with one or two —OR′ groups where each R′ is alkyl, as defined herein, and is independently selected. In some or any embodiments, alkoxyalkyl is C₁-C₆alkoxy-C₁-C₆alkyl.

The term “alkoxycarbonyl-NH-alkyl,” as used herein for a compound of Formula (I), and unless otherwise specified, refers to an alkyl group substituted with —NH—C(O)O(alkyl), wherein alkyl is as defined herein. In some or any embodiments, alkoxycarbonyl-NH-alkyl is C₁-C₆alkoxycarbonyl-NH—C₁-C₆alkyl.

As used herein “alkenyl,” as used herein for the GO-HA linker, means a straight or branched chain hydrocarbon having at least 2 carbon atoms, which contains at least one carbon-carbon double bond.

As used herein “alkynyl,” as used herein for the GO-HA linker, means a straight or branched chain hydrocarbon having at least 2 carbon atoms, which contains at least one carbon-carbon triple bond.

As used herein “amine” or “amino” as used herein for the GO-HA linker are represented by a formula -NA₁A₂, where A₁ and A₂ are, independently, hydrogen or alkyl, cycloalkyl, alkenyl, cycloalkenyl, alkynyl, cycloalkynyl, aryl, or heteroaryl group where each of these groups are as described herein for the GO-HA linker. In one or more embodiments amine (or amino) refers to any of NH₂, NH(alkyl), NH(aryl), N(alkyl)₂, N(alkyl)(aryl), and N(aryl)₂.

The term “aryl,” as used herein, and unless otherwise specified, refers to a monovalent C₆-C₁₅ carbocyclic ring system which comprises at least one aromatic ring wherein the aryl ring system is mono, di, or tricyclic. The aryl may be attached to the main structure through any of its rings, i.e. any aromatic or nonaromatic ring. In some or any embodiments, the aryl group may be a bridged (where chemically feasible) or non-bridged, spirocyclic (where chemically feasible) or not spirocyclic, and/or fused or not fused multicyclic group. In some or any embodiments, aryl is C₆-C₁₀ aryl. In some or any embodiments, aryl is C₆ aryl, i.e. phenyl. In some or any embodiments, aryl is phenyl, naphthyl, indanyl, fluorenyl, 6,7,8,9-tetrahydro-5H-benzo[7]annulenyl, or tetrahydronaphthyl. When aryl is substituted, it can be substituted on any ring, i.e. on any aromatic or nonaromatic ring comprised by aryl.

The term “haloalkyl,” as used herein, and unless otherwise specified, refers to an alkyl group substituted with 1, 2, 3, 4, or 5 halo groups. In some or any embodiments, the haloalkyl is a halo-C₁₋₆alkyl. In some or any embodiments, the haloalkyl is —CF₃, —CH₂F, —CHF₂, or —CH₂CF₃.

The terms “halogen” and “halo,” as used herein, and unless otherwise specified, are synonymous and refer to chloro, bromo, fluoro, or iodo.

The term “heteroaryl,” as used herein, and unless otherwise specified, refers to a monocyclic aromatic ring system or multicyclic aromatic ring system wherein one or more (in some or any embodiments, 1, 2, 3, or 4) of the ring atoms is a heteroatom independently selected from O, S(O)₀₋₂, NH, and N, and the remaining ring atoms are carbon atoms, and where the ring may be optionally substituted as described herein. The heteroaryl group is bonded to the rest of the molecule through any atom in the ring system, valency rules permitting. In some or any embodiments, each ring of a heteroaryl group can contain one or two O atoms, one or two S atoms, and/or one to four N atoms, or a combination thereof, provided that the total number of heteroatoms in each ring is four or less and each ring contains at least one carbon atom. In some or any embodiments, the heteroaryl has from 5 to 20, from 5 to 15, from 5 to 6 ring atoms, or from 5 to 10 ring atoms. When heteroaryl is substituted, it can be substituted on any ring. In one or more embodiments, heteroaryl is a 5- to 10-membered heteroaryl. In one or more embodiments, heteroaryl is a 5 or 6-membered heteroaryl. In one or more embodiments, heteroaryl is a 6-membered heteroaryl. In one or more embodiments, heteroaryl is,

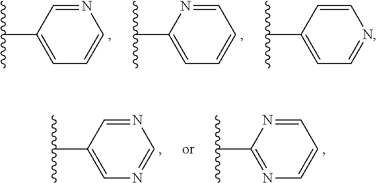

wherein

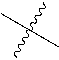

indicates the point of attachment of the heteroaryl to the rest of the molecule.

In some or any embodiments, monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl and triazolyl. In some or any embodiments, bicyclic heteroaryl groups include, but are not limited to, benzofuranyl, benzimidazolyl, benzoxazolyl, benzoisoxazolyl, benzopyranyl, benzothiadiazolyl, benzothiazolyl, benzoisothiazolyl, benzothienyl, benzotriazolyl, furopyridyl, thienopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, or quinazolinyl. In some or any embodiments, tricyclic heteroaryl groups include, but are not limited to, acridinyl, benzindolyl, carbazolyl, dibenzofuranyl, perimidinyl, phenanthrolinyl, phenanthridinyl, and phenazinyl. In some or any embodiments, heteroaryl is indolyl, furanyl, pyridinyl, pyrimidinyl, imidazolyl, or pyrazolyl; each of which is optionally substituted with 1, 2, 3, or 4 groups as defined throughout the specification, including in some embodiments with group(s) independently selected from C₁₋₆alkyl, hydroxy, halo, halo-C₁₋₆alkyl, C₁₋₆alkoxy, cyano, or phenyl.

The term “heterocyclic,” as used herein, and unless otherwise specified, refers to a monovalent monocyclic non-aromatic ring system or a monovalent multicyclic ring system that contains at least one non-aromatic ring; wherein one or more (in some or any embodiments, 1, 2, 3, or 4) of the monocyclic non-aromatic ring atoms is a heteroatom independently selected from O, S(O)₀₋₂, and N, and the remaining ring atoms are carbon atoms; and wherein one or more (in some or any embodiments, 1, 2, 3, or 4) of any of the ring atoms in the multicyclic ring system is a heteroatom(s) independently selected from O, S(O)₀₋₂, and N, and the remaining ring atoms are carbon. The term “heterocyclic” does not include fully aromatic ring(s), i.e. does not include imidazole, pyrimidine, pyridine, and the like. In some or any embodiments, the heterocyclic ring comprises one or two heteroatom(s) which are independently selected from nitrogen and oxygen. In some or any embodiments, the heterocyclic ring comprises one or two heteroatom(s) which are oxygen. In some or any embodiments, the heterocyclic ring comprises one or two heteroatom(s) which are nitrogen (where the nitrogen is substituted as described in any aspect or embodiment described herein). In some or any embodiments, heterocyclic is multicyclic and comprises one heteroatom in a non-aromatic ring, or comprises one heteroatom in an aromatic ring, or comprises two heteroatoms in an aromatic ring, or comprises two heteroatoms where one is in an aromatic ring and the other is in a non-aromatic ring. In some or any embodiments, the heterocyclic group has from 3 to 20, 3 to 15, 3 to 10, 3 to 8, 4 to 7, or 5 to 6 ring atoms. In one or more embodiments, the heterocyclic is a 4- to 10-membered heterocyclic. In one or more embodiments, the heterocyclic is a 5- to 10-membered heterocyclic. In some or any embodiments, the heterocyclic is a monocyclic, bicyclic, tricyclic, or tetracyclic ring system. In some or any embodiments, the heterocyclic group may be a bridged or non-bridged, spirocyclic or not spirocyclic, and/or fused or not fused multicyclic group. One or more of the nitrogen and sulfur atoms may be optionally oxidized, one or more of the nitrogen atoms may be optionally quaternized, one or more of the carbon atoms may be optionally replaced with

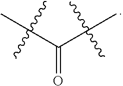 [SMILES: *C(*)=O]

Some rings may be partially or fully saturated, or aromatic provided that heterocyclic is not fully aromatic. The monocyclic and multicyclic heterocyclic rings may be attached to the main structure at any heteroatom or carbon atom which results in a stable compound. The multicyclic heterocyclic may be attached to the main structure through any of its rings, including any aromatic or nonaromatic ring, regardless of whether the ring contains a heteroatom. In some or any embodiments, heterocyclic is “heterocycloalkyl” which is 1) a saturated monovalent monocyclic group which contains at least one ring heteroatom, as described herein, or 2) a saturated monovalent bi- or tri-cyclic group in which at least one ring contains at least one heteroatom as described herein. In some or any embodiments, heterocyclic is 3- to 6-membered heterocycloalkyl. In some or any embodiments, heterocyclic is 3- to 8-membered heterocycloalkyl. In some or any embodiments, heterocyclic is 3- to 9-membered heterocycloalkyl. When heterocyclic and heterocycloalkyl are substituted, they can be substituted on any ring, i.e. on any aromatic or nonaromatic ring comprised by heterocyclic and heterocycloalkyl. In some or any embodiments, such heterocyclic includes, but are not limited to, azepinyl, benzodioxanyl, benzodioxolyl, 3,4-dihydro-2H-benzo[b][1,4]oxazinyl, 3,4-dihydro-2H-benzo[b][1,4]dioxepinyl, 1,3-dihydroisobenzofuranyl, benzofuranonyl, benzopyranonyl, benzopyranyl, dihydrobenzofuranyl, benzotetrahydrothienyl, 2,2-dioxo-1,3-dihydrobenzo[c]thienyl, benzothiopyranyl, benzoxazinyl, β-carbolinyl, chromanyl, chromonyl, cinnolinyl, coumarinyl, decahydroquinolinyl, decahydroisoquinolinyl, dihydro-benzimidazolonyl (including but not limited to 2-oxo-1,3-dihydro-2H-benzo[d]imidazol-1-yl), dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, 2,4-dioxo-imidazolidinyl, imidazolinyl, indolinyl, 2-oxo-indolinyl, isobenzotetrahydrofuranyl, isobenzotetrahydrothienyl, isochromanyl, isocoumarinyl, isoindolinyl, 1-oxo-isoindolinyl, 1,3-dioxo-isoindolinyl, isothiazolidinyl, isoxazolidinyl, 3-oxo-isoxazolidinyl, morpholinyl, 3,5-dioxo-morpholinyl, octahydroindolyl, octahydroisoindolyl, 1-oxo-octahydroisoindolyl, 1,3-dioxo-hexahydroisoindolyl, oxo-oxadiazolyl (including but not limited to 5-oxo-1,2,4-oxadiazol-3-yl), oxazolidinonyl, oxazolidinyl, oxiranyl, piperazinyl, 2,6-dioxo-piperazinyl, piperidinyl, 2,6-dioxo-piperidinyl, 4-piperidonyl, pyrazolidinyl, pyrazolinyl, pyrrolidinyl, pyrrolinyl, 2-oxopyrrolidinyl, 2,5-dioxopyrrolidinyl, quinuclidinyl, tetrahydrofuryl, tetrahydroisoquinolinyl, tetrahydropyranyl, tetrahydrothienyl, thiamorpholinyl, thiomorpholinyl, 3,5-dioxo-thiomorpholinyl, thiazolidinyl, 2,4-dioxo-thiazolidinyl, tetrahydroquinolinyl, phenothiazinyl, phenoxazinyl, xanthenyl, 1,3,5-trithianyl, or 1,3-dihydro-imidazopyridin-2-onyl. In some or any embodiments, heterocyclic is benzo-1,4-dioxanyl, benzodioxolyl, indolinyl, 2-oxo-indolinyl, pyrrolidinyl, piperidinyl, 2,3-dihydrobenzofuranyl, decahydroquinolinyl, dihydrocyclopentapyridyl, dihydropyranopyridyl, tetrahydronaphthyridyl, 2,2-dioxo-3,4-dihydro-thiopyrano-pyridyl, dihydrofuropyridyl, dihydropyrrolopyridyl, 2,2-dioxo-1,3-dihydro-thieno-pyridyl, or tetrahydrocyclopropacyclopentapyridyl, each of which is optionally substituted with 1, 2, 3, or 4 groups as defined throughout the specification, including in some or any embodiments with group(s) independently selected from halo, alkyl, and phenyl. In some embodiments, heterocycloalkyl is pyrrolidinyl. In some embodiments, heterocycloalkyl is an N-linked heterocycloalkyl.

The term “hydroxyalkyl” as used herein, unless otherwise specified, refers to an alkyl, as defined herein, substituted by 1, 2, or 3 hydroxy groups. In one or more embodiments, the hydroxy group is a primary, secondary, or tertiary alcohol. In one or more embodiments, the hydroxyalkyl group includes one to ten carbons, i.e., C₁ to C₁₀ hydroxyalkyl. In one or more embodiments, the hydroxyalkyl group includes one or two alcohol (hydroxy) groups, provided that they are not on the same carbon. In one or more embodiments, the hydroxyalkyl group is hydroxyC₁₋₆alkyl. In one or more embodiments, the hydroxyalkyl group is hydroxyC₁₋₃alkyl. In one or more embodiments, the hydroxyalkyl group is selected from the group consisting of hydroxymethyl, hydroxyethyl, hydroxypropyl, hydroxybutyl, hydroxypentyl, and hydroxyhexyl. In one or more embodiments, the hydroxyalkyl group is a C₁₋₆hydroxyalkyl. In one or more embodiments, the hydroxyalkyl group is selected from the group consisting of hydroxymethyl, 1-hydroxyethyl, 2-hydroxyethyl, 1-hydroxypropan-2-yl, and 2-hydroxypropan-2-yl.

The term “oxo” as used herein and unless otherwise specified, refers to a keto group (C═O). An oxo group that is a substituent of a nonaromatic carbon results in a conversion of a —CH₂— to —C═O. An oxo group that is a substituent of an aromatic carbon results in a conversion of —CH— to —C═O. When a substituent is oxo, then two hydrogens on the atom are replaced. When an oxo group substitutes aromatic moieties, the corresponding partially unsaturated ring replaces the aromatic ring. For example, a pyridyl group substituted by an oxo group is a pyridone. The person of ordinary skill in the art will appreciate that in some embodiments that such a group, e.g. pyridone and 2,4 (1H,3H)-dioxo-pyrimidinyl, can exist in its tautomeric form, e.g. hydroxypyridine and 2,4-dihydroxypyrimidinyl, respectively.

As used herein “regeneration” means the renewal or growth of destroyed or devitalized tissue from the remnant tissue. It is a reparative attempt of the body, and in the context of wound represents the migration, differentiation, or replication of cells or transformation of progenitor cells into the appropriate cell types for the respective tissue which may include sebaceous cells, hair follicles, nerve cells, chondrocytes.

As used herein “wound” means an injury to tissue or skin caused by scrapes, cuts, abrasion, surgical procedures (e.g., caused by minimally invasive surgery, laparoscopic surgery, robotic surgery, incisional biopsies, general surgery, and cosmetic surgery), denuded skin, burns, ulcers (e.g., diabetic ulcers, ulcers from vascular insufficiency, pressure sores, and burns), or other skin problems (e.g., allergies). Wound may range from superficial (e.g., affecting merely the epidermis) to more traumatic (e.g., lesions which affect layers of skin or tissue at depths which are beneath the epidermis). Wounds may be of any length or shape, e.g., in some embodiments, wounds are straight, jagged or curve.

In some embodiments, the term “pharmaceutically acceptable carrier” includes any and all and/or one or more solvents, co-solvents, complexing agents, dispersion media, coatings, antibacterial and antifungal agents, isotonic and absorption delaying agents, excipients, diluents, disintegrants, lubricants, adjuvants, and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index (Merck & Company, Rahway, N.J.), considerations for the inclusion of various components in pharmaceutical compositions are described, (e.g., Gilman et al. (Eds.), 2010, Goodman and Gilman's: The Pharmacological Basis of Therapeutics, 12th Ed., The McGraw-Hill Companies).

In some embodiments, the term “pharmaceutically acceptable salt” refers to salts that retain the biological effectiveness and properties of the compounds provided herein and which are not biologically or otherwise undesirable. In many cases, the compounds provided herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297.

In some embodiments, the term “pharmaceutically acceptable salt,” as used herein, and unless otherwise specified, refers to any salt of a compound provided herein which retains its biological properties and which is not toxic or otherwise desirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; and (2) base addition salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine tris(hydroxymethyl)-aminomethane, piperazine, tetramethylammonium hydroxide, and the like.

In some embodiments, pharmaceutically acceptable salts further include, in some or any embodiments, and without limitation, sodium, potassium, calcium, magnesium, ammonium, tetraalkylammonium salts and the like. When the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

In some embodiments, a “therapeutically effective amount” or “pharmaceutically effective amount” of a compound as provided herein, is one which is sufficient to achieve the desired effect and may vary according to the nature and severity of the disease condition, and the potency of the compound. “Therapeutically effective amount” is also intended to include one or more of the compositions of the present disclosure so as to result in the increased regeneration of tissue subject to a wound. The combination of compounds is preferably a synergistic combination. Synergy, as described in the art (for example, Chou, 2010, Canc. Res. 70(2):440-446), occurs when the effect of the compounds when administered in combination is greater than the additive effect of the compounds when administered alone as a single agent. In general, a synergistic effect is most clearly demonstrated at sub-optimal concentrations of the compounds. This amount can further depend upon the patient's height, weight, sex, age and medical history.

The term “mammal” specifically includes humans, cattle, horses, dogs, and cats, but also includes many other mammalian species like pigs, rats, mice, primates (e.g., a monkey such as a cynomolgous monkey, a chimpanzee and a human). In some embodiments, the mammal is a human.

The term “subject” refers to a mammal, as provided herein, as well as to a cell or biological sample.

The term “substantially free of” stereoisomers with respect to a composition refers to a composition that includes at least 85 or 90% by weight, in some or any embodiments 95%, 98%, 99% or 100% by weight, of a designated stereoisomer of a compound in the composition. In some or any embodiments, in the methods and compounds provided herein, the compounds are substantially free of stereoisomers.

Similarly, the term “isolated” with respect to a composition refers to a composition that includes at least 85, 90%, 95%, 98%, 99% to 100% by weight, of a specified compound, the remainder comprising other chemical species or stereoisomers.

The term “isotopic composition,” as used herein, and unless otherwise specified, refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopic enrichment,” as used herein, and unless otherwise specified, refers to the percentage of incorporation of an amount of a specific isotope at a given atom in a molecule in the place of that atom's natural isotopic abundance. In some or any embodiments, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

The term “isotopically enriched,” as used herein, and unless otherwise specified, refers to an atom having an isotopic composition other than the natural isotopic composition of that atom. “Isotopically enriched” may also refer to a compound containing at least one atom having an isotopic composition other than the natural isotopic composition of that atom.

As used herein, and unless otherwise specified, the term “IC₅₀” refers to an amount, concentration or dosage of a particular test compound that achieves a 50% inhibition of a maximal response in an assay that measures such response.

In some embodiments, the terms “therapeutic agent” and “therapeutic agents” refer to any agent(s) which can be used in the treatment or prevention of a disorder or one or more symptoms thereof. In some or any embodiments, the term “therapeutic agent” includes a compound provided herein. In some or any embodiments, a therapeutic agent is an agent which is known to be useful for, or has been or is currently being used for the treatment or prevention of a disorder or one or more symptoms thereof.

In some embodiments, “treating” or “treatment” of any condition or disorder refers, in some or any embodiments, to ameliorating a condition or disorder that exists in a subject. In another embodiment, “treating” or “treatment” includes ameliorating at least one physical parameter, which may be indiscernible by the subject. In yet another embodiment, “treating” or “treatment” includes modulating the condition or disorder, either physically (e.g., stabilization of a discernible symptom) or physiologically (e.g., stabilization of a physical parameter) or both. In yet another embodiment, “treating” or “treatment” includes delaying the onset of the condition or disorder. In yet another embodiment, “treating” or “treatment” includes the reduction or elimination of either the condition or one or more symptoms of the condition, or to retard the progression of the condition or of one or more symptoms of the condition, or to reduce the severity of the condition or of one or more symptoms of the condition.

### Compounds

Provided herein are compounds that can induce improved wound healing and tissue regeneration. Provided herein are compounds that can treat wounds and/or conditions associated with Wnt transcription products or Wnt signaling pathway activity, and in particular enhance tissue regeneration following treatment of a wound.

Further provided herein are compounds that can modulate the activity of the Wnt signaling pathway or Wnt transcription (products). The compounds can be formed as described herein and used for the treatment of conditions associated with Wnt transcription products or Wnt signaling pathway activity. In one or more embodiments, the condition associated with Wnt transcription products or Wnt signaling pathway activity is a chronic wound, an acute wound, an alkali-burned corneal wound, a burn, a lesion (including lesions caused by HPV and/or a virus selected from the Poxviridae family of viruses), an inflammatory dermatitis disease (including acne, psoriasis, rosacea, and scleroderma), a cartilage disease (including osteoarthritis, rheumatoid arthritis, internal derangement of the joints, and degenerative cartilage disease), a bone disease (including osteoporosis), organ fibrosis (including lung fibrosis, heart fibrosis, liver fibrosis, and kidney fibrosis), cancer (including melanoma, breast cancer, and prostate cancer), a de-nerved body part in need of reinnervation, tissue in need of regeneration (including damaged elastic cartilage), bacterial growth in need of inhibition, fungal growth in need of inhibition, tissue in need of neovascularization, osteoclast differentiation in need of inhibition, osteoblast differentiation disorders (where inhibition of osteoblast differentiation is needed), and/or bone destruction associated with breast cancer.

The present disclosure arises from the novel and unexpected finding of significant Wnt inhibition by the compound of Formula (I) (or any embodiments thereof, including in some embodiments, Compound 1, Compound 7, or Compound 8), and with some improved properties as compared to the prior art Wnt inhibitors, for example XAV939. Some improved characteristics may include improved wound healing and regeneration of tissue subject to a wound in mammals, both in the quantity of regrowth as well as the quality of the tissue regeneration and regrowth.

The aspects and embodiments described herein include the recited compounds as well as a pharmaceutically acceptable salt thereof and/or an isomer thereof. For instance, aspects and embodiments described herein include a single stereoisomer of mixture of stereoisomers thereof, and/or a pharmaceutically acceptable salt thereof.

Included herein, if chemically possible, are all stereoisomers of the compounds, including diastereomers and enantiomers. Also included are mixtures of possible stereoisomers in any ratio, including, but not limited to, racemic mixtures. Unless stereochemistry is explicitly indicated in a structure at a particular atom, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated. It will be apparent that certain structures recite specific stereochemistry at particular atoms.

A composition for inhibiting Wnt transcription products or Wnt signaling pathway activity comprising: a matrix component comprising a graphene oxide (GO) and hyaluronic acid (HA) conjugate (GO-HA), wherein GO and HA are covalently linked via a linker; polyethylene glycol (PEG), where the PEG is optional; a thickener, where the thickener is optional; a compound of any one of claims 1-39; and water, wherein the compound optionally constitutes from about 0.001 wt % to about 5 wt % of the total composition.

In one or more embodiments, the compound of Formula (I) is a potent inhibitor of the Wnt pathway.

Embodiment 1: In one or more embodiments of Formula (I), the compound is a potent inhibitor of the Wnt pathway, with a chemical name (4-(4-oxo-3,5,7,8-tetrahydro-4H-thiopyrano[4,3-d]pyrimidin-2-yl)phenyl)boronic acid (Compound 1). The structure of

Compound 1 is:

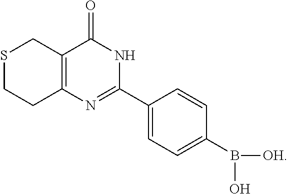 [SMILES: O=c1[nH]c(-c2ccc(B(O)O)cc2)nc2c1CSCC2]

In one or more embodiments, the compound of Formula (I) is Compound 1 or any prodrug, pharmaceutically acceptable salt, metabolite, polymorph, solvate, hydrate, stereoisomer, or tautomer thereof.

Embodiment 2: In one or more embodiments of Formula (I), the compound is a potent inhibitor of the Wnt pathway, with a chemical name 2-(4-(6-bromopyridin-3-yl) phenyl)-3,5,7,8-tetrahydro-4H-thiopyrano [4,3-d]pyrimidin-4-one (Compound 7). The structure of Compound 7 is:

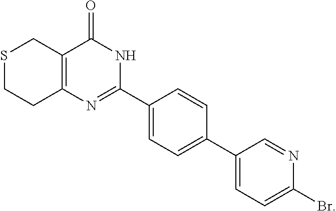 [SMILES: O=c1[nH]c(-c2ccc(-c3ccc(Br)nc3)cc2)nc2c1CSCC2]

In one or more embodiments, the compound of Formula (I) is Compound 7 or any prodrug, pharmaceutically acceptable salt, metabolite, polymorph, solvate, hydrate, stereoisomer, or tautomer thereof.

Embodiment 3: In one or more embodiments of Formula (I), the compound is a potent inhibitor of the Wnt pathway, with a chemical name 2-(4-(2-(2-hydroxyethoxy)propan-2-yl)phenyl)-3,5,7,8-tetrahydro-4H-thiopyrano[4,3-d]pyrimidin-4-one (Compound 8). The structure of Compound 8 is:

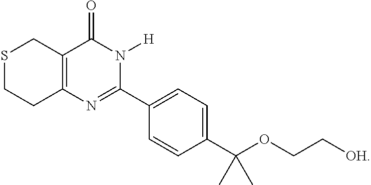 [SMILES: CC(C)(OCCO)c1ccc(-c2nc3c(c(=O)[nH]2)CSCC3)cc1]

In one or more embodiments, the compound of Formula (I) is Compound 8 or any prodrug, pharmaceutically acceptable salt, metabolite, polymorph, solvate, hydrate, stereoisomer, or tautomer thereof.

### Embodiment A

In one or more embodiments, provided is a compound of Formula (I), wherein R¹ and R^{1a} are independently selected from is hydrogen and deuterium.

### Embodiment B

In one or more embodiments, including any of the above embodiments, provided is a compound of Formula (I), wherein ring A is or

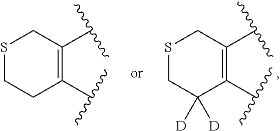 [SMILES: *C1=C(*)CSCC1 ; *C1=C(*)C([2H])([2H])CSC1]

and wherein [symbol] designates attachment to the remainder of the compound of Formula (I).

### Embodiments where R² is (a)

In one or more embodiments, including Embodiments A and B, provided is a compound of Formula (I), wherein R² is phenyl substituted with R³ and additionally optionally substituted with 1, 2, or 3 R^{3a} groups.

In one or more embodiments, including Embodiments A and B, provided is a compound of Formula (I), wherein
R³ is attached to the para-position of the phenyl ring; or
R³ and one R^{3a}, when on adjacent carbons, together with the carbons to which they are attached form ring (a-1) and
where the phenyl portion is optionally substituted with the remaining R^{3a} groups.

In one or more embodiments, including Embodiments A and B, provided is a compound of Formula (I), wherein when R² is (a), R³ is not halo or haloalkyl.

In one or more embodiments, including Embodiments A and B, provided is a compound of Formula (I), wherein when R² is (a), R³ is B(O)₂ or —(C₀-C₆alkylene)-O—R⁴.

In one or more embodiments, including Embodiments A and B, provided is a compound of Formula (I), wherein R² is selected from the group consisting of

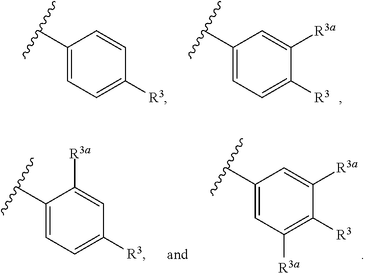

In one or more embodiments, R³ is not halo or haloalkyl. In one or more embodiments, R³ is B(O)₂ or —(C₀-C₆alkylene)-O—R⁴.

In one or more embodiments, including Embodiments A and B, provided is a compound of Formula (I), wherein R² is selected from the group consisting of

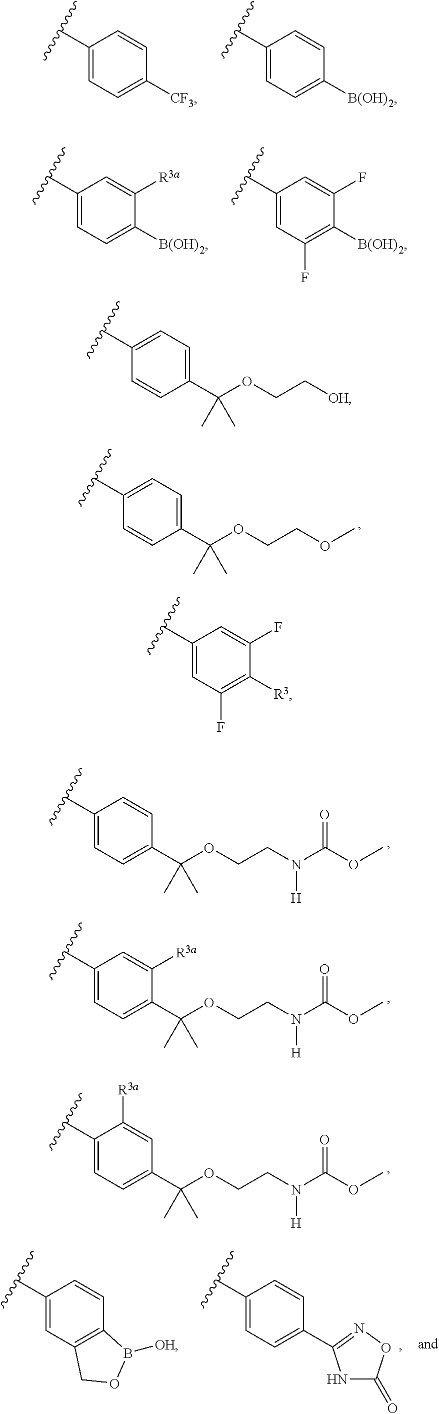

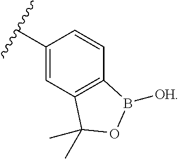 [SMILES: *c1ccc2c(c1)C(C)(C)OB2O]

In one or more embodiments, R² is not

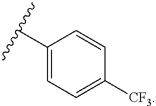 [SMILES: *c1ccc(C(F)(F)F)cc1]

In one or more embodiments, including Embodiments A and B, provided is a compound of Formula (I), wherein R² is selected from the group consisting of

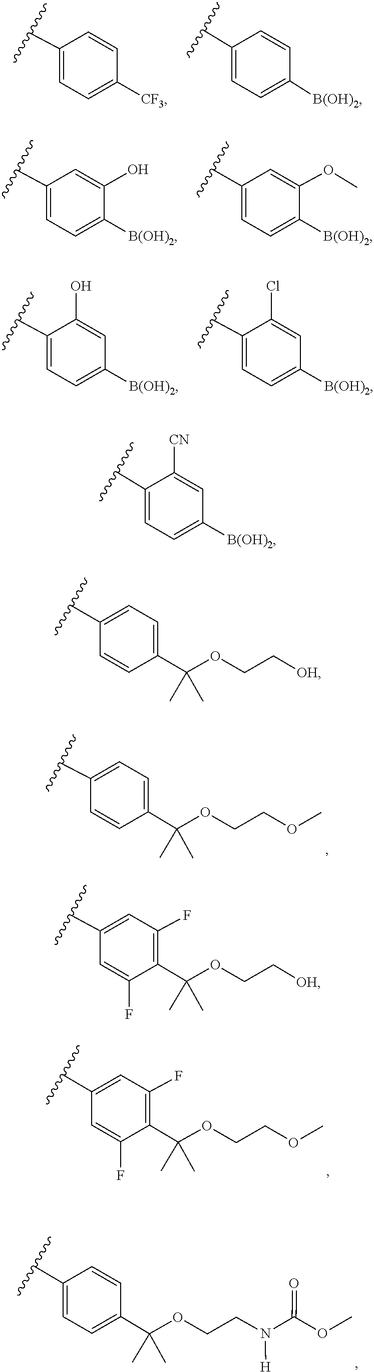

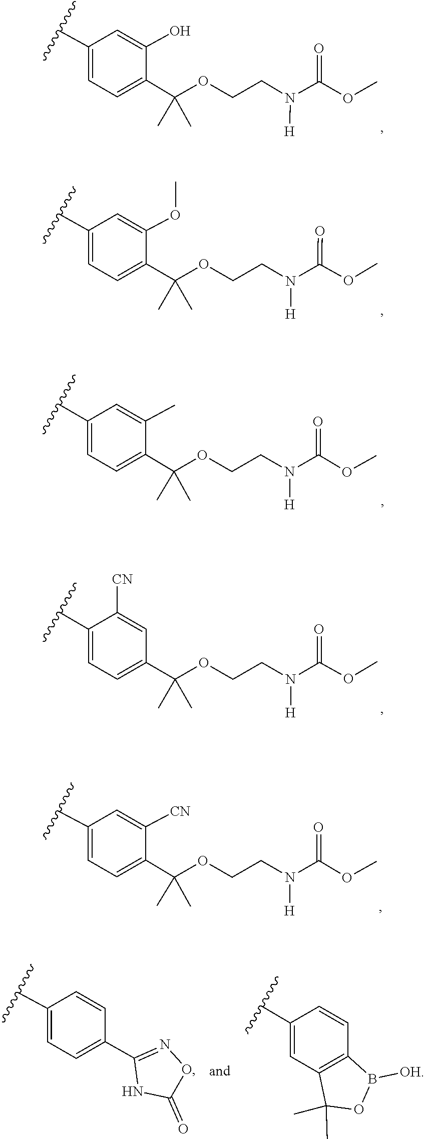

In one or more embodiments, R² is not

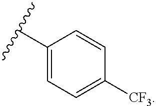 [SMILES: *c1ccc(C(F)(F)F)cc1]

### Embodiments where R2 is (b)

In one or more embodiments, including Embodiments A and B, provided is a compound of Formula (I), wherein R² is phenyl substituted with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups.

In one or more embodiments, including Embodiments A and B, provided is a compound of Formula (I), wherein R² is phenyl substituted at its para-position with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups; and when the 5- or 6-membered heteroaryl is a 6-membered heteroaryl then R³ is substituted on the para-position of the 6-membered heteroaryl.

In one or more embodiments, including Embodiments A and B, provided is a compound of Formula (I), wherein R² is selected from the group consisting of

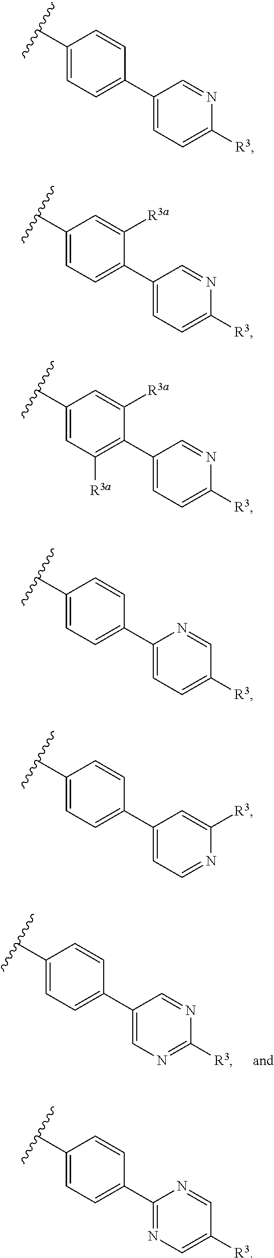

In one or more embodiments, including Embodiments A and B, provided is a compound of Formula (I), wherein R² is selected from the group consisting of

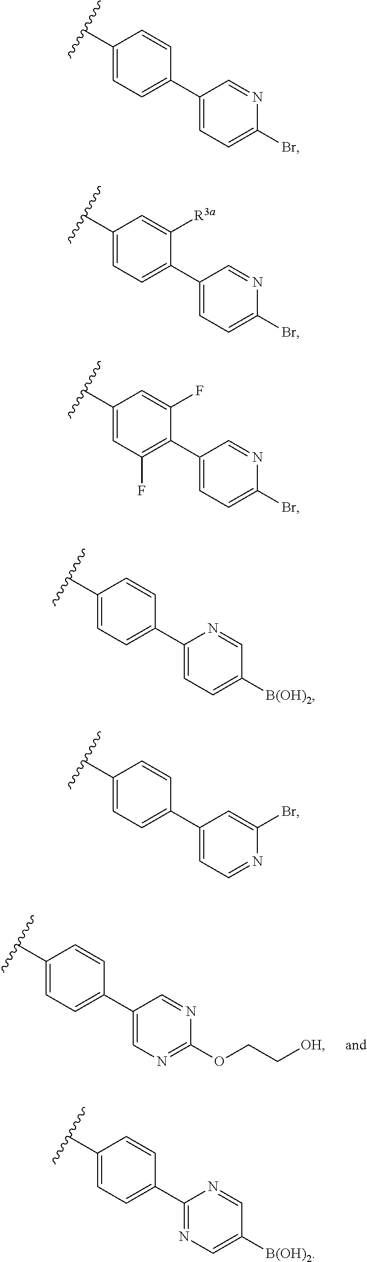

### Embodiments where R² is (c)

In one or more embodiments, including Embodiments A and B, provided is a compound of Formula (I), wherein R² is phenyl optionally substituted with 1, 2, or 3 R^{3a} groups and additionally substituted with -phenyl-R³ where the phenyl in -phenyl-R³ is optionally substituted with 1, 2, or 3 R^{3a} groups.

In one or more embodiments, including Embodiments A and B, provided is a compound of Formula (I), wherein R² is phenyl optionally substituted with 1, 2, or 3 R^{3a} groups and additionally substituted at its para-position with -phenyl-R³ where the phenyl in -phenyl-R³ is optionally substituted with 1, 2, or 3 R^{3a} groups and where the R³ is in the para-position of the phenyl in -phenyl-R³.

In one or more embodiments, including Embodiments A and B, provided is a compound of Formula (I), wherein R² is selected from the group consisting of

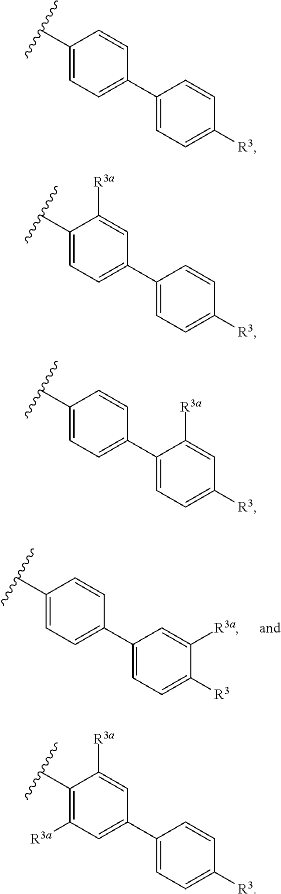

In one or more embodiments, including Embodiments A and B, provided is a compound of Formula (I), wherein R² is selected from the group consisting of

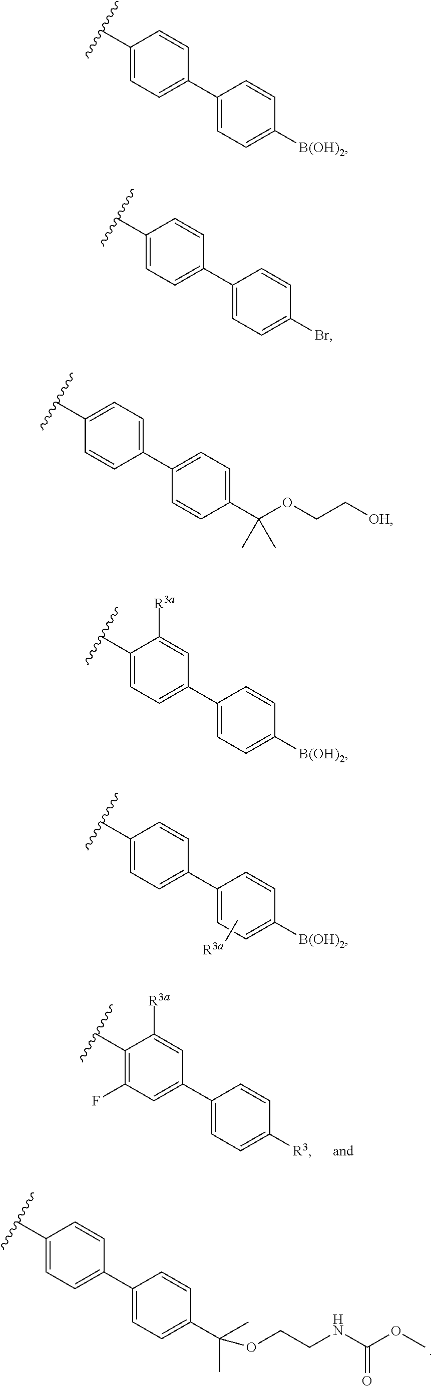

### Embodiments where R² is (d)

In one or more embodiments, including Embodiments A and B, provided is a compound of Formula (I), wherein R² is 5- to 6-membered heteroaryl substituted with R³ and additionally optionally substituted with 1, 2, or 3 R^{3a} groups; optionally wherein the R³ is at the para-position of the 6-membered heteroaryl.

In one or more embodiments, including Embodiments A and B, provided is a compound of Formula (I), wherein when R² is (d), R³ is not halo or haloalkyl.

In one or more embodiments, including Embodiments A and B, provided is a compound of Formula (I), wherein when R² is (d), R³ is B(O)₂ or —(C₀-C₆alkylene)-O—R⁴.

In one or more embodiments, including Embodiments A and B, provided is a compound of Formula (I), wherein when R² is (d), R³ is B(O)₂ or —(C₁-C₆alkylene)-O—R⁴.

### Embodiments where R² is (e)

In one or more embodiments, including Embodiments A and B, provided is a compound of Formula (I), wherein R² is 5- or 6-membered heteroaryl optionally substituted with 1, 2, or 3 R^{3a} groups and additionally substituted with -phenyl-R³ where the phenyl is additionally optionally substituted with 1, 2, or 3 R^{3a} groups; optionally wherein the -phenyl-R³ is at the para-position of the 6-membered heteroaryl; and optionally wherein the R³ is at the para-position of the phenyl.

### Embodiments where R² is (f)

In one or more embodiments, including Embodiments A and B, provided is a compound of Formula (I), wherein R² is 5- or 6-membered heteroaryl optionally substituted with 1, 2, or 3 R^{3a} groups and additionally substituted with -(5- or 6-membered heteroaryl)-R³ where the 5- or 6-membered heteroaryl in -(5- or 6-membered heteroaryl)-R³ is optionally substituted with 1, 2, or 3 R^{3a} groups; optionally wherein the -(6-membered heteroaryl)-R³ is at the para-position of the first 6-membered heteroaryl; and optionally wherein the R³ is at the para-position of the 6-membered heteroaryl to which it is attached.

In one or more embodiments, including Embodiments A and B, provided is a compound of Formula (I), wherein R² is

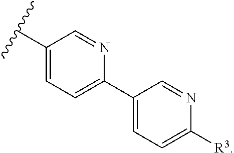

### Embodiments where R² is (g)

In one or more embodiments, including Embodiments A and B, provided is a compound of Formula (I), wherein R² is C₃-C₆cycloalkyl substituted with R³ and additionally optionally substituted with 1 or 2 R^{3a} groups.

In one or more embodiments, including Embodiments A and B, provided is a

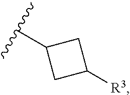

compound of Formula (I), wherein R² is R³, optionally wherein R³ is 5- to 10-membered heterocyclic optionally substituted with cyano.

### Embodiments where R² is (h)

In one or more embodiments, including Embodiments A and B, provided is a compound of Formula (I), wherein R² is C₃-C₆cycloalkyl substituted with 5- or 6-membered heteroaryl where the 5- or 6-membered heteroaryl is substituted with R³ and additionally optionally substituted with 1, 2, or 3 R^{3a} groups, and where the cycloalkyl is optionally substituted with 1 or 2 R^{3a} groups.

### Embodiments where R² is (i)

In one or more embodiments, including Embodiments A and B, provided is a compound of Formula (I), wherein R² is C₃-C₆cycloalkyl substituted with phenyl where the phenyl is substituted with R³ and additionally optionally substituted with 1, 2, or 3 R^{3a} groups, and where the cycloalkyl is optionally substituted with 1 or 2 R^{3a} groups.

### Embodiments where R2 is (j)

In one or more embodiments, including Embodiments A and B, provided is a compound of Formula (I), wherein R² is 3- to 8-membered heterocycloalkyl substituted with phenyl or 5- or 6-membered heteroaryl, where the phenyl and the 5- to 6-membered heteroaryl are substituted with R³ and additionally optionally substituted with 1, 2, or 3 R^{3a} groups.

In one or more embodiments, including Embodiments A and B, provided is a compound of Formula (I), wherein R² is selected from the group consisting of

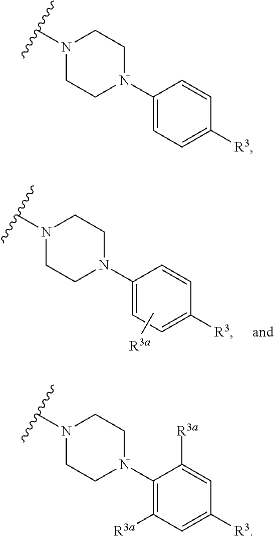

### Embodiments where R² is (k)

In one or more embodiments, including Embodiments A and B, provided is a compound of Formula (I), wherein R² is —CH═CH—R⁵ where R⁵ is phenyl or 5- or 6-membered heteroaryl, where the phenyl and the 5- or 6-membered heteroaryl are substituted with R³ and additionally optionally substituted with 1, 2, or 3 R^{3a} groups.

In one or more embodiments, including Embodiments A and B, provided is a compound of Formula (I), wherein R² is selected from the group consisting of

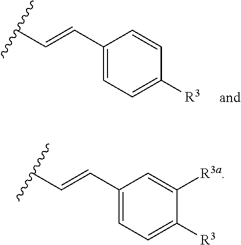

### Additional Embodiments

In one or more embodiments, including Embodiments A and B and any embodiments provided above when R² is (a)-(k), provided is a compound of Formula (I), wherein R³ is cyano, —B(OH)₂, or —(C₀-C₆alkylene)-O—R⁴. In one or more embodiments, provided is a compound of Formula (I), wherein R⁴ is hydroxy-C₁-C₆alkyl (in some embodiments, hydroxyethyl). In one or more embodiments, provided is a compound of Formula (I), wherein R⁴ is C₁-C₆alkoxy-C₁-C₆alkyl (in some embodiments, C₁-C₆alkoxy-C₂alkyl). In one or more embodiments, provided is a compound of Formula (I), wherein R⁴ is C₁-C₆alkoxycarbonyl-NH—C₁-C₆alkyl (in some embodiments, C₁-C₆alkoxycarbonyl-NH—C₂alkyl).

In one or more embodiments, including Embodiments A and B and any embodiments provided above when R² is (a)-(k), provided is a compound of Formula (I), wherein R³ is cyano.

In one or more embodiments, including Embodiments A and B and any embodiments provided above when R² is (a)-(k), provided is a compound of Formula (I), wherein R³ is —B(OH)₂.

In one or more embodiments, including Embodiments A and B and any embodiments provided above when R² is (a)-(k), provided is a compound of Formula (I), wherein R³ is —(C₀-C₆alkylene)-O—R⁴. In one or more embodiments, provided is a compound of Formula (I), wherein R⁴ is hydroxy-C₁-C₆alkyl (in some embodiments, hydroxyethyl). In one or more embodiments, provided is a compound of Formula (I), wherein R⁴ is C₁-C₆alkoxy-C₁-C₆alkyl (in some embodiments, C₁-C₆alkoxy-C₂alkyl). In one or more embodiments, provided is a compound of Formula (I), wherein R⁴ is C₁-C₆alkoxycarbonyl-NH—C₁-C₆alkyl (in some embodiments, C₁-C₆alkoxycarbonyl-NH—C₂alkyl).

In one or more embodiments, including Embodiments A and B and any embodiments provided above when R² is (a)-(k), provided is a compound of Formula (I), wherein R³ is —(C₁₋₆alkylene)-O—R⁴. In one or more embodiments, provided is a compound of Formula (I), wherein R⁴ is hydroxy-C₁-C₆alkyl (in some embodiments, hydroxyethyl). In one or more embodiments, provided is a compound of Formula (I), wherein R⁴ is C₁-C₆alkoxy-C₁-C₆alkyl (in some embodiments, C₁-C₆alkoxy-C₂alkyl). In one or more embodiments, provided is a compound of Formula (I), wherein R⁴ is C₁-C₆alkoxycarbonyl-NH—C₁-C₆alkyl (in some embodiments, C₁-C₆alkoxycarbonyl-NH—C₂alkyl).

In one or more embodiments, including Embodiments A and B and any embodiments provided above when R² is (b), (c), (e), or (f) provided is a compound of Formula (I), wherein R³ is halo, cyano, —B(OH)₂, or —(C₀-C₆alkylene)-O—R⁴. In one or more embodiments, provided is a compound of Formula (I), wherein R⁴ is hydroxy-C₁-C₆alkyl (in some embodiments, hydroxyethyl). In one or more embodiments, provided is a compound of Formula (I), wherein R⁴ is C₁-C₆alkoxy-C₁-C₆alkyl (in some embodiments, C₁-C₆alkoxy-C₂alkyl). In one or more embodiments, provided is a compound of Formula (I), wherein R⁴ is C₁-C₆alkoxycarbonyl-NH—C₁-C₆alkyl (in some embodiments, C₁-C₆alkoxycarbonyl-NH—C₂alkyl).

In one or more embodiments, including Embodiments A and B and any embodiments provided above,
when R² is (b), (c), (e), or (f) provided is a compound of Formula (I), wherein R³ is halo, cyano, —B(OH)₂, or —(C₀-C₆alkylene)-O—R⁴; or
when R² is (b), (c), (e), or (f) provided is a compound of Formula (I), wherein R³ is cyano, —B(OH)₂, or —(C₀-C₆alkylene)-O—R⁴.

In one or more embodiments, provided is a compound of Formula (I), wherein R⁴ is hydroxy-C₁-C₆alkyl (in some embodiments, hydroxyethyl). In one or more embodiments, provided is a compound of Formula (I), wherein R⁴ is C₁-C₆alkoxy-C₁-C₆alkyl (in some embodiments, C₁-C₆alkoxy-C₂alkyl). In one or more embodiments, provided is a compound of Formula (I), wherein R⁴ is C₁-C₆alkoxycarbonyl-NH—C₁-C₆alkyl (in some embodiments, C₁-C₆alkoxycarbonyl-NH—C₂alkyl).

In one or more embodiments, provided is a compound of Formula (I), selected from the group consisting of the Compounds 1-44 provided in Table 1.

In one or more embodiments, provided is a pharmaceutical composition comprising a compound of Formula (I); and comprising a pharmaceutically acceptable carrier. In one or more embodiments, provided is a pharmaceutical composition comprising a compound of Formula (I), or a single stereoisomer or mixture of stereoisomers thereof, a single tautomer or mixture of tautomers thereof, and/or a pharmaceutically acceptable salt thereof, comprising a pharmaceutically acceptable carrier.

In one or more embodiments, a pharmaceutical composition herein further comprises: a matrix component comprising a graphene oxide (GO) and hyaluronic acid (HA) conjugate (GO-HA), wherein GO and HA are covalently linked via a linker; polyethylene glycol (PEG), where the PEG is optional; a thickener, where the thickener is optional; and water, optionally wherein the compound optionally constitutes from about 0.001 wt % to about 5 wt % of the total composition.

In one or more embodiments, provided is a method for inhibiting Wnt transcription products or Wnt signaling pathway activity in a subject comprising contacting an effective amount of a compound of Formula (I) or a single stereoisomer or mixture of stereoisomers thereof, a single tautomer or mixture of tautomers thereof, and/or a pharmaceutically acceptable salt thereof, with the subject.

In one or more embodiments, provided is a method for treating a disease, disorder, or condition associated with Wnt transcription products or Wnt signaling pathway activity in a mammal, comprising administering a compound of Formula (I) or a single stereoisomer or mixture of stereoisomers thereof, a single tautomer or mixture of tautomers thereof, and/or a pharmaceutically acceptable salt thereof; or administering a pharmaceutical composition according to one or more embodiments herein to a subject in need thereof.

In one or more embodiments, provided is a method for stimulating regeneration of tissue at a wound in a subject in need thereof and wherein the wound is contacted with an effective amount of the compound of Formula (I) (or a single stereoisomer or mixture of stereoisomers thereof, a single tautomer or mixture of tautomers thereof, and/or a pharmaceutically acceptable salt thereof) or a pharmaceutical composition according to one or more embodiments herein.

In one or more embodiments, a disease, disorder, or condition (to be treated in one or more embodiments of a method herein) is selected from a chronic wound, an acute wound, an alkali-burned corneal wound, a burn, a lesion (including lesions caused by HPV and/or a virus selected from the Poxviridae family of viruses), an inflammatory dermatitis disease (including acne, psoriasis, rosacea, and scleroderma), a cartilage disease (including osteoarthritis, rheumatoid arthritis, internal derangement of the joints, and degenerative cartilage disease), a bone disease (including osteoporosis), organ fibrosis (including lung fibrosis, heart fibrosis, liver fibrosis, and kidney fibrosis), cancer (including melanoma, breast cancer, and prostate cancer), a de-nerved body part in need of reinnervation, tissue in need of regeneration (including damaged elastic cartilage), bacterial growth in need of inhibition, fungal growth in need of inhibition, tissue in need of neovascularization, osteoclast differentiation in need of inhibition, osteoblast differentiation disorders (where inhibition of osteoblast differentiation is needed), and/or bone destruction associated with breast cancer.

In one or more embodiments, provided is a method of inducing bacteriostasis associated with Wnt transcription products or Wnt signaling pathway activity, comprising administering XAV939 or tautomer thereof and/or pharmaceutically acceptable salt thereof, optionally as a pharmaceutical composition thereof, administering a compound of Formula (I), or a single stereoisomer or mixture of stereoisomers thereof, a single tautomer or mixture of tautomers thereof, and/or a pharmaceutically acceptable salt thereof; or administering a pharmaceutical composition according to one or more embodiments herein to a mammal in need thereof.

In one or more embodiments, provided is a compound, or a salt thereof, and/or a stereoisomer or mixture of stereoisomers according to any one of the following formulas:

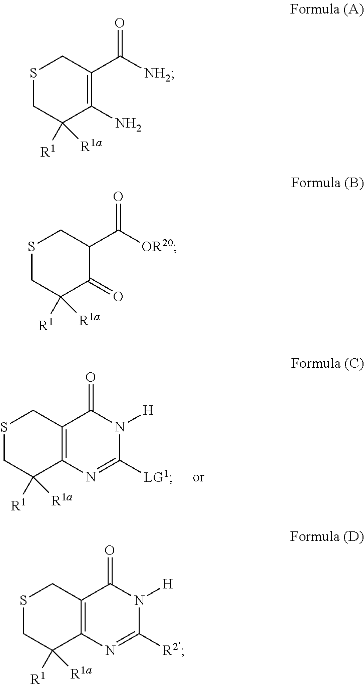

and wherein LG¹ is a leaving group, such as fluoro, chloro, bromo, iodo, triflate, mesylate, a triazole, a pyrazole, boronic acid, boronic ester, or aryl trifluoroborate; R¹ is hydrogen, deuterium, C₁-C₃alkyl, —OH, —O—C₁-C₃alkyl, —CH₂OH, or —B(OH)₂; R^{1a} is hydrogen, deuterium, or C₁-C₃alkyl; R²⁰ is alkyl, preferably methyl or ethyl, or CD₃; R^{2′} is (b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups, (c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups; (e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups; (f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups; (h1) C₃-C₆cycloalkyl substituted with NHl₂ or OH and additionally optionally substituted with 1 or 2 R^{3a} groups; or (i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and provided that the compound is not: methyl 4-oxotetrahydro-2H-thiopyran-3-carboxylate

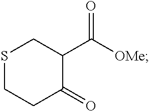 [SMILES: COC(=O)C1CSCCC1=O]

or a salt thereof and/or a stereoisomer or mixture of stereoisomers thereof.

LG¹ may be a suitable leaving group, for example, in a suitable nucleophilic aromatic substitution or a suitable cross coupling, not limited to a Suzuki-Miyaura coupling.

In one or more embodiments, provided is a method of preparing a compound of Formula (I), comprising: a) contacting a compound of Formula (A):

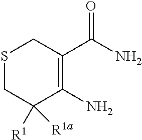

with R^{2′}—C(O)H; or b) contacting a compound of Formula (B):

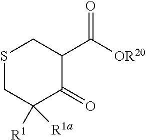

with R^{2′}—C(NH)NH₂, wherein R²⁰ is Me or CD₃; or c) contacting a compound of Formula (C):

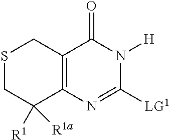

wherein LG¹ is fluoro, chloro, bromo, iodo, triflate, mesylate, a triazole, a pyrazole, boronic acid, boronic ester, or aryl trifluoroborate; and optionally isolating the compound of Formula (I); wherein R^{2′} is (b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups, (c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups; (e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups; (f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups; (h1) C₃-C₆cycloalkyl substituted with NHl₂ or OH and additionally optionally substituted with 1 or 2 R^{3a} groups; or (i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and provided that the compound is not: methyl 4-oxotetrahydro-2H-thiopyran-3-carboxylate

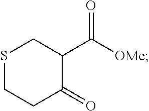 [SMILES: COC(=O)C1CSCCC1=O]

or a salt thereof and/or a stereoisomer or mixture of stereoisomers thereof. In one or more embodiments, methods of preparing a compound of Formula (I) include contacting under basic conditions.

In one or more embodiments of Formula (I), the compound is selected from any of Compounds 1-44 or a pharmaceutically acceptable salt thereof, from Table 1. In one or more embodiments of Formula (I), the compound is selected from any of Compounds 1-44, or an isomer thereof, from Table 1. In one or more embodiments of Formula (I), the compound is selected from any of Compounds 1-44, or a single stereoisomer of mixture of stereoisomers thereof, and/or a pharmaceutically acceptable salt thereof, from Table 1. In one or more embodiments of Formula (I), the compound is selected from any of Compounds 1-44, or a prodrug, pharmaceutically acceptable salt, metabolite, polymorph, solvate, hydrate, stereoisomer, or tautomer thereof, from Table 1.

TABLE 1
Compounds

| Compound | |
|---|---|
| Number | Structure |
| XAV939 | 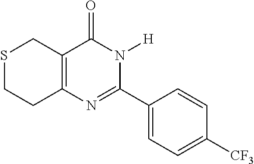 [SMILES: O=c1[nH]c(-c2ccc(C(F)(F)F)cc2)nc2c1CSCC2] |
| 1 | 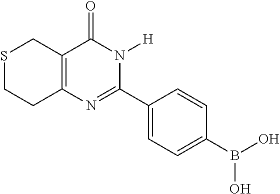 [SMILES: O=c1[nH]c(-c2ccc(B(O)O)cc2)nc2c1CSCC2] |
| 2 | 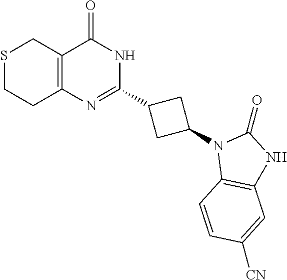 |
| 3 | 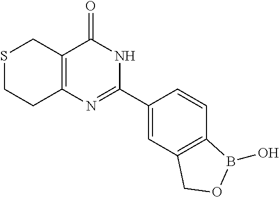 [SMILES: O=c1[nH]c(-c2ccc3c(c2)COB3O)nc2c1CSCC2] |
| 4 | 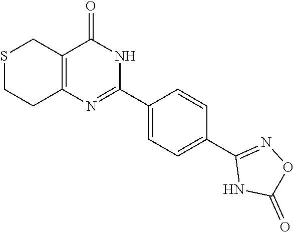 [SMILES: O=c1[nH]c(-c2ccc(-c3nc4c(c(=O)[nH]3)CSCC4)cc2)no1] |
| 5 | 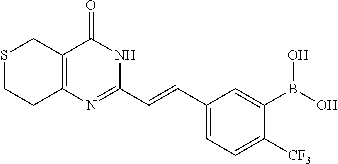 [SMILES: O=c1[nH]c(/C=C/c2ccc(C(F)(F)F)c(B(O)O)c2)nc2c1CSCC2] |
| 6 | 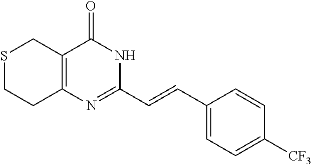 [SMILES: O=c1[nH]c(/C=C/c2ccc(C(F)(F)F)cc2)nc2c1CSCC2] |
| 7 | 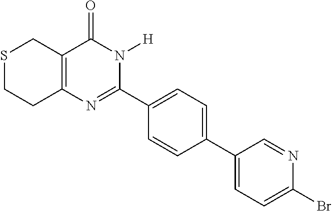 [SMILES: O=c1[nH]c(-c2ccc(-c3ccc(Br)nc3)cc2)nc2c1CSCC2] |
| 8 | 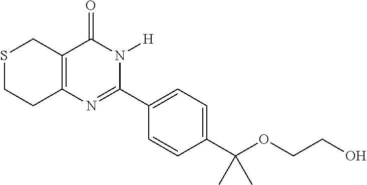 [SMILES: CC(C)(OCCO)c1ccc(-c2nc3c(c(=O)[nH]2)CSCC3)cc1] |
| 9 | 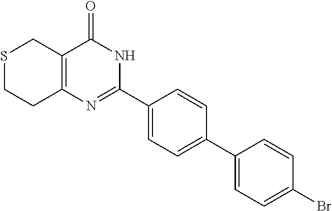 [SMILES: O=c1[nH]c(-c2ccc(-c3ccc(Br)cc3)cc2)nc2c1CSCC2] |
| 10 | 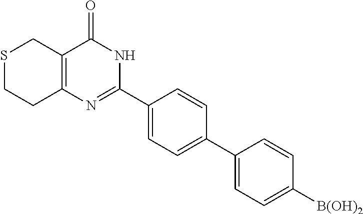 [SMILES: O=c1[nH]c(-c2ccc(-c3ccc(B(O)O)cc3)cc2)nc2c1CSCC2] |
| 11 | 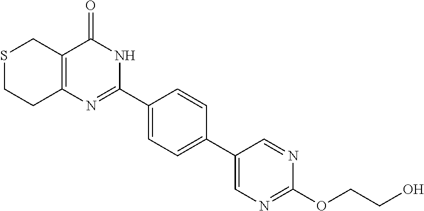 [SMILES: O=c1[nH]c(-c2ccc(-c3cnc(OCCO)nc3)cc2)nc2c1CSCC2] |
| 12 | 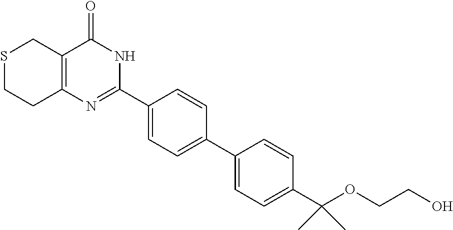 [SMILES: CC(C)(OCCO)c1ccc(-c2ccc(-c3nc4c(c(=O)[nH]3)CSCC4)cc2)cc1] |
| 13 | 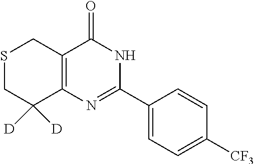 [SMILES: [2H]C1([2H])CSCc2c1nc(-c1ccc(C(F)(F)F)cc1)[nH]c2=O] |
| 14 | 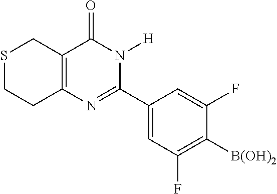 [SMILES: O=c1[nH]c(-c2cc(F)c(B(O)O)c(F)c2)nc2c1CSCC2] |
| 15 | 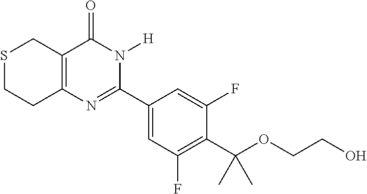 [SMILES: CC(C)(OCCO)c1c(F)cc(-c2nc3c(c(=O)[nH]2)CSCC3)cc1F] |
| 16 | 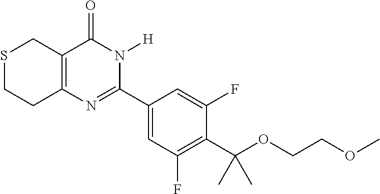 [SMILES: COCCOC(C)(C)c1c(F)cc(-c2nc3c(c(=O)[nH]2)CSCC3)cc1F] |
| 17 | 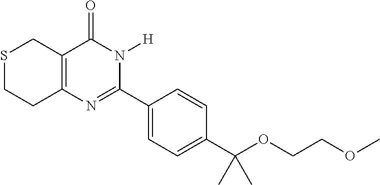 [SMILES: COCCOC(C)(C)c1ccc(-c2nc3c(c(=O)[nH]2)CSCC3)cc1] |
| 18 | 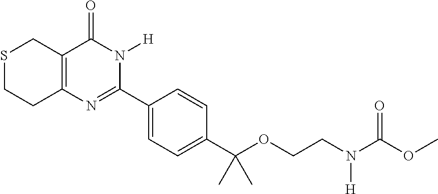 [SMILES: COC(=O)NCCOC(C)(C)c1ccc(-c2nc3c(c(=O)[nH]2)CSCC3)cc1] |
| 19 | 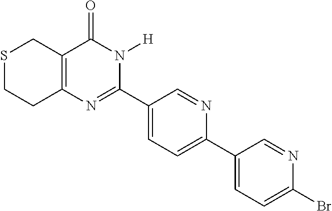 [SMILES: O=c1[nH]c(-c2ccc(-c3ccc(Br)nc3)nc2)nc2c1CSCC2] |
| 20 | 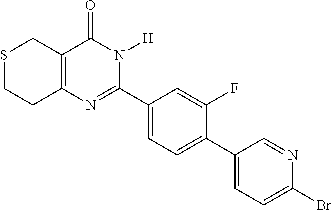 [SMILES: O=c1[nH]c(-c2ccc(-c3ccc(Br)nc3)c(F)c2)nc2c1CSCC2] |
| 21 | 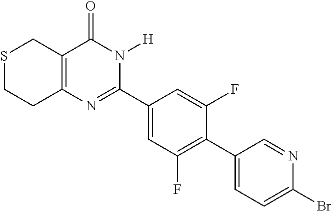 [SMILES: O=c1[nH]c(-c2cc(F)c(-c3ccc(Br)nc3)c(F)c2)nc2c1CSCC2] |
| 22 | 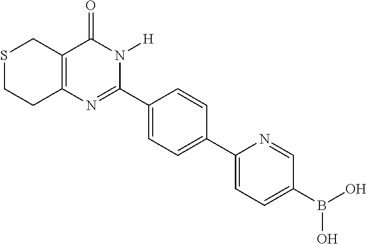 [SMILES: O=c1[nH]c(-c2ccc(-c3ccc(B(O)O)cn3)cc2)nc2c1CSCC2] |
| 23 | 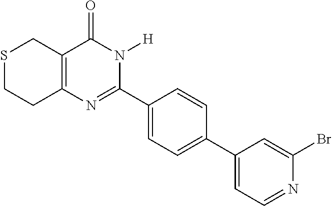 [SMILES: O=c1[nH]c(-c2ccc(-c3ccnc(Br)c3)cc2)nc2c1CSCC2] |
| 24 | 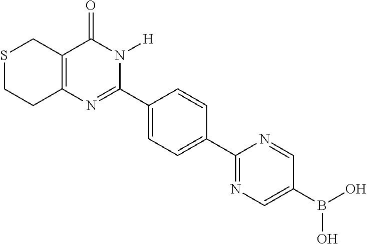 [SMILES: O=c1[nH]c(-c2ccc(-c3ncc(B(O)O)cn3)cc2)nc2c1CSCC2] |
| 25 | 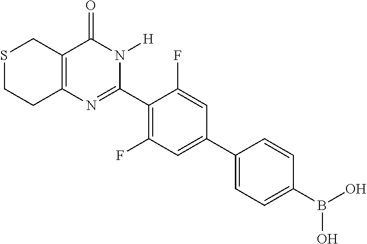 [SMILES: O=c1[nH]c(-c2c(F)cc(-c3ccc(B(O)O)cc3)cc2F)nc2c1CSCC2] |
| 26 | 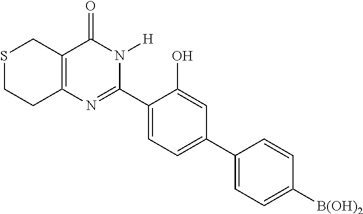 [SMILES: O=c1[nH]c(-c2ccc(-c3ccc(B(O)O)cc3)cc2O)nc2c1CSCC2] |
| 27 | 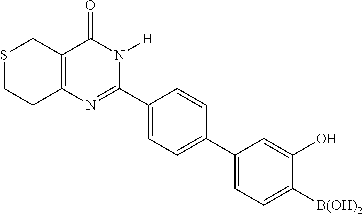 [SMILES: O=c1[nH]c(-c2ccc(-c3ccc(B(O)O)c(O)c3)cc2)nc2c1CSCC2] |
| 28 | 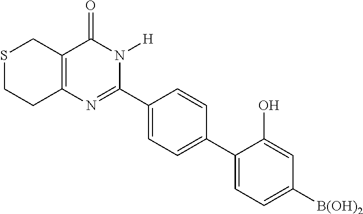 [SMILES: O=c1[nH]c(-c2ccc(-c3ccc(B(O)O)cc3O)cc2)nc2c1CSCC2] |
| 29 | 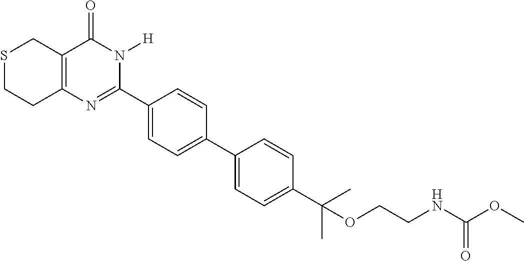 [SMILES: COC(=O)NCCOC(C)(C)c1ccc(-c2ccc(-c3nc4c(c(=O)[nH]3)CSCC4)cc2)cc1] |
| 30 | 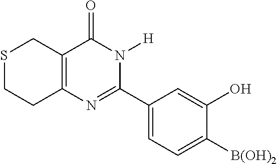 [SMILES: O=c1[nH]c(-c2ccc(B(O)O)c(O)c2)nc2c1CSCC2] |
| 31 | 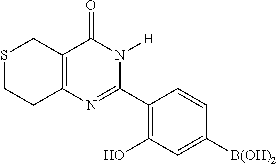 [SMILES: O=c1[nH]c(-c2ccc(B(O)O)cc2O)nc2c1CSCC2] |
| 32 | 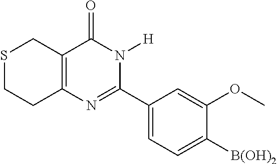 [SMILES: COc1cc(-c2nc3c(c(=O)[nH]2)CSCC3)ccc1B(O)O] |
| 33 | 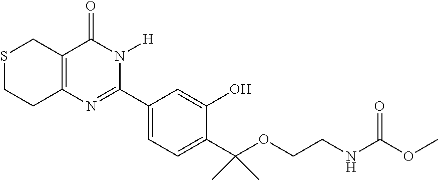 [SMILES: COC(=O)NCCOC(C)(C)c1ccc(-c2nc3c(c(=O)[nH]2)CSCC3)cc1O] |
| 34 | 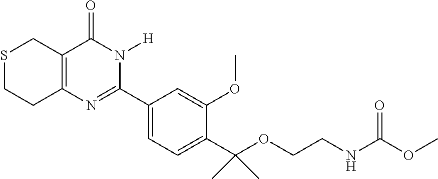 [SMILES: COC(=O)NCCOC(C)(C)c1ccc(-c2nc3c(c(=O)[nH]2)CSCC3)cc1OC] |
| 35 | 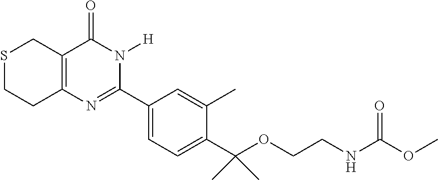 [SMILES: COC(=O)NCCOC(C)(C)c1ccc(-c2nc3c(c(=O)[nH]2)CSCC3)cc1C] |
| 36 | 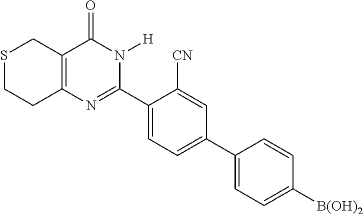 [SMILES: N#Cc1cc(-c2ccc(B(O)O)cc2)ccc1-c1nc2c(c(=O)[nH]1)CSCC2] |
| 37 | 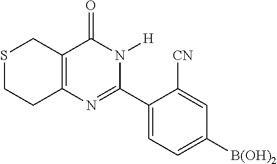 [SMILES: N#Cc1cc(B(O)O)ccc1-c1nc2c(c(=O)[nH]1)CSCC2] |
| 38 | 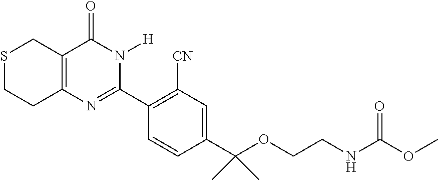 [SMILES: COC(=O)NCCOC(C)(C)c1ccc(-c2nc3c(c(=O)[nH]2)CSCC3)c(C#N)c1] |
| 39 | 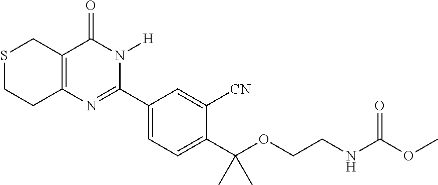 [SMILES: COC(=O)NCCOC(C)(C)c1ccc(-c2nc3c(c(=O)[nH]2)CSCC3)cc1C#N] |
| 40 | 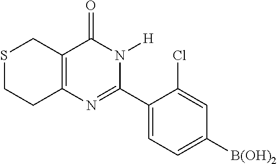 [SMILES: O=c1[nH]c(-c2ccc(B(O)O)cc2Cl)nc2c1CSCC2] |
| 41 | 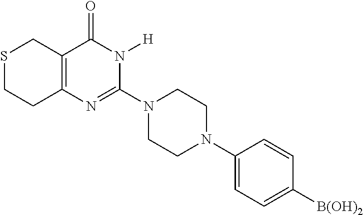 [SMILES: O=c1[nH]c(N2CCN(c3ccc(B(O)O)cc3)CC2)nc2c1CSCC2] |
| 42 | 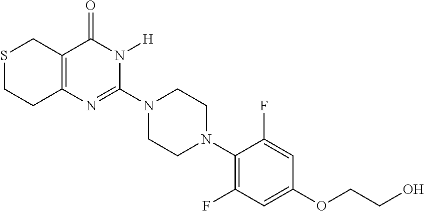 [SMILES: O=c1[nH]c(N2CCN(c3c(F)cc(OCCO)cc3F)CC2)nc2c1CSCC2] |
| 43 | 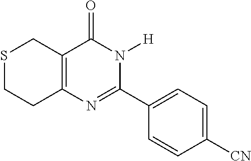 [SMILES: N#Cc1ccc(-c2nc3c(c(=O)[nH]2)CSCC3)cc1] |
| 44 | 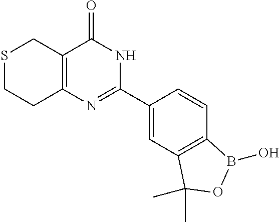 [SMILES: CC1(C)OB(O)c2ccc(-c3nc4c(c(=O)[nH]3)CSCC4)cc21] |

In one or more embodiments, provided herein is a compound according to any one of the following formulas:

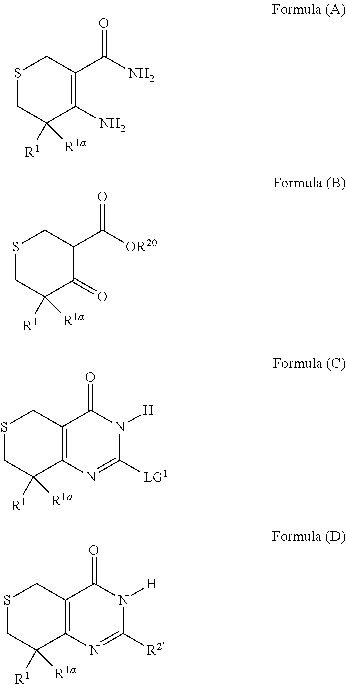

wherein
LG¹ is a leaving group, such as fluoro, chloro, bromo, iodo, triflate, mesylate, a triazole, a pyrazole, boronic acid, boronic ester, or aryl trifluoroborate;
R¹ is hydrogen, deuterium, C₁-C₃alkyl, —OH, —O—C₁-C₃alkyl, —CH₂OH, or —B(OH)₂;
R^{1a} is hydrogen, deuterium, or C₁-C₃alkyl;
R²⁰ is alkyl, preferably methyl or ethyl, or CD₃;
R^{2′} is
   (b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups,
   (c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups;
   (e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups;
   (f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups;
   (h1) C₃-C₆cycloalkyl substituted with NHl₂ or OH and additionally optionally substituted with 1 or 2 R^{3a} groups; or
   (i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and
provided that the compound is not:
   methyl 4-oxotetrahydro-2H-thiopyran-3-carboxylate

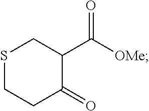 [SMILES: COC(=O)C1CSCCC1=O]

or a salt or enantiomer thereof.

In one or more embodiments, the compound is selected from any of the following compounds A1 to A6, or a prodrug, pharmaceutically acceptable salt, metabolite, polymorph, solvate, hydrate, stereoisomer, or tautomer thereof, from Table 2, which compounds are useful at least for making compounds of Formula (I).

TABLE 2
Intermediates

| Compound | |
|---|---|
| Number | Structure |
| A1 | 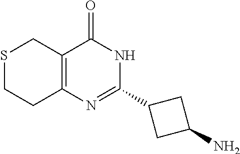 |
| A4 | 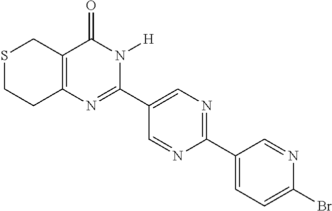 [SMILES: O=c1[nH]c(-c2cnc(-c3ccc(Br)nc3)nc2)nc2c1CSCC2] |
| A5 | 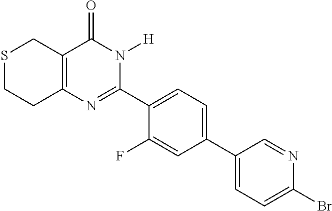 [SMILES: O=c1[nH]c(-c2ccc(-c3ccc(Br)nc3)cc2F)nc2c1CSCC2] |
| A6 | 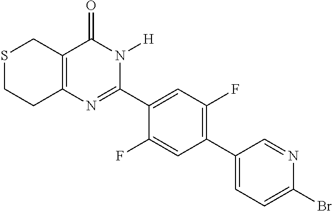 [SMILES: O=c1[nH]c(-c2cc(F)c(-c3ccc(Br)nc3)cc2F)nc2c1CSCC2] |

In some or any embodiments, provided herein are:
(a) compounds as described herein, e.g., of Formula (I) and Compounds 1-44 and pharmaceutically acceptable salts and compositions thereof,
(b) compounds as described herein, e.g., of Formula (I), and Compounds 1-44 and pharmaceutically acceptable salts and compositions thereof for use in stimulating regeneration of a wound in a mammal;
(c) compounds as described herein, e.g., of Formula (I), and Compounds 1-44 and pharmaceutically acceptable salts and compositions thereof for use in inhibiting Wnt transcription products or Wnt signaling pathway activity;
(d) processes for the preparation of compounds as described herein, e.g., of Formula (I) and Compounds 1-44 as described in more detail elsewhere herein;
(e) pharmaceutical formulations comprising a compound as described herein, e.g., of Formula (I) and Compounds 1-44 or a pharmaceutically acceptable salt thereof together with a pharmaceutically acceptable carrier (e.g. diluent);
(f) a method for the treatment of a condition associated with Wnt transcription products or Wnt signaling pathway activity in a mammal that includes the administration of a therapeutically effective amount of a compound as described herein, e.g., of Formula (I) and Compounds 1-44 or its pharmaceutically acceptable salt or composition;
(g) a method for the treatment of a wound in a mammal that includes the administration of a therapeutically effective amount of a compound as described herein, e.g., of Formula (I) and Compounds 1-44 or its pharmaceutically acceptable salt or composition;
(h) pharmaceutical formulations comprising a compound as described herein, e.g., of Formula (I) and Compounds 1-44 or a pharmaceutically acceptable salt thereof together with one or more other effective agents for treating a wound and/or condition modulated by Wnt transcription products or Wnt signaling pathway activity, optionally in a pharmaceutically acceptable carrier (e.g. diluent);
(i) a method for the treatment of a wound in a mammal that includes the administration of a therapeutically effective amount of a compound as described herein, e.g., of Formula (I) and Compounds 1-44 or its pharmaceutically acceptable salt or composition in combination and/or alternation with one or more agent for the treatment of a wound and/or conditions modulated by Wnt transcription products or Wnt signaling pathway activity;
(j) a method for the treatment of a condition associated with Wnt transcription products or Wnt signaling pathway activity in a mammal that includes the administration of a therapeutically effective amount of a compound as described herein, e.g., of Formula (I) and Compounds 1-44 or its pharmaceutically acceptable salt or composition in combination and/or alternation with one or more agent for the treatment of a wound; and
(k) use of any compound described herein, e.g., of Formula (I) and Compounds 1-44 or a composition comprising any compound described herein, e.g., of Formula (I) and Compounds 1-44 or a pharmaceutically acceptable salt or composition for the treatment of a wound associated with Wnt transcription products or Wnt signaling pathway activity described herein, optionally in combination and/or alternation with one or more agent for the treatment of a wound.

### Optically Active Compounds

It is appreciated that compounds provided herein have several chiral centers and may exist in and be isolated in optically active and racemic forms. It is to be understood that any racemic, optically-active, diastereomeric, tautomeric, or stereoisomeric form, mixture, or combination thereof, of a compound provided herein, which possess the useful properties described herein is within the scope of the invention. It being well known in the art how to prepare optically active forms (in some or any embodiments, by resolution of the racemic form by recrystallization techniques, by synthesis from optically-active starting materials, by chiral synthesis, or by chromatographic separation using a chiral stationary phase).

In some or any embodiments, methods to obtain optically active materials are known in the art, and include at least the following.
i) physical separation of crystals—a technique whereby macroscopic crystals of the individual stereoisomers are manually separated. This technique can be used if crystals of the separate stereoisomers exist, i.e., the material is a conglomerate, and the crystals are visually distinct;
ii) simultaneous crystallization—a technique whereby the individual stereoisomers are separately crystallized from a solution of the racemate, possible only if the latter is a conglomerate in the solid state;
iii) enzymatic resolutions—a technique whereby partial or complete separation of a racemate by virtue of differing rates of reaction for the stereoisomers with an enzyme; iv) enzymatic asymmetric synthesis—a synthetic technique whereby at least one step of the synthesis uses an enzymatic reaction to obtain a stereoisomerically pure or enriched synthetic precursor of the desired stereoisomer;
v) chemical asymmetric synthesis—a synthetic technique whereby the desired stereoisomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the stereoisomers with a chiral, non-racemic reagent or catalyst under kinetic conditions;
ix) stereospecific synthesis from non-racemic precursors—a synthetic technique whereby the desired stereoisomer is obtained from non-chiral starting materials and where the stereochemical integrity is not or is only minimally compromised over the course of the synthesis;
x) chiral liquid chromatography—a technique whereby the stereoisomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and stereoisomers are separated by virtue of their differing interactions in the gaseous mobile phase with a column containing a fixed non-racemic chiral adsorbent phase;
xii) extraction with chiral solvents—a technique whereby the stereoisomers are separated by virtue of preferential dissolution of one stereoisomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one stereoisomer of the racemate to pass through.

In some or any embodiments, provided is a composition of a compound that comprises a substantially pure designated stereoisomer of the compound. In some or any embodiments, in the methods and compounds, the compounds are substantially free of other stereoisomer. In some or any embodiments, a composition includes a compound that is at least 85%, 90%, 95%, 98%, 99% or 100% by weight, of the designated stereoisomer, the remainder comprising other chemical species or stereoisomers.

### Isotopically Enriched Compounds

Also provided herein are isotopically enriched compounds.

Isotopic enrichment (in some or any embodiments, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Isotopic enrichment of a drug can be used, in some or any embodiments, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrees the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g., Foster et al., Adv. Drug Res., vol. 14, pp. 1-36 (1985); Kushner et al., Can. J. Physiol. Pharmacol., vol. 77, pp. 79-88 (1999).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or 14C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

In some embodiments, the compounds described herein may be used as radiopharmaceuticals such as, for example, imaging agents. In one instance, radiopharmaceuticals are positron emission tomography (PET) imaging agents. In such embodiments, substitution of radionuclides (e.g., positron emitting isotopes) for atoms in the compounds allows for the syntheses of radiopharmaceuticals that can function as imaging agents. In some embodiments, radionuclides which can be substituted in the compounds described herein include, and are not limited to, ¹⁸F, ¹¹C, ¹³N, ¹¹, ⁷⁶Br, and ¹²⁴I. In some embodiments, the compound is isotopically enriched at one or more atoms, one atom, two atoms, or three atoms. In some embodiments, the compound is administered as an isotopic composition.

The animal body expresses a variety of enzymes for the purpose of eliminating foreign substances, such as therapeutic agents, from its circulation system. In some or any embodiments, such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### Preparation of Compounds

The compounds provided herein can be prepared, isolated or obtained by any method apparent to those of skill in the art. Compounds provided herein can be prepared according to the Exemplary Preparation Schemes provided below. Reaction conditions, steps and reactants not provided in the Exemplary Preparation Schemes would be apparent to, and known by, those skilled in the art.

Additional steps and reagents not provided in the Exemplary Preparation Scheme would be known to those of skill in the art. For example, intermediates and compounds could be prepared using the procedures known by one of ordinary skill in the art or as disclosed in U.S. Provisional Application Nos. 63/417,257; 63/418,947; and 63/418,956 (wherein the synthetic methods disclosed therein are herein incorporated by reference in their entirety). Exemplary methods of preparation are described in detail in the Examples herein.

In one or more embodiments, provided is a method of preparing a compound of Formula (I), comprising: a) contacting a compound of Formula (A):

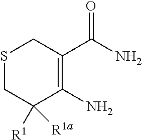

with R^{2′}—C(O)H; or b) contacting a compound of Formula (B):

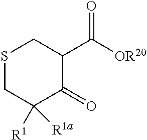

with R^{2′}—C(NH)NH₂, wherein R²⁰ is Me or CD₃; or c) contacting a compound of Formula (C):

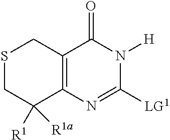

with R^{2′}—H, wherein LG¹ is fluoro, chloro, bromo, iodo, triflate, mesylate, a triazole, a pyrazole, boronic acid, boronic ester, or aryl trifluoroborate; and optionally isolating the compound of Formula (I); wherein R^{2′} is (b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups, (c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups; (e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups; (f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups; (h1) C₃-C₆cycloalkyl substituted with NHl₂ or OH and additionally optionally substituted with 1 or 2 R^{3a} groups; or (i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and provided that the compound is not: methyl 4-oxotetrahydro-2H-thiopyran-3-carboxylate

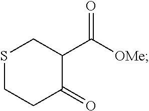 [SMILES: COC(=O)C1CSCCC1=O]

or a salt thereof and/or a stereoisomer or mixture of stereoisomers thereof. In one or more embodiments, methods of preparing a compound of Formula (I) include contacting under basic conditions; and all other groups are as defined herein in any embodiments; and optionally wherein R¹ and R^{1a} are independently hydrogen or alkyl.

One of skill will understand that the order of steps for any process described herein may be changed. Other variations will be apparent to one of skill in the art and all such variations are contemplated within the scope of embodiments presented herein.

### Pharmaceutical Compositions and Methods of Administration

The compounds provided herein can be formulated into pharmaceutical compositions using methods available in the art and those disclosed herein. Any of the compounds disclosed herein can be provided in the appropriate pharmaceutical composition and be administered by a suitable route of administration. Provided herein are pharmaceutical compositions comprising a compound of Formula (I), as described herein in some and any embodiments, and a pharmaceutically acceptable carrier.

In some embodiments, the composition is a topical composition.

The methods provided herein encompass administering pharmaceutical compositions containing at least one compound as described herein, including a compound of Formula (I) if appropriate in a salt form, either used alone or in the form of a combination with one or more compatible and pharmaceutically acceptable carriers, such as diluents or adjuvants, or with another agent for the treatment of wounds and/or conditions modulated by Wnt transcription products or Wnt signaling pathway activity.

In the compositions as described herein (including in the GO-HA pharmaceutical composition), other pharmaceutical or therapeutic compounds may be included in addition to the compound of Formula (I). In other words, the compositions (including in the GO-HA pharmaceutical composition) with the compound of Formula (I) present can also serve as a base dispersion medium in which other pharmaceutical or therapeutic agents, especially those which are hydrophobic, may be dispersed, e.g., for topical administration to a wound. These agents may include antifibrotic compounds such as pirfenidone, halofuginone, nintedanib, tocilizumab, rilonacept, etc., anti-cancer agents, anti-inflammatory agents, analgesics, antibiotics, Wnt inhibitors, Hedgehog pathway inhibitors, TGF-β inhibitors, LOX inhibitors, etc.

In some embodiments, the compositions can include a second medication or therapeutic agent to the wound, comprising one or more of: corticosteroid, a cytotoxic drug, an antibiotic, an antiseptic, nicotine, an anti-platelet drug, an NSAID, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, or nitric oxide. These therapeutic agents can be delivered as separate dosage forms from the compositions described herein, or may be included as additional components of the compositions described herein, hence delivered together with the compound of Formula (I)(or any embodiments thereof, including in some embodiments, Compound 1, Compound 7, or Compound 8).

In some or any embodiments, the second agent can be formulated or packaged with the compound provided herein. Of course, the second agent will only be formulated with the compound provided herein when, according to the judgment of those of skill in the art, such co-formulation should not interfere with the activity of either agent or the method of administration. In some or any embodiments, the compound provided herein and the second agent are formulated separately. They can be packaged together, or packaged separately, for the convenience of the practitioner of skill in the art.

In clinical practice the active agents provided herein may be administered by any conventional route, in particular parenterally, rectally, orally, by inhalation (e.g. in the form of aerosols), or topically.

The composition(s) of the present disclosure described herein can be administered by applying the composition(s) topically on the wound. If the composition is included in a medical device described herein which includes a substrate such as a patch or a pad, the medical device can be secured to the wound such that the composition contacts the wound.

Use may be made, as solid compositions for oral administration, of tablets, pills, hard gelatin capsules, powders or granules. In these compositions, the active product is mixed with one or more inert diluents or adjuvants, such as sucrose, lactose or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, in some or any embodiments, wetting, sweetening or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, in some or any embodiments, ethyl oleate. These compositions can also contain adjuvants, in particular wetting, isotonizing, emulsifying, dispersing and stabilizing agents. Sterilization can be carried out in several ways, in some or any embodiments, using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols or sprays, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, in some or any embodiments, dextran, mannitol or lactose. In one or more embodiments, a pharmaceutical composition provided herein is a spray.

In some or any embodiments, a composition provided herein is a pharmaceutical composition or a single unit dosage form. Pharmaceutical compositions and single unit dosage forms provided herein comprise a therapeutically effective amount of one or more therapeutic agents (e.g., a compound provided herein, or other therapeutic agent), and a typically one or more pharmaceutically acceptable carriers (e.g. excipients). In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the term “carrier” includes a diluent, disintegrant, lubricant, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Remington: The Science and Practice of Pharmacy; Pharmaceutical Press; 22 edition (Sep. 15, 2012).

Typical pharmaceutical compositions and dosage forms comprise one or more excipients. Suitable excipients are well-known to those skilled in the art of pharmacy, and in some or any embodiments, suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a mammal and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, in some or any embodiments, in the U.S. Pharmacopeia (USP 36-NF 31 S2). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, New York, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. In some or any embodiments, suitable packaging include, but are not limited to, hermetically sealed foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Further provided are pharmaceutical compositions and dosage forms that comprise one or more chemical permeation enhancer. In some or any embodiments, chemical permeation enhancers include but are not limited to ethanol, amides (such as Ozone, Laurocapram), alkyl and benzoate esters, fatty acid esters (such as isopropyl myristate, propylene glycol monocaprylate and propyleneglycomonolaurate), Transcutol (Registered name), fatty acids (oleic acid), glycols, pyrrolidone (N-methyl-2-pyrrolidone and 2-pyrrolidone, dimethylsulfoxide (DMSO), terpenes (such as essential oils comprising terpenes), phospholipids, and/or cyclodeextrines.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in some or any embodiments, in purified form, together with a suitable amount of carrier so as to provide the form for proper administration to the mammal. The formulation should suit the mode of administration. In a some or any embodiment, the pharmaceutical compositions or single unit dosage forms are sterile and in suitable form for administration to a mammal, in some or any embodiments, a human.

A pharmaceutical composition is formulated to be compatible with its intended route of administration. In some or any embodiments, routes of administration include, but are not limited to, parenteral, e.g., intrathecal, epidural, local or regional for peripheral nerve block, intravenous, intradermal, subcutaneous, intramuscular, subcutaneous, oral, buccal, sublingual, inhalation, intranasal, transdermal, topical (including administration to the eye, and in some embodiments to the cornea), transmucosal, intra-tumoral, intra-synovial, and rectal administration. In a specific embodiment, the composition is formulated in accordance with routine procedures as a pharmaceutical composition adapted for intravenous, subcutaneous, intramuscular, oral, intranasal, or topical (including administration to the eye, and in some embodiments to the cornea) administration to human beings. In a specific embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

In some or any embodiments, dosage forms include, but are not limited to: sprays, tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a mammal, including suspensions (e.g., aqueous or non-aqueous liquid suspensions, oil in water emulsions, or a water in oil liquid emulsions), solutions, and elixirs; liquid dosage forms suitable for parenteral administration to a mammal; and sterile solids (e.g., crystalline or amorphous solids) that can be reconstituted to provide liquid dosage forms suitable for parenteral administration to a mammal.

The composition, shape, and type of dosage forms provided herein will typically vary depending on their use. In some or any embodiments, a dosage form used in the initial treatment of the disease, disorder, or condition may contain larger amounts of one or more of the active ingredients it comprises than a dosage form used in the maintenance treatment of the same disease, disorder, or condition. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington: The Science and Practice of Pharmacy; Pharmaceutical Press; 22 edition (Sep. 15, 2012).

Generally, the ingredients of compositions are supplied either separately or mixed together in unit dosage form, in some or any embodiments, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: The Science and Practice of Pharmacy; Pharmaceutical Press; 22 edition (Sep. 15, 2012).

In some or any embodiments, the oral dosage forms are solid and prepared under anhydrous conditions with anhydrous ingredients, as described in detail herein. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. In some or any embodiments, excipients suitable for use in oral liquid or aerosol dosage forms include, but are not limited to, water, glycols, oils, alcohols, flavoring agents, preservatives, and coloring agents. In some or any embodiments, excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

In some or any embodiments, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

In some or any embodiments, one or more of the following can be used in the pharmaceutical composition: benzyl alcohol, butyl paraben, butylated hydroxy toluene, calcium carbonate, candelilla wax, colloidal silicone dioxide, calcium stearate, calcium disodium EDTA, copolyvidone or copovidone, calcium hydrogen phosphate dihydrate, crosspovidone, calcium phosphate (di and tri basic), emollients (glyceryl monostearate), iron oxide ivy yellow yellow, and iron.

In some or any embodiments, excipients that can be used in oral dosage forms include, but are not limited to, binders, fillers, disintegrants, and lubricants. Binders suitable for use in pharmaceutical compositions and dosage forms include, but are not limited to, copolyvidone or copovidone, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

In some or any embodiments, fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

In some or any embodiments, suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms include, but are not limited to, crosspovidone, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, in some or any embodiments, a syloid silica gel (AEROSIL 200, manufactured by W.R. Grace Co. of Baltimore, MD), a coagulated aerosol of synthetic silica (marketed by Degussa Co. of Plano, TX), CAB O SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

GO-HA Formulation(s) (with Compound(s) of Formula (I), and any Embodiments Thereof)

In one or more embodiments, a pharmaceutical composition (for example, for treating a wound) is provided, which includes: a matrix component comprising a conjugate of graphene oxide (GO) and hyaluronic acid (HA) where GO and HA are covalently linked via a linker; a compound of Formula (I) (or any embodiments thereof, including in some embodiments, Compound 1, Compound 7, or Compound 8); and water. The covalently-linked GO and HA is also referred to herein as GO-HA conjugate or simply GO-HA. The GO-HA conjugate can be made according to procedures known to a person of ordinary skill in the art, including those disclosed in US-2019-0105398-A1.

Graphene oxide (GO) as used herein refers to an oxidized form of graphene, which is a single layer form of graphite. GO can be obtained by treating graphite with strong oxidizers. GO contains carbon, oxygen, and hydrogen in various amounts, depending on how it is made. It can be of length of several hundreds of nanometers up to several micrometers, its planar direction, and about 0.7-1.2 nm in thickness. GO can include various oxygen containing moieties, such as oxygen epoxide groups, carboxylic acid (—COOH), phenol, etc., when prepared using sulfuric acid (e.g. Hummers method). An example GO structure is shown below.

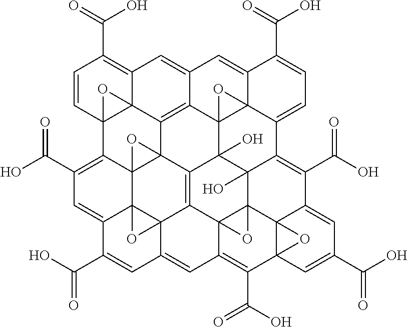

Hyaluronic acid (HA) is an anionic, highly hydrophilic, non-sulfated glycosaminoglycan, occurring naturally throughout the human body. It can be several thousands of carbohydrate units long, and can bind to water giving it a gel of stiff viscous quality. An example structure of HA is provided below:

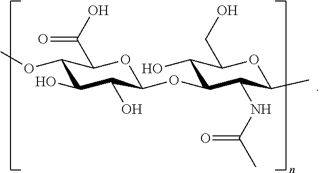

In a composition of present disclosure, the GO and HA are covalently linked to form a matrix component (or a carrier), which can serve to form a stable suspension the compound of Formula (I) (such as Compound 1, Compound 7, or Compound 8) as well as providing other simultaneous benefits to wound healing. The covalent linking can be accomplished by using a linker or linker moiety (“GO-HA linker”). In one or more embodiments, the GO-HA linker can include 2-25 carbons. In one or more embodiments, the GO-HA linker is linear. In one or more embodiments, the GO-HA linker is branched. The GO-HA linker can be saturated or unsaturated.

In one or more embodiments, the GO-HA linker can comprise a C₂-C₂₅ alkylene group, where the carbons and hydrogens in the alkylene group can be substituted by oxygen or other atoms or groups such as hydroxy, carboxy, amino, alkyl, alkoxy, alkenyl, alkynyl, nitro, etc. In one or more embodiments, the GO-HA linker can comprise one or more —CH₂CH₂O-units.

In one or more embodiments, the GO-HA linker comprises —Rₓ—Rₛ—R_{y}—, wherein Rₓ and R_{y} are each independently selected from the group consisting of —CO—, —COO—, —H—, —H—H—, —H—H—CO—, —CS—, —S—, —O—, and wherein Rₛ (which is also referred to as the spacer group in this application) can be an unsubstituted or substituted, saturated or unsaturated linear alkylene group having 2-20 backbone carbons. In one or more embodiments, both Rₓ and R_{y} are *—H—H—CO-(* denoting the ends of the linker distal to Rs).

In one or more embodiments, the spacer group in the GO-HA linker can be an unsubstituted or substituted, saturated or unsaturated linear alkylene group having 2-20 backbone carbons. For illustration and not limitation, the HA can be derivatized with one of the following spacer groups:

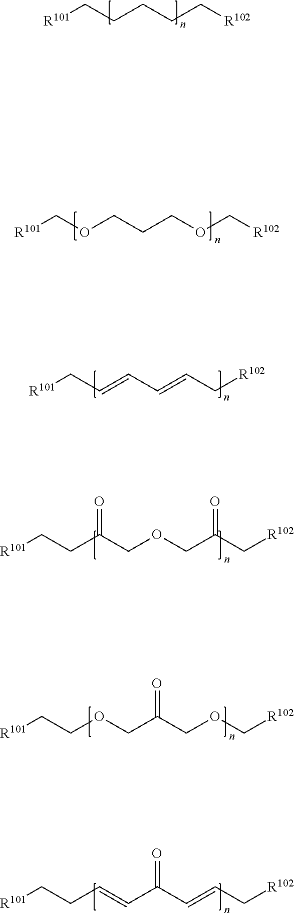

where R¹⁰¹ and R¹⁰² can be independently —CONHNH—, —S—, —NH—, —O—, or other nucleophiles, and n is an integer and can be for example, 1-20, e.g., 1, 2, 3, 4, 5, 6, 7, 8, 9, 10, etc. In one or more embodiments, the HA is derivatized with a spacer group comprising a dihydrazide (e.g. —NHNHC(O)-alkylene-CONHNH—), such as adipic acid dihydrazide (—NHNHC(O)(CH₂)₄CONHNH—).

In one or more embodiments of the GO-HA pharmaceutical composition, the weight ratio of the compound of Formula (I) (such as Compound 1, Compound 7, or Compound 8) to GO-HA can be from about 1:100 to 100:1, e.g., from about 1:2 to about 2:1. In one or more embodiments, in the GO-HA conjugate, the weight ratio of GO:HA can be from about 1:1 to about 1:20, or from about 1:6 to about 1:10.

In one or more embodiments, the GO-HA pharmaceutical composition further comprises pharmaceutical carriers (e.g. excipients), compounds, or materials which enable the compositions to be presented in topically administrable semi-solid aqueous gel forms. For example, carboxymethylcellulose can be used as a gel-forming agent. However, other cellulose derivatives such as microcrystalline cellulose as well as polysaccharides such as alginate, agarose, tragacanth, guar gum, and xanthum gum; are also suitable as gel-forming agents. The gel may, if required, be made thicker and/or stiffer by addition of a relatively resilient gel-forming material such as a cross-linked fibrous protein, e.g. gelatin or collagen cross-linked with formaldehyde. In one or more embodiments, the GO-HA pharmaceutical composition can be in a form of a cream, which can include those excipients suitable for a cream formulation, such as paraffin oil, vaseline, wax, organic esters such as cetyl palmitate, etc.

In one or more embodiments, the GO-HA pharmaceutical composition of the disclosure further comprises a thickener for desired viscosity of the composition for skin delivery. For example, the thickener can include hydroxypropyl cellulose (HPC). HPC can make the GO-HA pharmaceutical composition into a smooth film for easy application. It also reduces evaporation and allows the wound to stay moist longer, a factor that has been shown to improve healing and result in decreased scarring. There are different grades of UPC available according to molecular weights or viscosity of certain concentrations of UPC water solution.

In one or more embodiments of the GO-HA pharmaceutical composition, the compound of Formula (I) (such as Compound 1, Compound 7, or Compound 8) can constitute from about 0.001 wt % to about 5 wt % of the total composition (including water). In one or more embodiments of the GO-HA pharmaceutical composition, the compound of Formula (I) (such as Compound 1, Compound 7, or Compound 8) can constitute from about 0.01 wt % to about 2 wt %, from about 0.02 wt % to about 1 wt %, or from about 0.05 wt % to about 0.5 wt % of the total composition. In one or more embodiments, GO-HA constitutes from about 0.001 wt % to about 5 wt % of the total composition. In one or more embodiments, GO-HA can constitute from about 0.01 wt % to about 2 wt %, from about 0.02 wt % to about 1 wt %, or from about 0.05 wt % to about 0.5 wt % of the total composition.

In general, the GO-HA pharmaceutical composition overall can appear as a slightly dark or black viscous liquid. The compound of Formula (I) (such as Compound 1, Compound 7, or Compound 8) is evenly dispersed in the viscous suspension, which is stable at room temperature for months. In one or more embodiments, the composition further comprises a surfactant that enhances mixability or solubility of hydrophobic substances in water. In one or more examples, the surfactant can be a non-ionic hydrophilic material such as polyethylene glycol (PEG). The PEG can have a number-averaged molecular weight of from about 100 to about 10,000 Daltons, or about 200 to about 4000 Daltons, e.g., from about 200 to about 1000, from about 200 to about 800, from about 200 to about 500, from about 200 to about 400, from about 300 to about 400, from about 350 to about 450, about 200, about 250, about 300, about 350, about 400, about 450, about 500, about 550, about 600, about 650, about 700, about 750, about 800, about 850, about 900, about 950, about 1000 Daltons, etc. In one or more embodiments, the PEG can be present in the composition in an amount of from about 0.1 to about 20 wt % of that of the total composition. For example, the PEG can be from about 0.2 wt % to about 10 wt %, or from about 0.5 wt % to about 10 wt %, or from about 1 wt % to about 10 wt % of the total composition. Other non-ionic hydrophilic material such as copolymers of PEG and PPG (polypropylene glycol), e.g., poloxamers, can also be used. In one example, Poloxamer-188 (which has an average molecular weight of about 8400 Daltons) can be used.

In the GO-HA pharmaceutical compositions as described herein, other pharmaceutical or therapeutic compounds may be included in addition to a compound of Formula (I) (such as Compound 1, Compound 7, or Compound 8). In other words, the GO-HA pharmaceutical compositions with compound(s) of Formula (I) (such as Compound 1, Compound 7, or Compound 8) present can also serve as a base dispersion medium in which other pharmaceutical or therapeutic agents, especially those which are hydrophobic, may be dispersed, e.g., for topical administration to a wound. These agents may include antifibrotic compounds such as pirfenidone, halofuginone, nintedanib, tocilizumab, rilonacept, etc., anti-cancer agents, anti-inflammatory agents, analgesics, antibiotics, Wnt inhibitors, Hedgehog pathway inhibitors, TGF-β inhibitors, LOX inhibitors, etc.

### Delayed Release Dosage Forms

Active ingredients such as the compounds provided herein can be administered by controlled release means or by delivery devices that are well known to those of ordinary skill in the art. In some or any embodiments, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500; each of which is incorporated herein by reference in its entirety. Such dosage forms can be used to provide slow or controlled release of one or more active ingredients using, in some or any embodiments, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus encompassed herein are unit dosage forms suitable for oral administration such as, but not limited to, tablets, capsules, gel caps, and caplets that are adapted for controlled release.

All controlled release pharmaceutical products have a common goal of improving drug therapy over that achieved by their non-controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In some or any embodiments, the drug may be administered using intravenous infusion, an implantable osmotic pump, a transdermal patch, liposomes, or other modes of administration. In some or any embodiments, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a mammal at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the mammal.

### Parenteral Dosage Forms

In some or any embodiments, provided are parenteral dosage forms. Parenteral dosage forms can be administered to mammals by various routes including, but not limited to, subcutaneous, intravenous (including bolus injection), intramuscular, and intra-arterial. Because their administration typically bypasses the mammal's natural defenses against contaminants, parenteral dosage forms are typically, sterile or capable of being sterilized prior to administration to a mammal. In some or any embodiments, parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. In some or any embodiments, suitable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Transdermal, Topical & Mucosal Dosage Forms

Also provided are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington: The Science and Practice of Pharmacy; Pharmaceutical Press; 22 edition (Sep. 15, 2012); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable carriers (e.g., excipients and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington: The Science and Practice of Pharmacy; Pharmaceutical Press; 22 edition (Sep. 15, 2012).

Depending on the specific tissue to be treated, additional components may be used prior to, in conjunction with, or subsequent to treatment with active ingredients provided. In some or any embodiments, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Dosage and Unit Dosage Forms

In human therapeutics, the doctor will determine the posology which the doctor considers most appropriate according to the treatment needed (e.g. preventative or curative) and according to the age, weight, stage of the disease, disorder, or condition and other factors specific to the mammal to be treated. In some or any embodiments, topical doses are described as mg per cm² of treatment site (e.g. a wound) and are from about 0.001 to about 50 mg/cm², or from about 0.005 to about 50 mg/cm², or from about 0.01 to about 50 mg/cm², or from about 0.01 to about 40 mg/cm², or from about 0.01 to about 30 mg/cm², or from about 0.01 to about 20 mg/cm², or from about 0.01 to about 10 mg/cm², or from about 0.05 to about 10 mg/cm², or from about 0.05 to about 1 mg/cm².

In some or any embodiments, topical and non-topical doses are from about 1 to about 1000 mg per day for an adult, or from about 5 to about 250 mg per day or from about 10 to about 50 mg per day for an adult. In some or any embodiments, doses are from about 5 to about 400 mg per day or about 25 to about 200 mg per day per adult. In some or any embodiments, dose rates of from about 50 to about 500 mg per day are also contemplated. In some or any embodiments, doses for subcutaneous administration are from about 1 to about 50 mg per day, or from about 1 to about 25 mg per day, or from about 1 to about 10 mg per day, or from about 1 to about 20 mg per day, or from about 5 to about 25 mg per day, or from about 5 mg to about 20 mg per day, or from about 10 to about 20 mg per day. In some or any embodiments, doses for oral administration are from about 0.01 mg to about 100 mg per day, from about 0.01 to about 100 mg per day, or from about 0.01 mg to about 50 mg per day, from about 0.01 to about 25 mg per day, from about 0.01 to about 15 mg per day, from about 0.01 to about 10 mg per day, from about 0.05 to about 10 mg per day, from about 0.05 to about 5 mg per day, from about 0.05 to about 1 mg per day, from about 0.1 to about 100 mg per day, from about 0.1 to about 50 mg per day, from about 0.1 to about 25 mg per day, from about 0.1 to about 15 mg per day, from about 0.1 to about 10 mg per day, from about 0.1 to about 5 mg per day, or from about 0.5 mg to about 1 mg per day, or from about 10 mg to about 200 mg per day. In some or any embodiments, including any of the foregoing embodiments, the daily dose can be administered once a day. In some or any embodiments, including any of the foregoing embodiments, the daily dose can be divided and administered twice a day. In some or any embodiments, including any of the foregoing embodiments, the daily dose can be divided and administered three times a day.

In some embodiments, the mg/day amounts are for an adult. In further aspects, provided are methods of treating a disease, disorder, or condition associated with Wnt signaling pathway activity in a mammal by administering to a mammal in need thereof, a therapeutically or prophylactically effective amount of a compound provided herein, or a pharmaceutically acceptable salt thereof. The amount of the compound or composition which will be therapeutically or prophylactically effective in the treatment of a disorder or one or more symptoms thereof will vary with the nature and severity of the disease or condition, and the route by which the active ingredient is administered. The frequency and dosage will also vary according to factors specific for each mammal depending on the specific therapy (e.g., therapeutic or prophylactic agents) administered, the severity of the disorder, disease, or condition, the route of administration, as well as age, body, weight, response, and the past medical history of the mammal. Effective doses may be extrapolated from dose-response curves derived from in vitro or animal model test systems.

In some or any embodiments, exemplary doses of a composition include milligram or microgram amounts of the active compound per kilogram of mammal or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in some or any embodiments, the dosage administered to a mammal is 0.01 mg/kg to 3 mg/kg of the mammal's body weight, or 0.10 mg/kg to 3 mg/kg of the mammal's body weight, based on weight of the active compound. In some or any embodiments, the dosage administered to a mammal is between 0.20 mg/kg and 2.00 mg/kg, or between 0.30 mg/kg and 1.50 mg/kg of the mammal's body weight. In some embodiments, the dosage is administered subcutaneously to a mammal and is between about 0.01 mg/kg to 1 mg/kg (inclusive), or between about 0.03 mg/kg to 0.5 mg/kg (inclusive) of the mammal's body weight, based on weight of the active compound. In some embodiments, the dosage is administered orally to a mammal and is between about 0.10 mg/kg to 5 mg/kg (inclusive) of the mammal's body weight, or between about 0.10 mg/kg to 2 mg/kg (inclusive) of the mammal's body weight, based on weight of the active compound. In some or any embodiments, the recommended daily topical dose range of a composition provided herein for the conditions described herein lie within the range of from about 0.01 mg to about 100 mg per day, given as a single once-a-day dose or as divided doses (e.g. in two or three doses) throughout a day.

In some or any embodiments, the recommended daily dose range of a composition provided herein for the conditions described herein lie within the range of from about 0.1 mg to about 1000 mg per day, given as a single once-a-day dose or as divided doses throughout a day. In some or any embodiments, the daily dose is administered twice daily in equally divided doses. In some or any embodiments, the daily dose is administered thrice daily in equally divided doses. In some or any embodiments, the daily dose is administered four times daily in equally divided doses. In some or any embodiments, a daily dose range should be from about 0.01 mg to about 400 mg per day, from about 0.1 mg to about 250 mg per day, from about 10 mg to about 200 mg per day, in other embodiments, or from about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with the response of the mammal.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the herein described dosage amounts and dose frequency schedules. Further, when a mammal is administered multiple dosages of a composition provided herein, not all of the dosages need be the same. In some or any embodiments, the dosage administered to the mammal may be increased to improve the prophylactic or therapeutic effect of the composition or it may be decreased to reduce one or more side effects that a particular mammal is experiencing.

In some or any embodiment, the dosage of the composition provided herein, based on weight of the active compound, administered to prevent, treat, manage, or ameliorate a disorder, or one or more symptoms thereof in a mammal is about 0.01 mg/kg, about 0.1 mg/kg, about 1 mg/kg, about 2 mg/kg, about 3 mg/kg, about 4 mg/kg, about 5 mg/kg, about 6 mg/kg, about 10 mg/kg, or about 15 mg/kg or more of a mammal's body weight. In another embodiment, the dosage of the composition or a composition provided herein administered to prevent, treat, manage, or ameliorate a disorder, or one or more symptoms thereof in a mammal is a unit dose selected from about 0.01 mg/kg to about 100 mg/kg, selected from about 0.1 mg to about 200 mg, selected from about 0.1 mg to about 100 mg, selected from about 0.1 mg to about 50 mg, selected from about 0.1 mg to about 25 mg, selected from about 0.1 mg to about 20 mg, selected from about 0.1 mg to about 15 mg, selected from about 0.1 mg to about 10 mg, selected from about 0.1 mg to about 7.5 mg, selected from about 0.1 mg to about 5 mg, 0.1 to about 2.5 mg, selected from about 0.25 mg to about 20 mg, selected from about 0.25 to about 15 mg, selected from about 0.25 to 12 mg, selected from about 0.25 to about 10 mg, selected from about 0.25 mg to about 7.5 mg, selected from about 0.25 mg to about 5 mg, selected from about 0.5 mg to about 2.5 mg, 1 mg to about 20 mg, selected from about 1 mg to about 15 mg, selected from about 1 mg to about 12 mg, 1 mg to about 10 mg, selected from about 1 mg to about 7.5 mg, selected from about 1 mg to about 5 mg, or selected from about 1 mg to about 2.5 mg.

In some or any embodiments, a dose of a compound or composition provided herein can be administered to achieve a steady-state concentration of the active ingredient in blood or serum of the mammal. The steady-state concentration can be determined by measurement according to techniques available to those of skill or can be based on the physical characteristics of the mammal such as height, weight and age. In some or any embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In some or any embodiments, provided herein are unit dosages comprising a compound, or a pharmaceutically acceptable salt thereof, in a form suitable for administration. Such forms are described in detail herein. In some or any embodiments, the unit dosage comprises 1 to 1000 mg, 1 to 100 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

In some or any embodiments, dosages of the second agents to be used in a combination therapy are provided herein. In some or any embodiments, dosages lower than those which have been or are currently being used to treat the disease, disorder, or condition are used in the combination therapies provided herein. The recommended dosages of second agents can be obtained from the knowledge of those of skill in the art. For those second agents that are approved for clinical use, recommended dosages are described in, for example, Hardman et al., eds., 1996, Goodman & Gilman's The Pharmacological Basis Of Therapeutics 9^{th} Ed, Mc-Graw-Hill, New York; Physician's Desk Reference (PDR) 57^{th} Ed., 2003, Medical Economics Co., Inc., Montvale, NJ; which are incorporated herein by reference in their entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. In various embodiments, the therapies are administered no more than 24 hours apart or no more than 48 hours apart. In some or any embodiments, two or more therapies are administered within the same patient visit. In other embodiments, the compound provided herein and the second agent are administered concurrently.

In other embodiments, the compound provided herein and the second agent are administered at about 2 to 3 days apart, 2 to 4 days apart, at about 4 to 6 days apart, at about 1 week part, at about 1 to 2 weeks apart, or more than 2 weeks apart.

In some or any embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In some or any embodiments, a compound provided herein and a second agent are administered to a patient, in some or any embodiments, a mammal, such as a human, in a sequence and within a time interval such that the compound provided herein can act together with the other agent to provide an increased benefit than if they were administered otherwise. In some or any embodiments, the second active agent can be administered at the same time or sequentially in any order at different points in time; however, if not administered at the same time, they should be administered sufficiently close in time so as to provide the desired therapeutic or prophylactic effect. In some or any embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In some or any embodiments, the compound provided herein and the second agent are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some or any embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. In some or any embodiments, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In some or any embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered prior to or subsequent to administration of a second agent. Also contemplated are administration of a compound provided herein and a second agent by the same or different routes of administration, e.g., oral and parenteral. In some or any embodiments, when the compound provided herein is administered concurrently with a second agent that potentially produces adverse side effects including, but not limited to, toxicity, the second active agent can advantageously be administered at a dose that falls below the threshold that the adverse side effect is elicited.

### Kits

Also provided are kits for use in methods of treatment of disease, disorder, or condition associated with Wnt signaling pathway activity. The kits can include a compound or composition provided herein, a second agent or composition, and instructions providing information to a health care provider regarding usage for treating the disease, disorder, or condition associated with Wnt signaling pathway activity. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a mammal, a therapeutically or prophylactically effective plasma level of the compound or composition can be maintained in the mammal for at least 1 day. In some or any embodiments, a compound or composition can be included as a sterile aqueous pharmaceutical composition or dry powder (e.g., lyophilized) composition.

In some or any embodiments, suitable packaging is provided. As used herein, “packaging” includes a solid matrix or material customarily used in a system and capable of holding within fixed limits a compound provided herein and/or a second agent suitable for administration to a mammal. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Methods of Use

Provided herein is a method for inhibiting Wnt transcription signaling pathway in a mammal, which comprises contacting administration of an effect amount of a compound of Formula (I) including a single stereoisomer or mixture of stereoisomers thereof; and/or a pharmaceutically acceptable salt thereof.

Provided herein is a method for the treatment of a disease, disorder, or condition associated with Wnt transcription products or Wnt signaling pathway activity in a mammal, comprising the administration of a therapeutically or prophylactically effective amount of a compound of Formula (I) described herein or a pharmaceutical composition described herein. In one or more embodiments, the method is for treating a disease, disorder, or condition associated with Wnt transcription products or Wnt signaling pathway activity, comprising administering a compound of Formula (I) (or a single stereoisomer or mixture of stereoisomers thereof, a single tautomer or mixture of tautomers thereof, and/or a pharmaceutically acceptable salt thereof) or a pharmaceutical composition according to one or more embodiments to a mammal in need thereof.

In one or more embodiments, the method is for stimulating regeneration of tissue at a wound in a mammal in need thereof. In one or more embodiments, the method comprises contacting the wound with an effective amount of a compound of Formula (I) (or a single stereoisomer or mixture of stereoisomers thereof, a single tautomer or mixture of tautomers thereof, and/or a pharmaceutically acceptable salt thereof) or a pharmaceutical composition according to one or more embodiments. In some embodiments, the mammal is a human. In a group of embodiments, the disease, disorder, or condition is a wound.

In a group of embodiments, the disease, disorder, or condition is selected from a chronic wound, an acute wound, an alkali-burned corneal wound, a burn, a lesion (including lesions caused by HPV and/or a virus selected from the Poxviridae family of viruses), an inflammatory dermatitis disease (including acne, psoriasis, rosacea, and scleroderma), a cartilage disease (including osteoarthritis, rheumatoid arthritis, internal derangement of the joints, and degenerative cartilage disease), a bone disease (including osteoporosis), organ fibrosis (including lung fibrosis, heart fibrosis, liver fibrosis, and kidney fibrosis), cancer (including melanoma, breast cancer, and prostate cancer), a de-nerved body part in need of reinnervation, tissue in need of regeneration (including damaged elastic cartilage), bacterial growth in need of inhibition, fungal growth in need of inhibition, tissue in need of neovascularization, osteoclast differentiation in need of inhibition, osteoblast differentiation disorders (where inhibition of osteoblast differentiation is needed), and/or bone destruction associated with breast cancer

In some or any embodiments, provided herein are methods for treating a disease, disorder, or condition associated with Wnt transcription products or Wnt signaling pathway activity in a mammal in need thereof. In some or any embodiments, the methods encompass the step of administering to the subject in need thereof a therapeutically or prophylactically effective amount of a compound effective for the treatment of a disease, disorder, or condition associated with Wnt transcription products or Wnt signaling pathway activity in combination with a second agent effective for the treatment of a condition associated with Wnt transcription products or Wnt signaling pathway activity. The compound can be any compound as described herein, and the second agent can be any second agent described in the art or herein. In some or any embodiments, the compound is in the form of a pharmaceutical composition or dosage form, as described elsewhere herein.

In some or any embodiments, provided herein is a method of inhibiting Wnt transcription products or Wnt signaling pathway activity comprising contacting Wnt with a compound of Formula (I) or a compound selected from Compounds 1-44.

In one or more embodiments, the present disclosure provides for a method of improving the healing of a wound, the method comprising contacting the wound with an effective amount of the compositions of the present disclosure. The wound subject to an injury is contemplated to include, but not be limited to, those that arise from a surgical wounding caused by a physical impact that disrupts the structure and function of the skin (such as a laceration, abrasion, cut, scratch or puncture by a knife, scalpel, bullet, or other sharp or blunt objects). The present disclosure contemplates use on wounds arising by way of excessive (low or high) temperature such as a burn, ionizing radiation, chemotherapy, or unplanned acute injuries arising from accident or misadventure. The present disclosure contemplates use on chronic wounds arising as a consequence of an underlying condition, such as diabetic ulcerations.

The composition(s) of the present disclosure described herein can be administered by applying the composition(s) topically on the wound. If the composition is included in a medical device described herein which includes a substrate such as a patch or a pad, the medical device can be secured to the wound such that the composition contacts the wound.

In the preparation method of compositions used in the methods of the present disclosure, the spacer group can be an unsubstituted or substituted, saturated or unsaturated linear alkylene group having 2-20 backbone carbons. For illustration and not limitation, the reagent for derivatizing HA can be selected from the following:

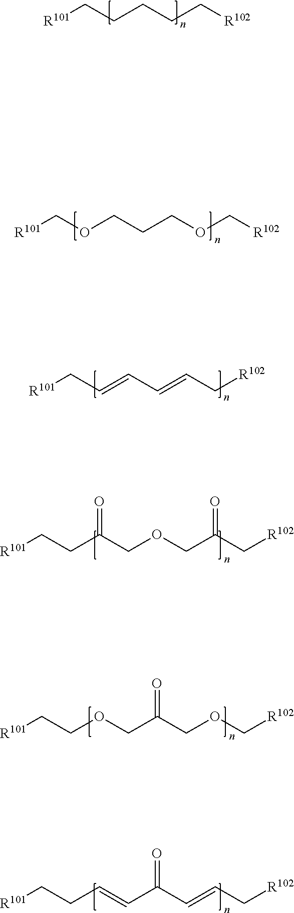

where R¹⁰¹ and R¹⁰² can be independently —CONHNH₂, —SH, —NH₂, —OH, or other nucleophiles, and n is an integer and can be for example, 1-20, e.g., 1, 2, 3, 4, 5, 6, 7, 8, 9, 10, etc. In one or more embodiments, the reagent for derivatizing HA can be a dihydrazide (e.g., —NHNHC(O)— alkylene-CONHNH₂), such as adipic acid dihydrazide (e.g., —NHNHC(O)(CH₂)₄CONHNH₂).

In one or more embodiments, a method for preparing a composition of the present disclosure includes obtaining GO-HA (e.g., by the methods herein), adding or dissolving the GO-HA conjugate in water to obtain a GO-HA water solution, and adding the compound of Formula (I) (or any embodiments thereof, including in some embodiments, Compound 1, Compound 7, or Compound 8) to the GO-HA water solution to form a mixture (GO-HA+compound of Formula (I), or any embodiments thereof, including in some embodiments, GO-HA+Compound 1, GO-HA+Compound 7, or GO-HA+Compound 8). In some examples, this is accomplished by adding or dissolving the compound of Formula (I) (or any embodiments thereof, including in some embodiments, Compound 1, Compound 7, or Compound 8) first in a non-ionic hydrophilic polymer, e.g., PEG-400 (or PEG 400, having an average molar mass of about 400), and then the compound of Formula (I) (or any embodiments thereof, including in some embodiments, Compound 1, Compound 7, or Compound 8) solution is added into the GO-HA conjugate water solution generating GO-HA+compound of Formula (I) (or any embodiments thereof, including in some embodiments, GO-HA+Compound 1, GO-HA+Compound 7, or GO-HA/Compound 8).

In some or any embodiments, the wound to be ameliorated, treated, repaired, or healed is selected from one or more of the group consisting of an acute wound, chronic wound, tear wound, abrasion wound, laceration wound, puncture wound, avulsion wound, skin cut, surgical wound, thermal wound, burn wound, ulcer, chemical wound, bite wound, stab wound, gunshot wound, other penetrating high velocity projectile wound, sting, electrical wound, chop wound, crush wound, poison wound, radiation wound, scalped wound, penetrating wound, incision wound, blunt force trauma wound, skin tear, internal wound, open wound, closed wound, excoriation, infected wound, weeping wound, non-healing wound, wound associated with dressing changes, amputation, necrotizing fasciitis wound, osteomyelitis wound, and post-trauma wound.

The present disclosure contemplates use with chronic wounds arising as a consequence of an underlying condition, such as diabetic ulcerations.

In some or any embodiments, the wound to be ameliorated, treated, repaired, or healed is a wound in an acute care setting, including post-surgery. In some or any embodiments, the wound to be ameliorated, treated, repaired, or healed is a wound in an acute care setting, including post-surgery and the compound is administered intravenously. In some or any embodiments, the wound to be ameliorated, treated, repaired, or healed is a surgical wound. In some or any embodiments, the wound to be ameliorated, treated, repaired, or healed is a surgical wound and the compound is administered topically (e.g., a spray). In some or any embodiments, the wound to be ameliorated, treated, repaired, or healed is an acute or chronic wound. In some or any embodiments, the wound to be ameliorated, treated, repaired, or healed is an acute or chronic wound and the compound is administered subcutaneously. In some or any embodiments, the wound to be ameliorated, treated, repaired, or healed is an acute or chronic wound and the compound is administered orally.

In a group of embodiments, the wound to be ameliorated, treated, repaired, or healed is a burn. In one or more embodiments, the burn is a thermal burn. In one or more embodiments, the burn is a chemical burn. In one or more embodiments, the burn is an electric burn. In one or more embodiments, the burn is a thermal burn. In one or more embodiments, the wound is a radiation burn. In one or more embodiments, the wound is a first degree burn. In one or more embodiments, the wound is a second degree burn. In one or more embodiments, the wound is a third degree burn.

In some or any embodiments, the compounds described herein are used for delaying the onset of a wound, or reducing the severity or duration of a wound. In some or any embodiments, the compounds described herein are used for the reduction of the severity or duration of a wound associated with Wnt transcription products or Wnt signaling pathway activity. In some embodiments, the compounds described herein are used for delaying or preventing onset of a wound.

In some or any embodiments, the compounds described herein are used for prevention of a wound or of a condition associated with Wnt transcription products or Wnt signaling pathway activity.

In some or any embodiments, the compounds described herein are used for treatment of a wound or of a condition associated with Wnt transcription products or Wnt signaling pathway activity.

### Assay Methods

Compounds can be assayed for efficacy in treating a disease, disorder, or condition associated with Wnt signaling pathway activity according to any assay known to those of skill in the art. Exemplary assay methods are provided elsewhere herein.

### Second Therapeutic Agents

In some or any embodiments, the compounds and compositions provided herein are useful in methods of treatment of a wound and/or a condition associated with Wnt transcription products and/or Wnt signaling pathway activity, that comprise further administration of a second agent effective for the treatment of a wound and/or a Wnt transcription related disorder and/or a condition associated with Wnt transcription products and/or Wnt signaling pathway activity. The second agent used in the method of treatment can be any agent known to those of skill in the art to be effective for the treatment of a wound and/or a Wnt transcription related disorder and/or a condition associated with Wnt transcription products and/or Wnt signaling pathway activity, including those currently approved by the United States Food and Drug Administration, or other similar body of a country foreign to the United States. Second medications (second agents) are previously described herein and can be used in the methods of treatment. In some or any embodiments, the second agent is a PARP inhibitor, silver, or a notich inhibitor.

In one or more embodiments, the second agent is one or more of: corticosteroid, a cytotoxic drug, an antibiotic, an antiseptic, nicotine, an anti-platelet drug, an NSAID, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, nitric oxide, an antifibrotic compounds (such as pirfenidone, halofuginone, nintedanib, tocilizumab, rilonacept, etc.), an anti-cancer agent, an anti-inflammatory agent, an analgesic, a Wnt inhibitor, a Hedgehog pathway inhibitor, a TGF-β inhibitor, and/or a LOX inhibitor, etc.

In some or any embodiments, a compound provided herein is administered in combination with one second agent. In further embodiments, a compound provided herein is administered in combination with two second agents. In still further embodiments, a compound provided herein is administered in combination with two or more second agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a mammal with a disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a mammal with a disorder.

As used herein, the term “synergistic” includes a combination of a compound provided herein and another therapy (e.g., a prophylactic or therapeutic agent) which has been or is currently being used to prevent, manage or treat a disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a mammal with a disorder. The ability to utilize lower dosages of a therapy (e.g., a prophylactic or therapeutic agent) and/or to administer said therapy less frequently reduces the toxicity associated with the administration of said therapy to a mammal without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The active compounds provided herein can be administered in combination or alternation with another therapeutic agent, in particular an agent effective in the treatment of a wound and/or a Wnt transcription related disorder and/or a condition associated with Wnt transcription products and/or Wnt signaling pathway activity. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the wound or Wnt-transcription-related disorder to be alleviated. It is to be further understood that for any particular mammal, specific dosage regimens and schedules should be adjusted over time according to the individual need and the professional judgment of the person administering or supervising the administration of the compositions.

### Examples

The following examples are provided for purpose of illustration of certain aspects of the description herein and should not be deemed to limit the disclosure in any way.

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); M (molar); M+1 (MS peak, presence of carbon-13 isotope in molecular ion+1 peak); mmol (millimoles); m/z (mass per charge); h, hr, or hrs (hours); min (minutes); eq (equivalent(s)); RT, R.T., or rt (room temperature); Rₜ or Rt (retention time); R_{f} or Rf (retention factor); v (V) or vol (volume); E (cis); Z (trans); MS (mass spectrometry); ESI (electrospray ionization); TLC (thin layer chromatography); HPLC (high pressure liquid chromatography); LC-MS (liquid chromatography-mass spectrometry); ¹H NMR (proton nuclear magnetic resonance); ACN or CH₃CN (acetonitrile); Ac₂O (acetic anhydride); AcOH (acetic acid); BPin (bis(pinacolato)); BBr₃ (boron tribromide); CDCl₃ (deuterated chloroform); CH₂Cl₂ or DCM (dichloromethane); CuBr₂ (copper (II) bromide); CN (cyanide or cyano); Cs₂CO₃ (cesium carbonate); DCM (dichloromethane); DMF (dimethylformamide); DMSO (dimethylsulfoxide); DMSO-d₆ (deuterated dimethylsulfoxide); EtOAc (ethyl acetate); FA (formic acid); H₂ (hydrogen gas); HCl (hydrochloride or hydrochloric acid); I₂ (iodine); K₂CO₃ (potassium carbonate); KOAc (potassium acetate); LDA (lithium diisopropylamide); LAH (lithium aluminum hydride); LHMIDS or LiHMDS (lithium bis(trimethylsilyl)amide); MeOH (methanol); MeOD (methanol-D); MeMgBr (methyl magnesium bromide); N₂ (nitrogen); NaH (sodium hydride); NH₂OH (hydroxylamine); Na₂SO₄ (sodium sulfate); NaHCO₃ (sodium bicarbonate); NaHMDS (sodium bis(trimethylsilyl)amide); NaOMe (sodium methoxide); NH₃ (ammonia); NH₄Cl (ammonium chloride); NH₄HCO₃ (ammonium bicarbonate); NMP (n-methyl-2-pyrrolidone); OMe (methoxy); Pd/C (palladium on carbon); PE (petroleum ether); Ph (phenyl); —Si(tert-Bu)(Ph)₂ and —Si^{t}BuPh₂ (tert-butyl-diphenylsilyl); SiO₂ (silicon dioxide); THE (tetrahydrofuran); TFA (trifluoroacetic acid); tBuONO (t-butyl nitrite); dppf (diphenylphosphino); TMS (trimethylsilyl); GO-HA (graphene oxide/hyaluronic acid); MWCO (molecular weight cutoff); RPM or rpm (revolutions per minute); N (normality) or N (newton); and CFU (colony-forming unit).

### Synthetic Examples

### Compound 1

### Synthesis of (4-(4-oxo-3,5,7,8-tetrahydro-4H-thiopyrano[4,3-d]pyrimidin-2-yl)phenyl)boronic acid (Compound 1)

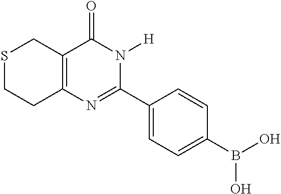 [SMILES: O=c1[nH]c(-c2ccc(B(O)O)cc2)nc2c1CSCC2]

### Synthesis of 2-(4-bromophenyl)-3,5,7,8-tetrahydro-4H-thiopyrano[4,3-d]pyrimidin-4-one

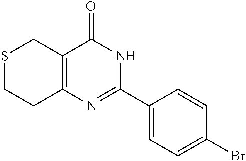 [SMILES: O=c1[nH]c(-c2ccc(Br)cc2)nc2c1CSCC2]

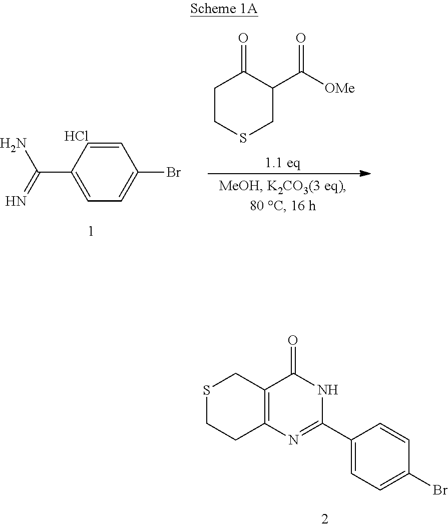 [SMILES: Cl ; COC(=O)C1CSCCC1=O ; N=C(N)c1ccc(Br)cc1 ; O=c1[nH]c(-c2ccc(Br)cc2)nc2c1CSCC2]

A mixture of 4-bromobenzimidamide hydrochloride (1) (22.0 g, 93.41 mmol, 1.0 eq), methyl 4-oxotetrahydro-2H-thiopyran-3-carboxylate (1.1 g, 102.76 mmol, 1.1 eq), and K₂CO₃ (38.73 g, 280.23 mmol, 3.0 eq) in MeOH (660 mL) was stirred at 80 ° C for 16 h. The mixture was cooled and concentrated in vacuo. The solid was triturated with water (500 mL), and then filtered and dried to afford 2-(4-bromophenyl)-3,5,7,8-tetrahydro-4H-thiopyrano[4,3-d]pyrimidin-4-one (2) (26.4 g, 81.7 mmol). LC-MS: calculated for C₁₃H₁₁BrN₂OS: 323.2; found: 325.0.

### Synthesis of 2-(4-(4,4,5,5-tetramethyl-1,3,2-dioxaborolan-2-yl)phenyl)-3,5,7,8-tetrahydro-4H-thiopyrano[4,3-d]pyrimidin-4-one

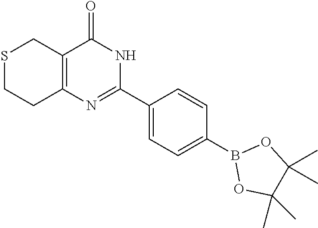 [SMILES: CC1(C)OB(c2ccc(-c3nc4c(c(=O)[nH]3)CSCC4)cc2)OC1(C)C]

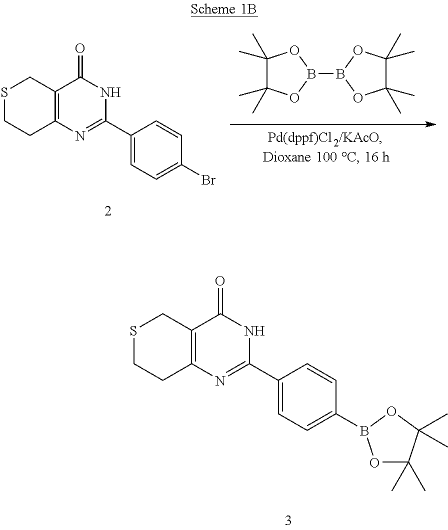

The degassed mixture of 2-(4-bromophenyl)-3,5,7,8-tetrahydro-4H-thiopyrano[4,3-d]pyrimidin-4-one (2) (20 g, 61.88 mmol, 1.0 eq), (BPin)₂ (31.43 g, 123.76 mmol, 2.0 eq), Pd(dppf)Cl₂ (2.26 g, 3.09 mmol, 0.05 eq), and KOAc (30.36 g, 309.40 mmol, 5.0 eq) in dioxane (200 mL) was stirred at 100° C. for 16 h under Argon. The mixture was concentrated in vacuo. The crude product was purified on a silica gel column (PE:EtOAc=5:1 to 1:1) to afford 2-(4-(4,4,5,5-tetramethyl-1,3,2-dioxaborolan-2-yl)phenyl)-3,5,7,8-tetrahydro-4H-thiopyrano[4,3-d]pyrimidin-4-one (3) (21.73 g, 58.72 mmol). LC-MS: calculated for C₁₉H₂₃BN₂O₃S: 370.1; found: 371.1.

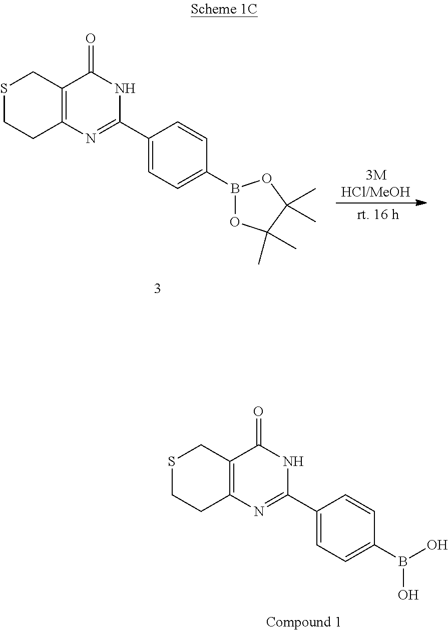 [SMILES: CC1(C)OB(c2ccc(-c3nc4c(c(=O)[nH]3)CSCC4)cc2)OC1(C)C ; O=c1[nH]c(-c2ccc(B(O)O)cc2)nc2c1CSCC2]

A solution of 2-(4-(4,4,5,5-tetramethyl-1,3,2-dioxaborolan-2-yl)phenyl)-3,5,7,8-tetrahydro-4H-thiopyrano[4,3-d]pyrimidin-4-one (3) (10.0 g, 370.27 mmol, 1.0 eq) in 3M HCl/MeOH (100 mL) was stirred at 25° C. for 16 h. The mixture was concentrated under vacuum to afford a crude product which was triturated with THE (150 mL) under reflux and filtered to afford (4-(4-oxo-3,5,7,8-tetrahydro-4H-thiopyrano[4,3-d]pyrimidin-2-yl)phenyl)boronic acid (Compound 1). LC-MS: calculated for C₁₃H₁₃BN₂O₃S: 288.1; found: 289.1. ¹H NMR (400 MHz, MeOD): δ 7.95 (s, 4H), 3.67 (s, 2H), 3.10 (m, 2H), 3.02 (m, 2H).

### Compound 2

### Synthesis of 2-oxo-1-((1r,3r)-3-(4-oxo-3,5,7,8-tetrahydro-4H-thiopyrano[4,3-d]pyrimidin-2-yl)cyclobutyl)-2,3-dihydro-1H-benzo[d]imidazole-5-carbonitrile (Compound 2)

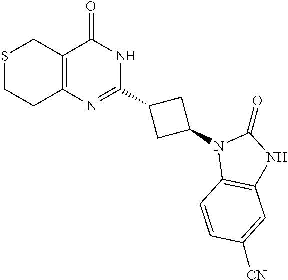 [SMILES: N#Cc1ccc2c(c1)[nH]c(=O)n2[C@H]1C[C@H](c2nc3c(c(=O)[nH]2)CSCC3)C1]

### Synthesis of (Z)-tert-butyl (3-hydroxycyclobutyl)carbamate

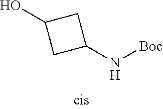 [SMILES: CC(C)(C)OC(=O)NC1CC(O)C1]

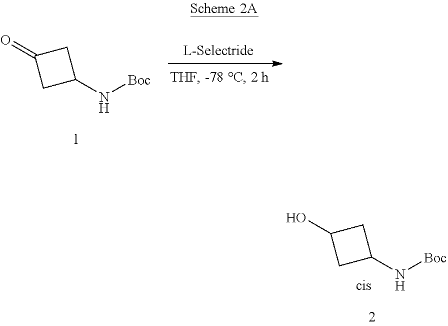

A solution of tert-butyl (3-oxocyclobutyl)carbamate (1) (2.5 g, 13.5 mmol, 1.0 eq) in 80 mL THF was cooled to −78° C. and treated with a 1 N solution of L-Selectride (16.2 mL, 16.2 mmol, 1.2 eq) in THF. After stirring for 1 h the reaction was quenched with 5 mL water and warmed to rt. The reaction mixture was concentrated and purified by silica gel chromatography (EA:PE=0~50%) provided the (Z)-tert-butyl (3-hydroxycyclobutyl)carbamate (2) (1.8 g). ¹H NMR (400 MHz, CDCl₃): δ (ppm): 4.68 (brs, 1H), 4.05-3.98 (m, 1H), 3.67-3.65 (m, 1H), 2.78-2.75 (m, 2H), 2.08 (brs, 1H), 1.81-1.78 (m, 2H), 1.44 (s, 9H).

### Synthesis of (Z)-3-((tert-butoxycarbonyl)amino)cyclobutyl methanesulfonate

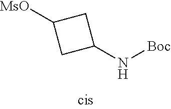 [SMILES: CC(C)(C)OC(=O)NC1CC(OS(C)(=O)=O)C1]

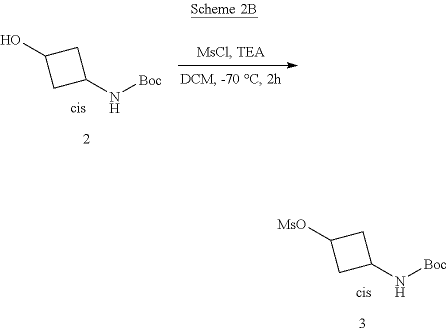

Methanesulfonyl chloride (1.3 g, 11.6 mmol, 1.2 eq) was added dropwise to a −70° C. solution of (Z)-tert-butyl (3-hydroxycyclobutyl)carbamate (2) (1.8 g, 9.6 mmol, 1.0 eq) and TEA (1.5 g, 14.8 mmol, 1.5 eq) in dichloromethane (60 mL). The resulting solution was stirred for 2 hours at −70° C., the mixture was diluted with 100 mL of water. The resulting solution was extracted with dichloromethane (3×60 mL) and the organic layers combined. The resulting mixture was washed with brine (100 mL), dried over anhydrous sodium sulfate and concentrated to give (Z)-3-((tert-butoxycarbonyl)amino)cyclobutyl methanesulfonate (3) (2.5 g). ¹H NMR (400 MHz, CDCl₃): δ (ppm): 4.75-4.67 (m, 2H), 3.85-3.82 (m, 1H), 2.98 (s, 3H), 2.93-2.88 (m, 2H), 2.22-2.14 (m, 2H), 1.44 (s, 9H).

### Synthesis of (E)-tert-butyl (3-cyanocyclobutyl)carbamate

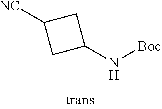 [SMILES: CC(C)(C)OC(=O)NC1CC(C#N)C1]

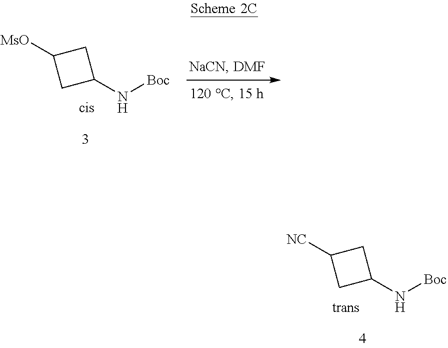

(Z)-3-((tert-butoxycarbonyl)amino)cyclobutyl methanesulfonate (3) (1.2 g, 4.5 mmol, 1.0 eq) in DMF (30 mL) was treated with NaCN (665.7 mg, 13.6 mmol, 3.0 eq) and the reaction was heated to 120° C. for 15 h, the mixture was diluted with 50 mL of water. The resulting solution was extracted with EA (2×60 mL) and the organic layers combined. The resulting mixture was washed with brine (60 mL), dried over anhydrous sodium sulfate and concentrated to give (E)-tert-butyl (3-cyanocyclobutyl)carbamate (4) (730.0 mg). ¹H NMR (400 MHz, CDCl₃): δ (ppm): 4.77 (br s, 1H), 4.41-4.39 (m, 1H), 3.06-3.01 (m, 1H), 2.74-2.41 (m, 2H), 2.27-2.24 (m, 2H), 1.45 (s, 9H).

### Synthesis of (E)-tert-butyl (3-(N-hydroxycarbamimidoyl)cyclobutyl)carbamate

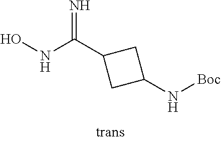 [SMILES: CC(C)(C)OC(=O)NC1CC(C(=N)NO)C1]

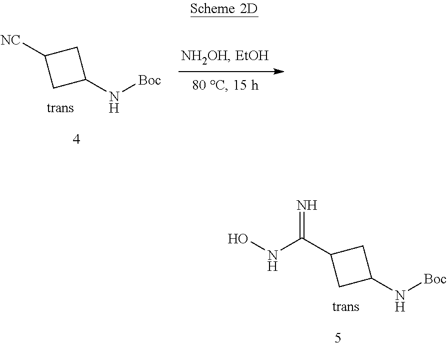

To a solution of (E)-tert-butyl (3-cyanocyclobutyl)carbamate (4) (730.0 mg, 3.7 mmol, 1.0 eq) in EtOH (25 mL) was added aqueous hydroxylamine (2.2 g/2.0 mL, 33.3 mmol, 9.0 eq) under N₂. The mixture was heated to 80° C. and stirred for 15 h. The reaction mixture was concentrated and purified by Prep-HPLC to give (E)-tert-butyl (3-(N-hydroxycarbamimidoyl)cyclobutyl)carbamate (5) (520.0 mg). LCMS (ESI): m/z 230.2 [M+H]⁺.

### Synthesis of (E)-tert-butyl (3-carbamimidoylcyclobutyl)carbamate

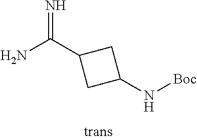 [SMILES: CC(C)(C)OC(=O)NC1CC(C(=N)N)C1]

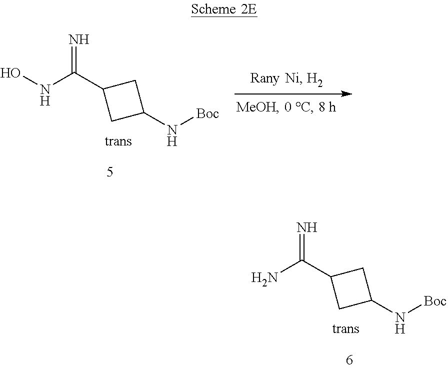

To a solution of (E)-tert-butyl (3-(N-hydroxycarbamimidoyl)cyclobutyl)carbamate (5) (520.0 mg, 2.3 mmol, 1.0 eq) in MeOH (100 mL) was added Raney-Ni (200 mg, 2.3 mmol, 1.0 eq) under N₂. The suspension was degassed under vacuum and purged with H₂ several times. The mixture was stirred under H₂ at 0° C. for 8 h. The reaction mixture was filtered, and the filtrate was concentrated under reduced pressure to give (E)-tert-butyl (3-carbamimidoylcyclobutyl)carbamate (6) (480.0 mg). LCMS (ESI): m/z 214.2 [M+H]⁺.

### Synthesis of (E)-tert-butyl ((1r,3r)-3-(4-oxo-3,5,7,8-tetrahydro-4H-thiopyrano[4,3-d]pyrimidin-2-yl)cyclobutyl)carbamate

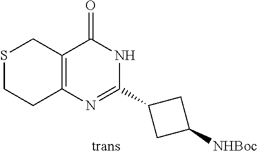

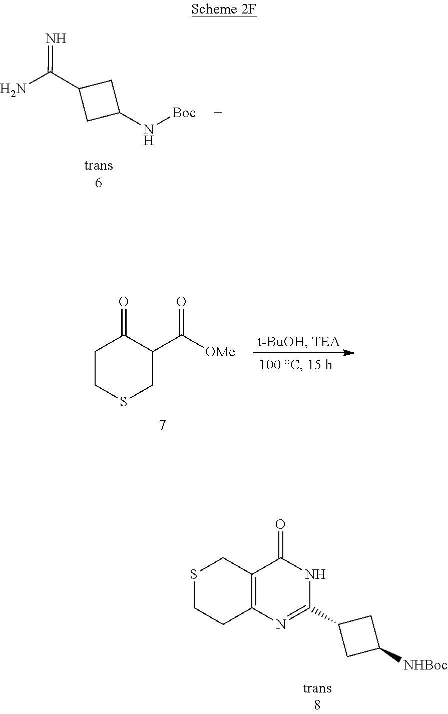 [SMILES: CC(C)(C)OC(=O)NC1CC(C(=N)N)C1 ; COC(=O)C1CSCCC1=O ; CC(C)(C)OC(=O)N[C@H]1C[C@H](c2nc3c(c(=O)[nH]2)CSCC3)C1]

To a solution of (E)-tert-butyl (3-carbamimidoylcyclobutyl)carbamate (6) (480.0 mg, 2.3 mmol, 1.0 eq) and methyl 4-oxotetrahydro-2H-thiopyran-3-carboxylate (7) (431.3 mg, 2.5 mmol, 1.1 eq) in t-BuOH (30 mL) was added TEA (1.3 g, 12.8 mmol, 5.6 eq) in one portion at 15° C. under N₂. The mixture was heated to 100° C. and stirred for 15 h. The reaction was concentrated in vacuo. The residue was purified by silica gel column chromatography (DCM/MeOH=100%~95%) to give (E)-tert-butyl ((1r,3r)-3-(4-oxo-3,5,7,8-tetrahydro-4H-thiopyrano[4,3-d]pyrimidin-2-yl)cyclobutyl)carbamate (8) (630.1 mg). LCMS (ESI): m/z 338.1 [M+H]⁺.

### Synthesis of (E)-2-((1r,3r)-3-aminocyclobutyl)-3,5,7,8-tetrahydro-4H-thiopyrano[4,3-d]pyrimidin-4-one

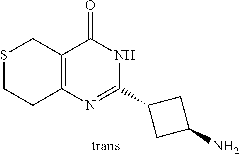

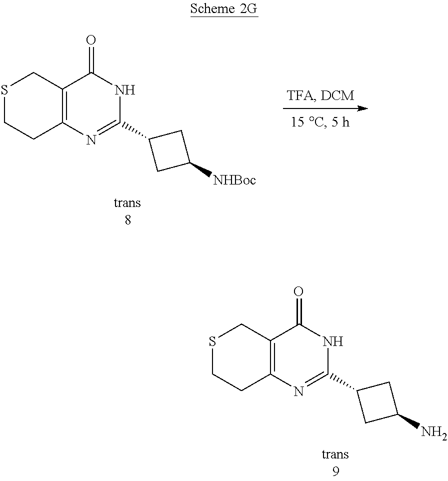

To a stirred solution of (8) (630 mg, 1.9 mmol, 1.0 eq) in DCM (30.0 mL) was added TFA (3.0 mL, 39.5 mmol, 20.8 eq) at 25° C. The reaction mixture was stirred at 25° C. under N₂ atmosphere for 5 h. The reaction mixture was concentrated in vacuum to give (E)-2-((1r,3r)-3-aminocyclobutyl)-3,5,7,8-tetrahydro-4H-thiopyrano[4,3-d]pyrimidin-4-one (9) (656.5 mg). LCMS (ESI): m/z 238.0 [M+H]⁺. ¹H-NMR (400 MHz, DMSO-d₆): δ (ppm): 8.41 (brs, 3H), 3.75-3.71 (m, 1H), 3.47 (s, 2H), 3.43-3.80 (m, 1H), 2.90-2.87 (m, 4H), 2.66-2.53 (m, 4H).

### Synthesis of (E)-3-nitro-4-(((1r,3r)-3-(4-oxo-3,5,7,8-tetrahydro-4H-thiopyrano[4,3-d]pyrimidin-2-yl)cyclobutyl)amino)benzonitrile

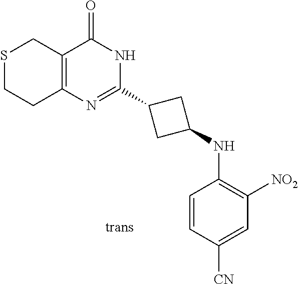 [SMILES: N#Cc1ccc(N[C@H]2C[C@H](c3nc4c(c(=O)[nH]3)CSCC4)C2)c([N+](=O)[O-])c1 ; "trans"]

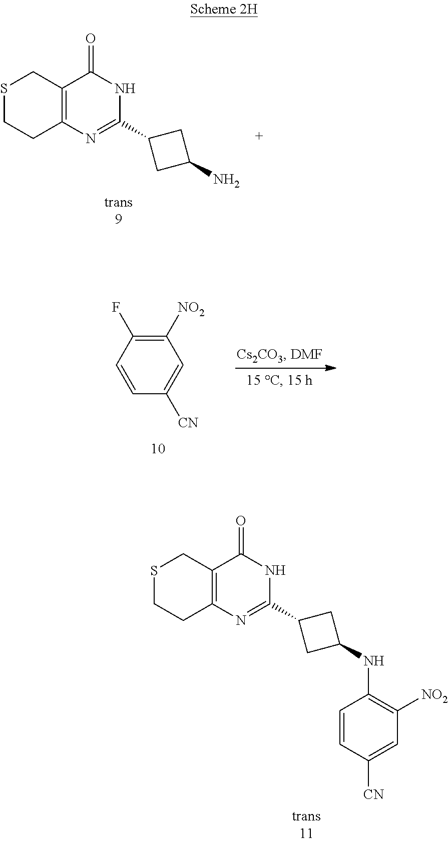 [SMILES: N[C@H]1C[C@H](c2nc3c(c(=O)[nH]2)CSCC3)C1 ; N#Cc1ccc(F)c([N+](=O)[O-])c1 ; N#Cc1ccc(N[C@H]2C[C@H](c3nc4c(c(=O)[nH]3)CSCC4)C2)c([N+](=O)[O-])c1]

To a solution of (9) (443.1 mg, 1.9 mmol, 1.0 eq) and 4-fluoro-3-nitrobenzonitrile (10) (248.3 mg, 1.5 mmol, 0.8 eq) in DMF (30 mL) was added Cs₂CO₃ (1.5 g, 4.6 mmol, 2.4 eq) in one portion at 15° C. under N₂. The reaction mixture was stirred at 15° C. under N₂ atmosphere for 15 h, the mixture was diluted with 50 mL of water. The resulting solution was extracted with EA (2×60 mL) and the organic layers combined. The resulting mixture was washed with brine (60 mL), dried over anhydrous sodium sulfate and concentrated to give (E)-3-nitro-4-(((1r,3r)-3-(4-oxo-3,5,7,8-tetrahydro-4H-thiopyrano[4,3-d]pyrimidin-2-yl)cyclobutyl)amino)benzonitrile (11) (500.9 mg). LCMS (ESI): m/z 384.0 [M+H]⁺.

### Synthesis of (E)-3-amino-4-(((1r,3r)-3-(4-oxo-3,5,7,8-tetrahydro-4H-thiopyrano[4,3-d]pyrimidin-2-yl)cyclobutyl)amino)benzonitrile

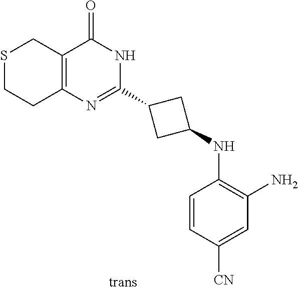 [SMILES: N#Cc1ccc(N[C@H]2C[C@H](c3nc4c(c(=O)[nH]3)CSCC4)C2)c(N)c1 ; "trans"]

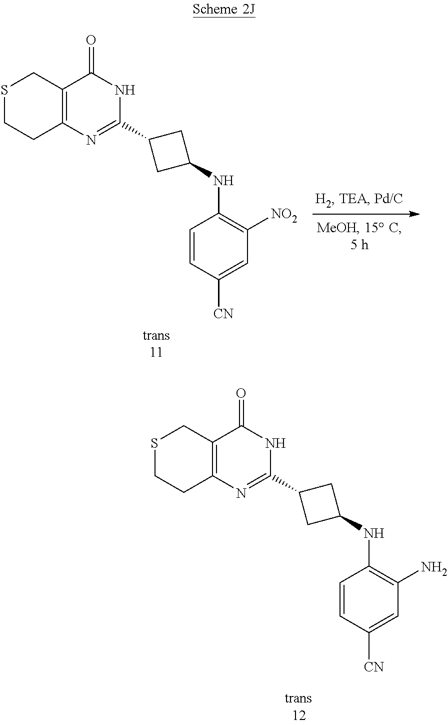

To a solution of (11) (200.0 mg, 0.52 mmol, 1.0 eq) and TEA (1.0 mL, 7.2 mmol, 13.8 eq) in MeOH (200 mL) was added Pd/C (200 mg) under N₂. The suspension was degassed under vacuum and purged with H₂ several times. The mixture was stirred under H₂ at 15° C. for 5 h. The reaction mixture was filtered, and the filtrate was concentrated under reduced pressure to give (E)-3-amino-4-(((1r,3r)-3-(4-oxo-3,5,7,8-tetrahydro-4H-thiopyrano[4,3-d]pyrimidin-2-yl)cyclobutyl)amino)benzonitrile (12) (180.0 mg). LCMS (ESI): m/z 354.2 [M+H]⁺.

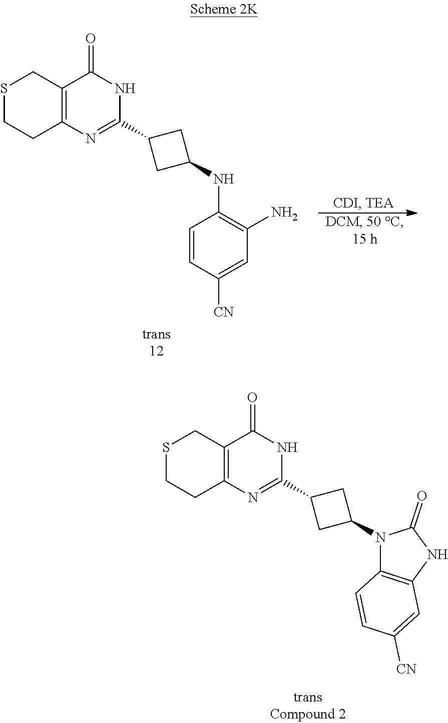 [SMILES: N#Cc1ccc(N[C@H]2C[C@H](c3nc4c(c(=O)[nH]3)CSCC4)C2)c(N)c1 ; N#Cc1ccc2c(c1)[nH]c(=O)n2[C@H]1C[C@H](c2nc3c(c(=O)[nH]2)CSCC3)C1]

(E)-3-amino-4-(((1r,3r)-3-(4-oxo-3,5,7,8-tetrahydro-4H-thiopyrano[4,3-d]pyrimidin-2-yl)cyclobutyl)amino)benzonitrile (12) (180.0 mg, 0.51 mmol, 1.0 eq) and TEA (3 mL, 21.7 mmol, 42.6 eq) in DCM (30 mL) was treated with CDI (918.3 mg, 5.7 mmol, 11.2 eq) and the reaction was heated to 50° C. for 15 h, the mixture was diluted with 50 mL of water. The resulting solution was extracted with EA (2×60 mL) and the organic layers combined. The resulting mixture was washed with brine (60 mL), dried over anhydrous sodium sulfate, the organic layers were concentrated and purified by Prep-HPLC to give (E)-2-oxo-1-((1r,3r)-3-(4-oxo-3,5,7,8-tetrahydro-4H-thiopyrano[4,3-d]pyrimidin-2-yl)cyclobutyl)-2,3-dihydro-1H-benzo[d]imidazole-5-carbonitrile (Compound 2) (102.0 mg). LCMS (ESI): m/z 380.0 [M+H]⁺. ¹H-NMR (400 MHz, DMSO-d₆): δ (ppm): 11.40 (s, 1H), 8.05 (d, J=8.4 Hz, 1H), 7.58 (dd, J=8.0 Hz, J=1.2 Hz, 1H), 7.40 (d, J=1.2 Hz, 1H), 5.02-4.92 (m, 1H), 3.47 (s, 2H), 3.31-3.24 (m, 1H), 3.10-3.02 (m, 2H), 2.93-2.85 (m, 4H), 2.71-2.63 (m, 2H).

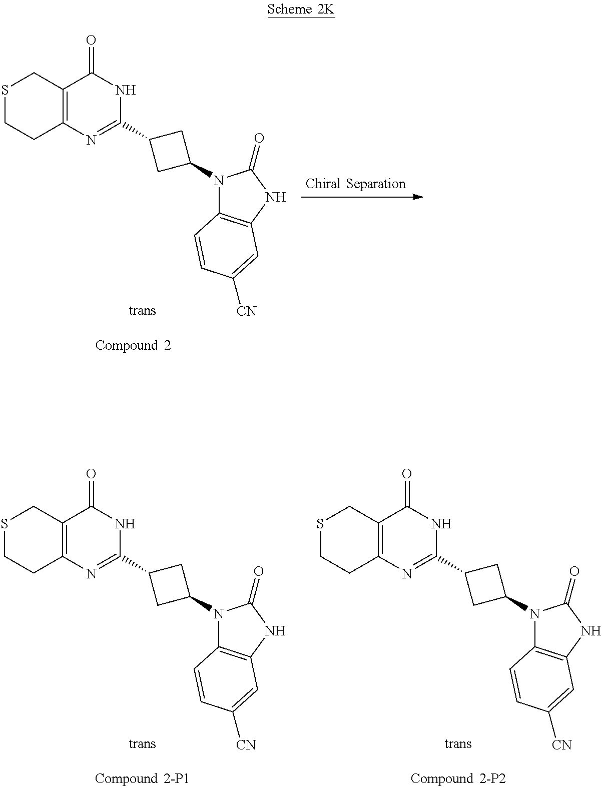

(Compound 2) (150 mg) was purified by Chiral-HPLC to give (Compound 2-P1) and (Compound 2-P2). LCMS (ESI): m/z 380.0 [M+H]⁺. ¹H-NMR (Compound 2-P1, 400 MHz, DMSO-d₆): δ (ppm): 7.49 (s, 2H), 7.38 (s, 1H), 5.10-5.04 (m, 1H), 3.58-3.50 (m, 1H), 3.47 (s, 2H), 3.17-3.09 (m, 2H), 2.93-2.85 (m, 4H), 2.71-2.63 (m, 2H). ¹H-NMR (Compound 2-P2, 400 MHz, DMSO-d₆): δ (ppm): 12.49 (s, 1H), 11.39 (s, 1H), 8.09 (d, J=8.4 Hz, 1H), 7.58 (dd, J=8.4 Hz, J=1.6 Hz, 1H), 7.40 (d, J=1.6 Hz, 1H), 5.02-4.92 (m, 1H), 3.47 (s, 2H), 3.31-3.24 (m, 1H), 3.10-3.01 (m, 2H), 2.93-2.90 (m, 4H), 2.71-2.63 (m, 2H).

### Compound 3

### Synthesis of 2-(1-hydroxy-1,3-dihydrobenzo[c][1,2]oxaborol-5-yl)-7,8-dihydro-3H-thiopyrano [4,3-d]pyrimidin-4 (5H)-one (Compound 3)

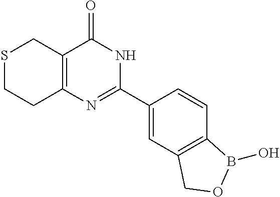 [SMILES: O=c1[nH]c(-c2ccc3c(c2)COB3O)nc2c1CSCC2]

### Synthesis of methyl 2-bromo-5-cyanobenzoate

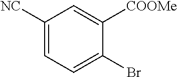 [SMILES: COC(=O)c1cc(C#N)ccc1Br]

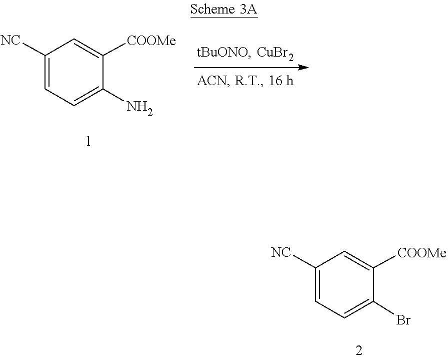

A mixture of CuBr₂ (3.04 g, 13.6 mmol, 1.2 eq) in CAN (50 mL) was added to tBuONO (1.64 g, 15.9 mmol, 1.4 eq) at 0° C. The mixture was stirred for 5 min. Methyl 2-amino-5-cyanobenzoate (1) (2.0 g, 11.4 mmol, 1 eq) was added in portions. The mixture was stirred for 16 h at room temperature and made acidic (pH=2) by the addition of 1 M HCl. The mixture was extracted with EtOAc (3×80 mL) and the combined organic extract was dried over Na₂SO₄, filtered, and evaporated to give methyl 2-bromo-5-cyanobenzoate (2) (2.7 g, crude).

### Synthesis of methyl 2-bromo-5-carbamimidoylbenzoate

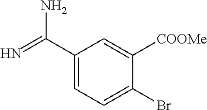 [SMILES: COC(=O)c1cc(C(=N)N)ccc1Br]

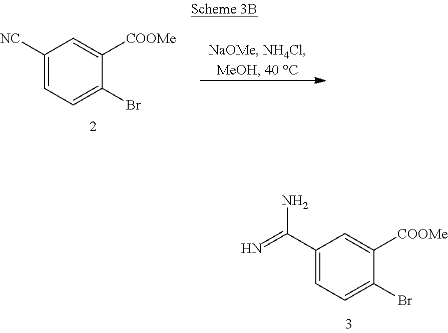 [SMILES: COC(=O)c1cc(C#N)ccc1Br ; COC(=O)c1cc(C(=N)N)ccc1Br]

A mixture of methyl 2-bromo-5-cyanobenzoate (2) (2.50 g, 10.4 mmol, 1.0 eq), ammonium chloride (0.38 g, 5.4 mmol, 2.5 eq), and sodium methoxide (0.22 g, 5.4 mmol, 2.5 eq) in MeOH (50 mL) was stirred at 40° C. for 16 h. The mixture was concentrated to afford methyl 2-bromo-5-carbamimidoylbenzoate (3) (crude). LC-MS (ESI) m/z calculated for C₉H₉BrN₂O₂+H⁺: 257.1; found: 256.9.

### Synthesis of methyl 2-bromo-5-(4-oxo-4,5,7,8-tetrahydro-3H-thiopyrano[4,3-d]pyrimidin-2-yl) benzoate

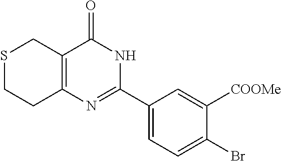 [SMILES: COC(=O)c1cc(-c2nc3c(c(=O)[nH]2)CSCC3)ccc1Br]

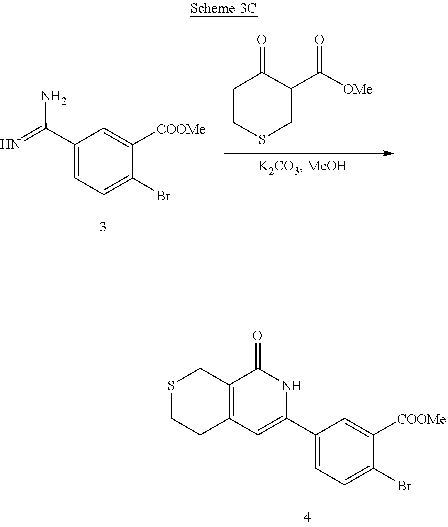

A mixture of methyl 2-bromo-5-carbamimidoylbenzoate (3) (2.5 g, 9.7 mmol, 1.0 eq), methyl 4-oxotetrahydro-2H-thiopyran-3-carboxylate (1.7 g, 9.7 mmol, 1.0 eq), and K₂CO₃ (4.0 g, 29.2 mmol, 3.0 eq) in MeOH (50 mL) was stirred at 70° C. for 16 h. The mixture was cooled and filtered. The filtrate was added into water (300 mL). The solid was filtered and dried to afford methyl 2-bromo-5-(4-oxo-4,5,7,8-tetrahydro-3H-thiopyrano[4,3-d]pyrimidin-2-yl) benzoate (4) (1.5 g, 3.9 mmol). LC-MS (ESI) m/z calculated for C₁₅H₁₃BrN₂O₃S+H⁺: 382.2; found: 383.0.

### Synthesis of methyl 5-(4-oxo-4,5,7,8-tetrahydro-3H-thiopyrano[4,3-d]pyrimidin-2-yl)-2-(4,4,5,5-tetramethyl-1,3,2-dioxaborolan-2-yl)benzoate

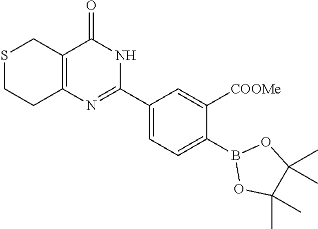 [SMILES: COC(=O)c1cc(-c2nc3c(c(=O)[nH]2)CSCC3)ccc1B1OC(C)(C)C(C)(C)O1]

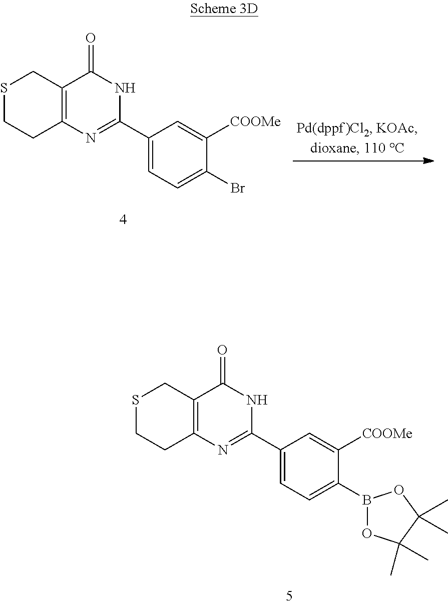 [SMILES: COC(=O)c1cc(-c2nc3c(c(=O)[nH]2)CSCC3)ccc1Br ; COC(=O)c1cc(-c2nc3c(c(=O)[nH]2)CSCC3)ccc1B1OC(C)(C)C(C)(C)O1]

The mixture of methyl 2-bromo-5-(4-oxo-4,5,7,8-tetrahydro-3H-thiopyrano[4,3-d]pyrimidin-2-yl) benzoate (4) (0.60 g, 1.57 mmol, 1.0 eq), (BPin)₂ (0.80 g, 3.15 mmol, 2.0 eq), Pd(dppf)Cl₂ (115 mg, 0.16 mmol, 0.1 eq), and KOAc (462 mg, 4.72 mmol, 3.0 eq) in dioxane (15 mL) was stirred at 110° C. for 16 h. The mixture was diluted with EtOAc (30 mL), washed with water (15 mL), dried over Na₂SO₄, and concentrated. The crude product was purified on a silica gel column (PE:EtOAc=5:1 to 1:1) to afford methyl 5-(4-oxo-4,5,7,8-tetrahydro-3H-thiopyrano[4,3-d]pyrimidin-2-yl)-2-(4,4,5,5-tetramethyl-1,3,2-dioxaborolan-2-yl)benzoate (5) (340 mg, 0.79 mmol). LC-MS (ESI) m/z calculated for C₂₁H₂₅BN₂O₅S+H⁺: 429.3; found: 429.1.

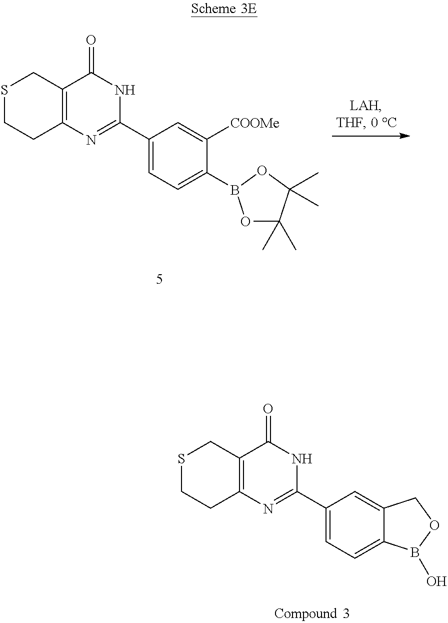

To a solution of methyl 5-(4-oxo-4,5,7,8-tetrahydro-3H-thiopyrano[4,3-d]pyrimidin-2-yl)-2-(4,4,5,5-tetramethyl-1,3,2-dioxaborolan-2-yl)benzoate (5) (0.34 g, 0.79 mmol, 1.0 eq) in THF (15 mL) was added LAH (60 mg, 1.60 mmol, 2.0 eq). The mixture was stirred at 0° C. for 1 h. The mixture was added to H₂O, filtered, and concentrated under vacuum to afford a crude product which was purified by flash chromatography (H₂O:CH₃CN=90:10 to 50:50) to provide 2-(1-hydroxy-1,3-dihydrobenzo[c][1,2]oxaborol-5-yl)-3,5,7,8-tetrahydro-4H-thiopyrano[4,3-d]pyrimidin-4-one (Compound 3). LC-MS (ESI) m/z calculated for C₁₄H₁₃BN₂O₃S+H⁺: 301.1; found: 301.0. ¹H NMR (400 MHz, MeOD): δ 8.09 (s, 1H), 8.03 (d, J=7.6 Hz, 1H), 7.84 (d, J=7.6 Hz, 1H), 3.54 (s, 2H), 5.07 (s, 2H), 2.95-2.85 (m, 4H).

### Compound 4 and Compound 43

### Synthesis of 3-(4-(4-oxo-3,5,7,8-tetrahydro-4H-thiopyrano[4,3-d]pyrimidin-2-yl)phenyl)-1,2,4-oxadiazol-5 (4H)-one (Compound 4)

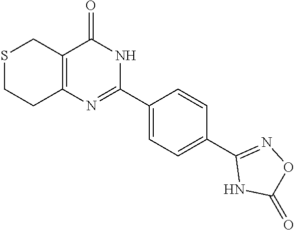 [SMILES: O=c1[nH]c(-c2ccc(-c3nc4c(c(=O)[nH]3)CSCC4)cc2)no1]

### Synthesis of 4-(4-oxo-3,5,7,8-tetrahydro-4H-thiopyrano[4,3-d]pyrimidin-2-yl)benzonitrile (Compound 43)

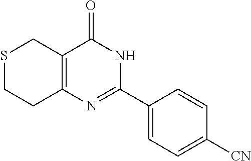 [SMILES: N#Cc1ccc(-c2nc3c(c(=O)[nH]2)CSCC3)cc1]

### Synthesis of 2-(4-bromophenyl)-3,5,7,8-tetrahydro-4H-thiopyrano[4,3-d]pyrimidin-4-one

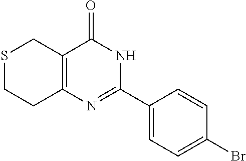 [SMILES: O=c1[nH]c(-c2ccc(Br)cc2)nc2c1CSCC2]

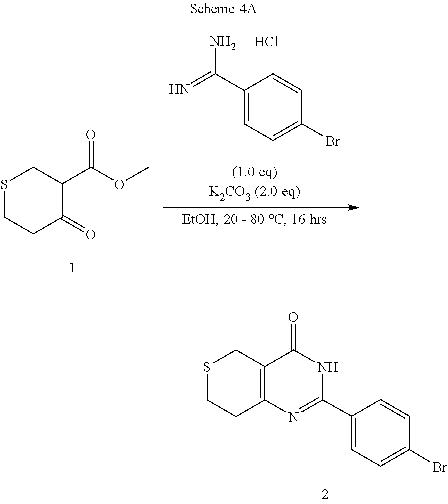

To a solution of methyl 4-oxotetrahydro-2H-thiopyran-3-carboxylate (1) (5.00 g, 28.7 mmol, 1.00 eq) and 4-bromobenzamidine hydrochloride (6.76 g, 28.7 mmol, 1.00 eq) in EtOH (50 mL) was added K₂CO₃ (7.93 g, 57.4 mmol, 2 eq) at 20° C. The mixture was stirred at 80° C. for 16 h. LC-MS showed that (1) was consumed completely. The reaction mixture was filtered and the filtrate concentrated under reduced pressure to give a residue that was suspended in water (100 mL), then stirred at 20° C. for 4 h. The mixture was filtered and the filter cake was dried under reduced pressure to give 2-(4-bromophenyl)-3,5,7,8-tetrahydro-4H-thiopyrano[4,3-d]pyrimidin-4-one (2) (6.60 g, crude) which was used directly in the next step without further purification. ¹H NMR (400 MHz, DMSO-d₆): δ 8.08-8.02 (m, 2H), 7.72-7.67 (m, 2H), 3.51 (s, 2H), 2.87 (qd, J=4.4, 8.4 Hz, 4H). LC-MS: 324.3+bromo isomers (M+1).

### Synthesis of 4-(4-oxo-3,5,7,8-tetrahydro-4H-thiopyrano[4,3-d]pyrimidin-2-yl)benzonitrile (Compound 43)

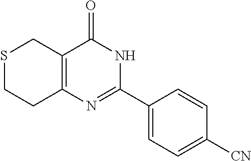 [SMILES: N#Cc1ccc(-c2nc3c(c(=O)[nH]2)CSCC3)cc1]

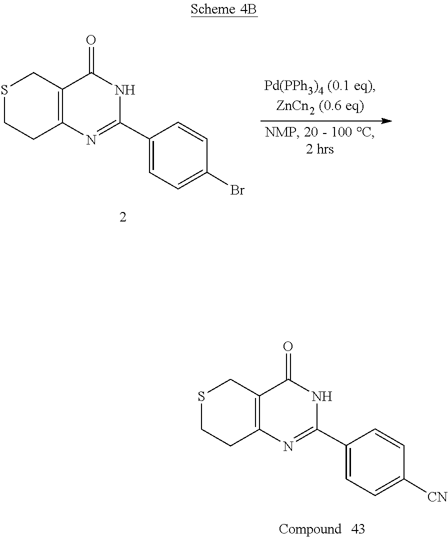

To a solution of 2-(4-bromophenyl)-3,5,7,8-tetrahydro-4H-thiopyrano[4,3-d]pyrimidin-4-one (2) (4.50 g, 13.9 mmol, 1 eq) in NMP (45 mL) were added, portion-wise, Zn(CN)₂ (981 mg, 8.35 mmol, 530 μL, 0.6 eq) and Pd(PPh₃)₄ (1.61 g, 1.39 mmol, 0.1 eq) at 20° C. under N₂. The mixture was stirred at 100° C. for 2 h. LC-MS showed that (2) was consumed completely. The resulting mixture was cooled to 20° C. and diluted with saturated Na₂CO₃ (120 mL) at 20° C. Then the mixture was extracted with EtOAc (40 mL×3). The combined organic layers were washed with brine, dried over Na₂SO₄, filtered, and concentrated under reduced pressure to give a residue that was purified by column chromatography (SiO₂, petroleum ether/ethyl acetate=100/1 to 0/1). 4-(4-oxo-3,5,7,8-tetrahydro-4H-thiopyrano[4,3-d]pyrimidin-2-yl)benzonitrile (Compound 43) (1.80 g, 6.68 mmol) was obtained. ¹H NMR (400 MHz, DMSO-d₆): δ 13.07-12.77 (m, 1H), 8.25 (br d, J=7.5 Hz, 2H), 8.00 (d, J=8.4 Hz, 2H), 3.55 (s, 2H), 2.91 (s, 4H). LC-MS: 270.1 (M+1).

### Synthesis of (Z)—N′-hydroxy-4-(4-oxo-3,5,7,8-tetrahydro-4H-thiopyrano[4,3-d]pyrimidin-2-yl)benzimidamide

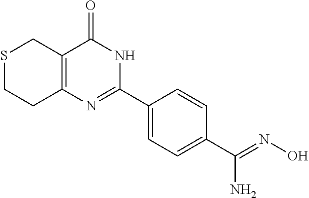 [SMILES: N/C(=N\O)c1ccc(-c2nc3c(c(=O)[nH]2)CSCC3)cc1]

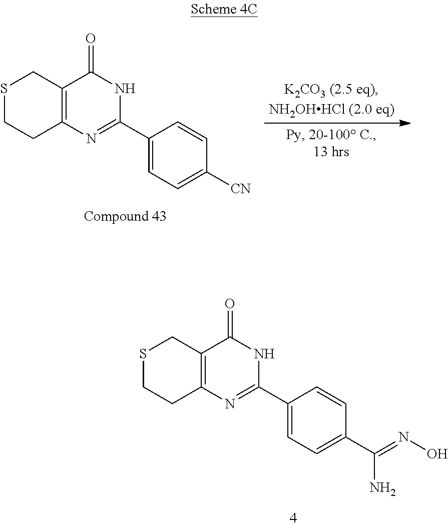 [SMILES: N#Cc1ccc(-c2nc3c(c(=O)[nH]2)CSCC3)cc1 ; N/C(=N\O)c1ccc(-c2nc3c(c(=O)[nH]2)CSCC3)cc1]

To a solution of 4-(4-oxo-3,5,7,8-tetrahydro-4H-thiopyrano[4,3-d]pyrimidin-2-yl)benzonitrile (Compound 43) (500 mg, 1.86 mmol, 1 eq) in pyridine (5 mL) was added NH₂OH·HCl (258 mg, 3.71 mmol, 2 eq) and K₂CO₃ (641 mg, 4.64 mmol, 2.50 eq) at 20° C. The mixture was stirred at 100° C. for 13 h. LC-MS showed that (Compound 43) was consumed. After cooling to 20° C., the reaction mixture was filtered and the filter cake dried and concentrated under reduced pressure to give a residue that was suspended in water (5.00 mL) and was stirred at 20° C. for 4 h. The mixture was filtered and the filter cake dried and concentrated under reduced pressure to give (Z)—N′-hydroxy-4-(4-oxo-3,5,7,8-tetrahydro-4H-thiopyrano[4,3-d]pyrimidin-2-yl)benzimidamide (4) (333 mg, crude), which was used in the next step without further purification. LC-MS: 302.9 (M+1).

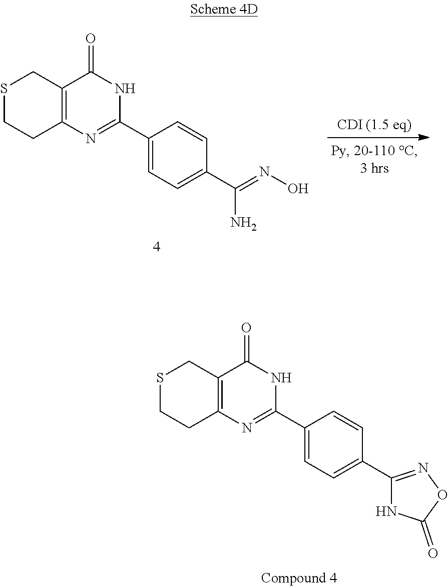 [SMILES: N/C(=N\O)c1ccc(-c2nc3c(c(=O)[nH]2)CSCC3)cc1 ; O=c1[nH]c(-c2ccc(-c3nc4c(c(=O)[nH]3)CSCC4)cc2)no1]

To a solution of (Z)—N′-hydroxy-4-(4-oxo-3,5,7,8-tetrahydro-4H-thiopyrano[4,3-d]pyrimidin-2-yl)benzimidamide (4) (333 mg, 1.10 mmol, 1 eq) in pyridine (3.30 mL) was added CDI (268 mg, 1.65 mmol, 1.50 eq) at 20° C. The mixture was stirred at 110° C. for 3 h. LC-MS showed that (4) was consumed completely. After cooling to 20° C., the mixture was filtered and the filter cake dried and concentrated under reduced pressure to give a residue that was stirred in DCM (2 mL) at RT for 6 h. The mixture was filtered and the filter cake dried and concentrated under reduced pressure to give 3-(4-(4-oxo-3,5,7,8-tetrahydro-4H-thiopyrano[4,3-d]pyrimidin-2-yl)phenyl)-1,2,4-oxadiazol-5 (4H)-one (Compound 4). ¹H NMR (400 MHz, DMSO-d₆): δ 13.26-12.46 (m, 1H), 8.23 (d, J=8.5 Hz, 2H), 7.94 (d, J=8.5 Hz, 2H), 7.18 (s, 1H), 3.55 (s, 2H), 2.91 (s, 4H). LC-MS: 329.1 (M+1).

### Compound 5

### Synthesis of (E)-(5-(2-(4-oxo-3,5,7,8-tetrahydro-4H-thiopyrano[4,3-d]pyrimidin-2-yl)vinyl)-2-(trifluoromethyl)phenyl)boronic acid (Compound 5)

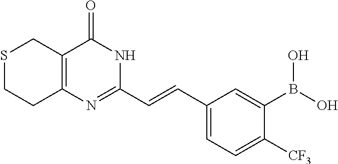 [SMILES: O=c1[nH]c(/C=C/c2ccc(C(F)(F)F)c(B(O)O)c2)nc2c1CSCC2]

### Synthesis of 3-(4,4,5,5-tetramethyl-1,3,2-dioxaborolan-2-yl)-4-(trifluoromethyl)benzaldehyde

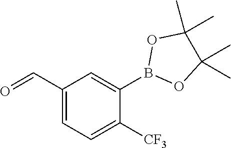 [SMILES: CC1(C)OB(c2cc(C=O)ccc2C(F)(F)F)OC1(C)C]

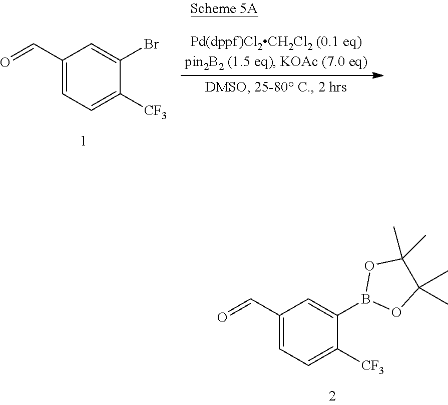 [SMILES: O=Cc1ccc(C(F)(F)F)c(Br)c1 ; CC1(C)OB(c2cc(C=O)ccc2C(F)(F)F)OC1(C)C]

To a solution of 3-bromo-4-(trifluoromethyl)benzaldehyde (1) (0.50 g, 1.98 mmol, 1.00 eq) and Pin₂B₂ (753 mg, 2.96 mmol, 1.50 eq) in DMSO (10.0 mL) was added KOAc (1.36 g, 13.8 mmol, 7.00 eq) and Pd(dppf)Cl₂·CH₂Cl₂ (161 mg, 198 mol, 0.10 eq) at 25° C. under N₂. The suspension was degassed in vacuo and purged with N₂ several times. The mixture was then warmed to 80° C. and stirred at 80° C. for 2 h. Thin-layer chromatography (TLC; petroleum ether/ethyl acetate=20/1) showed that 3-bromo-4-(trifluoromethyl)benzaldehyde (1) was consumed completely. The mixture was cooled to 25° C. and then diluted with EtOAc (5.00 mL). The mixture was filtered, and the filtrate was washed with water (5.00 mL×2) and brine (5.00 mL). The organic layer was dried over anhydrous Na₂SO₄, filtered, and concentrated under reduced pressure to give a residue. The residue was purified by Prep-TLC (SiO₂, Petroleum ether/Ethyl acetate=10/1). 3-(4,4,5,5-tetramethyl-1,3,2-dioxaborolan-2-yl)-4-(trifluoromethyl)benzaldehyde (2) (0.20 g, crude) was obtained and used in the next step without further purification.

### Synthesis of 2-methyl-3,5,7,8-tetrahydro-4H-thiopyrano[4,3-d]pyrimidin-4-one

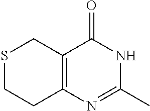

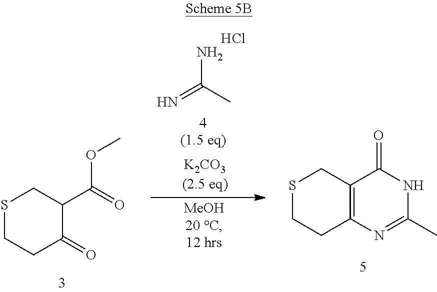

To a solution of acetamidine:hydrochloride (4) (814 mg, 8.61 mmol, 1.50 eq) in MeOH (10.0 mL) was added K₂CO₃ (1.98 g, 14.4 mmol, 2.50 eq) and methyl 4-oxotetrahydro-2H-thiopyran-3-carboxylate (3) (1.00 g, 5.74 mmol, 1.00 eq). The mixture was stirred at 20° C. for 12 h. TLC (petroleum ether/ethyl acetate=8/1) indicated methyl 4-oxotetrahydro-2H-thiopyran-3-carboxylate (3) was consumed completely. The mixture was filtered, and the filtrate was concentrated under reduced pressure to give a residue. 2-methyl-3,5,7,8-tetrahydro-4H-thiopyrano[4,3-d]pyrimidin-4-one (5) (1.00 g, crude) was obtained and used in the next step without further purification. The obtained compound was checked by ¹H NMR.

### Synthesis of (E)-2-(3-(4,4,5,5-tetramethyl-1,3,2-dioxaborolan-2-yl)-4-(trifluoromethyl)styryl)-3,5,7,8-tetrahydro-4H-thiopyrano[4,3-d]pyrimidin-4-one

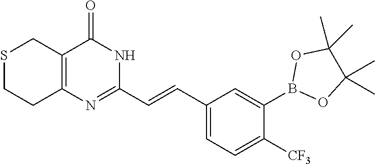 [SMILES: CC1(C)OB(c2cc(/C=C/c3nc4c(c(=O)[nH]3)CSCC4)ccc2C(F)(F)F)OC1(C)C]

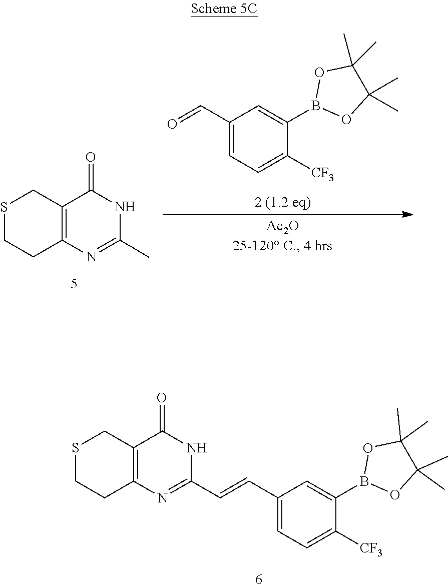 [SMILES: Cc1nc2c(c(=O)[nH]1)CSCC2 ; CC1(C)OB(c2cc(C=O)ccc2C(F)(F)F)OC1(C)C ; CC1(C)OB(c2cc(/C=C/c3nc4c(c(=O)[nH]3)CSCC4)ccc2C(F)(F)F)OC1(C)C]

To a solution of 2-methyl-3,5,7,8-tetrahydro-4H-thiopyrano[4,3-d]pyrimidin-4-one (5) (0.20 g, 666 μmol, 1.21 eq) in Ac₂O (1.00 mL) was added 3-(4,4,5,5-tetramethyl-1,3,2-dioxaborolan-2-yl)-4-(trifluoromethyl)benzaldehyde (2) (0.10 g, 549 μmol, 1.00 eq), ZnCl₂ (150 mg, 1.10 mmol, 51.4 uL, 2.00 eq), and AcOH (65.9 mg, 1.10 mmol, 62.8 uL, 2.00 eq) at 25° C. under N₂. The mixture was stirred at 120° C. for 4 h. LC-MS (ET48116-9-P1A) showed 3-(4,4,5,5-tetramethyl-1,3,2-dioxaborolan-2-yl)-4-(trifluoromethyl)-benzaldehyde (2) was consumed completely and one main peak with the desired m/z was detected. The mixture was cooled to 25° C. and then concentrated under reduced pressure to remove most of the Ac₂O. The resulting residue was dissolved in EtOAc (5.00 mL) and then washed with saturated NaHCO₃ (2.00 mL×2). The organic layer was separated and concentrated under reduced pressure to give a residue. The residue was purified by prep-TLC (SiO₂, petroleum ether/ethyl acetate=1/1). (E)-2-(3-(4,4,5,5-tetramethyl-1,3,2-dioxaborolan-2-yl)-4-(trifluoromethyl)styryl)-3,5,7,8-tetrahydro-4H-thiopyrano[4,3-d]pyrimidin-4-one (6) (0.25 g, crude) was obtained.

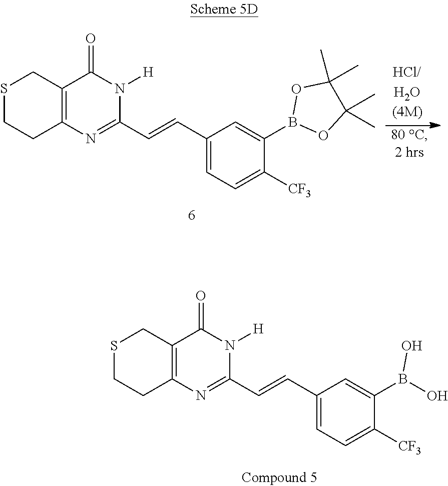 [SMILES: CC1(C)OB(c2cc(/C=C/c3nc4c(c(=O)[nH]3)CSCC4)ccc2C(F)(F)F)OC1(C)C ; O=c1[nH]c(/C=C/c2ccc(C(F)(F)F)c(B(O)O)c2)nc2c1CSCC2]

The mixture of (E)-2-(3-(4,4,5,5-tetramethyl-1,3,2-dioxaborolan-2-yl)-4-(trifluoromethyl)styryl)-3,5,7,8-tetrahydro-4H-thiopyrano[4,3-d]pyrimidin-4-one (6) (0.25 g, 538.44 mol, 1 eq) in HCl/H₂O (4 M, 2.50 mL) was stirred at 80° C. for 2 h. LC-MS showed starting material (6) was consumed completely. The reaction mixture was concentrated under reduced pressure to give a residue that was purified by prep-HPLC (column: Phenomenex luna C18 80*40 mm*3 m; mobile phase: [water (HCl)-ACN]; B %: 18%-48%, 7 min). (E)-(5-(2-(4-oxo-3,5,7,8-tetrahydro-4H-thiopyrano[4,3-d]pyrimidin-2-yl)vinyl)-2-(trifluoromethyl)phenyl)boronic acid (Compound 5) was obtained. LC-MS: 383.0 (M+1).

¹H NMR (400 MHz, DMSO-d₆): δ=7.89 (br d, J=16.4 Hz, 1H), 7.80-7.66 (m, 3H), 7.04 (br d, J=16.3 Hz, 1H), 3.51 (br s, 2H), 2.93-2.81 (m, 4H).

### Compound 6

### Synthesis of (E)-2-(4-(trifluoromethyl)styryl)-3,5,7,8-tetrahydro-4H-thiopyrano[4,3-d]pyrimidin-4-one (Compound 6)

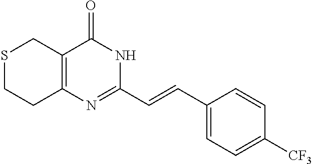 [SMILES: O=c1[nH]c(/C=C/c2ccc(C(F)(F)F)cc2)nc2c1CSCC2]

### Synthesis of 2-methyl-3,5,7,8-tetrahydro-4H-thiopyrano[4,3-d]pyrimidin-4-one

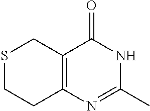

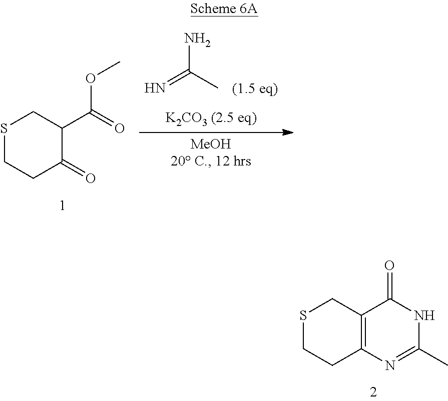

To a solution of acetamidine/hydrochloride (814 mg, 8.61 mmol, 1.50 eq) in MeOH (10.0 mL) was added K₂CO₃ (1.98 g, 14.4 mmol, 2.50 eq) and methyl 4-oxotetrahydro-2H-thiopyran-3-carboxylate (1) (1.00 g, 5.74 mmol, 1.00 eq). The mixture was stirred at 20° C. for 12 h. TLC (petroleum ether/ethyl acetate=8/1) indicated starting material (1) was consumed completely. The mixture was filtered, and the filtrate was concentrated under reduced pressure to give a residue. 2-methyl-3,5,7,8-tetrahydro-4H-thiopyrano[4,3-d]pyrimidin-4-one (2) (1.00 g, crude) was obtained and used in the next step without further purification.

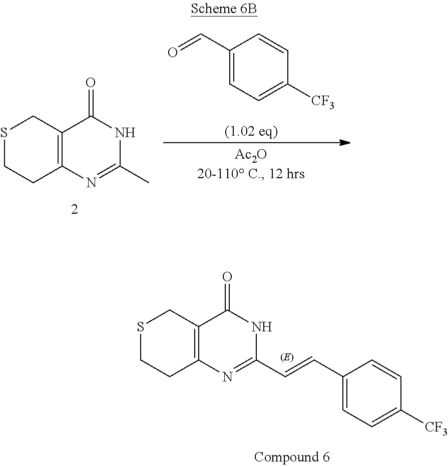 [SMILES: Cc1nc2c(c(=O)[nH]1)CSCC2 ; O=Cc1ccc(C(F)(F)F)cc1 ; O=c1[nH]c(/C=C/c2ccc(C(F)(F)F)cc2)nc2c1CSCC2]

To a solution of 2-methyl-3,5,7,8-tetrahydro-4H-thiopyrano[4,3-d]pyrimidin-4-one (2) (0.25 g, 1.37 mmol, 1.00 eq) in Ac₂O (2.50 mL) was added 4-(trifluoromethyl) benzaldehyde (244 mg, 1.40 mmol, 187 μL, 1.02 eq) at 20° C. The mixture was stirred at 110° C. for 12 h. The mixture was cooled to 20° C. and concentrated under reduced pressure to give a residue. The mixture was diluted with H₂O (2.00 mL) and extracted with EtOAc (2.00 mL×3). The organic layer was dried over anhydrous Na₂SO₄, filtered, and concentrated under reduced pressure to give a residue. Part of the residue was purified by prep-HPLC (column: Waters Xbridge BEH C18 100*30 mm*10 m; mobile phase: [water (10 mM NH₄HCO₃)-ACN]; B %: 40%-65%, 10 min). (E)-2-(4-(trifluoromethyl)styryl)-3,5,7,8-tetrahydro-4H-thiopyrano[4,3-d]pyrimidin-4-one (Compound 6) was obtained. LCMS: 339.1 (M+1). ¹H NMR (400 MHz, DMSO-d₆): δ=12.62-12.51 (m, 1H), 7.91-7.73 (m, 5H), 7.04-6.92 (m, 1H), 3.52-3.45 (m, 2H), 2.89-2.79 (m, 4H).

### Compound 7

### Synthesis of 2-(4-(6-bromopyridin-3-yl) phenyl)-3,5,7,8-tetrahydro-4H-thiopyrano [4,3-d]pyrimidin-4-one (Compound 7)

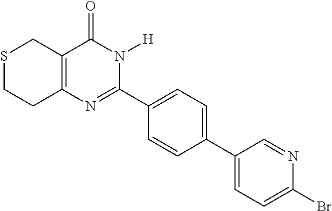 [SMILES: O=c1[nH]c(-c2ccc(-c3ccc(Br)nc3)cc2)nc2c1CSCC2]

### Synthesis of 4-(6-bromopyridin-3-yl)benzonitrile

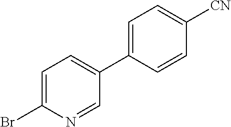 [SMILES: N#Cc1ccc(-c2ccc(Br)nc2)cc1]

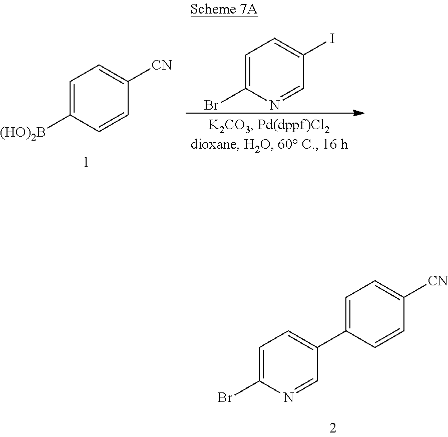 [SMILES: N#Cc1ccc(B(O)O)cc1 ; Brc1ccc(I)cn1 ; N#Cc1ccc(-c2ccc(Br)nc2)cc1]

A mixture of (4-cyanophenyl)boronic acid (1) (50.0 g, 340.0 mmol, 1.0 eq), 2-bromo-5-iodopyridine (96.6 g, 340.2 mmol, 1.0 eq), and K₂CO₃ (141.0 g, 1.02 mol, 3.0 eq) in dioxane (3.2 L) and H₂O (800 mL) was stirred at 60° C. for 16 h. The mixture was filtered and concentrated. The crude residue was purified through a silica gel column (PE:EtOAc=10:1 to 1:1) to afford 4-(6-bromopyridin-3-yl)benzonitrile (2) (63.0 g). LC-MS (ESI) m/z calculated for C₁₂H₇BrN₂+H⁺: 261.0; found: 261.1 (isotope of ⁸¹Br). This reaction is presented as Scheme 7A.

### Synthesis of 4-(6-bromopyridin-3-yl)-N-hydroxybenzimidamide

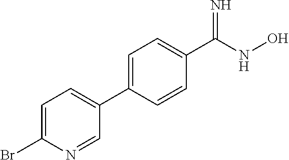 [SMILES: N=C(NO)c1ccc(-c2ccc(Br)nc2)cc1]

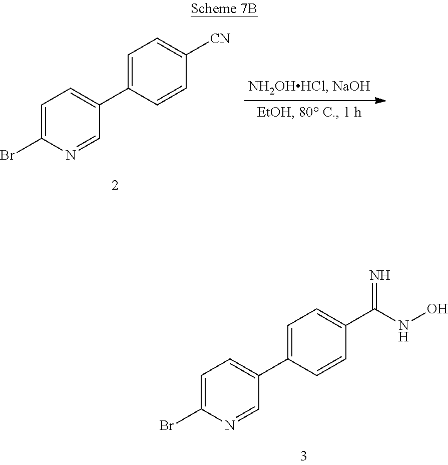 [SMILES: N#Cc1ccc(-c2ccc(Br)nc2)cc1 ; N=C(NO)c1ccc(-c2ccc(Br)nc2)cc1]

A mixture of 4-(6-bromopyridin-3-yl)benzonitrile (2) (60 g, 231.6 mmol, 1.0 eq), NH₂OH—HCl (40.2 g, 579 mmol, 2.5 eq), and NaOH (23.16 g, 579 mmol, 2.5 eq) in EtOH (500 mL) was stirred at 80° C. for 3 h. The mixture was diluted with EtOH (100 mL) and filtered. The filtrate was concentrated to afford 4-(6-bromopyridin-3-yl)-N-hydroxybenzimidamide (3) (40 g, crude). LC-MS (ESI) m/z calculated for C₁₂H₁₀BrN₃O+H⁺: 294.0; found: 294.1 (isotope of ⁸¹Br). This reaction is presented as Scheme 7B.

### Synthesis of 4-(6-bromopyridin-3-yl) benzimidamide

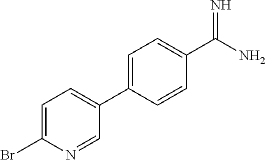 [SMILES: N=C(N)c1ccc(-c2ccc(Br)nc2)cc1]

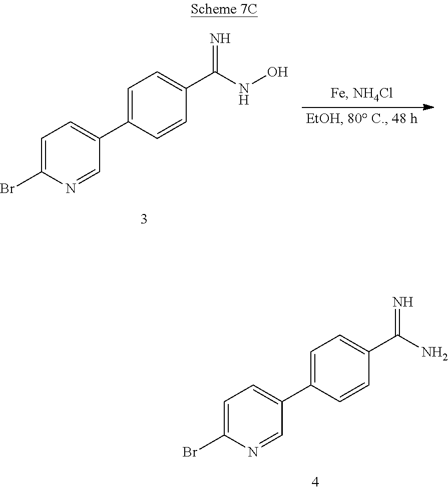 [SMILES: N=C(NO)c1ccc(-c2ccc(Br)nc2)cc1 ; N=C(N)c1ccc(-c2ccc(Br)nc2)cc1]

A mixture of 4-(6-bromopyridin-3-yl)-N-hydroxybenzimidamide (3) (40.0 g, 136.8 mmol, 1.0 eq), NH₄Cl (146.48 g, 3.460 mol, 20 eq), and Fe (230.04 g, 2.74 mol, 20 eq) in EtOH (600 mL) was stirred at 80° C. for 48 h. The mixture was cooled and filtered. The solvent was removed in vacuo to afford 4-(6-bromopyridin-3-yl) benzimidamide (4) (30.0 g, crude). LC-MS (ESI) m/z calculated for C₁₂H₁₀BrN₃+H⁺: 276.0; found: 276.1 (isotope of ⁷⁹Br). This reaction is presented as Scheme 7C.

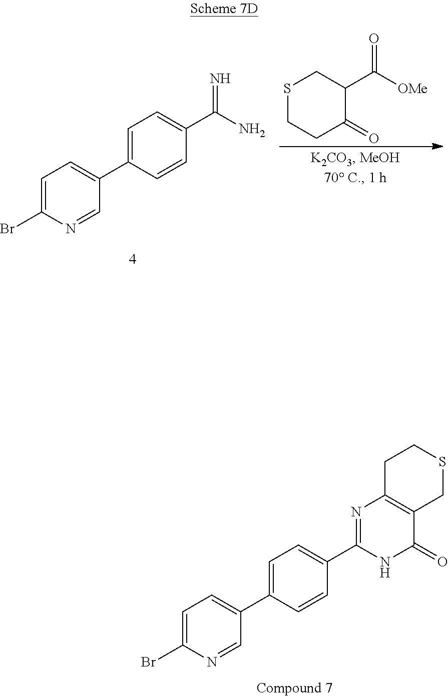

A mixture of 4-(6-bromopyridin-3-yl) benzimidamide (4) (30.0 g, 108.6 mmol, 1.0 eq), methyl 4-oxotetrahydro-2H-thiopyran-3-carboxylate (37.8 g, 217.2 mmol, 2 eq), and K₂CO₃ (45.0 g, 325.8 mmol, 3.0 eq) in MeOH (500 mL) was stirred at 70° C. for 4 h. The mixture was concentrated under reduced pressure. The residue was washed with H₂O (200 mL) and filtered. The solid was triturated in MeOH (150 mL), EtOAc (150 mL), and acetone (100 mL), respectively, to remove most of the impurities. The mixture was filtered off. The crude solid was triturated in aqueous NaOH (1% wt, 20 mL) and filtered. The solid was washed with water (50 mL) and acetone (100 mL) and dried under vacuum to afford 2-(4-(6-bromopyridin-3-yl) phenyl)-3,5,7,8-tetrahydro-4H-thiopyrano [4,3-d]pyrimidin-4-one (Compound 7). LC-MS (ESI) m/z calculated for C₁₈H₁₄BrN₃OS+H⁺: 402.0; found: 401.9 (isotope of ⁸¹Br). ¹H NMR (400 MHz, DMSO): δ 12.82 (brs, 1H), 8.81 (d, J=2.4 Hz, 1H), 8.22 (d, J=8.3 Hz, 2H), 8.14 (dd, J=8.4, 2.4 Hz, 1H), 7.91 (d, J=8.3 Hz, 2H), 7.77 (d, J=8.4 Hz, 1H), 3.54 (s, 2H), 2.91 (brs, 4H). This reaction is presented as Scheme 7D.

### Compound 8

### Synthesis of 2-(4-(2-(2-hydroxyethoxy)propan-2-yl)phenyl)-3,5,7,8-tetrahydro-4H-thiopyrano[4,3-d]pyrimidin-4-one (Compound 8)

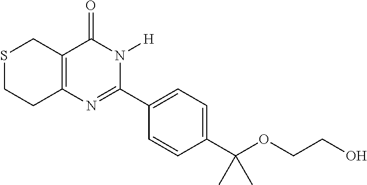 [SMILES: CC(C)(OCCO)c1ccc(-c2nc3c(c(=O)[nH]2)CSCC3)cc1]

### Synthesis of methyl 4-(4-oxo-3,5,7,8-tetrahydro-4H-thiopyrano[4,3-d]pyrimidin-2-yl)benzoate

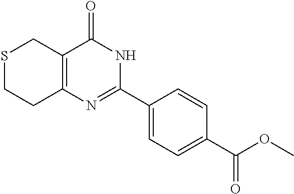 [SMILES: COC(=O)c1ccc(-c2nc3c(c(=O)[nH]2)CSCC3)cc1]

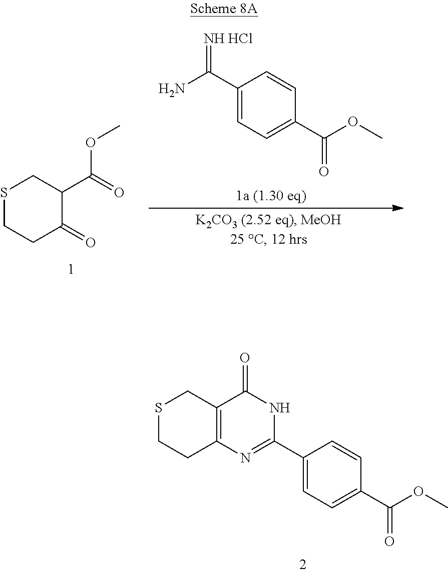

To the solution of methyl 4-oxotetrahydro-2H-thiopyran-3-carboxylate (1) (2 g, 11.48 mmol, 1 eq) in MeOH (20 mL) was added methyl 4-carbamimidoylbenzoate hydrochloride (1a) (3.20 g, 14.91 mmol, 1.30 eq) and K₂CO₃ (4.00 g, 28.94 mmol, 2.52 eq) at 25° C. The mixture was stirred at 25° C. for 12 h. The mixture was filtered, and the filter cake was concentrated under reduced pressure to give a residue. The residue was suspended in H₂O (40 mL) and stirred at 25° C. for 12 h. The mixture was filtered, and the filter cake was dried in vacuo to give methyl 4-(4-oxo-3,5,7,8-tetrahydro-4H-thiopyrano[4,3-d]pyrimidin-2-yl)benzoate (2) (1.5 g, crude). The crude product was used in the next step directly without further purification. This reaction is presented as Scheme 8A.

### Synthesis of 2-(4-(2-hydroxypropan-2-yl)phenyl)-3,5,7,8-tetrahydro-4H-thiopyrano[4,3-d]pyrimidin-4-one

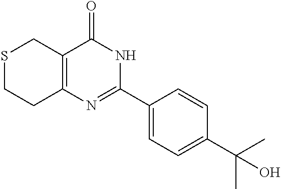 [SMILES: CC(C)(O)c1ccc(-c2nc3c(c(=O)[nH]2)CSCC3)cc1]

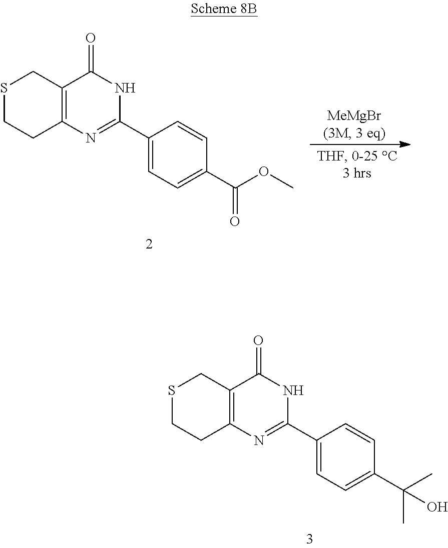 [SMILES: COC(=O)c1ccc(-c2nc3c(c(=O)[nH]2)CSCC3)cc1 ; CC(C)(O)c1ccc(-c2nc3c(c(=O)[nH]2)CSCC3)cc1]

To a solution of methyl 4-(4-oxo-3,5,7,8-tetrahydro-4H-thiopyrano[4,3-d]pyrimidin-2-yl)benzoate (2) (0.3 g, 992.24 mol, 1 eq) in THF (3 mL), MeMgBr (3 M, 992.24 μL, 3 eq) was added dropwise at 0° C. under N₂. The mixture was stirred at 25° C. for 3 h. The reaction mixture was quenched by the addition of NH₄Cl (5 mL), and the aqueous phase was extracted with 10 mL of DCM (5 mL×2). The combined organic layers were concentrated under reduced pressure to give a residue. 2-(4-(2-hydroxypropan-2-yl)phenyl)-3,5,7,8-tetrahydro-4H-thiopyrano[4,3-d]pyrimidin-4-one (3) (2 g, crude) was obtained, which was used in the next step directly without further purification. ¹H NMR: ET47629-1-P1A (400 MHz, CDCl₃): δ 12.54-12.27 (m, 1H), 8.07 (d, J=8.5 Hz, 2H), 7.56 (d, J=8.5 Hz, 2H), 3.62 (br s, 2H), 3.03-2.94 (m, 2H), 2.91-2.82 (m, 2H), 1.55 (s, 6H). This reaction is presented as Scheme 8B.

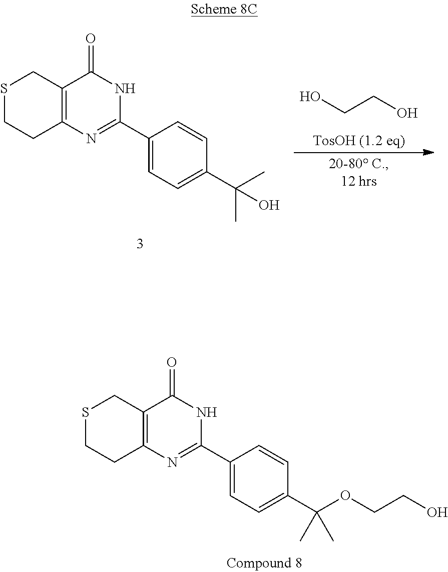

To a solution of 2-(4-(2-hydroxypropan-2-yl)phenyl)-3,5,7,8-tetrahydro-4H-thiopyrano[4,3-d]pyrimidin-4-one (3) (0.12 g, 396.84 mol, 1 eq) in ethylene glycol (5 mL) was added TosOH (82.00 mg, 476.21 mol, 1.2 eq) at 20° C. The mixture was stirred at 20° C. for 1 h, then warmed to 80° C. and stirred at 80° C. for 12 h. The mixture was purified directly by prep-HPLC (neutral condition, column: Waters Xbridge BEH C18 100*30 mm*10 m; mobile phase: [water (NH₄HCO₃)-ACN]; B %: 10%-50%, 10 min). 2-(4-(2-(2-hydroxyethoxy)propan-2-yl)phenyl)-3,5,7,8-tetrahydro-4H-thiopyrano[4,3-d]pyrimidin-4-one (Compound 8) was obtained. LC-MS: 347.2 (M+1). ¹H NMR: ET47430-4-P1A1 (400 MHz, CDCl₃): δ 8.14 (d, J=8.2 Hz, 2H), 7.60 (d, J=8.3 Hz, 2H), 3.80-3.69 (m, 4H), 3.34 (t, J=4.6 Hz, 2H), 3.14-3.06 (m, 2H), 3.01-2.93 (m, 2H), 1.62 (s, 6H). This reaction is presented as Scheme 8C.

### Compound 9

### Synthesis of 2-(4′-bromo-[1,1′-biphenyl]-4-yl)-3,5,7,8-tetrahydro-4H-thiopyrano[4,3-d]pyrimidin-4-one (Compound 9)

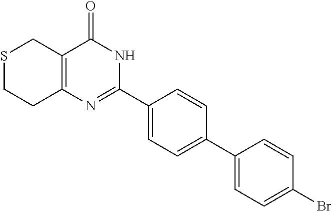 [SMILES: O=c1[nH]c(-c2ccc(-c3ccc(Br)cc3)cc2)nc2c1CSCC2]

### Synthesis of 4′-bromo-[1,1′-biphenyl]-4-carboximidamide

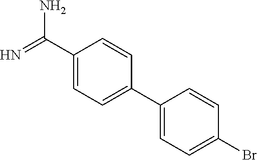 [SMILES: N=C(N)c1ccc(-c2ccc(Br)cc2)cc1]

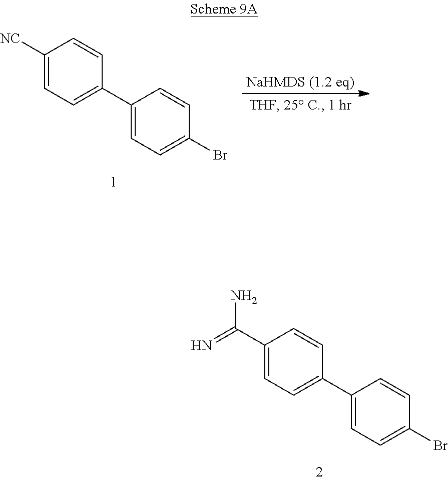 [SMILES: N#Cc1ccc(-c2ccc(Br)cc2)cc1 ; N=C(N)c1ccc(-c2ccc(Br)cc2)cc1]

To a mixture of 4′-bromo-[1,1′-biphenyl]-4-carbonitrile (1) (4 g, 15.5 mmol, 1 eq) in THF (40 mL) was added NaHMDS (1 M, 18.60 mL, 1.2 eq) dropwise at 25° C. under N₂. The mixture was stirred at 25° C. for 1 h, and a liquid was obtained. The reaction mixture was quenched by the addition of water (30 mL) at 25° C. The pH of the mixture was adjusted to 2~3 by addition of 1 M HCl. The aqueous layer was separated and evaporated to about 15% of its original volume, resulting in crystallization of the desired HCl-salts. The mixture was filtered, and the filter cake was dried in vacuo. 4′-bromo-[1,1′-biphenyl]-4-carboximidamide (2) (4.53 g, 14.5 mmol, HCl) was obtained. The crude product was used in the next step without further purification. ¹H NMR: (400 MHz, DMSO-d₆): δ 9.44 (s, 2H), 9.19 (s, 2H), 7.94 (s, 4H), 7.81-7.65 (m, 4H).

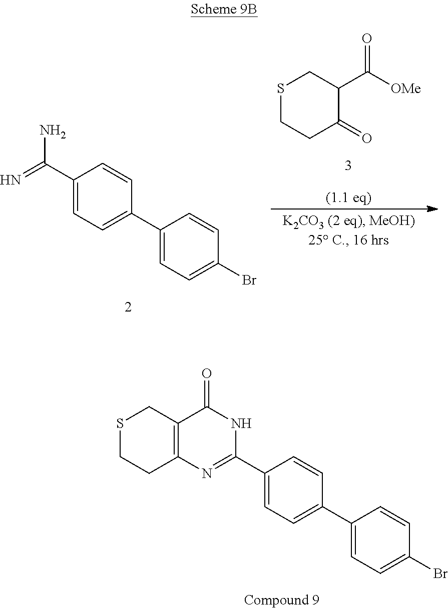 [SMILES: N=C(N)c1ccc(-c2ccc(Br)cc2)cc1 ; COC(=O)C1CSCCC1=O ; O=c1[nH]c(-c2ccc(-c3ccc(Br)cc3)cc2)nc2c1CSCC2]

To a solution of 4′-bromo-[1,1′-biphenyl]-4-carboximidamide (2) (0.066 g, 378.8 mol, 1.1 eq) in MeOH (1.8 mL) was added methyl 4-oxotetrahydro-2H-thiopyran-3-carboxylate (3) (107.3 mg, 344.4 mol, 1 eq, HCl) and K₂CO₃ (95.2 mg, 688.8 mol, 2 eq) at 25° C. The mixture was stirred at 25° C. for 16 h. The reaction mixture was filtered, and the filter cake was dried in vacuo to give a residue. The crude product was purified by precipitation from DMSO (3 mL). 2-(4′-bromo-[1,1′-biphenyl]-4-yl)-3,5,7,8-tetrahydro-4H-thiopyrano[4,3-d]pyrimidin-4-one (Compound 9) was obtained. LC-MS: 399.19 (M+1). ¹H NMR: ET48394-6-P1A (400 MHz, DMSO-d₆): δ 12.86-12.62 (m, 1H), 8.21 (d, J=8.3 Hz, 2H), 7.86-7.81 (m, 2H), 7.75-7.68 (m, 4H), 3.55 (s, 2H), 2.91 (s, 4H).

### Compound 10

### Synthesis of (4′-(4-oxo-3,5,7,8-tetrahydro-4H-thiopyrano[4,3-d]pyrimidin-2-yl)-[1,1′-biphenyl]-4-yl)boronic acid (Compound 10)

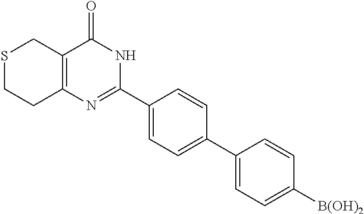 [SMILES: O=c1[nH]c(-c2ccc(-c3ccc(B(O)O)cc3)cc2)nc2c1CSCC2]

### Synthesis of 2-(4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one

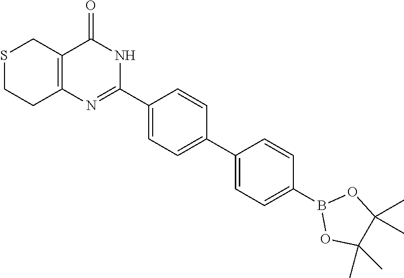 [SMILES: CC1(C)OB(c2ccc(-c3ccc(-c4nc5c(c(=O)[nH]4)CSCC5)cc3)cc2)OC1(C)C]

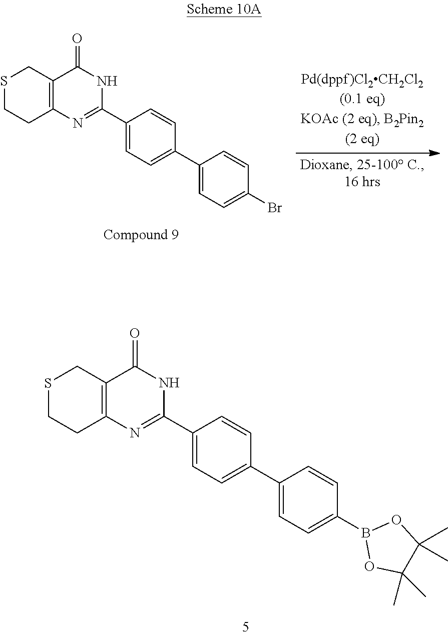 [SMILES: O=c1[nH]c(-c2ccc(-c3ccc(Br)cc3)cc2)nc2c1CSCC2 ; CC1(C)OB(c2ccc(-c3ccc(-c4nc5c(c(=O)[nH]4)CSCC5)cc3)cc2)OC1(C)C]

To a mixture of 2-(4′-bromo-[1,1′-biphenyl]-4-yl)-3,5,7,8-tetrahydro-4H-thiopyrano[4,3-d]pyrimidin-4-one (Compound 9) (0.31 g, 776.4 mol, 1 eq) and B₂Pin₂ (394.3 mg, 1.55 mmol, 2 eq) in dioxane (6 mL) was added KOAc (152.4 mg, 1.55 mmol, 2 eq) and Pd(dppf)Cl₂·CH₂Cl₂ (63.4 mg, 77.6 mol, 0.1 eq) in portions at 25° C. under N₂. The mixture was degassed under vacuum and purged three times with N₂. Then the reaction mixture was heated to 100° C. and stirred for 16 h. A suspension liquid was obtained. The crude product was triturated with H₂O (10 mL) at 25° C. for 2 h. The mixture was filtered, and the filter cake was washed with DCM (5 mL) and MeOH (5 mL). The filter cake was dried under reduced pressure to give 2-(4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (0.18 g) which was used in the next step without further purification.

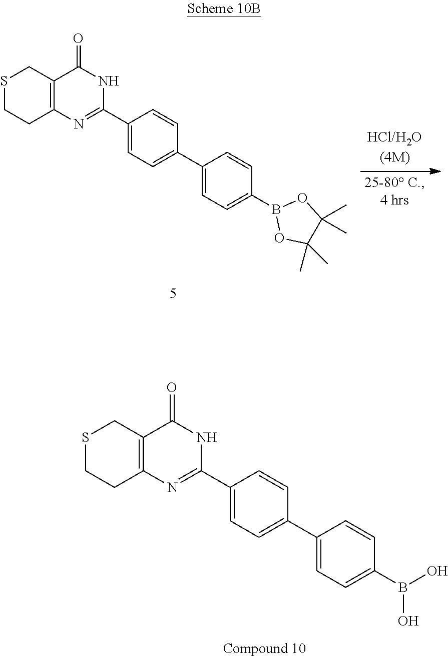 [SMILES: CC1(C)OB(c2ccc(-c3ccc(-c4nc5c(c(=O)[nH]4)CSCC5)cc3)cc2)OC1(C)C ; O=c1[nH]c(-c2ccc(-c3ccc(B(O)O)cc3)cc2)nc2c1CSCC2]

To a round-bottom flask was added 2-(4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (0.18 g, 403.2 mol, 1 eq) and HCl (4 M, 4 mL, 39.7 eq) at 25° C. The reaction mixture was stirred at 80° C. for 4 h. The mixture was filtered, and the filter cake was washed with DCM (5 mL) and MeOH (5 mL). The filter cake was dried in vacuo. (4′-(4-oxo-3,5,7,8-tetrahydro-4H-thiopyrano[4,3-d]pyrimidin-2-yl)-[1,1′-biphenyl]-4-yl)boronic acid (Compound 10) was obtained. LC-MS: 365.1 (M+1). ¹H NMR: ET48394-20 (400 MHz, DMSO-d₆): δ 8.20 (d, J=8.5 Hz, 2H), 7.89 (dd, J=8.3, 19.4 Hz, 4H), 7.74 (d, J=8.2 Hz, 2H), 3.55 (s, 2H), 2.92 (s, 4H).

### Compound 11

### Synthesis of 2-(4-(2-(2-hydroxyethoxy)pyrimidin-5-yl)phenyl)-3,5,7,8-tetrahydro-4H-thiopyrano[4,3-d]pyrimidin-4-one (Compound 11)

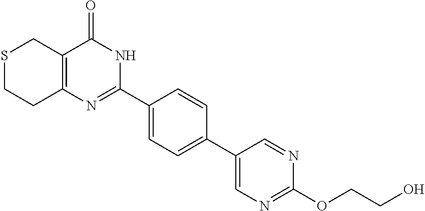 [SMILES: O=c1[nH]c(-c2ccc(-c3cnc(OCCO)nc3)cc2)nc2c1CSCC2]

### Synthesis of 4-(2-chloropyrimidin-5-yl)benzonitrile

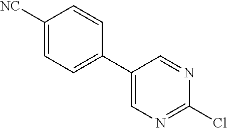 [SMILES: N#Cc1ccc(-c2cnc(Cl)nc2)cc1]

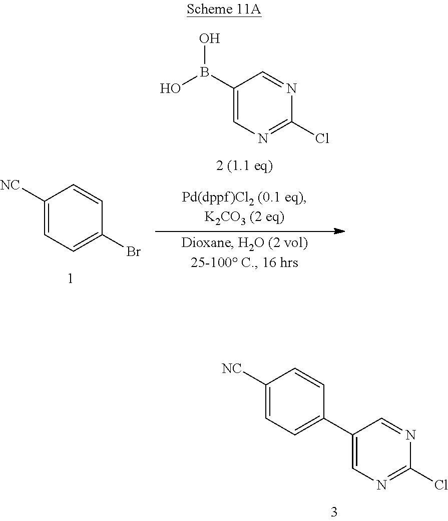 [SMILES: OB(O)c1cnc(Cl)nc1 ; N#Cc1ccc(Br)cc1 ; N#Cc1ccc(-c2cnc(Cl)nc2)cc1]

To a mixture of (2-chloropyrimidin-5-yl)boronic acid (2) (9.57 g, 60.43 mmol, 1.10 eq) and 4-bromobenzonitrile (1) (10.0 g, 54.9 mmol, 1.00 eq) in dioxane (200 mL) and H₂O (20.0 mL) was added K₂CO₃ (15.2 g, 110 mmol, 2.00 eq) and Pd(dppf)Cl₂·CH₂Cl₂ (4.49 g, 5.49 mmol, 0.10 eq) in one portion at 25° C. under N₂. The mixture was degassed with N₂ and then stirred at 100° C. for 16 h. A liquid was obtained. The reaction mixture was diluted with water (200 mL) and extracted with EtOAc 600 mL (200 mL×3). The combined organic layers were concentrated under reduced pressure to give a residue that was purified by flash silica gel chromatography (ISCO®; 200 g SepaFlash® Silica Flash Column, eluent of 0~20% ethyl acetate/petroleum ether gradient @100 mL/min, R_{f}=0.25). 4-(2-chloropyrimidin-5-yl)benzonitrile (3) (1.1 g) was obtained and used as a crude in the next step.

### Synthesis of 4-(2-(2-hydroxyethoxy)pyrimidin-5-yl)benzonitrile

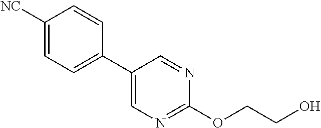 [SMILES: N#Cc1ccc(-c2cnc(OCCO)nc2)cc1]

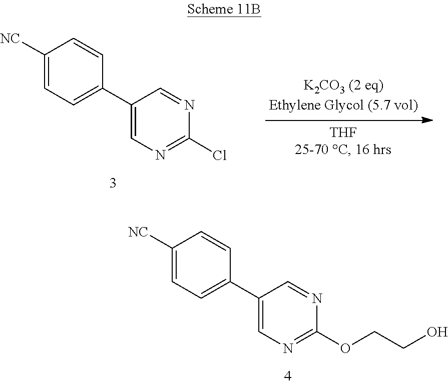 [SMILES: N#Cc1ccc(-c2cnc(Cl)nc2)cc1 ; N#Cc1ccc(-c2cnc(OCCO)nc2)cc1]

To a mixture of 4-(2-chloropyrimidin-5-yl)benzonitrile (3) (0.70 g, 3.25 mmol, 1.00 eq) in THE (4.00 mL) and ethylene glycol (4.00 mL) was added K₂CO₃ (897 mg, 6.49 mmol, 2.00 eq) in one portion at 25° C. under N₂. The mixture was stirred at 70° C. for 16 h. A suspension liquid was obtained. The reaction mixture was diluted with water (20 mL) and extracted with EtOAc 30 mL (10 mL×3). The combined organic layers were concentrated under reduced pressure to give a residue that was purified by flash silica gel chromatography (ISCO®; 40 g SepaFlash® Silica Flash Column, eluent of 0~50% ethyl acetate/petroleum ether gradient @40 mL/min, R_{f}=0.27). 4-(2-(2-hydroxyethoxy)pyrimidin-5-yl)benzonitrile (4) (540 mg, 2.24 mmol) was obtained and used crude in the next step.

### Synthesis of (Z)—N′-hydroxy-4-(2-(2-hydroxyethoxy)pyrimidin-5-yl)benzimidamide

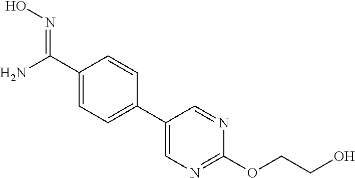 [SMILES: N/C(=N\O)c1ccc(-c2cnc(OCCO)nc2)cc1]

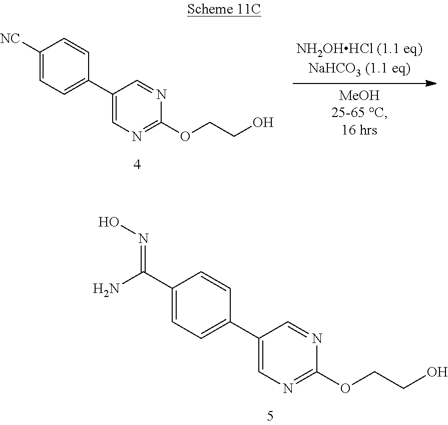 [SMILES: N#Cc1ccc(-c2cnc(OCCO)nc2)cc1 ; N/C(=N\O)c1ccc(-c2cnc(OCCO)nc2)cc1]

To a mixture of 4-(2-(2-hydroxyethoxy)pyrimidin-5-yl)benzonitrile (4) (0.54 g, 2.24 mmol, 1.00 eq) in MeOH (5.00 mL) was added NH₂OH·HCl (171 mg, 2.46 mmol, 1.10 eq) and NaHCO₃ (207 mg, 2.46 mmol, 95.8 μL, 1.10 eq) in one portion at 25° C. under N₂. The mixture was stirred at 65° C. for 16 h. A liquid was obtained. The reaction mixture was filtered and concentrated under reduced pressure to give a (Z)—N′-hydroxy-4-(2-(2-hydroxyethoxy)pyrimidin-5-yl)benzimidamide (5) (500 mg, crude) and used in the next step without further purification.

### Synthesis of 4-(2-(2-hydroxyethoxy)pyrimidin-5-yl)benzimidamide

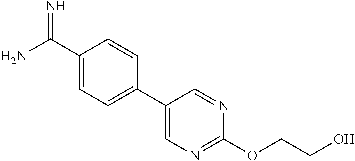 [SMILES: N=C(N)c1ccc(-c2cnc(OCCO)nc2)cc1]

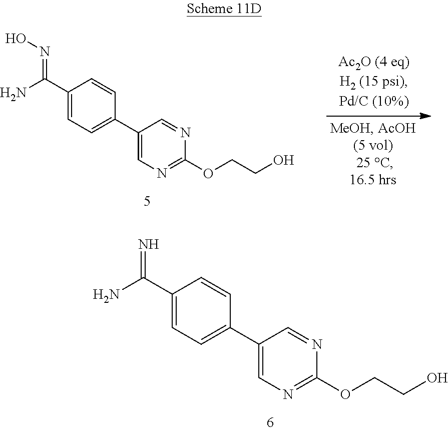 [SMILES: N/C(=N\O)c1ccc(-c2cnc(OCCO)nc2)cc1 ; N=C(N)c1ccc(-c2cnc(OCCO)nc2)cc1]

To a mixture of (Z)—N′-hydroxy-4-(2-(2-hydroxyethoxy)pyrimidin-5-yl)benzimidamide (5) (0.50 g, 1.82 mmol, 1.00 eq) in AcOH (2.62 g, 43.7 mmol, 2.50 mL) was added Ac₂O (744 mg, 7.29 mmol, 683 μL, 4.00 eq) in one portion at 25° C. under N₂. The mixture was stirred at 25° C. for 30 min. MeOH (20 mL) and Pd/C (0.2 g, 218.76 μmol) were then added. The resulting mixture was degassed three times with H₂ and stirred under H₂ (15 psi) at 25° C. for 16 h. The reaction mixture was filtered and concentrated under reduced pressure to give 4-(2-(2-hydroxyethoxy)pyrimidin-5-yl)benzimidamide (6) (500 mg, crude) and used in the next step without further purification.

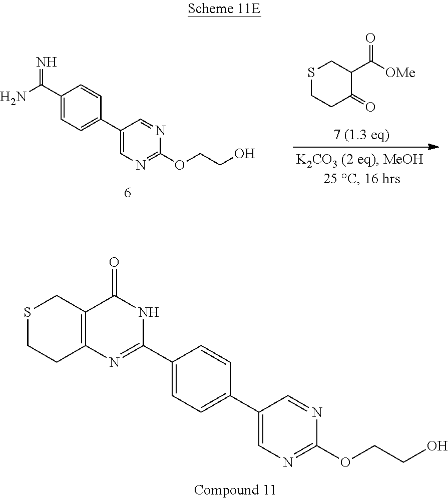 [SMILES: N=C(N)c1ccc(-c2cnc(OCCO)nc2)cc1 ; COC(=O)C1CSCCC1=O ; O=c1[nH]c(-c2ccc(-c3cnc(OCCO)nc3)cc2)nc2c1CSCC2]

To a mixture of 4-(2-(2-hydroxyethoxy)pyrimidin-5-yl)benzimidamide (6) (500 mg, 1.94 mmol, 1.00 eq) and methyl 4-oxotetrahydro-2H-thiopyran-3-carboxylate (7) (438 mg, 2.52 mmol, 1.30 eq) in MeOH (4.00 mL) was added K₂CO₃ (535 mg, 3.87 mmol, 2.00 eq) in one portion at 25° C. under N₂. The mixture was stirred at 25° C. for 16 h. The reaction mixture was filtered and concentrated under reduced pressure to give a residue that was purified by prep-HPLC (column: Waters Xbridge Prep OBD C18 150*40 mm*10 um; mobile phase: [water (NH₄HCO₃)-ACN]; B %: 10%-40%, 8 min). 2-(4-(2-(2-hydroxyethoxy)pyrimidin-5-yl)phenyl)-3,5,7,8-tetrahydro-4H-thiopyrano[4,3-d]pyrimidin-4-one (Compound 11) was obtained. ¹H NMR (400 MHz, DMSO-d6) δ=13.15-12.49 (m, 1H), 9.03 (s, 2H), 8.21 (br d, J=8.2 Hz, 2H), 7.91 (d, J=8.6 Hz, 2H), 4.93 (t, J=5.5 Hz, 1H), 4.42-4.36 (m, 2H), 3.76 (q, J=5.4 Hz, 2H), 3.54 (s, 2H), 2.95-2.86 (m, 4H). LCMS: 383 (M+1).

### Compound 12

### Synthesis of 2-(4′-(2-(2-hydroxyethoxy)propan-2-yl)-[1,1′-biphenyl]-4-yl)-3,5,7,8-tetrahydro-4H-thiopyrano[4,3-d]pyrimidin-4-one (Compound 12)

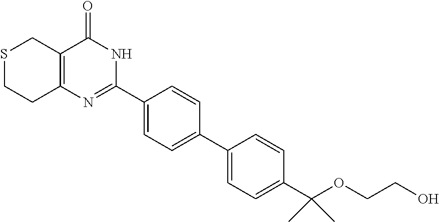 [SMILES: CC(C)(OCCO)c1ccc(-c2ccc(-c3nc4c(c(=O)[nH]3)CSCC4)cc2)cc1]

### Synthesis of 2-(4-bromophenyl)-3,5,7,8-tetrahydro-4H-thiopyrano[4,3-d]pyrimidin-4-one

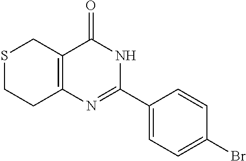 [SMILES: O=c1[nH]c(-c2ccc(Br)cc2)nc2c1CSCC2]

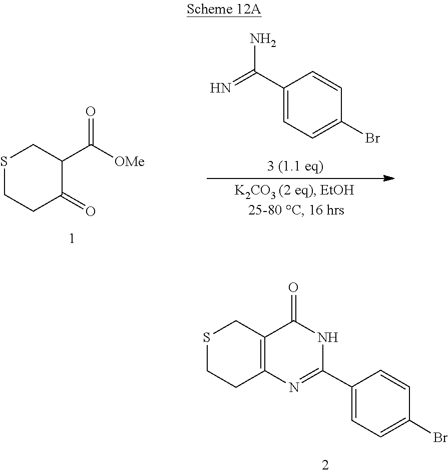 [SMILES: COC(=O)C1CSCCC1=O ; N=C(N)c1ccc(Br)cc1 ; O=c1[nH]c(-c2ccc(Br)cc2)nc2c1CSCC2]

To a mixture of methyl 4-oxotetrahydro-2H-thiopyran-3-carboxylate (1) (8.00 g, 45.9 mmol, 1.00 eq) and 4-bromobenzimidamide (3) (11.9 g, 50.5 mmol, 1.10 eq, HCl) in EtOH (100 mL) was added K₂CO₃ (12.7 g, 91.8 mmol, 2.00 eq) in one portion at 25° C. under N₂. The mixture was stirred at 80° C. for 16 h. The reaction mixture was filtered and concentrated under reduced pressure to give 2-(4-bromophenyl)-3,5,7,8-tetrahydro-4H-thiopyrano[4,3-d]pyrimidin-4-one (2) (15.6 g, crude) that was used in the next step without further purification. ¹H NMR: (400 MHz, DMSO-d6): δ 8.19-8.13 (m, 2H), 7.53-7.46 (m, 2H), 3.42 (s, 2H), 2.81-2.75 (m, 2H), 2.74-2.67 (m, 2H).

### Synthesis of 2-(4′-(2-hydroxypropan-2-yl)-[1,1′-biphenyl]-4-yl)-3,5,7,8-tetrahydro-4H-thiopyrano[4,3-d]pyrimidin-4-one

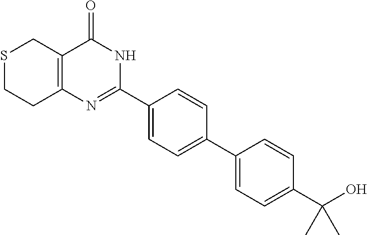 [SMILES: CC(C)(O)c1ccc(-c2ccc(-c3nc4c(c(=O)[nH]3)CSCC4)cc2)cc1]

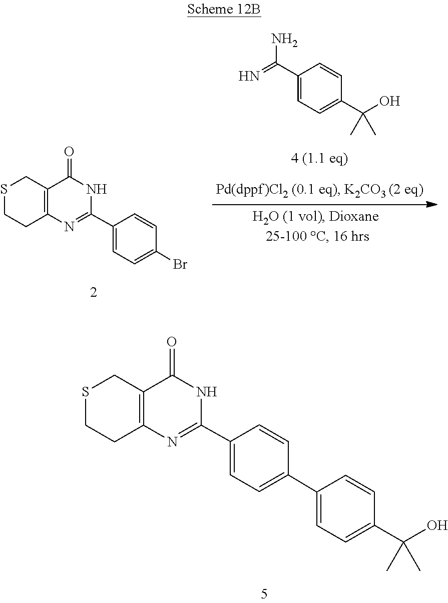 [SMILES: CC(C)(O)c1ccc(C(=N)N)cc1 ; O=c1[nH]c(-c2ccc(Br)cc2)nc2c1CSCC2 ; CC(C)(O)c1ccc(-c2ccc(-c3nc4c(c(=O)[nH]3)CSCC4)cc2)cc1]

To a mixture of 2-(4-bromophenyl)-3,5,7,8-tetrahydro-4H-thiopyrano[4,3-d]pyrimidin-4-one (2) (4.00 g, 12.4 mmol, 1.00 eq) and (4-(2-hydroxypropan-2-yl)phenyl)boronic acid (4) (2.67 g, 14.9 mmol, 1.20 eq) in dioxane (40.0 mL) and H₂O (5.00 mL) was added K₂CO₃ (3.42 g, 24.8 mmol, 2.00 eq) and Pd(dppf)Cl₂·CH₂Cl₂ (1.01 g, 1.24 mmol, 0.10 eq) in one portion at 25° C. under N₂. The mixture was degassed under vacuum, purged three times with N₂, and then heated to 100° C. and stirred for 16 h. The reaction mixture was diluted with EtOAc (20.0 mL) and then filtered. The filter cake was washed with water (10 mL) and MeOH (10 mL) to give 2-(4′-(2-hydroxypropan-2-yl)-[1,1′-biphenyl]-4-yl)-3,5,7,8-tetrahydro-4H-thiopyrano[4,3-d]pyrimidin-4-one (5) (2.10 g, 5.55 mmol, crude) and used in the next step without further purification. ¹H NMR: (400 MHz, DMSO-d6): δ=13.13-12.31 (m, 1H), 8.51-7.75 (m, 4H), 7.75-7.15 (m, 4H), 5.07 (br d, J=3.2 Hz, 1H), 3.54 (br s, 2H), 2.90 (br s, 4H), 1.78-0.88 (m, 6H).

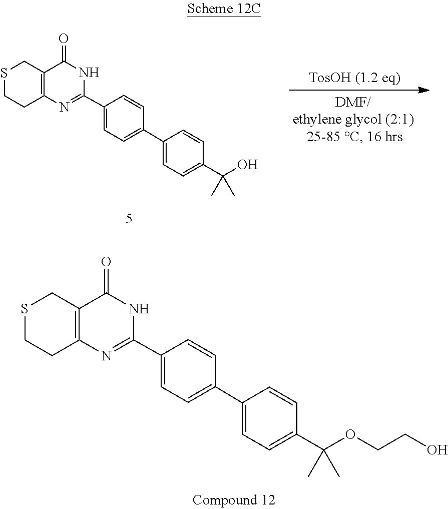 [SMILES: CC(C)(O)c1ccc(-c2ccc(-c3nc4c(c(=O)[nH]3)CSCC4)cc2)cc1 ; CC(C)(OCCO)c1ccc(-c2ccc(-c3nc4c(c(=O)[nH]3)CSCC4)cc2)cc1]

To a mixture of 2-(4′-(2-hydroxypropan-2-yl)-[1,1′-biphenyl]-4-yl)-3,5,7,8-tetrahydro-4H-thiopyrano[4,3-d]pyrimidin-4-one (5) (1.50 g, 3.96 mmol, 1.00 eq) in DMF (10.0 mL) was added TosOH (819 mg, 4.76 mmol, 1.20 eq) and ethylene glycol (5.55 g, 89.4 mmol, 5.00 mL, 22.6 eq) in one portion at 25° C. under N₂. The mixture was stirred at 85° C. for 16 h. The mixture was cooled to RT and filtered. The filter cake was dissolved in DMSO and then purified directly by prep-HPLC (column: Phenomenex Luna 80 * 30 mm*3 um; mobile phase: [water (HCl)-MEOH]; B %: 40%-65%, 8 min) to give 2-(4′-(2-(2-hydroxyethoxy)propan-2-yl)-[1,1′-biphenyl]-4-yl)-3,5,7,8-tetrahydro-4H-thiopyrano[4,3-d]pyrimidin-4-one (Compound 12). LCMS: 423.2 (M+1). ¹H NMR: (400 MHz, CHLOROFORM-d): δ 11.47-10.94 (m, 1H), 8.14 (d, J=8.4 Hz, 2H), 7.76 (d, J=8.5 Hz, 2H), 7.67-7.61 (m, 2H), 7.54 (d, J=8.4 Hz, 2H), 3.80-3.69 (m, 4H), 3.41-3.31 (m, 2H), 3.10-3.03 (m, 2H), 2.99-2.91 (m, 2H), 1.66-1.62 (m, 6H).

### Compound 13

### Synthesis of 2-(4-(trifluoromethyl)phenyl)-3,5,7,8-tetrahydro-4H-thiopyrano[4,3-d]pyrimidin-4-one-8,8-d2 (Compound 13)

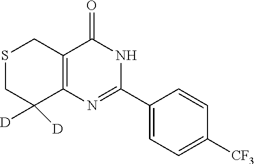 [SMILES: [2H]C1([2H])CSCc2c1nc(-c1ccc(C(F)(F)F)cc1)[nH]c2=O]

### Synthesis of methyl-d3 4-oxotetrahydro-2H-thiopyran-3-carboxylate-3,5,5-d3

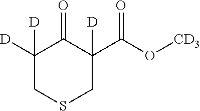 [SMILES: [2H]C([2H])([2H])OC(=O)C1([2H])CSCC([2H])([2H])C1=O]

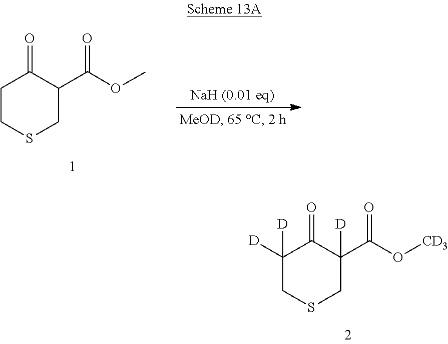 [SMILES: COC(=O)C1CSCCC1=O ; [2H]C([2H])([2H])OC(=O)C1([2H])CSCC([2H])([2H])C1=O]

A mixture of methyl 4-oxotetrahydro-2H-thiopyran-3-carboxylate (1) (0.9 g, 5.16 mmol, 1.0 eq) and NaH (2.1 mg, 0.051 mmol, 0.01 eq) in MeOD (13.5 mL) was stirred at 65° C. for 2 h. Methyl-d3 4-oxotetrahydro-2H-thiopyran-3-carboxylate-3,5,5-d3 (2) was obtained as a crude product in MeOD solution. ¹H NMR (400 MHz, MeOD): δ 3.23-3.07 (m, 1H), 2.95 (s, 1H), 2.76 (s, 1H), 2.73 (s, 2H).

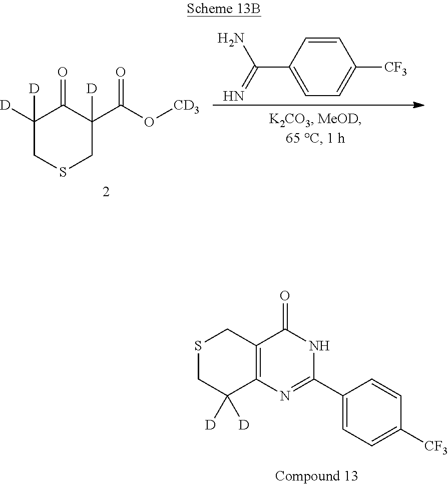 [SMILES: [2H]C([2H])([2H])OC(=O)C1([2H])CSCC([2H])([2H])C1=O ; N=C(N)c1ccc(C(F)(F)F)cc1 ; [2H]C1([2H])CSCc2c1nc(-c1ccc(C(F)(F)F)cc1)[nH]c2=O]

A mixture of (2) (0.90 g, 5.07 mmol, 1.11 eq), and 4-(trifluoromethyl)benzimidamide (0.861 g, 4.56 mmol, 1.0 eq) in MeOD (12 mL) was stirred at 65° C. for 1 h. The mixture was concentrated to a half volume, then added to H₂O (50 mL). The precipitate was filtered and washed with H₂O (30 mL) and MeOH/H₂O (20 mL, 1:1, v/v), and dried in vacuo to afford 2-(4-(trifluoromethyl)phenyl)-3,5,7,8-tetrahydro-4H-thiopyrano[4,3-d]pyrimi-din-4-one-8,8-d2 (Compound 13). LC-MS (ESI) m/z calculated for C₄H₉D₂F₃N₂OS+H⁺: 315.1; found: 315.1. ¹H NMR (400 MHz, DMSO-d₆): δ 8.29 (d, J=8 Hz, 2H), 7.88 (d, J=8 Hz, 2H), 3.54 (s, 2H), 2.89 (s, 2H). ¹⁹F NMR (375 MHz, DMSO-d₆): δ 61.33 (s, 3F).

### Compound 14

### Synthesis of (2,6-difluoro-4-(4-oxo-3,5,7,8-tetrahydro-4H-thiopyrano[4,3-d]pyrimidin-2-yl)phenyl)boronic acid (Compound 14)

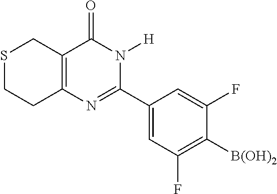 [SMILES: O=c1[nH]c(-c2cc(F)c(B(O)O)c(F)c2)nc2c1CSCC2]

### Synthesis of 3,5-difluorobenzimidamide

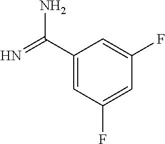 [SMILES: N=C(N)c1cc(F)cc(F)c1]

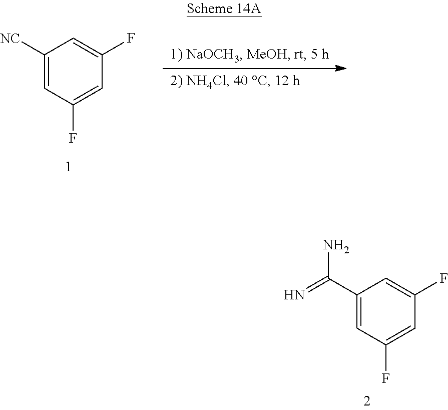 [SMILES: N#Cc1cc(F)cc(F)c1 ; N=C(N)c1cc(F)cc(F)c1]

To a solution of 3,5-difluorobenzonitrile (1) (5.0 g, 36.0 mmol, 1.0 eq) in MeOH (50 mL) was added MeONa (3.9 g, 71.9 mmol, 2.0 eq); the mixture was stirred at 20° C. for 4 h. To the mixture was added NH₄Cl (3.9 g, 71.9 mmol, 2.0 eq), and then the mixture was stirred at 40° C. for 16 h. It was cooled to RT, filtered, and then the filtrate was concentrated to give 3,5-difluorobenzimidamide (2) (7 g, crude). LC-MS 157.4 [M+H]⁺.

### Synthesis of 2-(3,5-difluorophenyl)-3,5,7,8-tetrahydro-4H-thiopyrano[4,3-d]pyrimidin-4-one

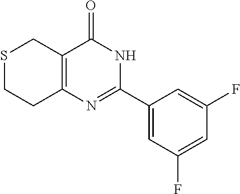 [SMILES: O=c1[nH]c(-c2cc(F)cc(F)c2)nc2c1CSCC2]

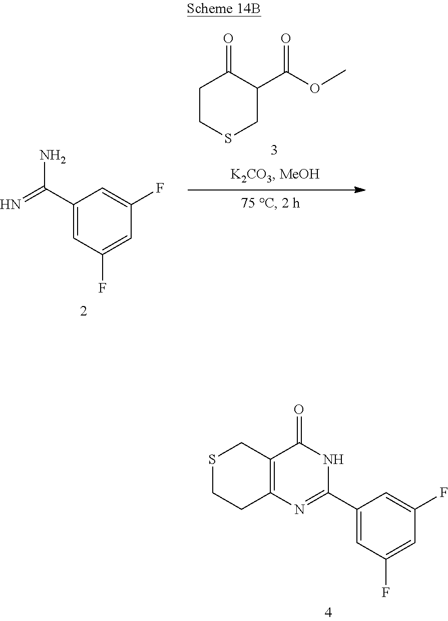 [SMILES: COC(=O)C1CSCCC1=O ; N=C(N)c1cc(F)cc(F)c1 ; O=c1[nH]c(-c2cc(F)cc(F)c2)nc2c1CSCC2]

To a mixture of 3,5-difluorobenzimidamide (2) (7 g, 44.9 mmol, 1.0 eq) and methyl 4-oxotetrahydro-2H-thiopyran-3-carboxylate (3) (6.2 g, 35.9 mmol, 0.8 eq) in MeOH (50 mL) was added K₂CO₃ (12.4 g, 89.7 mmol, 2.0 eq) under N₂. The mixture was stirred at 75° C. for 2 h. The reaction mixture was concentrated to give 2-(3,5-difluorophenyl)-3,5,7,8-tetrahydro-4H-thiopyrano[4,3-d]pyrimidin-4-one (4), which was used directly for the next step. LC-MS 281.1 [M+H]⁺.

### Synthesis of 2-(3,5-difluorophenyl)-4-((2-methoxyethoxy)methoxy)-7,8-dihydro-5H-thiopyrano[4,3-d]pyrimidine

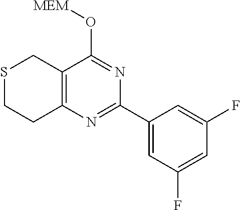

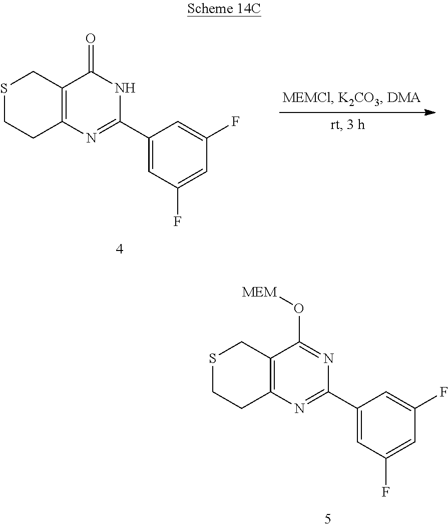

To a mixture of 2-(3,5-difluorophenyl)-3,5,7,8-tetrahydro-4H-thiopyrano[4,3-d]pyrimidin-4-one (4) (crude, 1.0 eq) in DMA (50 mL) was added 1-(chloromethoxy)-2-methoxyethane (11.2 g, 89.7 mmol, 2.0 eq). The reaction mixture was stirred at RT for 3 h and then poured into H₂O (300 mL) and extracted with EtOAc (2×100 mL). The combined organic layer was washed with brine (300 mL), dried over Na₂SO₄, filtered, and concentrated. The residue was purified by column chromatography on silica gel eluted with 0-30% EtOAc/hexane to afford 2-(3,5-difluorophenyl)-4-((2-methoxyethoxy)methoxy)-7,8-dihydro-5H-thiopyrano[4,3-d]pyrimidine (5) (6 g). LC-MS: 369.2 [M+H]⁺.

### Synthesis of (2,6-difluoro-4-(4-((2-methoxyethoxy)methoxy)-7,8-dihydro-5H-thiopyrano[4,3-d]pyrimidin-2-yl)phenyl)boronic acid

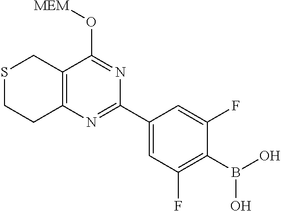

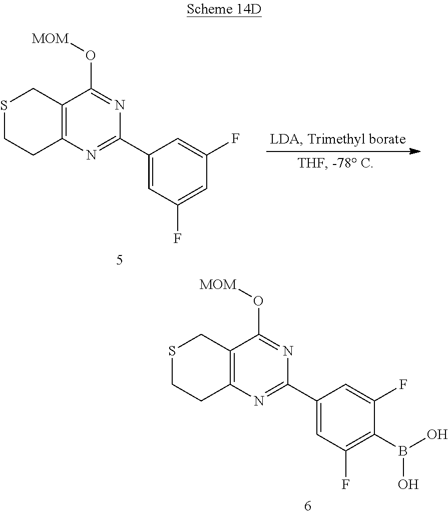

To a mixture of 2-(3,5-difluorophenyl)-4-((2-methoxyethoxy)methoxy)-7,8-dihydro-5H-thiopyrano[4,3-d]pyrimidine (5) (400 mg, 1.4 mmol, 1.0 eq) in dry THF (10 mL) cooled at −78° C. was added LDA (2 M, 1.1 mL, 1.5 eq) dropwise; the mixture was stirred at −78° C. for 1 h. Trimethyl borate was added (191 mg, 1.9 mmol, 1.3 eq) and then the mixture was stirred at −78° C. for 3 h. The mixture was quenched with NH₄Cl solution (30 mL) and extracted with EtOAc (2×20 mL). The combined organic layer was washed with brine (50 mL), dried over Na₂SO₄, filtered, and concentrated. The residue was purified by reverse-phase chromatography (0-50% acetonitrile/0.05% formic acid) to afford (2,6-difluoro-4-(4-((2-methoxyethoxy)methoxy)-7,8-dihydro-5H-thiopyrano[4,3-d]pyrimidin-2-yl)phenyl)boronic acid (6) (200 mg). LC-MS: 413.4 [M+H]⁺.

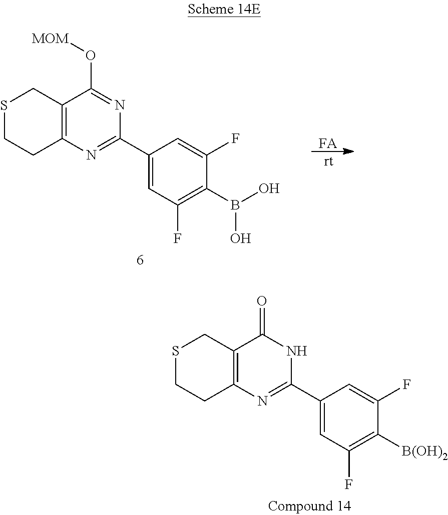

A mixture of (2,6-difluoro-4-(4-((2-methoxyethoxy)methoxy)-7,8-dihydro-5H-thiopyrano[4,3-d]pyrimidin-2-yl)phenyl)boronic acid (6) (200 mg, 0.48 mmol, 1.0 eq) in FA (3 mL) was stirred at RT for 2 h. It was then concentrated and purified by Prep-HPLC to afford (2,6-difluoro-4-(4-oxo-3,5,7,8-tetrahydro-4H-thiopyrano[4,3-d]pyrimidin-2-yl)phenyl)boronic acid (Compound 14). LC-MS 325.1 [M+H]⁺. ¹H NMR (400 MHz, DMSO-d₆): δ 12.85 (s, 1H), 8.86 (s, 2H), 7.72 (d, J=7.2 Hz, 2H), 3.54 (s, 2H), 2.90 (s, 4H).

### Compound 15

### Synthesis of 2-(3,5-difluoro-4-(2-(2-hydroxyethoxy)propan-2-yl)phenyl)-3,5,7,8-tetrahydro-4H-thiopyrano[4,3-d]pyrimidin-4-one (Compound 15)

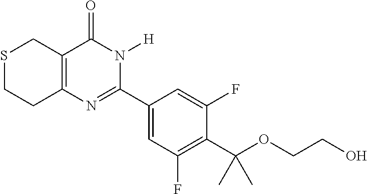 [SMILES: CC(C)(OCCO)c1c(F)cc(-c2nc3c(c(=O)[nH]2)CSCC3)cc1F]

### Synthesis of methyl 4-cyano-2,6-difluorobenzoate

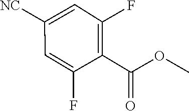

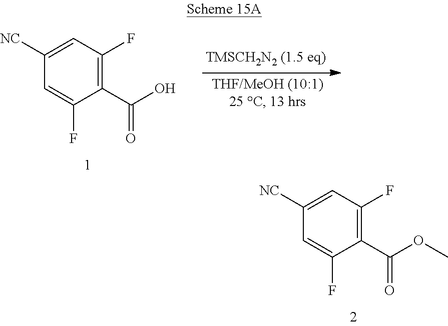

To a solution of 4-cyano-2,6-difluorobenzoic acid (1) (5.00 g, 27.31 mmol, 1.0 eq) in THF, (50 mL)/MeOH (50 mL) TMSCHN₂ (2 M, 20.48 mL, 1.5 eq) was added dropwise at 25° C. The mixture was stirred at 25° C. for 13 h. TLC indicated (petroleum ether/ethyl acetate=5/1, R_{f} (Cpd.1)=0.33) (1) was consumed completely. The reaction mixture was concentrated under reduced pressure to give a residue. Methyl 4-cyano-2,6-difluorobenzoate (2) (4.1 g, crude) was obtained.

### Synthesis of methyl 4-carbamimidoyl-2,6-difluorobenzoate

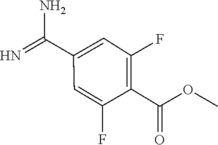

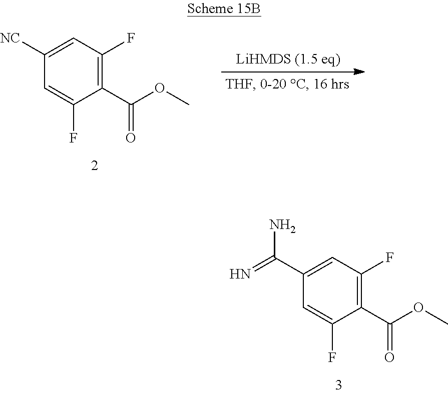

To a solution of methyl 4-cyano-2,6-difluorobenzoate (2) (2.00 g, 10.15 mmol, 1.0 eq) in THF (20 mL) was added LiHMDS (1 M, 15.22 mL, 1.5 eq) at 0° C. The mixture was stirred at 20° C. for 16 h. LC-MS (ET68120-10-P1A1) showed that (2) was consumed completely, and the desired mass was detected. The reaction mixture was quenched by addition of HCl/dioxane (4 mol/L, 8 mL) at 0° C., and then concentrated under reduced pressure to give a residue. The residue was triturated with MeOH (40 mL) and the mixture was stirred at 20° C. for 1 h. Then the mixture was filtered, and the filter cake was dried under reduced pressure. Methyl 4-carbamimidoyl-2,6-difluorobenzoate (3) (2.6 g, crude, HCl) was used directly in the next step without further purification.

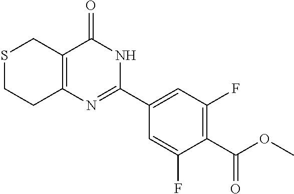 [SMILES: COC(=O)c1c(F)cc(-c2nc3c(c(=O)[nH]2)CSCC3)cc1F]

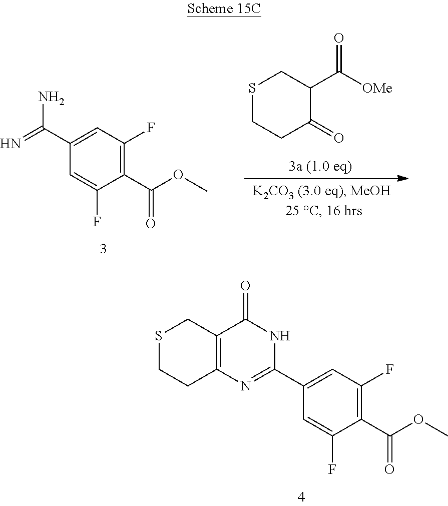 [SMILES: COC(=O)c1c(F)cc(C(=N)N)cc1F ; COC(=O)C1CSCCC1=O ; COC(=O)c1c(F)cc(-c2nc3c(c(=O)[nH]2)CSCC3)cc1F]

To a solution of methyl 4-carbamimidoyl-2,6-difluorobenzoate (3) (470 mg, 1.88 mmol, 1.0 eq, HCl) and methyl 4-oxotetrahydro-2H-thiopyran-3-carboxylate (3a) (327 mg, 1.88 mmol, 1.0 eq) in MeOH (4.7 mL) was added K₂CO₃ (778 mg, 5.63 mmol, 3.0 eq) at 25° C. The mixture was stirred at 25° C. for 16 h. LC-MS showed that (3) was consumed completely, and one peak with the desired mass was detected. The reaction mixture was filtered, and the filtrate was concentrated under reduced pressure to give a residue. The residue was purified by prep-HPLC (neutral condition; column: Waters Xbridge Prep OBD C18 150×40 mm×10 um; mobile phase: [water (NH₄HCO₃)-ACN]; B %: 10%-40%, 8 mins). Methyl 2,6-difluoro-4-(4-oxo-3,5,7,8-tetrahydro-4H-thiopyrano[4,3-d]pyrimidin-2-yl)benzoate (4) (180 mg) was obtained.

### Synthesis of 2-(3,5-difluoro-4-(2-hydroxypropan-2-yl)phenyl)-3,5,7,8-tetrahydro-4H-thiopyrano[4,3-d]pyrimidin-4-one

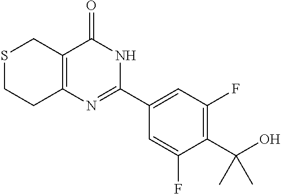 [SMILES: CC(C)(O)c1c(F)cc(-c2nc3c(c(=O)[nH]2)CSCC3)cc1F]

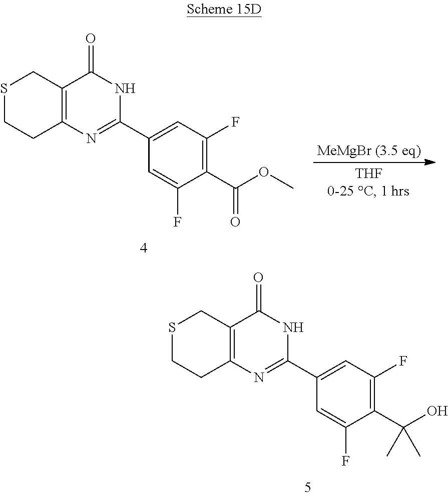 [SMILES: COC(=O)c1c(F)cc(-c2nc3c(c(=O)[nH]2)CSCC3)cc1F ; CC(C)(O)c1c(F)cc(-c2nc3c(c(=O)[nH]2)CSCC3)cc1F]

To a solution of methyl 2,6-difluoro-4-(4-oxo-3,5,7,8-tetrahydro-4H-thiopyrano[4,3-d]pyrimidin-2-yl)benzoate (4) (300 mg, 887 μmol, 1.0 eq) in THE (5.0 mL) was added MeMgBr (3 M, 1.03 mL, 3.5 eq) at 0° C. The mixture was stirred at 25° C. for 1 h. TLC (petroleum ether/ethyl acetate=1/1, R_{f}(Cpd.4)=0.60) indicated that (4) was consumed completely, and two new spots formed. LC-MS (ET65158-9-P1A1) showed that (4) was consumed completely and one main peak with the desired MS was detected. The reaction mixture was quenched by the addition of NH₄Cl (15 ml) at 0° C., and then extracted with 45 mL of EtOAc (15 mL×3). The combined organic layers were dried over Na₂SO₄, filtered, and concentrated under reduced pressure to give a residue. The residue was purified by Prep-TLC (petroleum ether/ethyl acetate=1/1). 2-(3,5-difluoro-4-(2-hydroxypropan-2-yl)phenyl)-3,5,7,8-tetrahydro-4H-thiopyrano[4,3-d]pyrimidin-4-one (5) (150 mg, crude) was obtained and used immediately in the next step. LC-MS (ET65158-9-P1A1, product: Rₜ=0.575 mins).

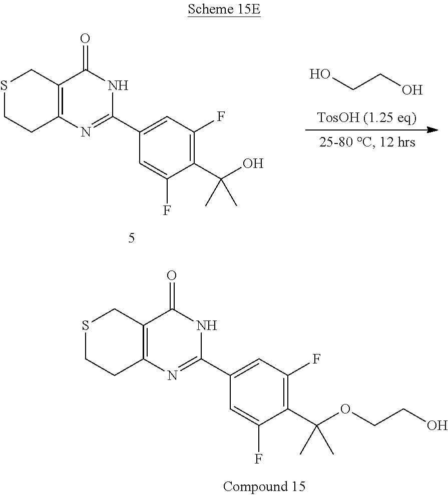

To a solution of 2-(3,5-difluoro-4-(2-hydroxypropan-2-yl)phenyl)-3,5,7,8-tetrahydro-4H-thiopyrano[4,3-d]pyrimidin-4-one (5) (20.0 mg, 59.11 mol, 1.0 eq) in ethylene glycol (1.59 g, 25.62 mmol, 1.43 mL, 433.41 eq) was added TosOH (12.7 mg, 73.88 mol, 1.25 eq) at 25° C. The mixture was stirred at 80° C. for 12 h. TLC (petroleum ether/ethyl acetate=0/1, R_{f}(Cpd.5)=0.53) indicated that (5) was consumed completely, and two new spots formed. After cooling to 25° C., the mixture was concentrated under reduced pressure to give a residue. The residue was purified by prep-TLC (SiO₂, petroleum ether/ethyl acetate=0/1) to provide 2-(3,5-difluoro-4-(2-(2-hydroxyethoxy)propan-2-yl)phenyl)-3,5,7,8-tetrahydro-4H-thiopyrano[4,3-d]pyrimidin-4-one (Compound 15) was obtained. LC-MS: 383.1 (M+1). ¹H NMR (400 MHz, DMSO-d₆): δ=7.78-7.70 (m, 2H), 4.49-4.45 (m, 1H) 3.47-3.43 (m, 4H), 3.21-3.17 (m, 2H), 2.85~2.75 (m, 4H), 1.637 (s, 6H).

### Compound 16

### Synthesis of 2-(3,5-difluoro-4-(2-(2-methoxyethoxy)propan-2-yl)phenyl)-3,5,7,8-tetrahydro-4H-thiopyrano[4,3-d]pyrimidin-4-one (Compound 16)

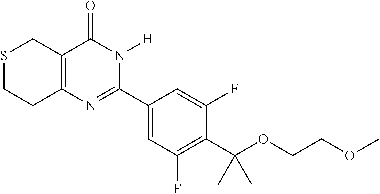 [SMILES: COCCOC(C)(C)c1c(F)cc(-c2nc3c(c(=O)[nH]2)CSCC3)cc1F]

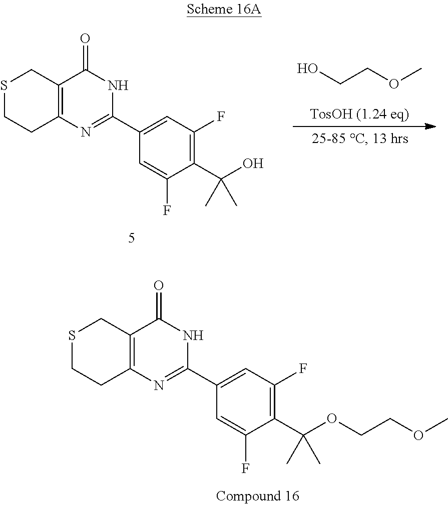

Synthesis of 2-(3,5-difluoro-4-(2-hydroxypropan-2-yl)phenyl)-3,5,7,8-tetrahydro-4H-thiopyrano[4,3-d]pyrimidin-4-one (5) is shown in Schemes 15A-15D.

To a solution of 2-(3,5-difluoro-4-(2-hydroxypropan-2-yl)phenyl)-3,5,7,8-tetrahydro-4H-thiopyrano[4,3-d]pyrimidin-4-one (5) (50.0 mg, 147 μmol, 1.0 eq) in 2-methoxyethanol (0.5 mL) was added TosOH (31.6 mg, 183 μmol, 1.24 eq) at 25° C. and then heated to 85° C. and stirred for 13 h. LC-MS showed ~17% of (5) remained. Several new peaks were shown on LC-MS, and the desired compound was detected. After cooling to 25° C., the reaction mixture was purified by prep-HPLC (neutral condition; column: Waters Xbridge BEH C18 100×30 mm×10 um; mobile phase: [water (NH₄HCO₃)-can]; B %: 25%-55%, 8 mins) to provide 2-(3,5-difluoro-4-(2-(2-methoxyethoxy)propan-2-yl)phenyl)-3,5,7,8-tetrahydro-4H-thiopyrano[4,3-d]pyrimidin-4-one (Compound 16). LC-MS: 397.1 (M+1). ¹H NMR (400 MHz, DMSO-d₆): δ=7.81~7.75 (m, 2H), 3.549 (s, 2H) 3.53~3.38 (m, 2H), 3.35-3.25 (m, 2H), 3.22 (s, 3H), 2.906 (m, 4H), 1.653 (s, 6H).

### Compound 17

### Synthesis of 2-(4-(2-(2-methoxyethoxy)propan-2-yl)phenyl)-3,5,7,8-tetrahydro-4H-thiopyrano[4,3-d]pyrimidin-4-one (Compound 17)

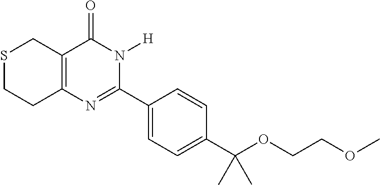 [SMILES: COCCOC(C)(C)c1ccc(-c2nc3c(c(=O)[nH]2)CSCC3)cc1]

### Synthesis of 1-bromo-4-(2-(2-methoxyethoxy)propan-2-yl)benzene

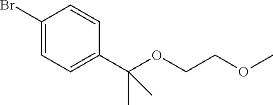

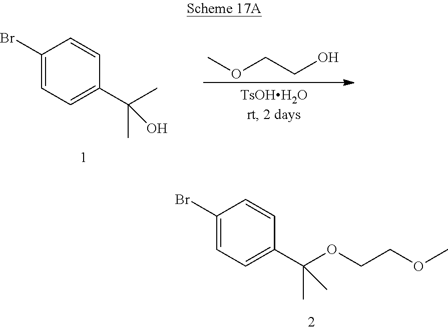

To solution of 2-(4-bromophenyl)propan-2-ol (1) (2.6 g, 12.2 mmol, 1.0 eq) in 2-methoxyethan-1-ol (20 mL) was added TsOH H₂O (2.3 g, 12.2 mmol, 1 eq). The mixture was stirred at RT for 2 days, then quenched with water (100 mL) and extracted with EtOAc (3×60 mL). The organic layers were concentrated and the residue was purified by column chromatography on silica gel eluted with 0-30% EtOAc/hexane to afford 1-bromo-4-(2-(2-methoxyethoxy)propan-2-yl)benzene (2) (2.7 g). ¹H NMR (400 MHz, DMSO-d₆): δ 7.54-7.51 (m, 2H), 7.38-7.35 (m, 2H), 3.42-3.40 (m, 2H), 3.24-3.22 (m, 5H), 1.44 (s, 6H).

### Synthesis of 4-(2-(2-methoxyethoxy)propan-2-yl)benzonitrile

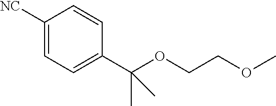 [SMILES: COCCOC(C)(C)c1ccc(C#N)cc1]

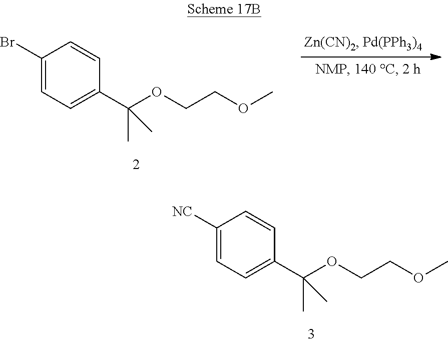 [SMILES: COCCOC(C)(C)c1ccc(Br)cc1 ; COCCOC(C)(C)c1ccc(C#N)cc1]

To a solution of 1-bromo-4-(2-(2-methoxyethoxy)propan-2-yl)benzene (2) (1.5 g, 3.7 mmol, 1 eq) in NMP (10 mL) was added Zn(CN)₂ (260 mg, 2.2 mmol, 0.6 eq) and Pd(PPh₃)₄ (430 mg, 0.37 mmol, 0.1 eq). The resulting solution was stirred for 2 h at 140° C. under Ar. The resulting mixture was cooled to RT, diluted with water (50 mL), and extracted with EtOAc (3×60 mL). The organic layers were combined, washed with brine (100 mL), dried over anhydrous sodium sulfate, and concentrated. The residue was purified by column chromatography on silica gel eluted with 0-40% EtOAc/hexane to give 4-(2-(2-methoxyethoxy)propan-2-yl)benzonitrile (3) (600 mg). ¹H NMR (400 MHz, DMSO-d₆): δ 7.81 (d, J=8.4 Hz, 2H), 7.62 (d, J=8.4 Hz, 2H), 3.44 (t, J=5.2 Hz, 2H), 3.28-3.25 (m, 5H), 1.47 (s, 6H).

### Synthesis of 4-(2-(2-methoxyethoxy)propan-2-yl)benzimidamide

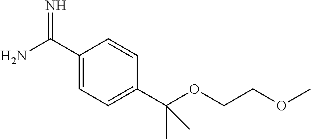 [SMILES: COCCOC(C)(C)c1ccc(C(=N)N)cc1]

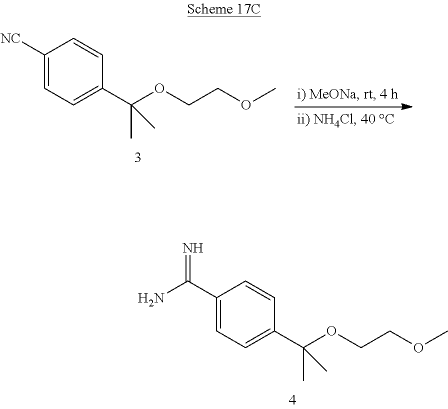 [SMILES: COCCOC(C)(C)c1ccc(C#N)cc1 ; COCCOC(C)(C)c1ccc(C(=N)N)cc1]

To a solution of 4-(2-(2-methoxyethoxy)propan-2-yl)benzonitrile (3) (0.6 g, 2.7 mmol, 1.0 eq) in MeOH (5 mL) was added MeONa (295 mg, 5.5 mmol, 2.0 eq); the mixture was stirred at RT for 4 h. NH₄Cl (287 mg, 5.5 mmol, 2.0 eq) was added and then the mixture was stirred at 40° C. for 16 h. The mixture was cooled to RT, filtered, and then the filtrate was concentrated to give 4-(2-(2-methoxyethoxy)propan-2-yl)benzimidamide (4) (285 mg, crude). LC-MS: 237.2 [M+H]⁺.

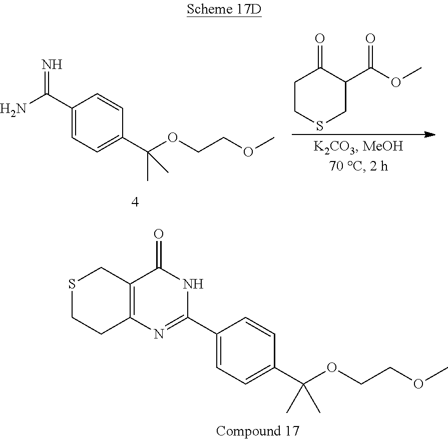

To a solution of 4-(2-(2-methoxyethoxy)propan-2-yl)benzimidamide (4) (150 mg, 0.7 mmol, 1.0 eq) in MeOH (5 mL) was added K₂CO₃ (180 mg, 1.3 mmol, 2.0 eq) and methyl 4-oxotetrahydro-2H-thiopyran-3-carboxylate (80 mg, 0.5 mmol, 0.7 eq) under N₂. The mixture was stirred at 70° C. for 2 h. It was then concentrated and purified by Prep-HPLC to give 2-(4-(2-(2-methoxyethoxy)propan-2-yl)phenyl)-3,5,7,8-tetrahydro-4H-thiopyrano[4,3-d]pyrimidin-4-one (Compound 17). LC-MS: 361.1 [M+H]⁺. ¹H NMR (400 MHz, DMSO-d₆): δ 12.70 (s, 1H), 8.06 (d, J=8.6 Hz, 2H), 7.54 (d, J=8.6 Hz, 2H), 3.52 (s, 2H), 3.46-3.41 (m, 2H), 3.25 (d, J=5.0 Hz, 5H), 2.88 (dd, J=7.6, 4.0 Hz, 4H), 1.48 (s, 6H).

### Compound 18 Synthesis of methyl (2-((2-(4-(4-oxo-3,5,7,8-tetrahydro-4H-thiopyrano[4,3-d]pyrimidin-2-yl)phenyl)propan-2-yl)oxy)ethyl)carbamate (Compound 18)

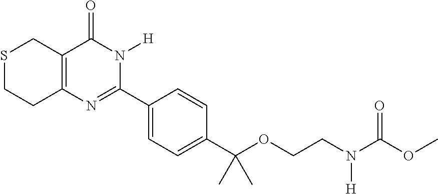 [SMILES: COC(=O)NCCOC(C)(C)c1ccc(-c2nc3c(c(=O)[nH]2)CSCC3)cc1]

### Synthesis of 1-bromo-4-(2-(2-nitroethoxy)propan-2-yl)benzene

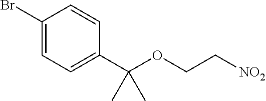 [SMILES: CC(C)(OCC[N+](=O)[O-])c1ccc(Br)cc1]

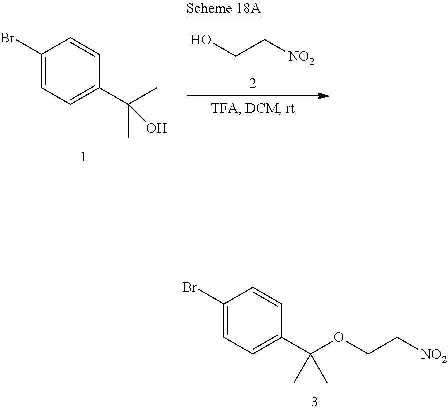 [SMILES: CC(C)(O)c1ccc(Br)cc1 ; O=[N+]([O-])CCO ; CC(C)(OCC[N+](=O)[O-])c1ccc(Br)cc1]

To a solution of 2-(4-bromophenyl)propan-2-ol (1) (2.6 g, 12.2 mmol, 1.0 eq) in DCM (20 mL) was added TFA (2.3 g, 12.2 mmol, 1.0 eq) and 2-nitroethan-1-ol (2) (10 mL). The mixture was stirred at RT for 3 days. The reaction was quenched with water (100 mL) and extracted with EtOAc (3×60 mL). The organic layers were concentrated, and the residue was purified by column chromatography on silica gel eluted with 0-30% EtOAc/hexane to afford 1-bromo-4-(2-(2-nitroethoxy)propan-2-yl)benzene (3) (2.7 g). ¹H NMR (400 MHz, CDCl3): δ 7.80 (d, J=8.8 Hz, 2H), 7.31 (d, J=8.4 Hz, 2H), 4.55 (t, J=4.8 Hz, 2H), 3.69 (t, J=4.8 Hz, 2H), 1.51 (s, 6H).

### Synthesis of 2-((2-(4-bromophenyl)propan-2-yl)oxy)ethan-1-amine

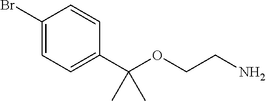

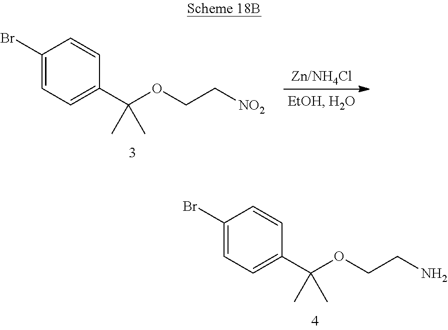 [SMILES: CC(C)(OCC[N+](=O)[O-])c1ccc(Br)cc1 ; CC(C)(OCCN)c1ccc(Br)cc1]

To a solution of 1-bromo-4-(2-(2-nitroethoxy)propan-2-yl)benzene (3) (1.5 g, 3.7 mmol, 1.0 eq) in EtOH/H₂O (10 mL/2 mL) was added Zn (1.2 g, 18.5 mmol, 5.0 eq) and NH₄Cl (1.0 g, 18.5 mmol, 5.0 eq). The resulting mixture was stirred for 12 h at 80° C. under Ar. The reaction mixture was cooled to RT, diluted with water (50 mL), and extracted with EtOAc (3×60 mL). The organic layers were combined, washed with brine (100 mL), dried over anhydrous sodium sulfate, and concentrated to give 2-((2-(4-bromophenyl)propan-2-yl)oxy)ethan-1-amine (4) (1.2 g, crude) LC-MS: 258.0, 260.0 [M+H]⁺.

### Synthesis of methyl (2-((2-(4-bromophenyl)propan-2-yl)oxy)ethyl)carbamate

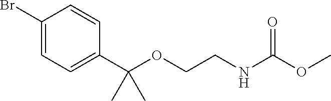 [SMILES: COC(=O)NCCOC(C)(C)c1ccc(Br)cc1]

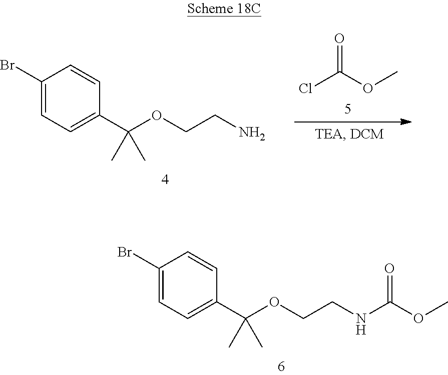

To a solution of 2-((2-(4-bromophenyl)propan-2-yl)oxy)ethan-1-amine (4) (1.2 g, 4.7 mmol, 1.0 eq) in DCM (10 mL) was added TEA (707 mg, 7.0 mmol, 1.5 eq), followed by the addition of methyl carbonochloridate (5) (395 mg, 4.2 mmol, 0.9 eq). The mixture was stirred at RT for one day. The reaction was concentrated and the residue was purified by column chromatography on silica gel eluted with 0-30% EtOAc/hexane to afford methyl (2-((2-(4-bromophenyl)propan-2-yl)oxy)ethyl)carbamate (6) (1.0 g). LC-MS: 357.0, 359.0 [M+H+MeCN]⁺.

### Synthesis of methyl (2-((2-(4-cyanophenyl)propan-2-yl)oxy)ethyl)carbamate

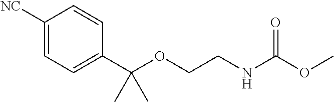 [SMILES: COC(=O)NCCOC(C)(C)c1ccc(C#N)cc1]

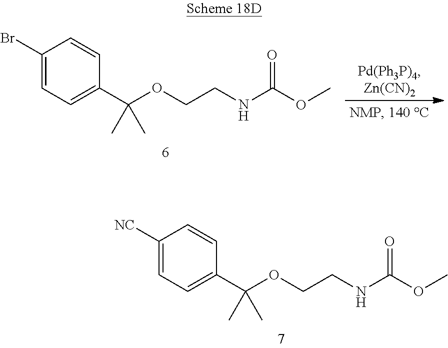 [SMILES: COC(=O)NCCOC(C)(C)c1ccc(Br)cc1 ; COC(=O)NCCOC(C)(C)c1ccc(C#N)cc1]

To a solution of methyl (2-((2-(4-bromophenyl)propan-2-yl)oxy)ethyl)carbamate (6) (1.0 g, 3.7 mmol, 1 eq) in NMP (10 mL) was added Zn(CN)₂ (260 mg, 2.2 mmol, 0.6 eq), followed by Pd(PPh₃)₄ (430 mg, 0.37 mmol, 0.1 eq). The resulting solution was stirred for 2 h at 140° C. under Ar. The resulting mixture was cooled to RT, diluted with water (50 mL), and extracted with EtOAc (3×20 mL). The organic layers were combined, washed with brine (100 mL), dried over anhydrous sodium sulfate, and concentrated. The residue was purified by column chromatography on silica gel eluted with 0-40% EtOAc/hexane to give methyl (2-((2-(4-cyanophenyl)propan-2-yl)oxy)ethyl)carbamate (7) (600 mg). ¹H NMR (400 MHz, DMSO): δ 7.86-7.76 (m, 2H), 7.64-7.58 (m, 2H), 7.25-7.11 (m, 1H), 3.33 (s, 3H), 3.15-3.04 (m, 4H), 1.46 (s, 6H).

### Synthesis of methyl (2-((2-(4-carbamimidoylphenyl)propan-2-yl)oxy)ethyl)carbamate

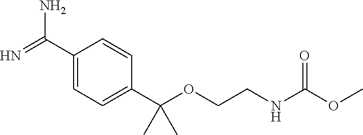 [SMILES: COC(=O)NCCOC(C)(C)c1ccc(C(=N)N)cc1]

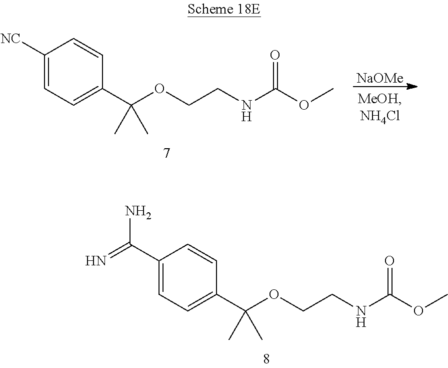 [SMILES: COC(=O)NCCOC(C)(C)c1ccc(C#N)cc1 ; COC(=O)NCCOC(C)(C)c1ccc(C(=N)N)cc1 ; "NaOMe"]

To a solution of methyl (2-((2-(4-cyanophenyl)propan-2-yl)oxy)ethyl)carbamate (7) (0.6 g, 2.7 mmol, 1.0 eq) in MeOH (5 mL) was added MeONa (295 mg, 5.5 mmol, 2.0 eq); the mixture was stirred at RT for 4 h. NH₄Cl (287 mg, 5.5 mmol, 2.0 eq) was added and the mixture was stirred at 40° C. for 16 h. The mixture was cooled to RT, filtered, and the filtrate was concentrated to give methyl (2-((2-(4-carbamimidoylphenyl)propan-2-yl)oxy)ethyl)carbamate (8) (285 mg, crude). LC-MS: 280.2 [M+H]⁺.

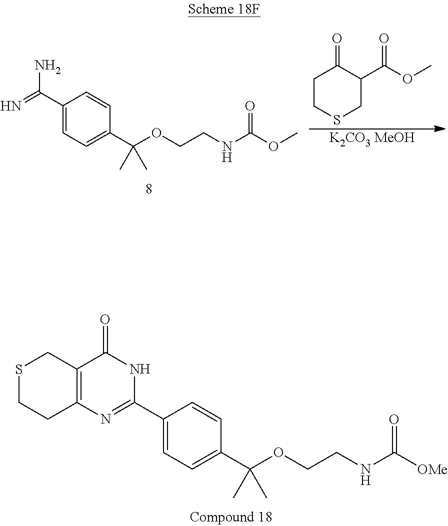

To a solution of methyl (2-((2-(4-carbamimidoylphenyl)propan-2-yl)oxy)ethyl)carbamate (8) (285 mg, 0.7 mmol, 1.0 eq) in MeOH (5 mL) was added K₂CO₃ (180 mg, 1.3 mmol, 2.0 eq) and methyl 4-oxotetrahydro-2H-thiopyran-3-carboxylate (80 mg, 0.5 mmol, 0.7 eq) under N₂. The mixture was stirred at 70° C. for 2 h, and then concentrated and purified by Prep-HPLC to give methyl (2-((2-(4-(4-oxo-3,5,7,8-tetrahydro-4H-thiopyrano[4,3-d]pyrimidin-2-yl)phenyl)propan-2-yl)oxy)ethyl)carbamate (Compound 18). LC-MS: 404.2 [M+H]⁺. ¹H NMR (400 MHz, DMSO): δ 12.71 (s, 1H), 8.05 (d, J=8.2 Hz, 2H), 7.56 (d, J=8.4 Hz, 2H), 7.16 (s, 1H), 3.51 (m, 5H), 3.12 (dd, J=12.5, 5.0 Hz, 4H), 2.89 (d, J=4.0 Hz, 4H), 1.47 (s, 6H).

### Compound 19

### Synthesis of 2-(6′-bromo-[2,3′-bipyridin]-5-yl)-3,5,7,8-tetrahydro-4H-thiopyrano[4,3-d]pyrimidin-4-one (Compound 19)

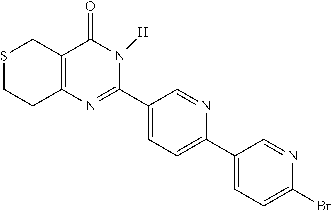 [SMILES: O=c1[nH]c(-c2ccc(-c3ccc(Br)nc3)nc2)nc2c1CSCC2]

### Synthesis of 6′-bromo-[2,3′-bipyridine]-5-carbonitrile

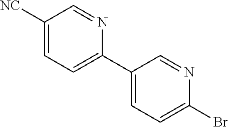 [SMILES: N#Cc1ccc(-c2ccc(Br)nc2)nc1]

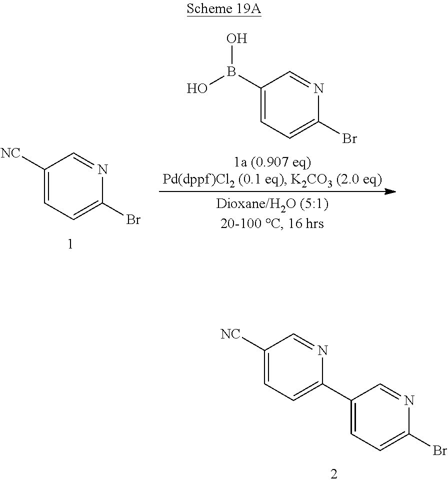 [SMILES: OB(O)c1ccc(Br)nc1 ; N#Cc1ccc(Br)nc1 ; N#Cc1ccc(-c2ccc(Br)nc2)nc1]

To a solution of 6-bromonicotinonitrile (1) (2.00 g, 10.93 mmol, 1.0 eq) and (6-bromopyridin-3-yl)boronic acid (1a) (2.00 g, 9.91 mmol, 9.07e-1 eq) in dioxane (20 mL)/H₂O (4 mL) was added K₂CO₃ (3.02 g, 21.86 mmol, 2.0 eq) and Pd(dppf)Cl₂ (799.65 mg, 1.09 mmol, 0.1 eq) at 20° C. under N₂. The mixture was stirred at 100° C. for 16 h under N₂. After cooling to 25° C., the mixture was filtered and then the filtrate was concentrated under reduced pressure to give a residue that was purified by column chromatography (SiO₂, petroleum ether/ethyl acetate=50/1 to 0/1). 6′-bromo-[2,3′-bipyridine]-5-carbonitrile (2) (730 mg) was obtained. ¹H NMR: (400 MHz, CDCl₃): δ=8.92 (dd, J=1.8, 12.3 Hz, 2H), 8.19 (dd, J=2.5, 8.3 Hz, 1H), 8.00 (dd, J=2.0, 8.3 Hz, 1H), 7.81 (d, J=8.3 Hz, 1H), 7.58 (d, J=8.3 Hz, 1H)

### Synthesis of 6′-bromo-[2,3′-bipyridine]-5-carboximidamide

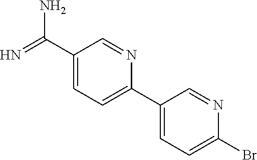 [SMILES: N=C(N)c1ccc(-c2ccc(Br)nc2)nc1]

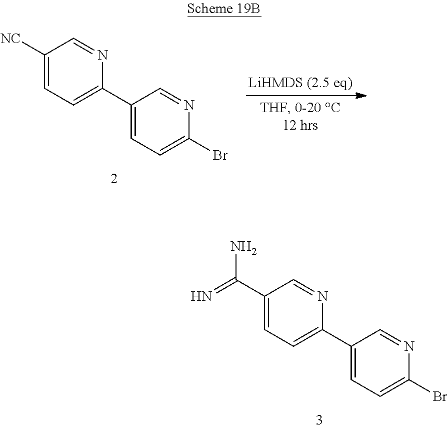 [SMILES: N#Cc1ccc(-c2ccc(Br)nc2)nc1 ; N=C(N)c1ccc(-c2ccc(Br)nc2)nc1]

To a solution of 6′-bromo-[2,3′-bipyridine]-5-carbonitrile (2) (280 mg, 1.08 mmol, 1.0 eq) in THF (3 mL) was added LiHMDS (1 M, 2.69 mL, 2.5 eq) at 0° C. under N₂. The mixture was warmed to 20° C. and stirred at 20° C. for 12 h. The reaction mixture was quenched by addition of HCl/dioxane (4 mol/L, 1 mL) at 0° C. and then concentrated under reduced pressure to give a residue. The residue was suspended in MeOH (7 mL), and the mixture stirred at 20° C. for 1 hr. Then the mixture was filtered, and the filter cake was dried under reduced pressure to give a 6′-bromo-[2,3′-bipyridine]-5-carboximidamide (3) (350 mg, crude, HCl) and used immediately in the next step.

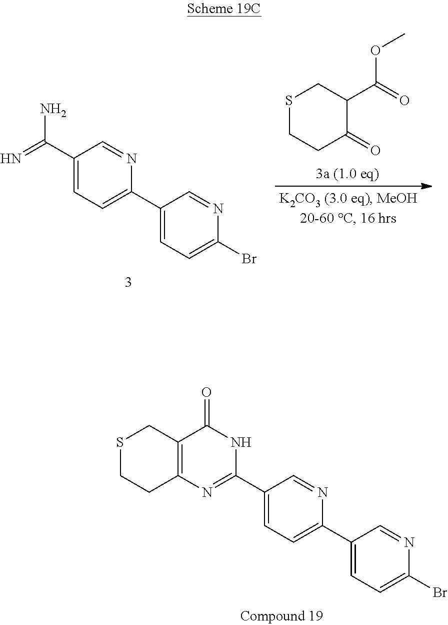 [SMILES: N=C(N)c1ccc(-c2ccc(Br)nc2)nc1 ; COC(=O)C1CSCCC1=O ; O=c1[nH]c(-c2ccc(-c3ccc(Br)nc3)nc2)nc2c1CSCC2]

To a solution of 6′-bromo-[2,3′-bipyridine]-5-carboximidamide (3) (350 mg, 1.12 mmol, 1.0 eq, HCl) in MeOH (3.5 mL) was added methyl 4-oxotetrahydro-2H-thiopyran-3-carboxylate (3a) (194.45 mg, 1.12 mmol, 1.0 eq) and K₂CO₃ (462.79 mg, 3.35 mmol, 3.0 eq) at 20° C. The mixture was stirred at 60° C. for 16 h. The mixture was filtered, and concentrated under reduced pressure to give a residue that was purified by prep-HPLC (neutral condition; column: Phenomenex C18 75*30 mm*3 um; mobile phase: [water (NH₄HCO₃)-ACN]; B %: 25%-40%, 10 mins). 2-(6′-bromo-[2,3′-bipyridin]-5-yl)-3,5,7,8-tetrahydro-4H-thiopyrano[4,3-d]pyrimidin-4-one (Compound 19) was obtained. LC-MS: 401 (M+1). ¹H NMR: (400 MHz, DMSO-d₆): =9.27 (d, J=1.3 Hz, 1H), 8.94 (d, J=2.2 Hz, 1H), 8.43 (dd, J=1.9, 8.3 Hz, 1H), 8.27 (dd, J=2.3, 8.4 Hz, 1H), 7.90 (d, J=8.3 Hz, 1H), 7.61 (d, J=8.4 Hz, 1H), 3.28 (s, 2H), 2.61 (br dd, J=5.0, 15.4 Hz, 4H).

### Compound 20

### Synthesis of 2-(4-(6-bromopyridin-3-yl)-3-fluorophenyl)-3,5,7,8-tetrahydro-4H-thiopyrano[4,3-d]pyrimidin-4-one (Compound 20)

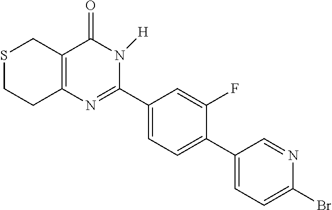 [SMILES: O=c1[nH]c(-c2ccc(-c3ccc(Br)nc3)c(F)c2)nc2c1CSCC2]

### Synthesis of 4-(6-bromopyridin-3-yl)-3-fluorobenzonitrile

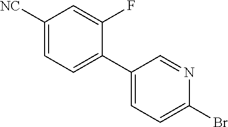 [SMILES: N#Cc1ccc(-c2ccc(Br)nc2)c(F)c1]

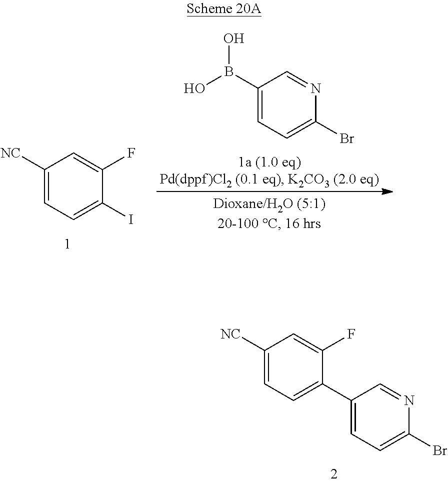 [SMILES: OB(O)c1ccc(Br)nc1 ; N#Cc1ccc(I)c(F)c1 ; N#Cc1ccc(-c2ccc(Br)nc2)c(F)c1]

To a solution of (6-bromopyridin-3-yl)boronic acid (1a) (408.52 mg, 2.02 mmol, 1.0 eq) in dioxane (5 mL)/H₂O (1 mL) was added 3-fluoro-4-iodobenzonitrile (1) (0.5 g, 2.02 mmol, 1.0 eq), K₂CO₃ (559.52 mg, 4.05 mmol, 2.0 eq), and Pd(dppf)Cl₂ (148.11 mg, 202.42 mol, 0.1 eq) at 20° C. under N₂. The mixture was stirred at 100° C. for 16 h under N₂. After cooling to 20° C., the mixture was filtered, and concentrated under reduced pressure to give a residue that was purified by column chromatography (SiO₂, petroleum ether/ethyl acetate=20/1 to 0/1). 4-(6-bromopyridin-3-yl)-3-fluorobenzonitrile (2) (200 mg, crude) was obtained and used as is in the next step. ¹H NMR: (400 MHz, CDCl₃): δ=8.48 (s, 1H), 7.68 (td, J=1.9, 8.3 Hz, 1H), 7.56 (s, 1H), 7.50 (br d, J=7.5 Hz, 2H), 7.47-7.45 (m, 1H).

### Synthesis of 4-(6-bromopyridin-3-yl)-3-fluorobenzimidamide

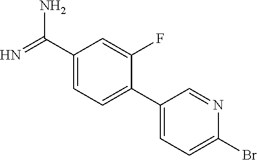 [SMILES: N=C(N)c1ccc(-c2ccc(Br)nc2)c(F)c1]

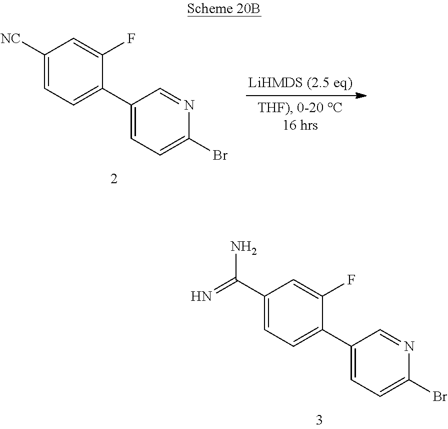 [SMILES: N#Cc1ccc(-c2ccc(Br)nc2)c(F)c1 ; N=C(N)c1ccc(-c2ccc(Br)nc2)c(F)c1]

To a solution of 4-(6-bromopyridin-3-yl)-3-fluorobenzonitrile (2) (200 mg, 721.78 mol, 1.0 eq) in THE (2 mL) was added LiHMDS (1 M, 1.80 mL, 2.5 eq) at 0° C. under N₂. The mixture was warmed to 20° C. and stirred at 20° C. for 16 h. The reaction mixture was quenched by addition of HCl/dioxane (4 mol/L, 0.6 mL) at 0° C. and then concentrated under reduced pressure to give a residue that was suspended in MeOH (3 ml), and the mixture stirred at 20° C. for 1 h. Then the mixture was filtered, and the filter cake was dried under reduced pressure to give 4-(6-bromopyridin-3-yl)-3-fluorobenzimidamide (3) (0.11 g, crude) that was used in the next step without further purification.

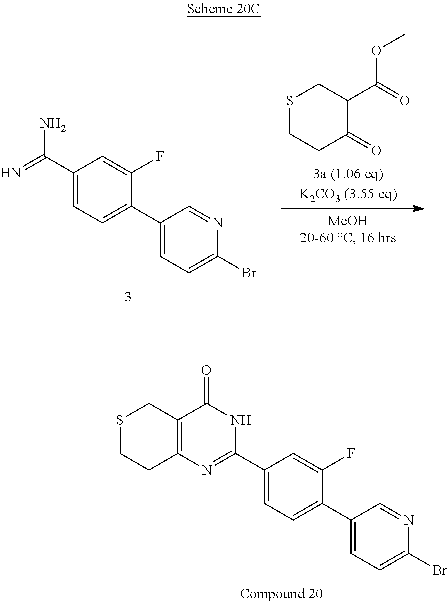 [SMILES: N=C(N)c1ccc(-c2ccc(Br)nc2)c(F)c1 ; COC(=O)C1CSCCC1=O ; O=c1[nH]c(-c2ccc(-c3ccc(Br)nc3)c(F)c2)nc2c1CSCC2]

To a solution of methyl 4-oxotetrahydro-2H-thiopyran-3-carboxylate (3a) (68.75 mg, 394.62 μmol, 1.06 eq) and 4-(6-bromopyridin-3-yl)-3-fluorobenzimidamide (3) (0.11 g, 373.99 mol, 1.0 eq) in MeOH (2 mL) was added K₂CO₃ (183.33 mg, 1.33 mmol, 3.55 eq) at 20° C. The mixture was stirred at 60° C. for 16 h. After cooling to 20° C., the mixture was filtered, and then the filtrate was concentrated under reduced pressure to give a residue that was purified by prep-HPLC (neutral condition; column: Waters Xbridge Prep OBD C18 150*40 mm*10 m; mobile phase: [water (NH₃H₂O+NH₄HCO₃)-ACN]; B %: 30%-60%, 8 mins). 2-(4-(6-bromopyridin-3-yl)-3-fluorophenyl)-3,5,7,8-tetrahydro-4H-thiopyrano[4,3-d]pyrimidin-4-one (Compound 20) was obtained. LC-MS: 417+bromo isomers (M+1).

¹H NMR: (400 MHz, DMSO-d₆): δ 8.66 (s, 1H), 8.13-8.06 (m, 2H), 8.06-8.00 (m, 1H), 7.86-7.77 (m, 2H), 3.55 (s, 2H), 2.91 (s, 4H).

### Compound 21

### Synthesis of 2-(4-(6-bromopyridin-3-yl)-3,5-difluorophenyl)-3,5,7,8-tetrahydro-4H-thiopyrano[4,3-d]pyrimidin-4-one (Compound 21)

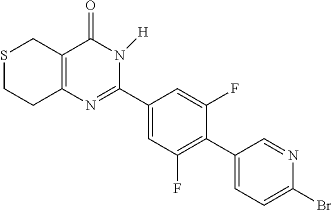 [SMILES: O=c1[nH]c(-c2cc(F)c(-c3ccc(Br)nc3)c(F)c2)nc2c1CSCC2]

### Synthesis of 3,5-difluoro-4-iodobenzonitrile

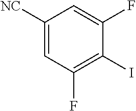 [SMILES: N#Cc1cc(F)c(I)c(F)c1]

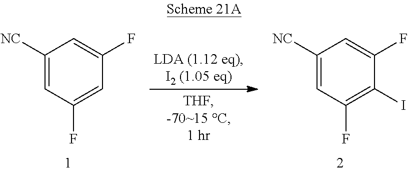 [SMILES: N#Cc1cc(F)cc(F)c1 ; N#Cc1cc(F)c(I)c(F)c1]

To a solution of 3,5-difluorobenzonitrile (1) (2.00 g, 14.07 mmol, 1.0 eq) in THF (20 mL), LDA (2 M, 7.86 mL, 1.12 eq) was added dropwise at −70° C. Then I₂ (3.76 g, 14.80 mmol, 2.98 mL, 1.05 eq) in THF (10 mL) at −70° C. was added at −70° C. The reaction mixture was slowly warmed to 15° C. then stirred at 15° C. for 1 hr. The mixture was quenched with 10% sodium thiosulfite solution (15 ml). The reaction mixture was extracted with a 1/1 mixture of ethyl acetate/hexanes (7 mL×3). The combined organic phase was dried over Na₂SO₄ and concentrated in vacuo. The residue was purified by prep-HPLC (neutral condition; column: Welch Xtimate C18 250*70 mm #10 m; mobile phase: [water (NH₄HCO₃)-ACN]; B %: 40%-70%, 20 mins). 3,5-difluoro-4-iodobenzonitrile (2) (2.00 g) was obtained. H NMR: (400 MHz, CDCl₃): δ=7.23-7.18 (m, 2H).

### Synthesis of 4-(6-bromopyridin-3-yl)-3,5-difluorobenzonitrile

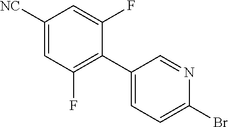 [SMILES: N#Cc1cc(F)c(-c2ccc(Br)nc2)c(F)c1]

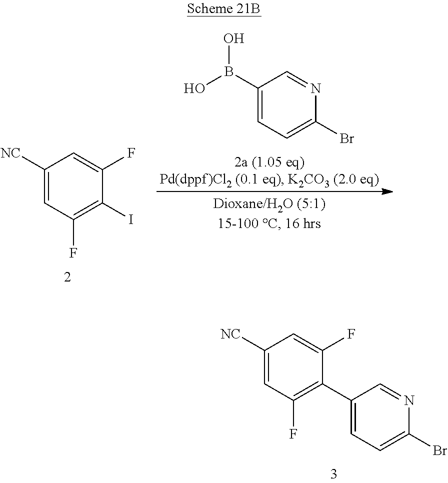 [SMILES: OB(O)c1ccc(Br)nc1 ; N#Cc1cc(F)c(I)c(F)c1 ; N#Cc1cc(F)c(-c2ccc(Br)nc2)c(F)c1]

To a solution of (6-bromopyridin-3-yl)boronic acid (2a) (1.60 g, 7.93 mmol, 1.05 eq) and 3,5-difluoro-4-iodobenzonitrile (2) (2.00 g, 11.32 mmol, 1.0 eq) in dioxane (20 mL)/H₂O (4 mL) was added K₂CO₃ (2.09 g, 15.09 mmol, 2.0 eq) and Pd(dppf)Cl₂ (552.23 mg, 0.75 mmol, 0.1 eq) at 15° C. under N₂. The mixture heated to 100° C. for 16 h under N₂. After cooling to 15° C., the mixture was filtered, and then the filtrate was concentrated under reduced pressure to give a residue. The residue was purified by column chromatography (SiO₂, petroleum ether/ethyl acetate=50/1 to 0/1). 4-(6-bromopyridin-3-yl)-3,5-difluorobenzonitrile (3) (620 mg, 210 mmol) was obtained. ¹H NMR: (400 MHz, CDCl₃): δ=8.43 (s, 1H), 7.63-7.55 (m, 2H), 7.34-7.26 (m, 2H).

### Synthesis of 4-(6-bromopyridin-3-yl)-3,5-difluorobenzimidamide

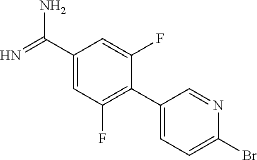 [SMILES: N=C(N)c1cc(F)c(-c2ccc(Br)nc2)c(F)c1]

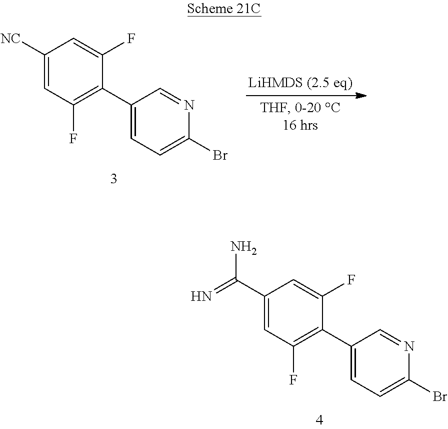 [SMILES: N#Cc1cc(F)c(-c2ccc(Br)nc2)c(F)c1 ; N=C(N)c1cc(F)c(-c2ccc(Br)nc2)c(F)c1]

To a solution of 4-(6-bromopyridin-3-yl)-3,5-difluorobenzonitrile (3) (500 mg, 1.69 mmol, 1.0 eq) in THF (5 mL) was added LiHMDS (1 M, 4.24 mL, 2.5 eq) at 0° C. under N₂. The mixture was warmed to 20° C. and stirred for 16 h. The reaction mixture was quenched by addition of HCl/dioxane (4 mol/L, 2.0 mL) at 0° C., and then concentrated under reduced pressure to give a residue. The residue was suspended in MeOH (8 ml), and the mixture was stirred at 20° C. for 1 hr. Then the mixture was filtered, and the filtrate was concentrated under reduced pressure to give 4-(6-bromopyridin-3-yl)-3,5-difluorobenzimidamide (4) (540 mg, crude, HCl). The compound was used immediately in the next step.

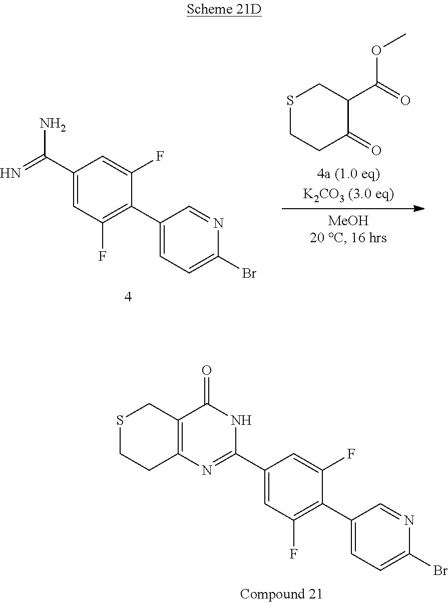 [SMILES: N=C(N)c1cc(F)c(-c2ccc(Br)nc2)c(F)c1 ; COC(=O)C1CSCCC1=O ; O=c1[nH]c(-c2cc(F)c(-c3ccc(Br)nc3)c(F)c2)nc2c1CSCC2]

To a solution of 4-(6-bromopyridin-3-yl)-3,5-difluorobenzimidamide (4) (440 mg, 1.41 mmol, 1.0 eq) in MeOH (4.4 mL) was added methyl 4-oxotetrahydro-2H-thiopyran-3-carboxylate (4a) (245.60 mg, 1.41 mmol, 1.0 eq) and K₂CO₃ (584.52 mg, 4.23 mmol, 3.0 eq) at 20° C. The mixture was stirred at 20° C. for 16 h. The mixture was filtered, and the filtrate was concentrated under reduced pressure to give a residue. The residue was purified by prep-HPLC (neutral condition; column: NP-1; mobile phase: [Heptane-EtOH]; B %: 10%-70%, mins). 2-(4-(6-bromopyridin-3-yl)-3,5-difluorophenyl)-3,5,7,8-tetrahydro-4H-thiopyrano[4,3-d]pyrimidin-4-one (Compound 21) was obtained. LC-MS: 438+bromo isomers (M+1). ¹H NMR: (400 MHz, DMSO-d₆): =13.06-12.89 (m, 1H), 8.64 (s, 1H), 8.10-8.00 (m, 3H), 7.93 (d, J=8.4 Hz, 1H), 3.62 (s, 2H), 2.98 (s, 4H).

### Compound 22

### Synthesis of (6-(4-(4-oxo-3,5,7,8-tetrahydro-4H-thiopyrano[4,3-d]pyrimidin-2-yl)phenyl)pyridin-3-yl)boronic acid (Compound 22)

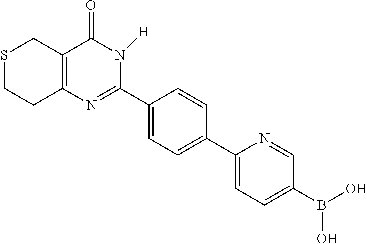 [SMILES: O=c1[nH]c(-c2ccc(-c3ccc(B(O)O)cn3)cc2)nc2c1CSCC2]

### Synthesis of 4-(5-bromopyridin-2-yl)benzonitrile

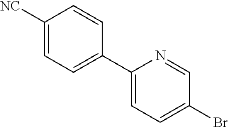 [SMILES: N#Cc1ccc(-c2ccc(Br)cn2)cc1]

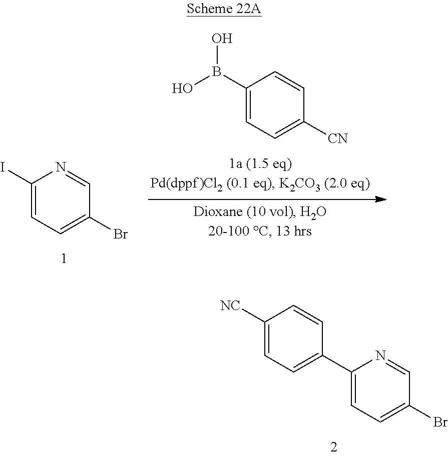 [SMILES: N#Cc1ccc(B(O)O)cc1 ; Brc1ccc(I)nc1 ; N#Cc1ccc(-c2ccc(Br)cn2)cc1]

To a solution of 5-bromo-2-iodopyridine (1) (0.5 g, 1.76 mmol, 1.0 eq) and (4-cyanophenyl)boronic acid (1a) (388.19 mg, 2.64 mmol, 1.5 eq) in dioxane (5 mL)/H₂O (1 mL) was added Pd(dppf)Cl₂ (128.87 mg, 176.12 μmol, 0.1 eq) and K₂CO₃ (486.83 mg, 3.52 mmol, 2.0 eq) at 20° C. under N₂. The mixture was stirred at 100° C. for 13 h. After cooling to 20° C., the mixture was filtered, and then the filtrate was concentrated under reduced pressure to give a residue. The residue was purified by column chromatography (SiO₂, petroleum ether/ethyl acetate=50/1 to 0/1). 4-(5-bromopyridin-2-yl)benzonitrile (2) (400 mg, crude) was obtained and used immediately in the next step. ¹H NMR: (400 MHz, CDCl₃): δ=8.71 (d, J=2.1 Hz, 1H), 8.06-8.00 (m, 2H), 7.87.

### Synthesis of 4-(5-bromopyridin-2-yl)benzimidamide

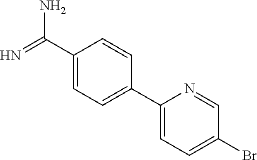 [SMILES: N=C(N)c1ccc(-c2ccc(Br)cn2)cc1]

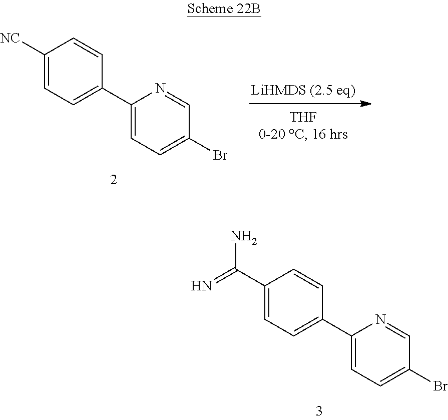 [SMILES: N#Cc1ccc(-c2ccc(Br)cn2)cc1 ; N=C(N)c1ccc(-c2ccc(Br)cn2)cc1]

To a solution of 4-(5-bromopyridin-2-yl)benzonitrile (2) (100 mg, 385.95 μmol, 1.0 eq) in THE (1 mL) was added LiHMDS (1 M, 964.87 uL, 2.5 eq) at 0° C. under N₂. The mixture was warmed to 20° C. and stirred for 16 h. The reaction mixture was quenched by addition of HCl/dioxane (4 mol/L, 0.3 mL) at 0° C., and then concentrated under reduced pressure to give a residue. The residue was suspended in MeOH (2 ml), and the mixture was stirred at 20° C. for 1 h. Then the mixture was filtered, and the filtrate was concentrated under reduced pressure to give a residue. 4-(5-bromopyridin-2-yl)benzimidamide (3) (110 mg, crude, HCl salt) was obtained and used in the next step without further purification.

### Synthesis of 2-(4-(5-bromopyridin-2-yl)phenyl)-3,5,7,8-tetrahydro-4H-thiopyrano[4,3-d]pyrimidin-4-one

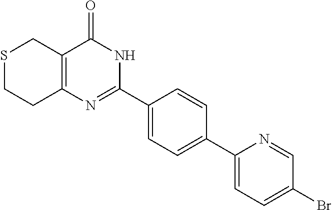 [SMILES: O=c1[nH]c(-c2ccc(-c3ccc(Br)cn3)cc2)nc2c1CSCC2]

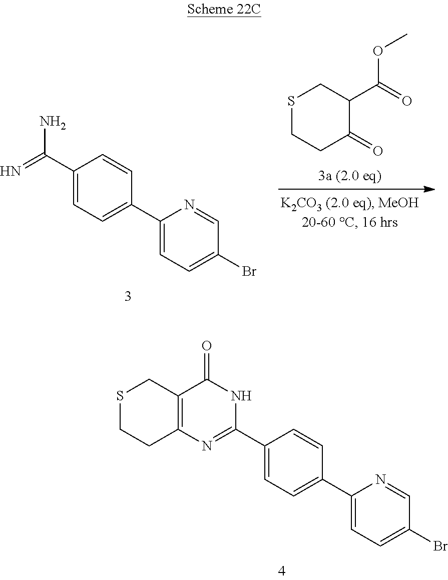 [SMILES: N=C(N)c1ccc(-c2ccc(Br)cn2)cc1 ; COC(=O)C1CSCCC1=O ; O=c1[nH]c(-c2ccc(-c3ccc(Br)cn3)cc2)nc2c1CSCC2]

To a solution of 4-(5-bromopyridin-2-yl)benzimidamide (3) (100 mg, 362.15 mol, 1.0 eq) in MeOH (1 mL) was added methyl 4-oxotetrahydro-2H-thiopyran-3-carboxylate (3a) (126.18 mg, 724.29 μmol, 2.0 eq) and K₂CO₃ (100.10 mg, 724.29 mol, 2.0 eq) at 20° C. The mixture was stirred at 60° C. for 16 h. The mixture was filtered, and the filter cake was dried under reduced pressure to give 2-(4-(5-bromopyridin-2-yl)phenyl)-3,5,7,8-tetrahydro-4H-thiopyrano[4,3-d]pyrimidin-4-one (4) (238 mg, crude) that was used immediately in the next step. ¹H NMR: (400 MHz, CDCl₃): =8.75-8.69 (m, 1H), 8.14-8.04 (m, 4H), 7.90-7.83 (m, 1H), 7.68-7.61 (m, 1H), 3.65-3.62 (m, 2H), 3.04-2.97 (m, 2H), 2.92-2.86 (m, 2H).

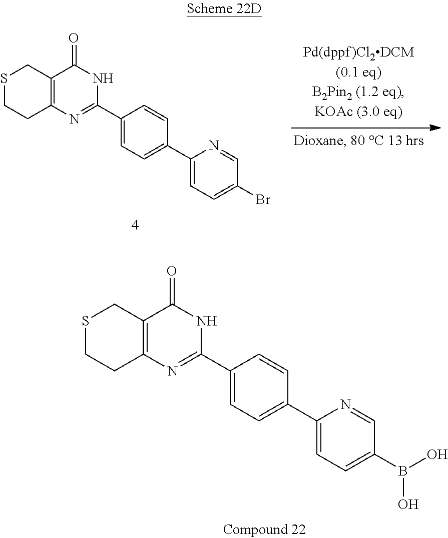 [SMILES: O=c1[nH]c(-c2ccc(-c3ccc(Br)cn3)cc2)nc2c1CSCC2 ; O=c1[nH]c(-c2ccc(-c3ccc(B(O)O)cn3)cc2)nc2c1CSCC2]

To a solution of 2-(4-(5-bromopyridin-2-yl)phenyl)-3,5,7,8-tetrahydro-4H-thiopyrano[4,3-d]pyrimidin-4-one (4) (238 mg, 594.57 mol, 1.0 eq) in dioxane (3 mL) was added B₂Pin₂ (181.18 mg, 713.48 μmol, 1.2 eq), KOAc (175.06 mg, 1.78 mmol, 3.0 eq), and Pd(dppf)Cl₂·DCM (48.55 mg, 59.46 mol, 0.1 eq) under N₂. The mixture was stirred at 80° C. for 13 h. After cooling to 25° C., the mixture was filtered, and the filtrate was concentrated under reduced pressure to give a residue. The residue was purified by prep-HPLC (neutral condition; column: Waters Xbridge Prep OBD C18 150*40 mm*10 m; mobile phase: [water (NH₄HCO₃)-ACN]; B %: 15%-45%, 8 mins). (6-(4-(4-oxo-3,5,7,8-tetrahydro-4H-thiopyrano[4,3-d]pyrimidin-2-yl)phenyl)pyridin-3-yl)boronic acid (Compound 22) was obtained. LC-MS: 366.1 (M+1). ¹H NMR: (400 MHz, DMSO-d₆): δ=12.83 (br s, 1H), 9.01 (s, 1H), 8.41 (s, 2H), 8.30-8.20 (m, 5H), 8.06 (d, J=8.0 Hz, 1H), 3.55 (s, 2H), 2.92 (s, 4H).

### Compound 23

### Synthesis of 2-(4-(2-bromopyridin-4-yl)phenyl)-3,5,7,8-tetrahydro-4H-thiopyrano[4,3-d]pyrimidin-4-one (Compound 23)

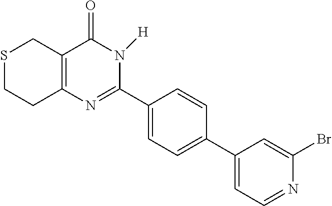 [SMILES: O=c1[nH]c(-c2ccc(-c3ccnc(Br)c3)cc2)nc2c1CSCC2]

### Synthesis of 4-(2-bromopyridin-4-yl)benzonitrile

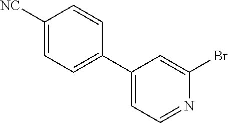 [SMILES: N#Cc1ccc(-c2ccnc(Br)c2)cc1]

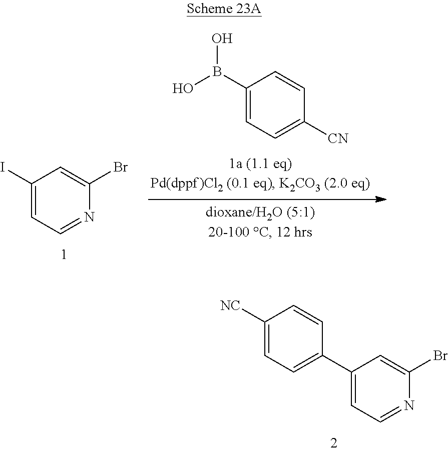 [SMILES: N#Cc1ccc(B(O)O)cc1 ; Brc1cc(I)ccn1 ; N#Cc1ccc(-c2ccnc(Br)c2)cc1]

To a solution of 2-bromo-4-iodopyridine (1) (2.50 g, 8.81 mmol, 1.00 eq) and (4-cyanophenyl)boronic acid (1a) (1.42 g, 9.69 mmol, 1.10 eq) in dioxane (25.0 mL) and H₂O (5.00 mL) was added K₂CO₃ (2.43 g, 17.6 mmol, 2.00 eq) at 20° C. The suspension was degassed and purged three times with N₂. Then to the mixture was added Pd(dppf)Cl₂ (644 mg, 881 μmol, 0.10 eq) at 20° C. under N₂. The suspension was degassed and purged three times with N₂. The mixture was stirred under N₂ at 100° C. for 12 h. The reaction mixture was diluted with H₂O (10.0 mL) and extracted with EtOAc 60.0 mL (20.0 mL×3). The combined organic layers were dried over Na₂SO₄, filtered, and concentrated under reduced pressure to give a residue. The residue was purified by column chromatography (SiO₂, petroleum ether/ethyl acetate=10/1 to 1/1). 4-(2-bromopyridin-4-yl)benzonitrile (2) (950 mg) was obtained. ¹H NMR: (400 MHz, DMSO-d6): δ=8.57 (d, J=5.3 Hz, 1H), 8.17-8.03 (m, 5H), 7.91 (dd, J=1.7, 5.2 Hz, 1H).

### Synthesis of 4-(2-bromopyridin-4-yl)benzimidamide

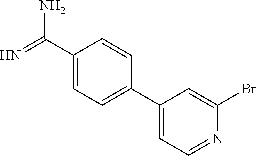 [SMILES: N=C(N)c1ccc(-c2ccnc(Br)c2)cc1]

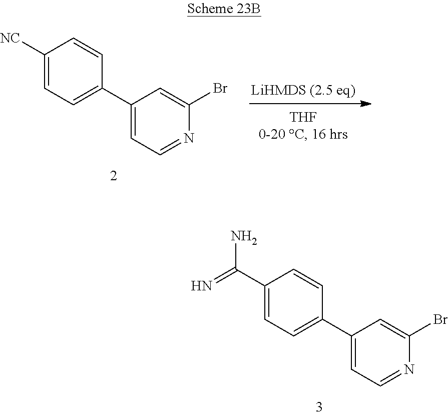 [SMILES: N#Cc1ccc(-c2ccnc(Br)c2)cc1 ; N=C(N)c1ccc(-c2ccnc(Br)c2)cc1]

To a solution of 4-(2-bromopyridin-4-yl)benzonitrile (2) (400 mg, 1.54 mmol, 1.00 eq) in THF (4.00 mL), LiHMDS (1 M, 3.09 mL, 2.00 eq) was added dropwise at 0° C. The mixture was stirred at 20° C. for 16 h. The reaction mixture was diluted with 4 N HCl (4.00 mL). The combined organic layers were concentrated under reduced pressure to give a residue. The residue was suspended in MeOH (20.0 mL) and stirred at 20° C. for 1 hr. Then the mixture was filtered. The filtrate was concentrated under reduced pressure to give a residue. 4-(2-bromopyridin-4-yl)benzimidamide (3) (500 mg, crude) was obtained and used in the next step without further purification.

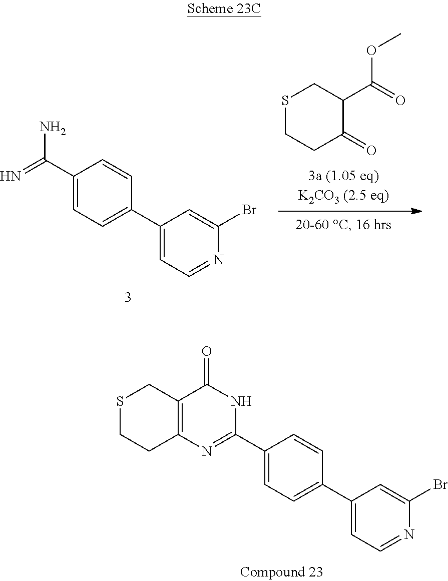 [SMILES: N=C(N)c1ccc(-c2ccnc(Br)c2)cc1 ; COC(=O)C1CSCCC1=O ; O=c1[nH]c(-c2ccc(-c3ccnc(Br)c3)cc2)nc2c1CSCC2]

To a solution of 4-(2-bromopyridin-4-yl)benzimidamide (3) (250 mg, 905 mol, 1.00 eq) and methyl 4-oxotetrahydro-2H-thiopyran-3-carboxylate (3a) (166 mg, 951 mol, 1.05 eq) in MeOH (2.50 mL) was added K₂CO₃ (313 mg, 2.26 mmol, 2.50 eq) at 20° C. The mixture was stirred at 60° C. for 16 h. The reaction mixture was filtered. The filter cake was slurried by MeOH (0.50 mL) and H₂O (1.00 mL) and then filtered. The filter cake was dried in vacuo to give a residue. The residue was purified by prep-HPLC (column: Phenomenex luna C18 80*40 mm*3 m; mobile phase: [water (HCl)-ACN]; B %: 35%-65%, 7 min). 2-(4-(2-bromopyridin-4-yl)phenyl)-3,5,7,8-tetrahydro-4H-thiopyrano[4,3-d]pyrimidin-4-one (Compound 23) was obtained. ¹H NMR (400 MHz, DMSO-d6): δ=8.48 (d, J=5.3 Hz, 1H), 8.27 (d, J=8.4 Hz, 2H), 8.07 (s, 1H), 7.99 (d, J=8.4 Hz, 2H), 7.87 (dd, J=1.2, 5.2 Hz, 1H), 3.54 (s, 2H), 2.89 (s, 4H). LC-MS: 404.1 (M+1).

### Compound 24

### Synthesis of (2-(4-(4-oxo-3,5,7,8-tetrahydro-4H-thiopyrano[4,3-d]pyrimidin-2-yl)phenyl)pyrimidin-5-yl)boronic acid (Compound 24)

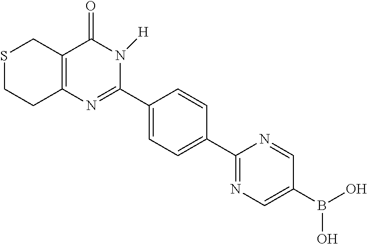 [SMILES: O=c1[nH]c(-c2ccc(-c3ncc(B(O)O)cn3)cc2)nc2c1CSCC2]

### Synthesis of 4-(5-bromopyrimidin-2-yl)benzonitrile

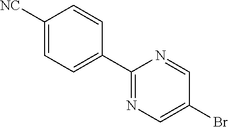 [SMILES: N#Cc1ccc(-c2ncc(Br)cn2)cc1]

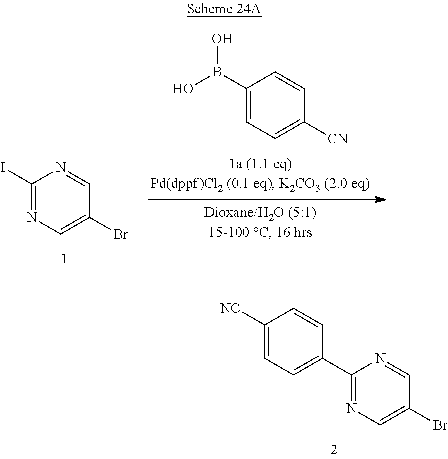 [SMILES: N#Cc1ccc(B(O)O)cc1 ; Brc1cnc(I)nc1 ; N#Cc1ccc(-c2ncc(Br)cn2)cc1]

To a solution of 5-bromo-2-iodopyrimidine (1) (2.50 g, 8.78 mmol, 1.00 eq) and (4-cyanophenyl)boronic acid (1a) (1.42 g, 9.65 mmol, 1.10 eq) in dioxane (25.0 mL) and H₂O (5.00 mL) was added K₂CO₃ (2.43 g, 17.6 mmol, 2.00 eq) at 15° C. The suspension was degassed and purged three times with N₂. Then to the mixture was added Pd(dppf)Cl₂ (642 mg, 878 mol, 0.10 eq) at 15° C. under N₂. The suspension was degassed and purged three times with N₂. The mixture was stirred under N₂ at 100° C. for 16 h. The reaction mixture was diluted with H₂O (10.0 mL) and filtered. The filtrate was extracted with EtOAc (10 mL×2). The combined organic layers were dried over Na₂SO₄, filtered, and concentrated under reduced pressure to give a residue. The residue was suspended in MTBE (10.0 mL), stirred at 15° C. for 16 h, then filtered. The filter cake was dried under reduced pressure to give a residue. 4-(5-bromopyrimidin-2-yl)benzonitrile (2) (2.00 g, crude) was obtained and used immediately in the next step. ¹H NMR (400 MHz, DMSO-d6): δ=9.16 (s, 2H), 8.49 (br d, J=8.3 Hz, 2H), 8.01 (br d, J=8.3 Hz, 2H).

### Synthesis of 4-(5-bromopyrimidin-2-yl)benzimidamide

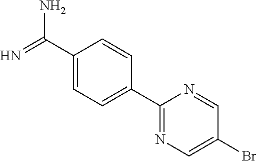 [SMILES: N=C(N)c1ccc(-c2ncc(Br)cn2)cc1]

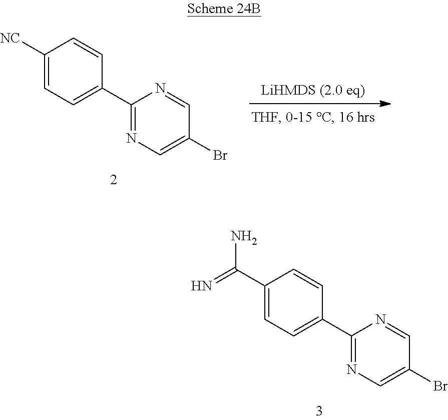 [SMILES: N#Cc1ccc(-c2ncc(Br)cn2)cc1 ; N=C(N)c1ccc(-c2ncc(Br)cn2)cc1]

To a solution of 4-(5-bromopyrimidin-2-yl)benzonitrile (2) (0.80 g, 3.08 mmol, 1.00 eq) in THF (8.00 mL) was added LiHMDS (1 M, 6.15 mL, 2.00 eq) at 0° C. The mixture was stirred at 15° C. for 16 h. The reaction mixture was diluted with 4 N HCl/dioxane (6.00 mL). The combined organic layers were concentrated under reduced pressure to give a residue. The residue was suspended in MeOH (8.00 mL) and stirred at 15° C. for 2 h, then filtered. The filtrate was concentrated under reduced pressure to give a residue. 4-(5-bromopyrimidin-2-yl)benzimidamide (3) (0.8 g, crude) was obtained which was used in the next step without further purification.

### Synthesis of 2-(4-(5-bromopyrimidin-2-yl)phenyl)-3,5,7,8-tetrahydro-4H-thiopyrano[4,3-d]pyrimidin-4-one

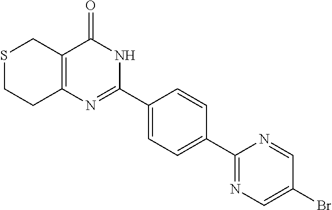 [SMILES: O=c1[nH]c(-c2ccc(-c3ncc(Br)cn3)cc2)nc2c1CSCC2]

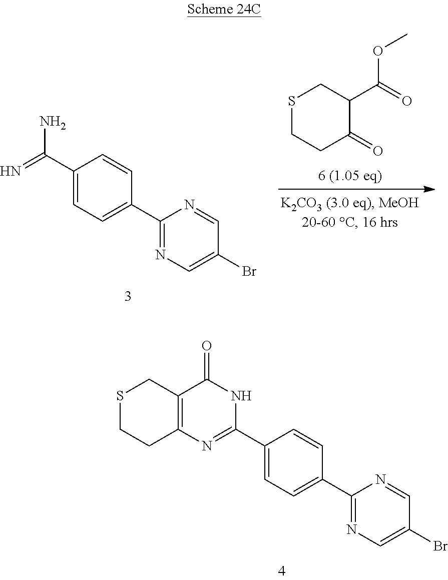 [SMILES: N=C(N)c1ccc(-c2ncc(Br)cn2)cc1 ; COC(=O)C1CSCCC1=O ; O=c1[nH]c(-c2ccc(-c3ncc(Br)cn3)cc2)nc2c1CSCC2]

To a solution of 4-(5-bromopyrimidin-2-yl)benzimidamide (3) (0.80 g, 2.89 mmol, 1.00 eq) and methyl 4-oxotetrahydrothiopyran-3-carboxylate (6) (528 mg, 3.03 mmol, 1.05 eq) in MeOH (16.0 mL) was added K₂CO₃ (1.20 g, 8.66 mmol, 3.00 eq) at 25° C. The mixture was stirred at 60° C. for 16 h. The reaction mixture was filtered. The filtrate was concentrated under reduced pressure. The resulting solid was suspended in water (5 mL) and stirred at 20° C. for 3 h. Then the mixture was filtered, and the filter cake was dried in vacuo to give 2-(4-(5-bromopyrimidin-2-yl)phenyl)-3,5,7,8-tetrahydro-4H-thiopyrano[4,3-d]pyrimidin-4-one (4) (300 mg, crude). The crude product was used in the next step directly without further purification.

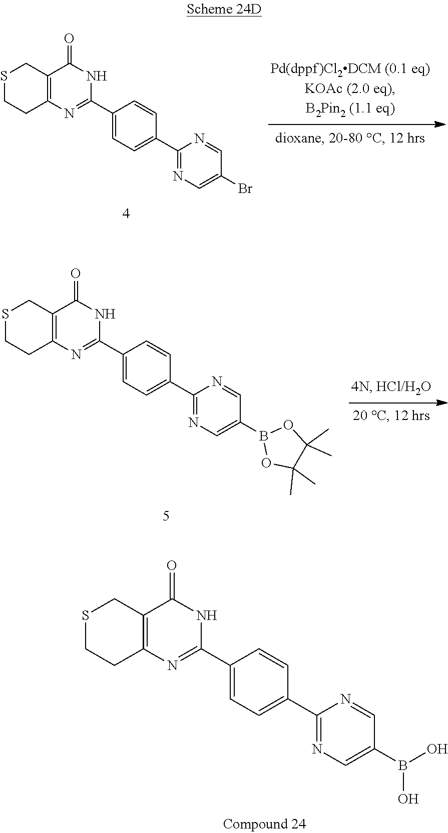 [SMILES: O=c1[nH]c(-c2ccc(-c3ncc(Br)cn3)cc2)nc2c1CSCC2 ; CC1(C)OB(c2cnc(-c3ccc(-c4nc5c(c(=O)[nH]4)CSCC5)cc3)nc2)OC1(C)C ; O=c1[nH]c(-c2ccc(-c3ncc(B(O)O)cn3)cc2)nc2c1CSCC2]

To a solution of 2-(4-(5-bromopyrimidin-2-yl)phenyl)-3,5,7,8-tetrahydro-4H-thiopyrano[4,3-d]pyrimidin-4-one (4) (0.1 g, 249.20 mol, 1 eq) in dioxane (2 mL) was added 4,4,5,5-tetramethyl-2-(4,4,5,5-tetramethyl-1,3,2-dioxaborolan-2-yl)-1,3,2-dioxaborolane (69.61 mg, 274.12 mol, 1.1 eq), KOAc (48.91 mg, 498.41 mol, 2 eq), and Pd(dppf)Cl₂·CH₂Cl₂ (20.35 mg, 24.92 mol, 0.1 eq) at 20° C. under N₂. The mixture was stirred at 80° C. for 12 h under N₂. The mixture was concentrated under reduced pressure to give a residue. The residue was suspended in MeCN/H₂O (1:1, 3 mL) and stirred at 20° C. for 2 h. Then the mixture was filtered, and the filter cake was dried in vacuo to give 2-(4-(5-(4,4,5,5-tetramethyl-1,3,2-dioxaborolan-2-yl)pyrimidin-2-yl)phenyl)-3,5,7,8-tetrahydro-4H-thiopyrano[4,3-d]pyrimidin-4-one (5) (50 mg, crude). The crude product was suspended in HCl/H₂O (4 N, 3 mL) and stirred at 20° C. for 12 h. Then the mixture was diluted with DMSO (5 mL) and purified by prep-HPLC (column: Phenomenex luna C18 250 * 50 mm*10 m; mobile phase: [water (HCl)-ACN]; B %: 15%-45%, 10 min). (2-(4-(4-oxo-3,5,7,8-tetrahydro-4H-thiopyrano[4,3-d]pyrimidin-2-yl)phenyl)pyrimidin-5-yl)boronic acid (Compound 24) was obtained. LCMS: 367 (M+1). ¹H NMR: (400 MHz, DMSO-d6): δ=9.16 (s, 2H), 8.54 (d, J=8.5 Hz, 2H), 8.25 (d, J=8.5 Hz, 2H), 3.55 (s, 2H), 2.92 (s, 4H).

### Compound 25

### Synthesis of (3′,5′-difluoro-4′-(4-oxo-3,5,7,8-tetrahydro-4H-thiopyrano[4,3-d]pyrimidin-2-yl)-[1,1′-biphenyl]-4-yl)boronic acid (Compound 25)

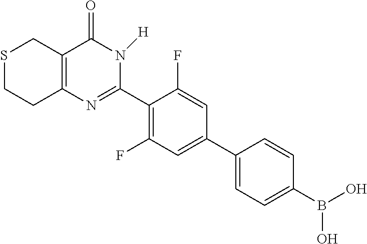 [SMILES: O=c1[nH]c(-c2c(F)cc(-c3ccc(B(O)O)cc3)cc2F)nc2c1CSCC2]

### Synthesis of 4′-bromo-3,5-difluoro-[1,1′-biphenyl]-4-carbonitrile

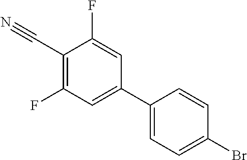 [SMILES: N#Cc1c(F)cc(-c2ccc(Br)cc2)cc1F]

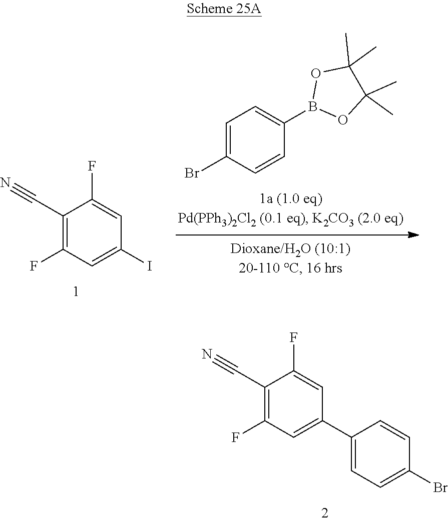 [SMILES: N#Cc1c(F)cc(I)cc1F ; CC1(C)OB(c2ccc(Br)cc2)OC1(C)C ; N#Cc1c(F)cc(-c2ccc(Br)cc2)cc1F]

To a mixture of 2,6-difluoro-4-iodobenzonitrile (1) (5 g, 22.94 mmol, 1 eq) and 2-(4-bromophenyl)-4,4,5,5-tetramethyl-1,3,2-dioxaborolane (1a) (6.49 g, 22.94 mmol, 1 eq) in dioxane (100 mL) and H₂O (10 mL) was added K₂CO₃ (6.34 g, 45.87 mmol, 2 eq) and Pd(PPh₃)₂Cl₂ (1.61 g, 2.29 mmol, 0.1 eq) at 20° C. under N₂. The mixture was degassed and purged three times with N₂, and then the mixture was stirred at 110° C. for 16 h under an N₂ atmosphere. The mixture was cooled to 20° C. and then diluted with water (100 mL). Then the mixture was extracted with EtOAc 300 mL (100 mL×3). The combined organic layers were washed with brine (100 mL), dried over Na₂SO₄, filtered, and concentrated under reduced pressure to give a residue that was purified by column chromatography (SiO₂, petroleum ether/ethyl acetate=50/1 to 0/1). 4′-bromo-3,5-difluoro-[1,1′-biphenyl]-4-carbonitrile (2) (1.8 g) was obtained. ¹H NMR: (400 MHz, CDCl₃): δ=7.57 (d, J=8.6 Hz, 2H), 7.39-7.33 (m, 2H), 7.20-7.17 (m, 2H).

### Synthesis of 4′-bromo-3,5-difluoro-[1,1′-biphenyl]-4-carboximidamide

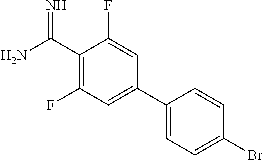 [SMILES: N=C(N)c1c(F)cc(-c2ccc(Br)cc2)cc1F]

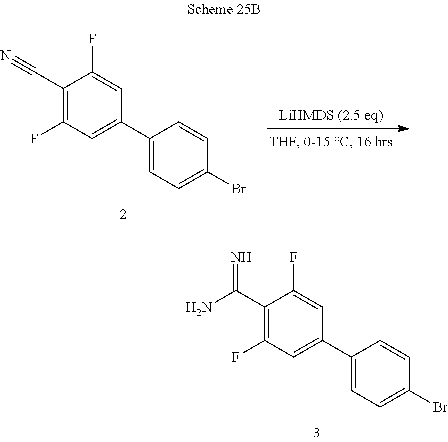 [SMILES: N#Cc1c(F)cc(-c2ccc(Br)cc2)cc1F ; N=C(N)c1c(F)cc(-c2ccc(Br)cc2)cc1F]

To a solution of 4′-bromo-3,5-difluoro-[1,1′-biphenyl]-4-carbonitrile (2) (0.5 g, 1.70 mmol, 1 eq) in THF (5 mL), LiHMDS (1 M, 4.25 mL, 2.5 eq) was added dropwise at 0° C. The mixture was stirred at 15° C. for 16 h. The reaction mixture was quenched by dropwise addition of 4 N HCl/dioxane (10 mL). The mixture was concentrated under reduced pressure to give a residue. The residue was suspended in MeOH (10 mL) and stirred at 20° C. for 1 hr. Then the mixture was filtered, and the filtrate was concentrated under reduced pressure to give a residue. 4′-bromo-3,5-difluoro-[1,1′-biphenyl]-4-carboximidamide (3) (528 mg, crude) was obtained which was used in the next step without further purification.

### Synthesis of 2-(4′-bromo-3,5-difluoro-[1,1′-biphenyl]-4-yl)-3,5,7,8-tetrahydro-4H-thiopyrano[4,3-d]pyrimidin-4-one

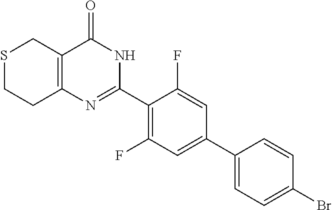 [SMILES: O=c1[nH]c(-c2c(F)cc(-c3ccc(Br)cc3)cc2F)nc2c1CSCC2]

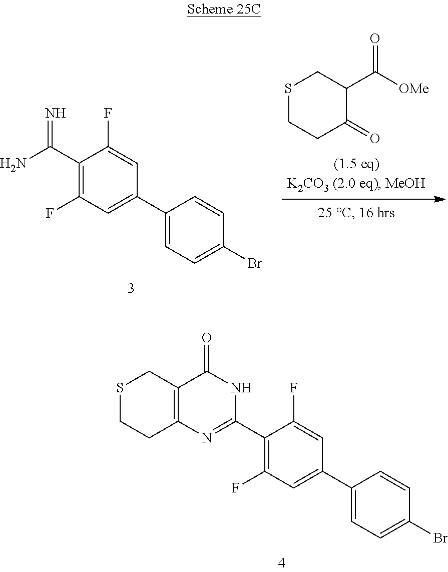 [SMILES: N=C(N)c1c(F)cc(-c2ccc(Br)cc2)cc1F ; COC(=O)C1CSCCC1=O ; O=c1[nH]c(-c2c(F)cc(-c3ccc(Br)cc3)cc2F)nc2c1CSCC2]

To a mixture of 4′-bromo-3,5-difluoro-[1,1′-biphenyl]-4-carboximidamide-HCl (3) (528 mg, 1.52 mmol, 1 eq) in MeOH (10 mL) was added K₂CO₃ (419.88 mg, 3.04 mmol, 2 eq) and methyl 4-oxotetrahydrothiopyran-3-carboxylate (396.97 mg, 2.28 mmol, 1.5 eq) at 25° C. under N₂. Then the mixture was stirred at 25° C. for 16 h under an N₂ atmosphere. The reaction mixture was filtered, and the filtrate was concentrated under reduced pressure to give a residue. The residue was triturated with water (10 mL) at 25° C. for 1 hr and then filtered. The filter cake was dried in vacuo to give 2-(4′-bromo-3,5-difluoro-[1,1′-biphenyl]-4-yl)-3,5,7,8-tetrahydro-4H-thiopyrano[4,3-d]pyrimidin-4-one (4) (0.9 g, crude) which was used directly in the next step.

### Synthesis of 2-(3,5-difluoro-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one

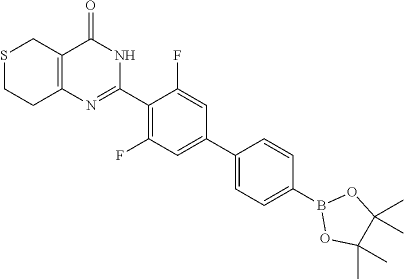 [SMILES: CC1(C)OB(c2ccc(-c3cc(F)c(-c4nc5c(c(=O)[nH]4)CSCC5)c(F)c3)cc2)OC1(C)C]

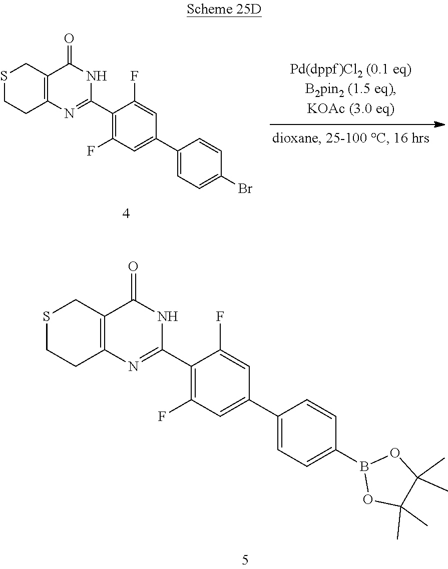 [SMILES: O=c1[nH]c(-c2c(F)cc(-c3ccc(Br)cc3)cc2F)nc2c1CSCC2 ; CC1(C)OB(c2ccc(-c3cc(F)c(-c4nc5c(c(=O)[nH]4)CSCC5)c(F)c3)cc2)OC1(C)C]

To a mixture of 2-(4′-bromo-3,5-difluoro-[1,1′-biphenyl]-4-yl)-3,5,7,8-tetrahydro-4H-thiopyrano[4,3-d]pyrimidin-4-one (4) (150 mg, 344.60 mol, 1 eq) in dioxane (3 mL) was added B₂Pin₂ (131.26 mg, 516.90 mol, 1.5 eq) and KOAc (101.46 mg, 1.03 mmol, 3 eq) at 25° C. The mixture was degassed and purged three times with N₂ and then Pd(dppf)Cl₂ (28.14 mg, 34.46 mol, 0.1 eq) was added at 25° C. under N₂. The mixture was stirred at 100° C. for 16 h under a N₂ atmosphere. After cooling to 20° C., the reaction mixture was filtered and concentrated under reduced pressure to give a residue. The crude product was triturated with water (2 mL) and acetonitrile (2 ml) at 25° C. for 1 hr. The mixture was filtered, and the filter cake was dried in vacuo. 2-(3,5-difluoro-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (120 mg) was obtained.

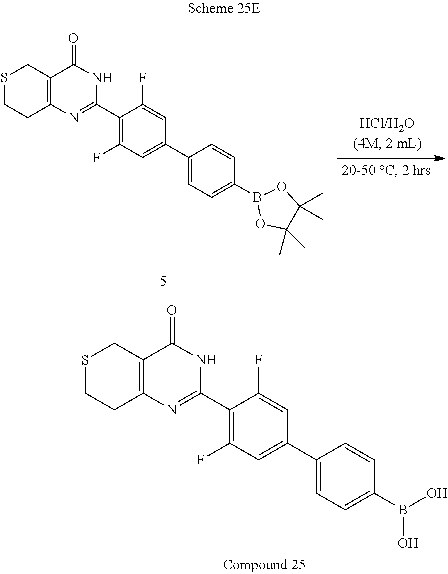 [SMILES: CC1(C)OB(c2ccc(-c3cc(F)c(-c4nc5c(c(=O)[nH]4)CSCC5)c(F)c3)cc2)OC1(C)C ; O=c1[nH]c(-c2c(F)cc(-c3ccc(B(O)O)cc3)cc2F)nc2c1CSCC2]

To a stirred solution of HCl/H₂O (4 M, 2 mL), 2-(3,5-difluoro-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (120 mg, 248.78 mol, 1 eq) was added portion-wise at 20° C. under N₂. Then the mixture was stirred at 50° C. for 2 h under an N₂ atmosphere. The mixture was concentrated under reduced pressure to give a residue. The residue was purified by prep-HPLC (column: Waters Xbridge BEH C18 100*30 mm*10 m; mobile phase: [water (NH₄HCO₃)-ACN]; B %: 25%-55%, 8 min). (3′,5′-difluoro-4′-(4-oxo-3,5,7,8-tetrahydro-4H-thiopyrano[4,3-d]pyrimidin-2-yl)-[1,1′-biphenyl]-4-yl)boronic acid (Compound 25) was obtained. LCMS: 401 (M+1). ¹H NMR: (400 MHz, DMSO-d6): δ=13.30-12.89 (m, 1H), 8.19 (s, 2H), 7.98-7.86 (m, 2H), 7.84-7.77 (m, 2H), 7.73-7.65 (m, 2H), 3.55 (br s, 2H), 2.97-2.81 (m, 4H).

### Compound 26

### Synthesis of (3′-hydroxy-4′-(4-oxo-3,5,7,8-tetrahydro-4H-thiopyrano[4,3-d]pyrimidin-2-yl)-[1,1′-biphenyl]-4-yl)boronic acid (Compound 26)

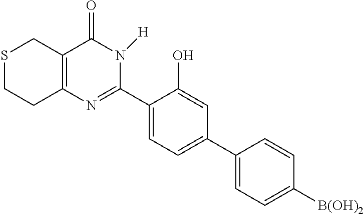 [SMILES: O=c1[nH]c(-c2ccc(-c3ccc(B(O)O)cc3)cc2O)nc2c1CSCC2]

Synthesis of 4′-bromo-3-methoxy-[1,1′-biphenyl]-4-carbonitrile

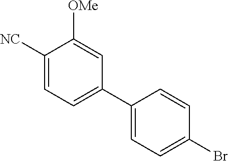 [SMILES: COc1cc(-c2ccc(Br)cc2)ccc1C#N]

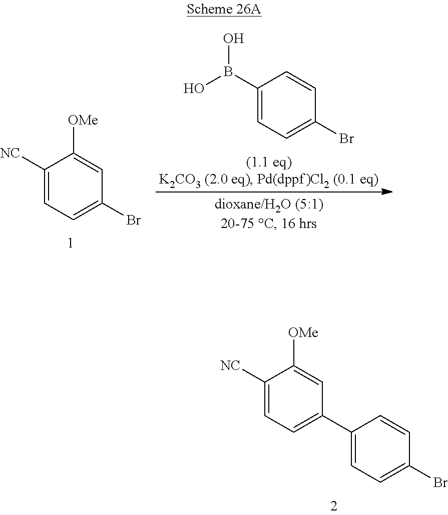 [SMILES: COc1cc(Br)ccc1C#N ; OB(O)c1ccc(Br)cc1 ; COc1cc(-c2ccc(Br)cc2)ccc1C#N]

To a solution of 4-bromo-2-methoxybenzonitrile (1) (10.0 g, 47.2 mmol, 1.00 eq) and (4-bromophenyl)boronic acid (10.4 g, 51.9 mmol, 1.10 eq) in dioxane (50.0 mL) and H₂O (10.0 mL) was added K₂CO₃ (13.0 g, 94.3 mmol, 2.00 eq) and Pd(dppf)Cl₂ (3.45 g, 4.72 mmol, 0.10 eq) at 20° C. The mixture was heated to 75° C. for 16 h. TLC (petroleum ether/ethyl acetate=5/1, R_{f}=0.53) indicated that (1) was consumed completely. The reaction mixture was diluted with EtOAc (50.0 mL) and H₂O (20.0 mL). The organic layer was separated from the mixture and the aqueous phase was extracted with EtOAc (50.0 mL×3). The combined organic layers were dried over Na₂SO₄, filtered, and concentrated under reduced pressure to give a residue that was purified by column chromatography (SiO₂, petroleum ether/ethylacetate=10/1 to 0/1) to provide 4′-bromo-3-methoxy-[1,1′-biphenyl]-4-carbonitrile (2) (3.30 g, 8.02 mmol). The compound was used without further purification. ¹H NMR (400 MHz, DMSO-d₆). δ=7.86 (d, J=8.1 Hz, 1H), 7.84-7.72 (m, 4H), 7.52 (d, J=1.1 Hz, 1H), 7.45 (dd, J=1.4, 8.0 Hz, 1H), 4.08 (s, 3H).

### Synthesis of 4′-bromo-3-methoxy-[1,1′-biphenyl]-4-carboximidamide

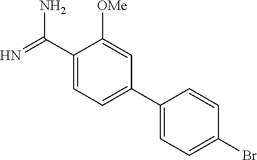 [SMILES: COc1cc(-c2ccc(Br)cc2)ccc1C(=N)N]

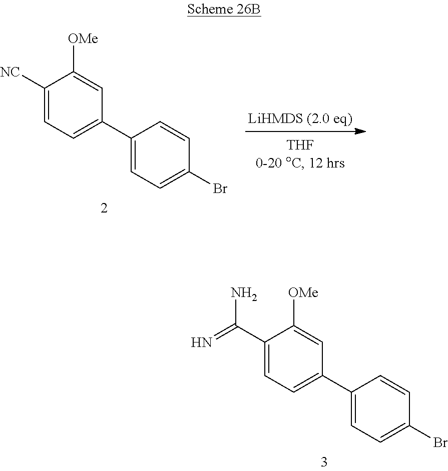 [SMILES: COc1cc(-c2ccc(Br)cc2)ccc1C#N ; COc1cc(-c2ccc(Br)cc2)ccc1C(=N)N]

To a solution of 4′-bromo-3-methoxy-[1,1′-biphenyl]-4-carbonitrile (2) (2.00 g, 6.94 mmol, 1.00 eq) in THF (20.0 mL), LiHMDS (1 M, 13.9 mL, 2.00 eq) was added dropwise at 0° C. and then allowed to warm to 20° C. and stirred for 12 h. The reaction mixture was quenched with HCl/dioxane (13.0 mL) at 0° C., and then concentrated under reduced pressure to give a residue. The residue was triturated with MeOH (10 mL) and filtered. The filter cake was dried in vacuo to give a residue. 4′-bromo-3-methoxy-[1,1′-biphenyl]-4-carboximidamide (3) (3.00 g, crude) was obtained. The crude product was used in the next step without further purification. LC-MS: 306 (M+1)

### Synthesis of 2-(4′-bromo-3-methoxy-[1,1′-biphenyl]-4-yl)-3,5,7,8-tetrahydro-4H-thiopyrano[4,3-d]pyrimidin-4-one

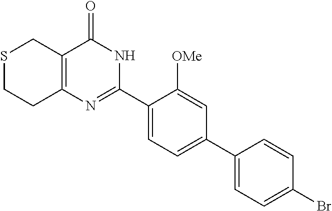 [SMILES: COc1cc(-c2ccc(Br)cc2)ccc1-c1nc2c(c(=O)[nH]1)CSCC2]

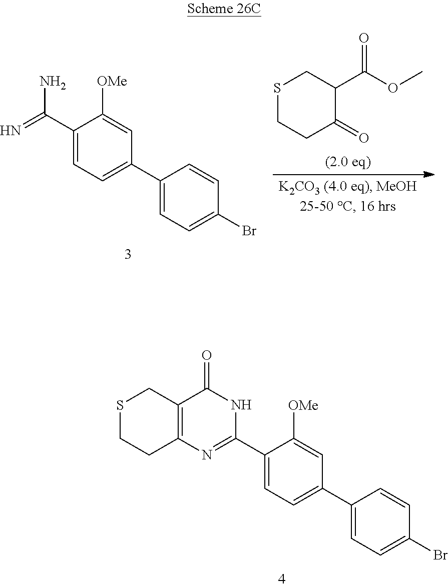 [SMILES: COc1cc(-c2ccc(Br)cc2)ccc1C(=N)N ; COC(=O)C1CSCCC1=O ; COc1cc(-c2ccc(Br)cc2)ccc1-c1nc2c(c(=O)[nH]1)CSCC2]

To a solution of 4′-bromo-3-methoxy-[1,1′-biphenyl]-4-carboximidamide (3) (3.00 g, 9.83 mmol, 1.00 eq) and methyl 4-oxotetrahydrothiopyran-3-carboxylate (3.43 g, 19.6 mmol, 2.00 eq) in MeOH (45.0 mL) was added K₂CO₃ (5.43 g, 39.3 mmol, 4.00 eq) at 25° C. and then heated to 50° C. and stirred for 16 h. LC-MS showed that (3) was consumed. The reaction mixture was filtered, and the filter cake was dried under reduced pressure to give a residue. The residue was triturated with H₂O (30.0 mL) and stirred at 20° C. for 16 h. Then the mixture was filtered, and the filter cake was dried under reduced pressure to give 2-(4′-bromo-3-methoxy-[1,1′-biphenyl]-4-yl)-3,5,7,8-tetrahydro-4H-thiopyrano[4,3-d]pyrimidin-4-one (4) (1.5 g, crude), which was used in the next step without further purification. LC-MS: 430.2 (and bromo isomers) (M+1).

### Synthesis of 2-(3-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one

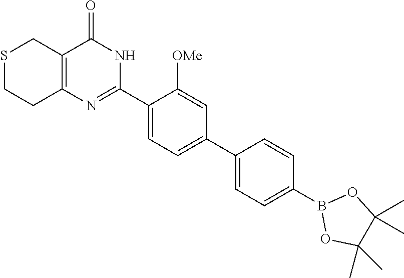 [SMILES: COc1cc(-c2ccc(B3OC(C)(C)C(C)(C)O3)cc2)ccc1-c1nc2c(c(=O)[nH]1)CSCC2]

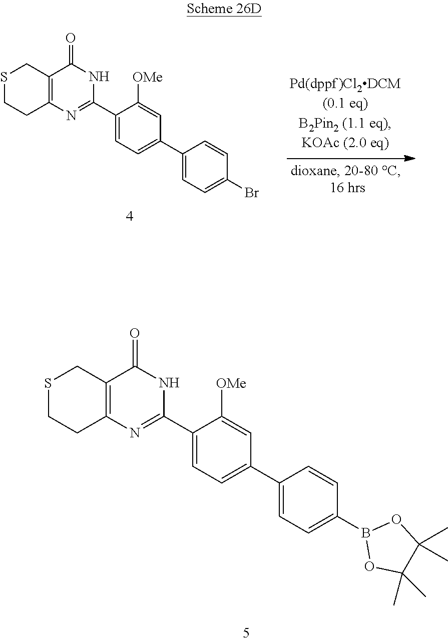 [SMILES: COc1cc(-c2ccc(Br)cc2)ccc1-c1nc2c(c(=O)[nH]1)CSCC2 ; COc1cc(-c2ccc(B3OC(C)(C)C(C)(C)O3)cc2)ccc1-c1nc2c(c(=O)[nH]1)CSCC2]

To a solution of 2-(4′-bromo-3-methoxy-[1,1′-biphenyl]-4-yl)-3,5,7,8-tetrahydro-4H-thiopyrano[4,3-d]pyrimidin-4-one (4) (1.00 g, 2.33 mmol, 1.00 eq) and Pin₂B₂ (650 mg, 2.56 mmol, 1.10 eq) in dioxane (50.0 mL) was added KOAc (457 mg, 4.66 mmol, 2.00 eq) and Pd(dppf)Cl₂·CH₂Cl₂ (190 mg, 233 mol, 0.10 eq) at 20° C. The mixture was heated to 80° C. and stirred for 16 h. LC-MS showed (4) was consumed completely and one main peak with the desired mass was detected. The mixture was filtered, and the filtrate was concentrated under reduced pressure to provide 2-(3-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (1.60 g, crude). The crude product was used in the next step without further purification. LC-MS: 477.2 (M+1).

### Synthesis of (3′-methoxy-4′-(4-oxo-3,5,7,8-tetrahydro-4H-thiopyrano[4,3-d]pyrimidin-2-yl)-[1,1′-biphenyl]-4-yl)boronic acid

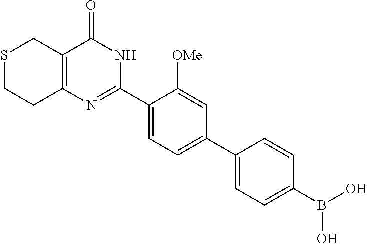 [SMILES: COc1cc(-c2ccc(B(O)O)cc2)ccc1-c1nc2c(c(=O)[nH]1)CSCC2]

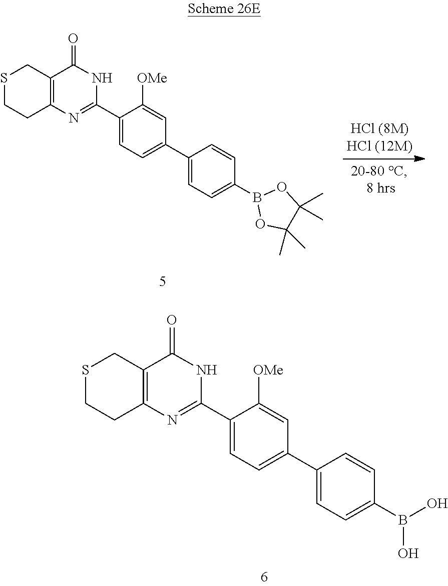 [SMILES: COc1cc(-c2ccc(B3OC(C)(C)C(C)(C)O3)cc2)ccc1-c1nc2c(c(=O)[nH]1)CSCC2 ; COc1cc(-c2ccc(B(O)O)cc2)ccc1-c1nc2c(c(=O)[nH]1)CSCC2]

A mixture of 2-(3-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (1.60 g, 3.36 mmol, 1.00 eq) in HCl (8 M, 32.0 mL, 76.2 eq) was heated to 80° C. and stirred for 16 h. HCl (12 M, 8.00 mL, 28.6 eq) was added to the mixture at 20° C., and then heated to 80° C. for 4 h. LC-MS showed (5) was consumed completely and one main peak with the desired mass was detected. The reaction mixture was filtered, then the filter cake was dried under reduced pressure to give (3′-methoxy-4′-(4-oxo-3,5,7,8-tetrahydro-4H-thiopyrano[4,3-d]pyrimidin-2-yl)-[1,1′-biphenyl]-4-yl)boronic acid (6) (710 mg, crude). The crude product was used in the next step without further purification. LC-MS: 395.2 (M+1).

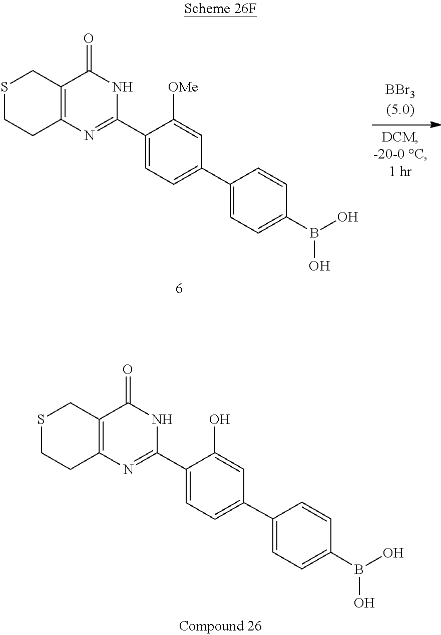 [SMILES: COc1cc(-c2ccc(B(O)O)cc2)ccc1-c1nc2c(c(=O)[nH]1)CSCC2 ; O=c1[nH]c(-c2ccc(-c3ccc(B(O)O)cc3)cc2O)nc2c1CSCC2]

To a solution of (3′-methoxy-4′-(4-oxo-3,5,7,8-tetrahydro-4H-thiopyrano[4,3-d]pyrimidin-2-yl)-[1,1′-biphenyl]-4-yl)boronic acid (6) (710 mg, 1.80 mmol, 1.00 eq) in DCM (21.6 mL), BBr₃ (1 M in DCM, 9.00 mL, 5.00 eq) was added dropwise at −20° C. The mixture was heated to 20° C. and stirred for 1 h. LC-MS showed that (6) was consumed completely and one peak with the desired mass was detected. The reaction mixture was quenched by the addition of ice water (20.0 mL) at 0° C. The mixture was filtered, and the filter cake was dried under reduced pressure to give a residue that was purified by prep-HPLC (column: Phenomenex Gemini NXC18 (75×30 mm×3 um); mobile phase: [H₂O (0.05% NH₃H₂O+10 mM NH₄HCO₃)-ACN]; gradient: 15%-65% B over 8.0 min) to give (3′-hydroxy-4′-(4-oxo-3,5,7,8-tetrahydro-4H-thiopyrano[4,3-d]pyrimidin-2-yl)-[1,1′-biphenyl]-4-yl)boronic acid (Compound 26). LC-MS: 381.2 (M+1). ¹H NMR (400 MHz, DMSO-d₆): δ=8.27 (d, J=8.1 Hz, 1H), 8.13 (s, 2H), 7.90 (d, J=8.0 Hz, 2H), 7.71 (d, J=8.0 Hz, 2H), 7.32-7.19 (m, 2H), 3.55 (s, 2H), 2.93 (s, 4H).

### Compound 27

### Synthesis of (3-hydroxy-4′-(4-oxo-3,5,7,8-tetrahydro-4H-thiopyrano[4,3-d]pyrimidin-2-yl)-[1,1′-biphenyl]-4-yl)boronic acid (Compound 27)

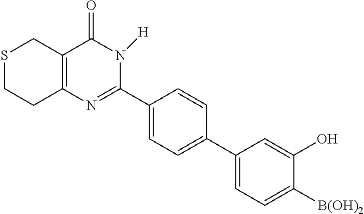 [SMILES: O=c1[nH]c(-c2ccc(-c3ccc(B(O)O)c(O)c3)cc2)nc2c1CSCC2]

### Synthesis of 4′-bromo-3′-methoxy-[1,1′-biphenyl]-4-carbonitrile

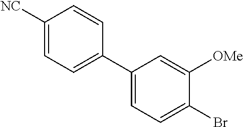 [SMILES: COc1cc(-c2ccc(C#N)cc2)ccc1Br]

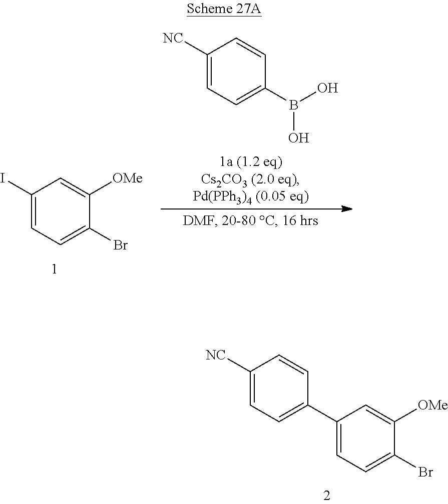 [SMILES: N#Cc1ccc(B(O)O)cc1 ; COc1cc(I)ccc1Br ; COc1cc(-c2ccc(C#N)cc2)ccc1Br]

To a solution of 1-bromo-4-iodo-2-methoxybenzene (1) (8 g, 25.56 mmol, 1 eq) and (4-cyanophenyl)boronic acid (1a) (4.51 g, 30.7 mmol, 1.20 eq) in DMF (160 mL) was added Cs₂CO₃ (16.7 g, 51.1 mmol, 2.00 eq) and Pd(PPh₃)₄ (1.48 g, 1.28 mmol, 0.05 eq) at 25° C. The mixture was heated to 80° C. and stirred for 16 h under N₂. LC-MS showed that (1) was consumed completely. The reaction mixture was diluted with EtOAc and H₂O. The organic layer was separated from the mixture and the aqueous phase was extracted with EtOAc. The combined organic layers were dried over Na₂SO₄, filtered, and concentrated under reduced pressure to give a residue. The residue was purified by column chromatography (SiO₂, petroleum ether/ethyl acetate=10/1 to 0/1) to provide 4′-bromo-3′-methoxy-[1,1′-biphenyl]-4-carbonitrile (2) (4.50 g, 15.6 mmol). ¹H NMR (400 MHz, DMSO-d6): δ=8.00 (s, 4H), 7.74 (d, J=8.2 Hz, 1H), 7.46 (d, J=2.0 Hz, 1H), 7.31 (dd, J=2.1, 8.2 Hz, 1H), 4.02 (s, 3H).

### Synthesis of 4′-bromo-3′-methoxy-[1,1′-biphenyl]-4-carboximidamide

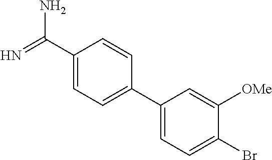 [SMILES: COc1cc(-c2ccc(C(=N)N)cc2)ccc1Br]

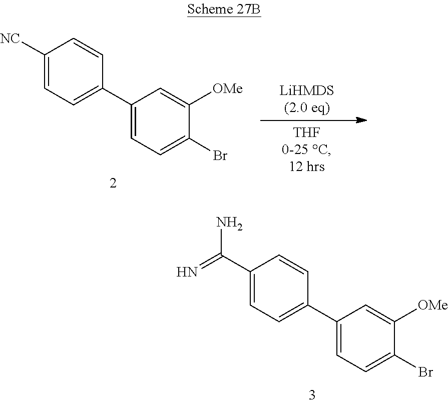 [SMILES: COc1cc(-c2ccc(C#N)cc2)ccc1Br ; COc1cc(-c2ccc(C(=N)N)cc2)ccc1Br]

To a solution of 4′-bromo-3′-methoxy-[1,1′-biphenyl]-4-carbonitrile (2) (2.00 g, 6.94 mmol, 1.00 eq) in THF (20.0 mL), was added dropwise LiHMDS (1 M, 13.9 mL, 2.00 eq) at 0° C. The mixture was warmed to 25° C. and stirred for 12 h. LC-MS showed that (2) was consumed completely. The reaction mixture was quenched by dropwise addition of HCl/dioxane (4M, 8.00 mL) at 0° C., then concentrated under reduced pressure to give a residue. The residue was suspended in MeOH (20.0 mL), and the mixture stirred at 25° C. for 1 h. Then the mixture was filtered, and the filtrate was concentrated under reduced pressure to give 4′-bromo-3′-methoxy-[1,1′-biphenyl]-4-carboximidamide (3) (3.00 g, crude) and used directly in the next step without further purification. LC-MS: 306.1+bromo isomers (M+1).

### Synthesis of 2-(4′-bromo-3′-methoxy-[1,1′-biphenyl]-4-yl)-3,5,7,8-tetrahydro-4H-thiopyrano[4,3-d]pyrimidin-4-one

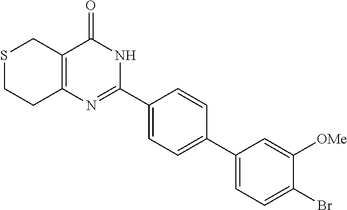 [SMILES: COc1cc(-c2ccc(-c3nc4c(c(=O)[nH]3)CSCC4)cc2)ccc1Br]

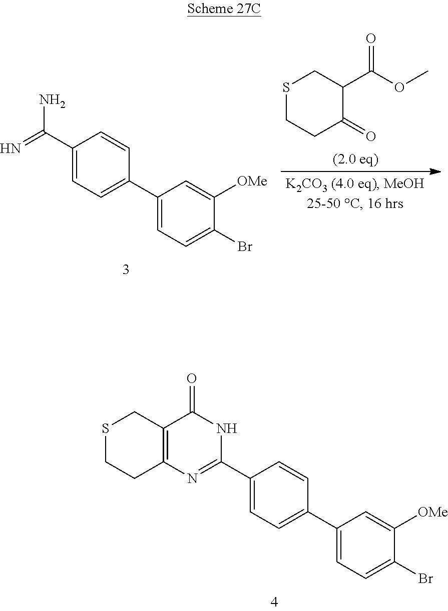 [SMILES: COc1cc(-c2ccc(C(=N)N)cc2)ccc1Br ; COC(=O)C1CSCCC1=O ; COc1cc(-c2ccc(-c3nc4c(c(=O)[nH]3)CSCC4)cc2)ccc1Br]

To a solution of 4′-bromo-3′-methoxy-[1,1′-biphenyl]-4-carboximidamide (3) (1.00 g, 3.28 mmol, 1.00 eq) and methyl 4-oxotetrahydrothiopyran-3-carboxylate (1.14 g, 6.55 mmol, 2 eq) in MeOH (15.0 mL) was added K₂CO₃ (1.81 g, 13.1 mmol, 4.00 eq) at 25° C. The mixture was heated to 50° C. and stirred for 16 h. LC-MS showed (3) was consumed completely. The reaction mixture was filtered, and the filter cake was dried under reduced pressure to give a residue. H₂O (10.0 mL) was added to the residue to form a slurry that was stirred at 20° C. for 16 h. Then the mixture was filtered, and the filter cake was dried under reduced pressure to give 2-(4′-bromo-3′-methoxy-[1,1′-biphenyl]-4-yl)-3,5,7,8-tetrahydro-4H-thiopyrano[4,3-d]pyrimidin-4-one (4) (1.50 g, crude). LC-MS: 430.2 and bromo isomers (M+1). ¹H NMR (400 MHz, DMSO-d6): δ=12.76 (br s, 1H), 8.26-8.20 (m, J=8.4 Hz, 2H), 7.90-7.82 (m, J=8.4 Hz, 2H), 7.68 (d, J=8.3 Hz, 1H), 7.42 (d, J=1.9 Hz, 1H), 7.27 (dd, J=2.0, 8.3 Hz, 1H), 3.98 (s, 3H), 3.53 (s, 2H), 2.89 (s, 4H).

### Synthesis of 2-(3′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one

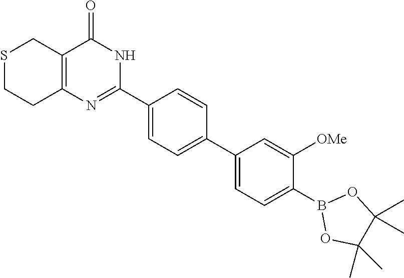 [SMILES: COc1cc(-c2ccc(-c3nc4c(c(=O)[nH]3)CSCC4)cc2)ccc1B1OC(C)(C)C(C)(C)O1]

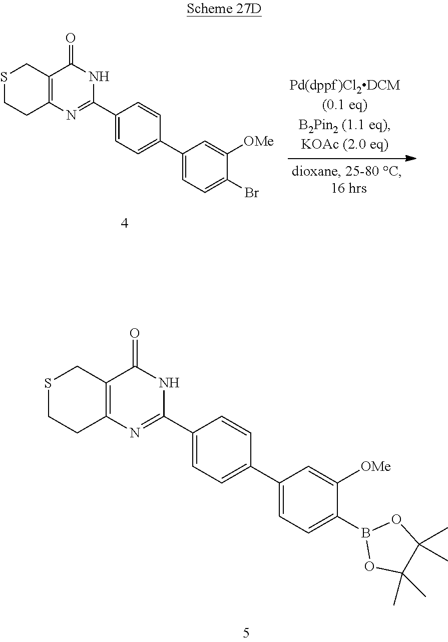 [SMILES: COc1cc(-c2ccc(-c3nc4c(c(=O)[nH]3)CSCC4)cc2)ccc1Br ; COc1cc(-c2ccc(-c3nc4c(c(=O)[nH]3)CSCC4)cc2)ccc1B1OC(C)(C)C(C)(C)O1]

To a solution of 2-(4′-bromo-3′-methoxy-[1,1′-biphenyl]-4-yl)-3,5,7,8-tetrahydro-4H-thiopyrano[4,3-d]pyrimidin-4-one (4) (1.00 g, 2.33 mmol, 1.00 eq) and B₂Pin₂ (651 mg, 2.56 mmol, 1.10 eq) in dioxane (30.0 mL) was added KOAc (457 mg, 4.66 mmol, 2.00 eq) and Pd(dppf)Cl₂·DCM (190 mg, 233 mol, 0.10 eq) at 25° C. The mixture was heated to 80° C. and stirred under N₂ for 16 h. LC-MS showed that (4) was consumed completely. The mixture was filtered, and the filtrate was concentrated under reduced pressure to give a residue. 2-(3′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (1.00 g, crude) was obtained, which was used directly in the next step without further purification. LC-MS: 477.1 (M+1).

### Synthesis of (3-methoxy-4′-(4-oxo-3,5,7,8-tetrahydro-4H-thiopyrano[4,3-d]pyrimidin-2-yl)-[1,1′-biphenyl]-4-yl)boronic acid

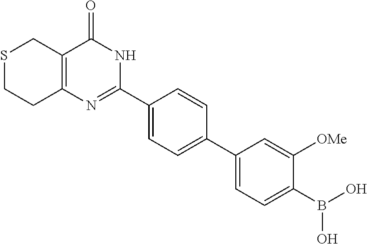 [SMILES: COc1cc(-c2ccc(-c3nc4c(c(=O)[nH]3)CSCC4)cc2)ccc1B(O)O]

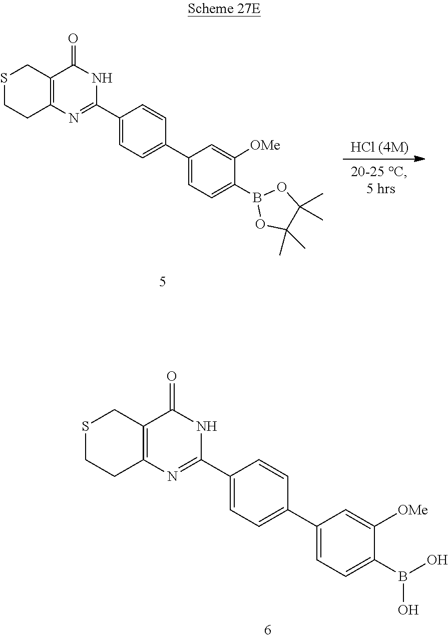 [SMILES: COc1cc(-c2ccc(-c3nc4c(c(=O)[nH]3)CSCC4)cc2)ccc1B1OC(C)(C)C(C)(C)O1 ; COc1cc(-c2ccc(-c3nc4c(c(=O)[nH]3)CSCC4)cc2)ccc1B(O)O]

2-(3′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (1.00 g, 2.10 mmol, 1.00 eq) was added portion-wise to a stirred solution of HCl/H₂O (4 M, 20.0 mL, 38.1 eq) at 20° C. The mixture was stirred at 25° C. for 5 h. LC-MS showed that (5) was consumed. The reaction mixture was filtered and the filter cake dried under reduced pressure to give (3-methoxy-4′-(4-oxo-3,5,7,8-tetrahydro-4H-thiopyrano[4,3-d]pyrimidin-2-yl)-[1,1′-biphenyl]-4-yl)boronic acid (6) (250 mg, crude), which was used as is in the next step. LC-MS: 395.2 (M+1).

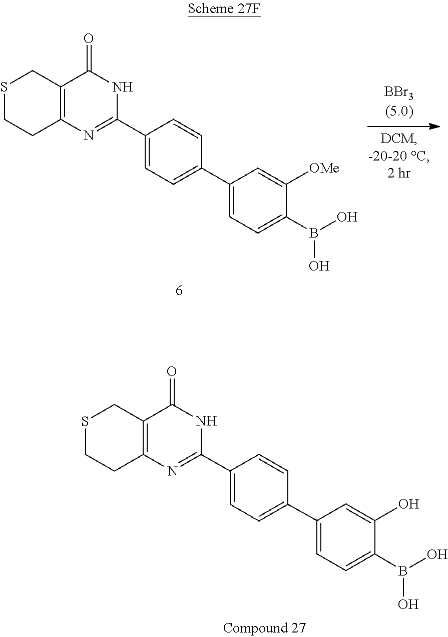 [SMILES: COc1cc(-c2ccc(-c3nc4c(c(=O)[nH]3)CSCC4)cc2)ccc1B(O)O ; O=c1[nH]c(-c2ccc(-c3ccc(B(O)O)c(O)c3)cc2)nc2c1CSCC2]

To a solution of (3-methoxy-4′-(4-oxo-3,5,7,8-tetrahydro-4H-thiopyrano[4,3-d]pyrimidin-2-yl)-[1,1′-biphenyl]-4-yl)boronic acid (6) (250 mg, 634 mol, 1.00 eq) in DCM (7.50 mL), was added BBr₃ (1 M, 3.17 mL, 5.00 eq) dropwise at −20° C. The mixture was stirred at 20° C. for 2 h. LC-MS showed that (6) was consumed completely. The reaction mixture was cooled to 0° C. and quenched with ice water (10.0 mL) and stirred at 0° C for 10 min. The mixture was filtered and the filter cake dried under reduced pressure to give a residue. The residue was purified by prep-HPLC (column: Phenomenex luna C18 100×40 mm×5 um; mobile phase: [H₂O (0.04% HCl)-ACN]; gradient: 10%-50% B over 8.0 min) to provide (3-hydroxy-4′-(4-oxo-3,5,7,8-tetrahydro-4H-thiopyrano[4,3-d]pyrimidin-2-yl)-[1,1′-biphenyl]-4-yl)boronic acid (Compound 27). LC-MS: 381.2 (M+1). ¹H NMR (400 MHz, DMSO-d6): δ=8.24-8.07 (m, 2H), 7.94-7.84 (m, 1H), 7.83-7.74 (m, 2H), 7.37-7.04 (m, 2H), 3.55 (s, 2H), 2.92 (s, 4H).

### Compound 28

### Synthesis of (2-hydroxy-4′-(4-oxo-3,5,7,8-tetrahydro-4H-thiopyrano[4,3-d]pyrimidin-2-yl)-[1,1′-biphenyl]-4-yl)boronic acid (Compound 28)

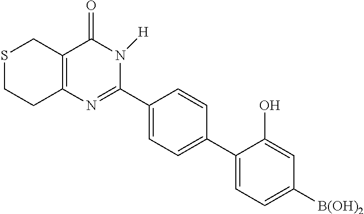 [SMILES: O=c1[nH]c(-c2ccc(-c3ccc(B(O)O)cc3O)cc2)nc2c1CSCC2]

### Synthesis of 4′-bromo-2′-methoxy-[1,1′-biphenyl]-4-carbonitrile

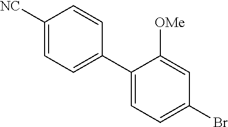 [SMILES: COc1cc(Br)ccc1-c1ccc(C#N)cc1]

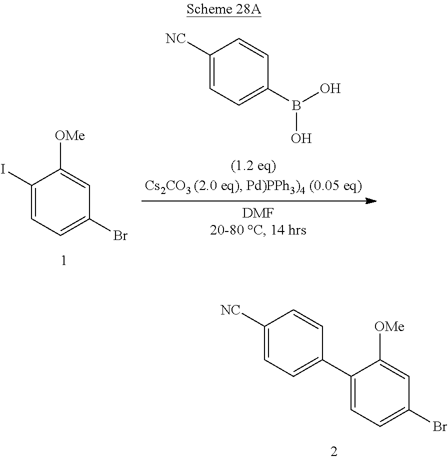 [SMILES: COc1cc(Br)ccc1I ; N#Cc1ccc(B(O)O)cc1 ; COc1cc(Br)ccc1-c1ccc(C#N)cc1]

To a solution of 4-bromo-1-iodo-2-methoxybenzene (1) (8.00 g, 25.6 mmol, 1.00 eq) and (4-cyanophenyl)boronic acid (4.51 g, 30.7 mmol, 1.20 eq) in DMF (80.0 mL) was added Cs₂CO₃ (16.7 g, 51.1 mmol, 2.00 eq) and Pd(PPh₃)₄ (1.48 g, 1.28 mmol, 0.05 eq) at 20° C. under N₂. The mixture was stirred at 80° C. for 14 h under N₂. TLC (petroleum ether/ethyl acetate=8/1, R_{f}=0.54) indicated that (1) was consumed completely. The reaction mixture was diluted with EtOAc (50.0 mL) and H₂O (150 mL). The layers were separated and the aqueous phase was extracted with EtOAc (50.0 mL×3). The combined organic layers were dried over Na₂SO₄, filtered, and the filtrate was concentrated under reduced pressure to give a residue that was purified by column chromatography (SiO₂, petroleum ether/ethyl acetate=10/1 to 0/1) to give 4′-bromo-2′-methoxy-[1,1′-biphenyl]-4-carbonitrile (2) (6.10 g, 21.2 mmol). ¹H NMR (400 MHz, DMSO-d6): δ=7.93 (d, J=8.4 Hz, 2H), 7.73 (d, J=8.4 Hz, 2H), 7.42 (d, J=1.4 Hz, 1H), 7.38-7.30 (m, 2H), 3.92-3.83 (m, 3H).

### Synthesis of 4′-bromo-2′-methoxy-[1,1′-biphenyl]-4-carboximidamide

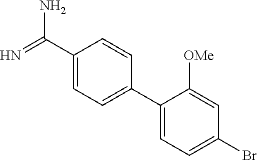 [SMILES: COc1cc(Br)ccc1-c1ccc(C(=N)N)cc1]

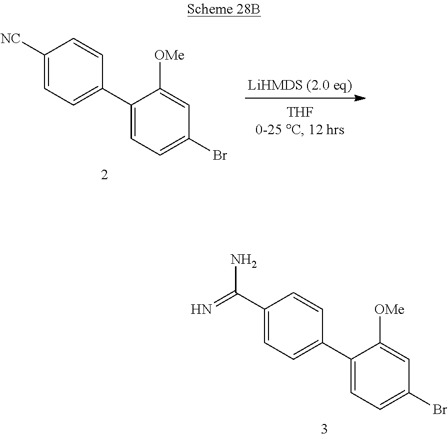 [SMILES: COc1cc(Br)ccc1-c1ccc(C#N)cc1 ; COc1cc(Br)ccc1-c1ccc(C(=N)N)cc1]

To a solution of 4′-bromo-2′-methoxy-[1,1′-biphenyl]-4-carbonitrile (2) (1.00 g, 3.47 mmol, 1.00 eq) in THF (10.0 mL), LiHMDS (1 M, 6.94 mL, 2.00 eq) was added dropwise at 0° C. The mixture was stirred at 25° C. for 12 h. LC-MS showed that (2) was consumed completely. The reaction mixture was quenched by dropwise addition of HCl/dioxane (6 mL) at 0° C., then concentrated under reduced pressure to give a residue that was suspended in MeOH (20 mL) and the mixture stirred at 25° C. for 1 h. The mixture was filtered and the filtrate was concentrated under reduced pressure to give 4′-bromo-2′-methoxy-[1,1′-biphenyl]-4-carboximidamide (3) (2.00 g, crude), which was used in the next step without further purification. LC-MS: 306.2+bromo isomers (M+1).

### Synthesis of 2-(4′-bromo-2′-methoxy-[1,1′-biphenyl]-4-yl)-3,5,7,8-tetrahydro-4H-thiopyrano[4,3-d]pyrimidin-4-one

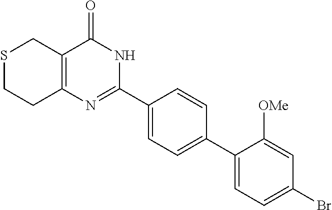 [SMILES: COc1cc(Br)ccc1-c1ccc(-c2nc3c(c(=O)[nH]2)CSCC3)cc1]

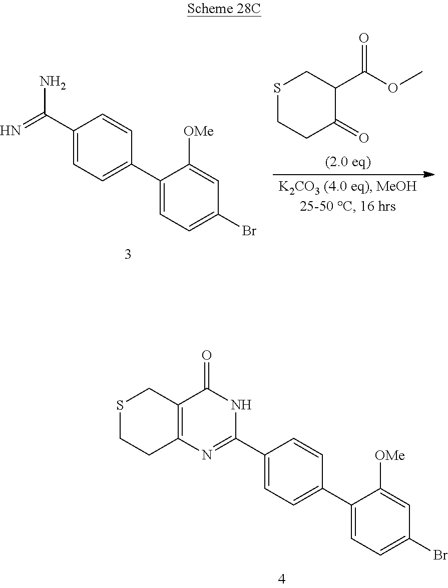 [SMILES: COc1cc(Br)ccc1-c1ccc(C(=N)N)cc1 ; COC(=O)C1CSCCC1=O ; COc1cc(Br)ccc1-c1ccc(-c2nc3c(c(=O)[nH]2)CSCC3)cc1]

To a solution of 4′-bromo-2′-methoxy-[1,1′-biphenyl]-4-carboximidamide (3) (1.00 g, 3.28 mmol, 1.00 eq) and methyl 4-oxotetrahydrothiopyran-3-carboxylate (1.14 g, 6.55 mmol, 2.00 eq) in MeOH (10 mL) was added K₂CO₃ (1.81 g, 13.1 mmol, 4.00 eq) at 25° C. The mixture was stirred at 50° C. for 16 h. LC-MS showed that (3) was consumed completely. The reaction mixture was filtered and filter cake dried under reduced pressure to give a residue. H₂O (15 mL) was added to make a slurry and the mixture stirred at 20° C. for 16 h. The mixture was filtered and the filter cake dried under reduced pressure to give 2-(4′-bromo-2′-methoxy-[1,1′-biphenyl]-4-yl)-3,5,7,8-tetrahydro-4H-thiopyrano[4,3-d]pyrimidin-4-one (4) (2.00 g, crude). The crude product was used in the next step without further purification. LC-MS: 430.3+bromo isomers (M+1).

### Synthesis of 2-(2′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one

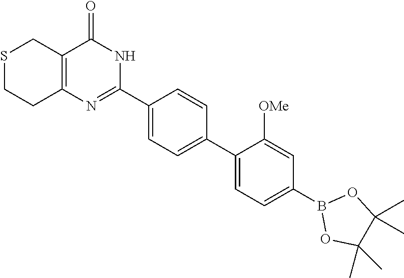 [SMILES: COc1cc(B2OC(C)(C)C(C)(C)O2)ccc1-c1ccc(-c2nc3c(c(=O)[nH]2)CSCC3)cc1]

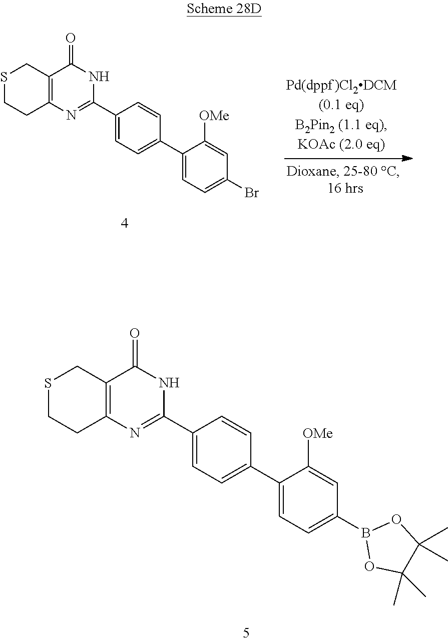 [SMILES: COc1cc(Br)ccc1-c1ccc(-c2nc3c(c(=O)[nH]2)CSCC3)cc1 ; COc1cc(B2OC(C)(C)C(C)(C)O2)ccc1-c1ccc(-c2nc3c(c(=O)[nH]2)CSCC3)cc1]

To a solution of 2-(4′-bromo-2′-methoxy-[1,1′-biphenyl]-4-yl)-3,5,7,8-tetrahydro-4H-thiopyrano[4,3-d]pyrimidin-4-one (4) (400 mg, 932 mol, 1 eq) and B₂Pin₂ (260 mg, 1.02 mmol, 1.10 eq) in dioxane (20 mL) was added KOAc (183 mg, 1.86 mmol, 2.00 eq) and Pd(dppf)Cl₂·DCM (76.1 mg, 93.17 mol, 0.1 eq) at 25° C. The mixture was stirred under N₂ at 80° C. for 16 h. LC-MS showed that (4) was consumed completely. The mixture was filtered and the filtrate was concentrated under reduced pressure to give 2-(2′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (700 mg, crude). The crude product was used in the next step without further purification. LC-MS: 477.2 (M+1).

### Synthesis of (2-methoxy-4′-(4-oxo-3,5,7,8-tetrahydro-4H-thiopyrano[4,3-d]pyrimidin-2-yl)-[1,1′-biphenyl]-4-yl)boronic acid

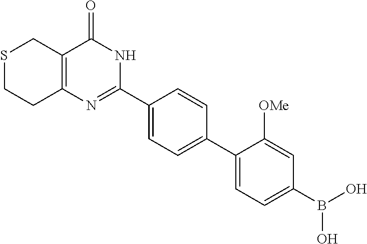 [SMILES: COc1cc(B(O)O)ccc1-c1ccc(-c2nc3c(c(=O)[nH]2)CSCC3)cc1]

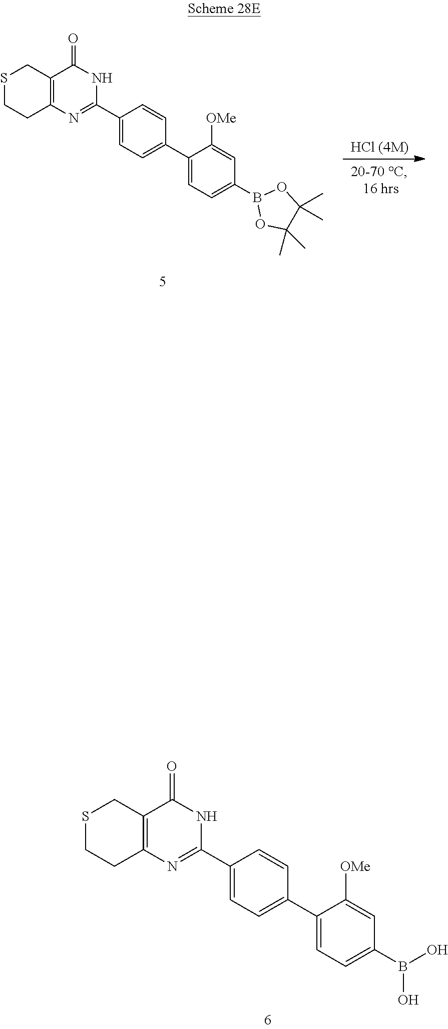 [SMILES: COc1cc(B2OC(C)(C)C(C)(C)O2)ccc1-c1ccc(-c2nc3c(c(=O)[nH]2)CSCC3)cc1 ; COc1cc(B(O)O)ccc1-c1ccc(-c2nc3c(c(=O)[nH]2)CSCC3)cc1]

2-(2′-methoxy-4′-(4,4,5,5-tetramethyl-1,3,2-dioxaborolan-2-yl)-[1,1′-biphenyl]-4-yl)-3,5,7,8-tetrahydro-4H-thiopyrano[4,3-d]pyrimidin-4-one (5) (400 mg, 840 mol, 1.0 eq) was added portion-wise to a stirred solution of HCl (4M, 20 mL, 95.3 eq) at 20° C. The mixture was stirred at 70° C. for 16 h. LC-MS showed that (5) was consumed completely. The reaction mixture was filtered, and the filtrate concentrated under reduced pressure to give (2-methoxy-4′-(4-oxo-3,5,7,8-tetrahydro-4H-thiopyrano[4,3-d]pyrimidin-2-yl)-[1,1′-biphenyl]-4-yl)boronic acid (6) (490 mg, crude). The crude product was used in the next step without further purification. LC-MS: 395.1 (M+1).

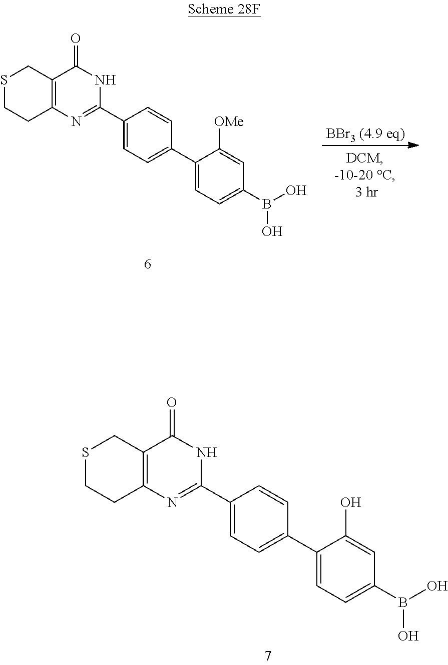 [SMILES: COc1cc(B(O)O)ccc1-c1ccc(-c2nc3c(c(=O)[nH]2)CSCC3)cc1 ; O=c1[nH]c(-c2ccc(-c3ccc(B(O)O)cc3O)cc2)nc2c1CSCC2]

To a solution of (2-methoxy-4′-(4-oxo-3,5,7,8-tetrahydro-4H-thiopyrano[4,3-d]pyrimidin-2-yl)-[1,1′-biphenyl]-4-yl)boronic acid (6) (200 mg, 507 mol, 1 eq) in DCM (3 mL), tribromoborane (1 M, 2.49 mL, 4.9 eq) was added dropwise at −10° C. The mixture was warmed to 20° C. and stirred for 3 h. LC-MS showed that (6) was consumed completely. The reaction mixture was filtered and the filter cake dried under reduced pressure to give a residue that was purified by prep-HPLC (column: Phenomenex luna C18 100×40 mm×5 um; mobile phase: [H₂O (0.04% HCl)-ACN]; gradient: 5%-45% B over 8.0 min). (2-hydroxy-4′-(4-oxo-3,5,7,8-tetrahydro-4H-thiopyrano[4,3-d]pyrimidin-2-yl)-[1,1′-biphenyl]-4-yl)boronic acid (Compound 28) was obtained. ¹H NMR (400 MHz, DMSO-d6): δ=9.69-9.32 (m, 1H), 8.12 (d, J=8.5 Hz, 2H), 7.73 (d, J=8.4 Hz, 2H), 7.39-7.37 (m, 1H), 7.36-7.28 (m, 2H), 3.64-3.59 (m, 2H), 2.91 (s, 4H). LC-MS: 381.1 (M+1).

### Compound 29

### Synthesis of methyl (2-((2-(4′-(4-oxo-3,5,7,8-tetrahydro-4H-thiopyrano[4,3-d]pyrimidin-2-yl)-[1,1′-biphenyl]-4-yl)propan-2-yl)oxy)ethyl)carbamate (Compound 29)

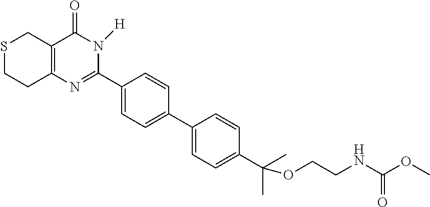 [SMILES: COC(=O)NCCOC(C)(C)c1ccc(-c2ccc(-c3nc4c(c(=O)[nH]3)CSCC4)cc2)cc1]

### Synthesis of 2-(4-bromophenyl)-3,5,7,8-tetrahydro-4H-thiopyrano[4,3-d]pyrimidin-4-one

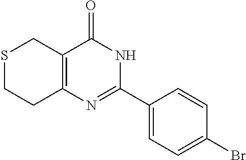 [SMILES: O=c1[nH]c(-c2ccc(Br)cc2)nc2c1CSCC2]

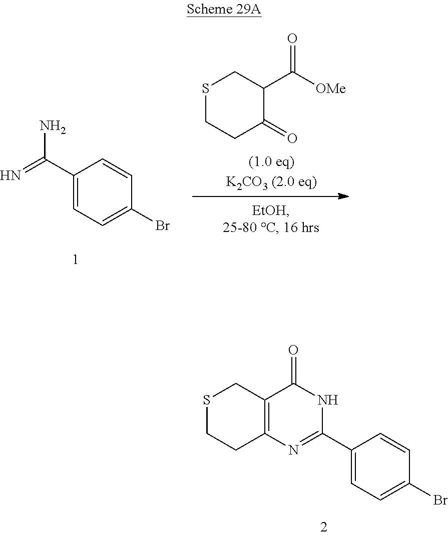 [SMILES: N=C(N)c1ccc(Br)cc1 ; COC(=O)C1CSCCC1=O ; O=c1[nH]c(-c2ccc(Br)cc2)nc2c1CSCC2]

To a solution of 4-bromobenzimidamide (1) (5 g, 21.2 mmol, 1.00 eq) in EtOH (50 mL) was added methyl 4-oxotetrahydrothiopyran-3-carboxylate (3.70 g, 21.2 mmol, 1.00 eq) and K₂CO₃ (5.87 g, 42.5 mmol, 2.00 eq) at 25° C. The mixture was stirred at 80° C. for 16 h. The reaction mixture was filtered, and the filtrate was concentrated under reduced pressure to give a residue. 2-(4-bromophenyl)-3,5,7,8-tetrahydro-4H-thiopyrano[4,3-d]pyrimidin-4-one (2) (7 g, crude) was obtained and used without further purification. ¹H NMR: (400 MHz, DMSO-d6): δ=8.21-8.11 (m, 2H), 7.53-7.46 (m, 2H), 3.42 (s, 2H), 2.83-2.64 (m, 4H).

### Synthesis of 2-(4-(4,4,5,5-tetramethyl-1,3,2-dioxaborolan-2-yl)phenyl)-3,5,7,8-tetrahydro-4H-thiopyrano[4,3-d]pyrimidin-4-one

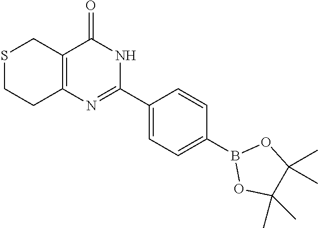 [SMILES: CC1(C)OB(c2ccc(-c3nc4c(c(=O)[nH]3)CSCC4)cc2)OC1(C)C]

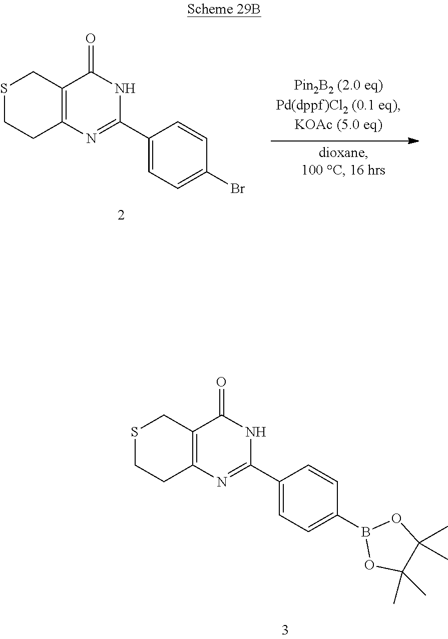 [SMILES: O=c1[nH]c(-c2ccc(Br)cc2)nc2c1CSCC2 ; CC1(C)OB(c2ccc(-c3nc4c(c(=O)[nH]3)CSCC4)cc2)OC1(C)C]

To a solution of 2-(4-bromophenyl)-3,5,7,8-tetrahydro-4H-thiopyrano[4,3-d]pyrimidin-4-one (2) (7 g, 21.7 mmol, 1.00 eq) in dioxane (70 mL) was added Pin₂B₂ (11.0 g, 43.3 mmol, 2.00 eq), KOAc (10.6 g, 108 mmol, 5.00 eq), and Pd(dppf)Cl₂ (1.58 g, 2.17 mmol, 0.10 eq) at 25° C. under N₂. The mixture was degassed three times with N₂. The mixture was stirred at 100° C. for 16 h. The reaction mixture was concentrated under reduced pressure to give a crude product. The crude product was triturated with H₂O (150 mL) at 20° C. for 2 h. The mixture was filtered, and the filter cake was concentrated under reduced pressure to give a residue. 2-(4-(4,4,5,5-tetramethyl-1,3,2-dioxaborolan-2-yl)phenyl)-3,5,7,8-tetrahydro-4H-thiopyrano[4,3-d]pyrimidin-4-one (3) (9 g, crude) was obtained and used immediately in the next step.

### Synthesis of methyl (2-((2-(4′-(4-oxo-3,5,7,8-tetrahydro-4H-thiopyrano[4,3-d]pyrimidin-2-yl)-[1,1′-biphenyl]-4-yl)propan-2-yl)oxy)ethyl)carbamate

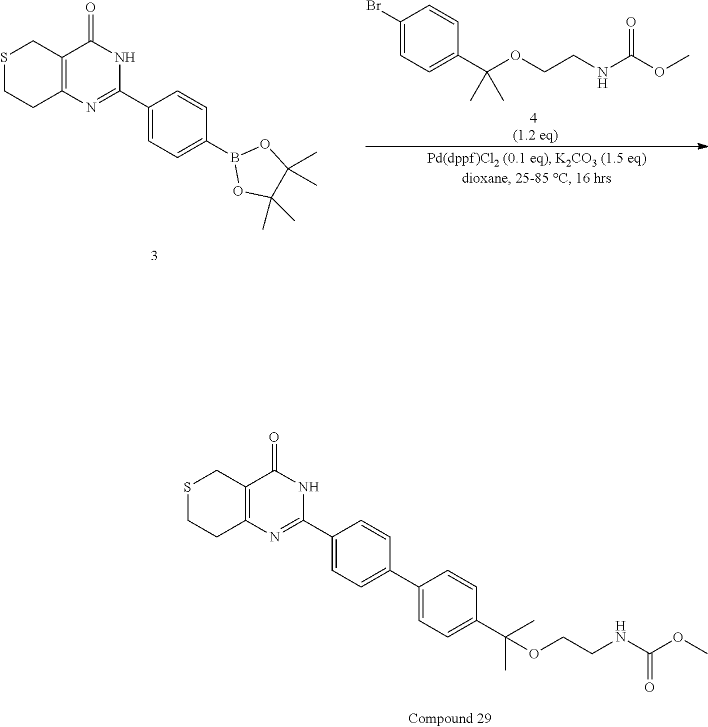 [SMILES: CC1(C)OB(c2ccc(-c3nc4c(c(=O)[nH]3)CSCC4)cc2)OC1(C)C ; COC(=O)NCCOC(C)(C)c1ccc(Br)cc1 ; COC(=O)NCCOC(C)(C)c1ccc(-c2ccc(-c3nc4c(c(=O)[nH]3)CSCC4)cc2)cc1]

To a solution of 2-(4-(4,4,5,5-tetramethyl-1,3,2-dioxaborolan-2-yl)phenyl)-3,5,7,8-tetrahydro-4H-thiopyrano[4,3-d]pyrimidin-4-one (3) (0.8 g, 2.16 mmol, 1.00 eq) in dioxane (16 mL) was added methyl (2-((2-(4-bromophenyl)propan-2-yl)oxy)ethyl)carbamate (4) (820 mg, 2.59 mmol, 1.20 eq), Pd(dppf)Cl₂ (158 mg, 216 mol, 0.10 eq), and K₂CO₃ (1.49 g, 10.8 mmol, 5 eq) at 25° C. under N₂. The mixture was degassed three times with N₂. The mixture was stirred at 85° C. for 16 h. The reaction mixture was concentrated under reduced pressure and the resultant residue diluted with H₂O (20 mL) and extracted with ethyl acetate 60 mL (20 mL×3). The combined organic layers were washed with brine (30 mL×1), dried over Na₂SO₄, filtered, and concentrated under reduced pressure. The residue was purified by prep-HPLC (column: Phenomenex Luna C18 75*30 mm*3 m; mobile phase: [NaHCO₃-ACN]; gradient: 40%-70% B over 16.0 min). Methyl (2-((2-(4′-(4-oxo-3,5,7,8-tetrahydro-4H-thiopyrano[4,3-d]pyrimidin-2-yl)-[1,1′-biphenyl]-4-yl)propan-2-yl)oxy)ethyl)carbamate (Compound 29) was obtained. LCMS: 480.1 (M+1). ¹H NMR: (400 MHz, DMSO-d6): δ=8.19 (d, J=8.4 Hz, 2H), 7.82 (d, J=8.5 Hz, 2H), 7.72 (d, J=8.4 Hz, 2H), 7.57-7.47 (m, 2H), 7.17-7.11 (m, 1H), 3.53 (s, 2H), 3.49 (s, 3H), 3.18-3.08 (m, 4H), 2.90 (br s, 4H), 1.49 (s, 6H).

### Compound 30

### Synthesis of (2-hydroxy-4-(4-oxo-3,5,7,8-tetrahydro-4H-thiopyrano[4,3-d]pyrimidin-2-yl)phenyl)boronic acid (Compound 30)

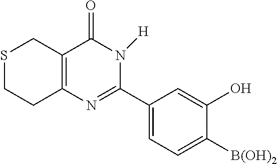 [SMILES: O=c1[nH]c(-c2ccc(B(O)O)c(O)c2)nc2c1CSCC2]

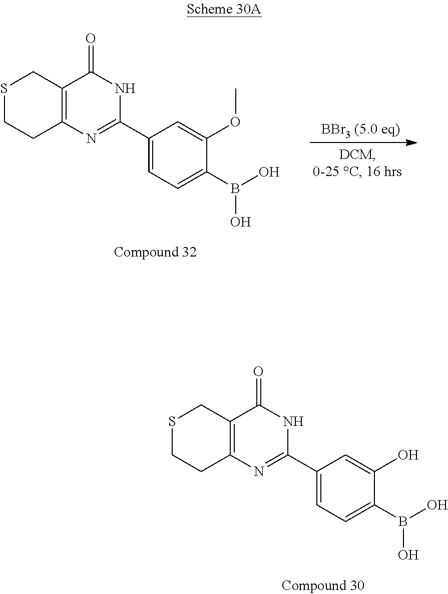 [SMILES: COc1cc(-c2nc3c(c(=O)[nH]2)CSCC3)ccc1B(O)O ; O=c1[nH]c(-c2ccc(B(O)O)c(O)c2)nc2c1CSCC2]

The synthesis of (2-methoxy-4-(4-oxo-3,5,7,8-tetrahydro-4H-thiopyrano[4,3-d]pyrimidin-2-yl)phenyl)boronic acid (Compound 32) is shown in Schemes 32A-D.

To a solution of (2-methoxy-4-(4-oxo-3,5,7,8-tetrahydro-4H-thiopyrano[4,3-d]pyrimidin-2-yl)phenyl)boronic acid (Compound 32) (0.30 g, 943 mol, 1.00 eq) in DCM (3 mL), was added BBr₃ (1.18 g, 4.71 mmol, 454 μL, 5.00 eq) dropwise at 0° C. and then stirred at 25° C. for 16 h. LC-MS showed that (Compound 32) was consumed. The reaction mixture was quenched by dropwise addition of H₂O (5 mL at 25° C.) and extracted with ethyl acetate (3 mL×3). The combined organic layers were washed and concentrated under reduced pressure to give a residue that was purified by prep-HPLC (column: Phenomenex Luna C_{8 250×50} mm×10 μm; mobile phase: [H₂O (0.04% HCl)-ACN]; gradient: 5%-40% B over 10.0 min). (2-hydroxy-4-(4-oxo-3,5,7,8-tetrahydro-4H-thiopyrano[4,3-d]pyrimidin-2-yl)phenyl)boronic acid (Compound 30) was obtained.

¹H NMR (400 MHz, DMSO-d6): δ=10.30-9.99 (m, 1H), 7.73 (d, J=7.8 Hz, 1H), 7.55-7.45 (m, 2H), 3.52 (s, 2H), 2.92-2.88 (m, 4H).

### Compound 31

### Synthesis of (3-hydroxy-4-(4-oxo-3,5,7,8-tetrahydro-4H-thiopyrano[4,3-d]pyrimidin-2-yl)phenyl)boronic acid (Compound 31)

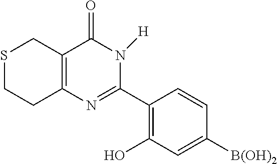 [SMILES: O=c1[nH]c(-c2ccc(B(O)O)cc2O)nc2c1CSCC2]

### Synthesis of 4-bromo-2-methoxybenzimidamide

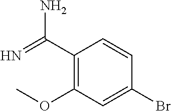

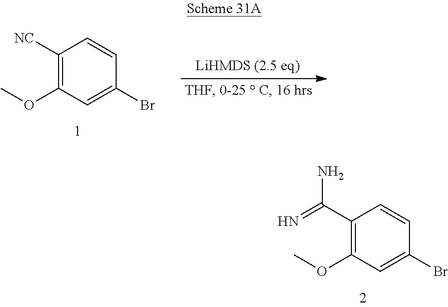

To a solution of 4-bromo-2-methoxybenzonitrile (1) (3.00 g, 14.2 mmol, 1.00 eq) in THF (30 mL), was added LiHMDS (1 M, 35.37 mL, 2.50 eq) dropwise at 0° C. and then stirred at 25° C. for 16 h. LC-MS showed that (1) was consumed. The reaction mixture was quenched by addition of 4 M HCl/dioxane (40 mL). The combined organic layers were concentrated under reduced pressure to give a residue which was suspended in MeOH (30 mL) and stirred at 15° C. for 2 h, then filtered. The filtrate was concentrated under reduced pressure to give 4-bromo-2-methoxybenzimidamide (2) (3.50 g, crude) was obtained. The crude product was used in the next step without further purification. ¹H NMR (400 MHz, DMSO-d6): δ=7.65-7.55 (m, 1H), 7.52-7.44 (m, 1H), 7.38-7.32 (m, 1H), 3.56 (s, 3H).

### Synthesis of 2-(4-bromo-2-methoxyphenyl)-3,5,7,8-tetrahydro-4H-thiopyrano[4,3-d]pyrimidin-4-one

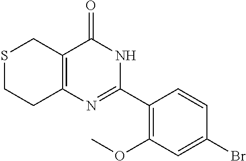 [SMILES: COc1cc(Br)ccc1-c1nc2c(c(=O)[nH]1)CSCC2]

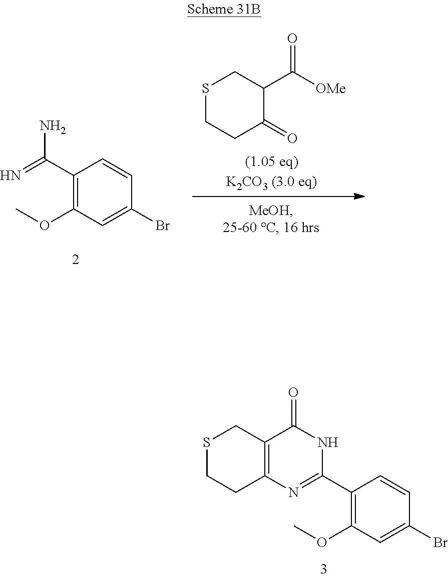 [SMILES: COc1cc(Br)ccc1C(=N)N ; COC(=O)C1CSCCC1=O ; COc1cc(Br)ccc1-c1nc2c(c(=O)[nH]1)CSCC2]

To a solution of 4-bromo-2-methoxybenzimidamide (2) (3.50 g, 15.3 mmol, 1.00 eq) in MeOH (35 mL) was added K₂CO₃ (6.34 g, 45.8 mmol, 3.00 eq) and methyl 4-oxotetrahydrothiopyran-3-carboxylate (2.79 g, 16.0 mmol, 1.05 eq) at 25° C. and then stirred at 60° C. for 16 h. LC-MS showed (2) was consumed. The reaction mixture was filtered, and the filtrate was concentrated under reduced pressure to give a residue that was purified by re-crystallization from MTBE (30 mL) at 25° C. 2-(4-bromo-2-methoxyphenyl)-3,5,7,8-tetrahydro-4H-thiopyrano[4,3-d]pyrimidin-4-one (3) (3.00 g, crude) was obtained. ¹H NMR (400 MHz, DMSO-d6): δ=7.24 (d, J=8.1 Hz, 1H), 7.16 (s, 1H), 7.11-7.07 (m, 1H), 3.72 (s, 3H), 3.45 (s, 2H), 2.74-2.58 (m, 4H).

### Synthesis of 2-(2-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one

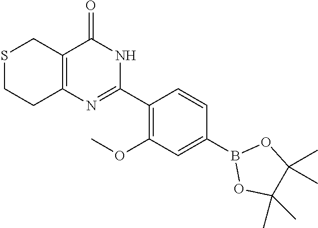 [SMILES: COc1cc(B2OC(C)(C)C(C)(C)O2)ccc1-c1nc2c(c(=O)[nH]1)CSCC2]

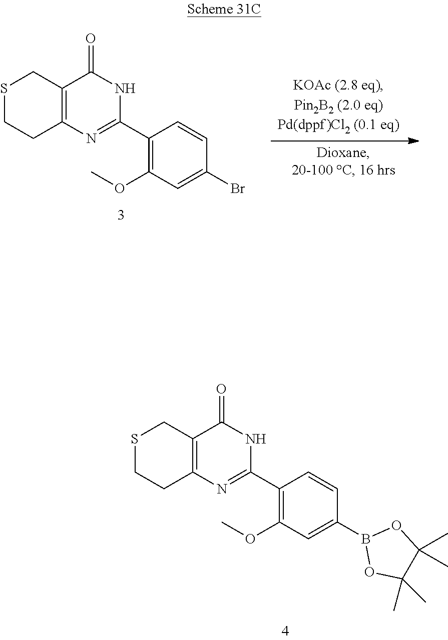 [SMILES: COc1cc(Br)ccc1-c1nc2c(c(=O)[nH]1)CSCC2 ; COc1cc(B2OC(C)(C)C(C)(C)O2)ccc1-c1nc2c(c(=O)[nH]1)CSCC2]

To a solution of 2-(4-bromo-2-methoxyphenyl)-3,5,7,8-tetrahydro-4H-thiopyrano[4,3-d]pyrimidin-4-one (3) (3.00 g, 8.49 mmol, 1.00 eq) in dioxane (30 mL) was added Pin₂B₂ (4.31 g, 17.0 mmol, 2.00 eq), K₂CO₃ (3.29 g, 23.8 mmol, 2.80 eq), and Pd(dppf)Cl₂ (621 mg, 849 mol, 0.10 eq) at 20° C. under N₂. The suspension was degassed and purged three times with N₂. The mixture was stirred under N₂ at 100° C. for 16 h. LC-MS showed that (3) was consumed. The reaction mixture was concentrated under reduced pressure to remove solvent and then diluted with H₂O (30 mL) and extracted with ethyl acetate (20 mL×3). The combined organic layers were washed with brine (30 mL), dried over Na₂SO₄, filtered, and concentrated under reduced pressure to give a residue. The crude product was triturated with MTBE at 25° C. for 120 min. 2-(2-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one (4) (2.5 g, crude) was obtained. The crude product was used in the next step without further purification. LC-MS: 401.2 (M+1).

### Synthesis of (3-methoxy-4-(4-oxo-3,5,7,8-tetrahydro-4H-thiopyrano[4,3-d]pyrimidin-2-yl)phenyl)boronic acid

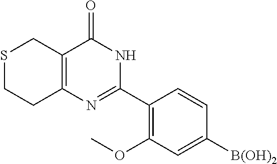 [SMILES: COc1cc(B(O)O)ccc1-c1nc2c(c(=O)[nH]1)CSCC2]

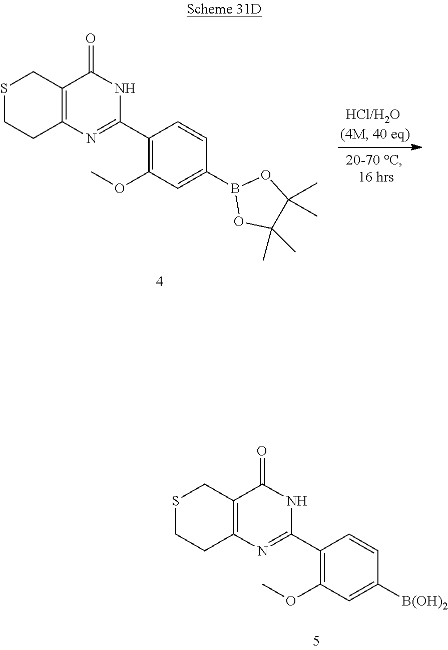 [SMILES: COc1cc(B2OC(C)(C)C(C)(C)O2)ccc1-c1nc2c(c(=O)[nH]1)CSCC2 ; COc1cc(B(O)O)ccc1-c1nc2c(c(=O)[nH]1)CSCC2]

A mixture of 2-(2-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one (4) (1.50 g, 3.75 mmol, 1.00 eq) in HCl/H₂O (4 M, 37.5 mL, 40.0 eq) was degassed and purged three times with N₂ at 20° C., and then stirred at 70° C. for 16 h under N₂. LC-MS showed (4) was consumed. The reaction mixture was concentrated under reduced pressure to give a residue that was triturated with MTBE at 25° C. for 120 min. The mixture was filtered, and the solvent was concentrated under reduced pressure to give a residue that was purified by prep-HPLC (column: Waters Xbridge Prep OBD C18 150×40 mm×10 um; mobile phase: [water (NH₄HCO₃)-ACN]; gradient: 10%-45% B over 8 min) to provide (3-methoxy-4-(4-oxo-3,5,7,8-tetrahydro-4H-thiopyrano[4,3-d]pyrimidin-2-yl)phenyl)boronic acid (5) (1.00 g, 3.14 mmol). ¹H NMR (400 MHz, DMSO-d6): δ=8.27 (s, 2H), 7.61-7.52 (m, 2H), 7.46 (d, J=7.8 Hz, 1H), 3.86 (s, 3H), 3.51 (s, 2H), 2.91-2.81 (m, 4H).

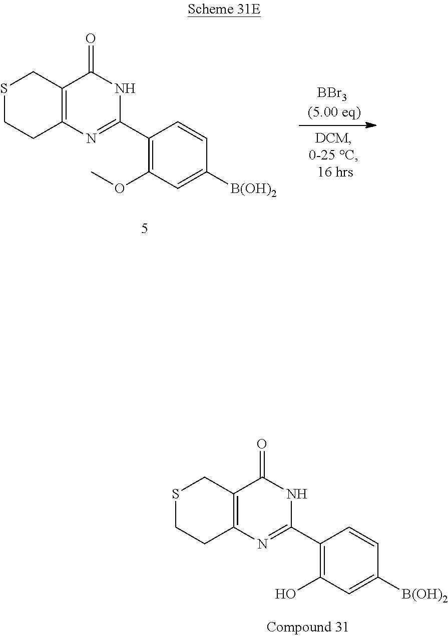 [SMILES: COc1cc(B(O)O)ccc1-c1nc2c(c(=O)[nH]1)CSCC2 ; O=c1[nH]c(-c2ccc(B(O)O)cc2O)nc2c1CSCC2]

To a solution of (3-methoxy-4-(4-oxo-3,5,7,8-tetrahydro-4H-thiopyrano[4,3-d]pyrimidin-2-yl)phenyl)boronic acid (5) (0.9 g, 2.83 mmol, 1 eq) in DCM (9 mL), was added BBr₃ (3.54 g, 14.1 mmol, 1.36 mL, 5.00 eq) dropwise at 0° C. and then stirred at 25° C. for 16 h. LC-MS showed that (5) was consumed. The reaction mixture was quenched by the addition of H₂O (5 mL at 25° C.) and extracted with ethyl acetate (3 mL×3). The combined organic layers were washed and concentrated under reduced pressure to give a residue that was purified by prep-HPLC (column: Phenomenex luna C18 100×40 mm×5 um; mobile phase: [H₂O (0.04% HCl)-ACN]; gradient: 5%-35% B over 8.0 min) to provide (3-hydroxy-4-(4-oxo-3,5,7,8-tetrahydro-4H-thiopyrano[4,3-d]pyrimidin-2-yl)phenyl)boronic acid (Compound 31). LCMS: 305.1 (M=1). ¹H NMR (400 MHz, DMSO-d6): δ=8.08 (br d, J=7.9 Hz, 1H), 7.78-7.43 (m, 2H), 7.37 (s, 1H), 7.31 (br d, J=7.7 Hz, 1H), 3.54 (br s, 2H), 2.98-2.84 (m, 4H).

### Compound 32

### Synthesis of (2-methoxy-4-(4-oxo-3,5,7,8-tetrahydro-4H-thiopyrano[4,3-d]pyrimidin-2-yl)phenyl)boronic acid (Compound 32)

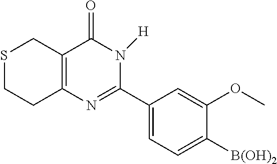 [SMILES: COc1cc(-c2nc3c(c(=O)[nH]2)CSCC3)ccc1B(O)O]

### Synthesis of 4-bromo-3-methoxybenzimidamide

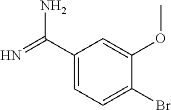

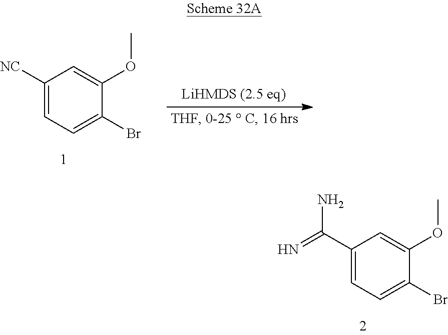

To a solution of 4-bromo-3-methoxybenzonitrile (1) (3.00 g, 14.2 mmol, 1.00 eq) in THF (30 mL), was added LiHMDS (1.00M, 35.4 mL, 2.50 eq) dropwise at 0° C. The mixture was warmed to 25° C. and stirred for 16 h. LC-MS showed that (1) was consumed. The reaction mixture was quenched by dropwise addition of 4 M HCl/dioxane (30 mL). The combined organic layers were concentrated under reduced pressure to give a residue. The residue was suspended in MeOH (30 mL) and stirred at 15° C. for 2 h, filtered, and concentrated under reduced pressure to give 4-bromo-3-methoxybenzimidamide (2) (4.00 g, crude). The crude product was used in the next step without further purification. ¹H NMR (400 MHz, DMSO-d6): 6=7.81 (d, J=8.3 Hz, 1H), 7.74 (d, J=1.4 Hz, 1H), 7.45 (dd, J=1.6, 8.3 Hz, 1H), 3.99 (s, 3H).

### Synthesis of 2-(4-bromo-3-methoxyphenyl)-3,5,7,8-tetrahydro-4H-thiopyrano[4,3-d]pyrimidin-4-one

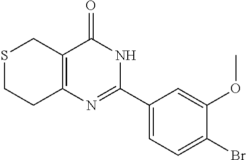 [SMILES: COc1cc(-c2nc3c(c(=O)[nH]2)CSCC3)ccc1Br]

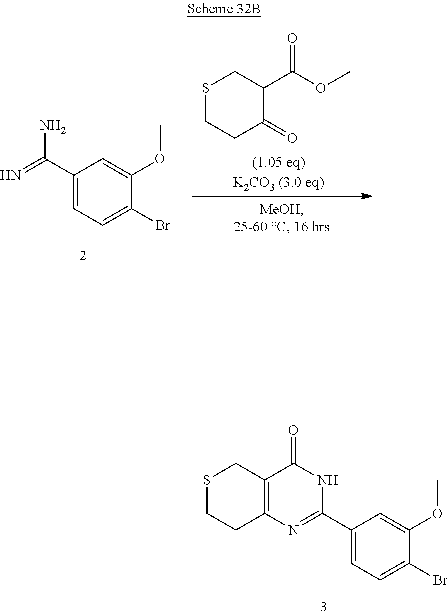

To a solution of 4-bromo-3-methoxybenzimidamide (2) (2.00 g, 8.73 mmol, 1.00 eq) in MeOH (20 mL) was added methyl 4-oxotetrahydrothiopyran-3-carboxylate (1.60 g, 9.17 mmol, 1.05 eq) and K₂CO₃ (3.62 g, 26.2 mmol, 3.00 eq) at 25° C. The mixture was stirred at 60° C. for 16 h. LC-MS showed that (2) was consumed completely. The reaction mixture was filtered, and the filtrate was concentrated under reduced pressure to give 2-(4-bromo-3-methoxyphenyl)-3,5,7,8-tetrahydro-4H-thiopyrano[4,3-d]pyrimidin-4-one (3) (3.00 g, crude) was obtained. The crude product was used in the next step without further purification.

¹H NMR (400 MHz, DMSO-d6): δ=13.16-12.30 (m, 1H), 7.78 (s, 1H), 7.73-7.68 (m, 1H), 7.66-7.60 (m, 1H), 3.95 (s, 3H), 3.52 (s, 2H), 2.88 (br s, 4H).

### Synthesis of 2-(3-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one

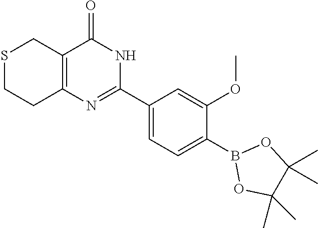 [SMILES: COc1cc(-c2nc3c(c(=O)[nH]2)CSCC3)ccc1B1OC(C)(C)C(C)(C)O1]

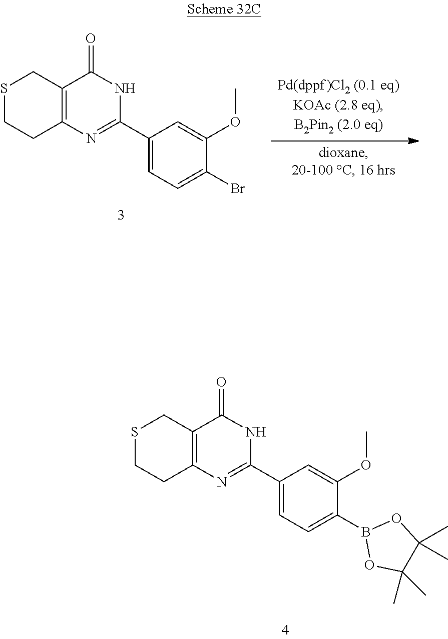 [SMILES: COc1cc(-c2nc3c(c(=O)[nH]2)CSCC3)ccc1Br ; COc1cc(-c2nc3c(c(=O)[nH]2)CSCC3)ccc1B1OC(C)(C)C(C)(C)O1]

To a solution of 2-(4-bromo-3-methoxyphenyl)-3,5,7,8-tetrahydro-4H-thiopyrano[4,3-d]pyrimidin-4-one (3) (2.00 g, 5.66 mmol, 1.00 eq) in dioxane (20 mL) was added B₂Pin₂ (2.88 g, 11.3 mmol, 2.00 eq), KOAc (1.56 g, 15.85 mmol, 2.80 eq), and Pd(dppf)Cl₂ (414 mg, 566 mol, 0.10 eq) at 25° C. The suspension was degassed and purged three times with N₂. The mixture was stirred at 100° C. for 16 h. LC-MS showed that (3) was consumed. The reaction mixture was concentrated under reduced pressure to provide a residue that was diluted with H₂O (40 mL) and extracted with ethyl acetate (20 mL×3). The combined organic layers were washed with brine (30 mL), dried over Na₂SO₄, filtered, and concentrated under reduced pressure to give 2-(3-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one (4) (2.50 g, crude) was obtained. The crude product was used in the next step without further purification. LC-MS: 401.3 (M+1).

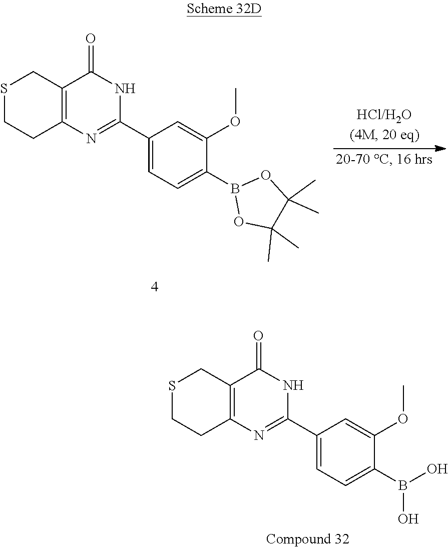 [SMILES: COc1cc(-c2nc3c(c(=O)[nH]2)CSCC3)ccc1B1OC(C)(C)C(C)(C)O1 ; COc1cc(-c2nc3c(c(=O)[nH]2)CSCC3)ccc1B(O)O]

A mixture of 2-(3-methoxy-4-(4,4,5,5-tetramethyl-1,3,2-dioxaborolan-2-yl)phenyl)-3,5,7,8-tetrahydro-4H-thiopyrano[4,3-d]pyrimidin-4-one (4) (1.00 g, 2.50 mmol, 1.00 eq) in HCl/H₂O (4 M, 12.5 mL, 20 eq) was degassed and purged three times with N₂, and then stirred at 70° C. for 16 h under N₂. LC-MS showed that (4) was consumed. The reaction mixture was concentrated under reduced pressure to give a residue that was triturated with MTBE at 25° C. for 120 min. The mixture was then filtered, and the filter cake dried under reduced pressure to give a residue that was purified by prep-HPLC (column: Waters Xbridge BEH C18 250×50 mm×10 μm; mobile phase: [water (NH₄HCO₃)-ACN]; B %: 5%-45%, 10 min). (2-methoxy-4-(4-oxo-3,5,7,8-tetrahydro-4H-thiopyrano[4,3-d]pyrimidin-2-yl)phenyl)boronic acid (Compound 32) was obtained. ¹H NMR (400 MHz, DMSO-d6): δ=13.14-12.16 (m, 1H), 7.96-7.81 (m, 2H), 7.74-7.53 (m, 3H), 3.89 (br s, 3H), 3.53 (br s, 2H), 2.89 (br s, 4H).

### Compound 33

### Synthesis of methyl (2-((2-(2-hydroxy-4-(4-oxo-3,5,7,8-tetrahydro-4H-thiopyrano[4,3-d]pyrimidin-2-yl)phenyl)propan-2-yl)oxy)ethyl)carbamate (Compound 33)

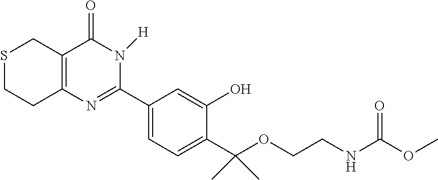 [SMILES: COC(=O)NCCOC(C)(C)c1ccc(-c2nc3c(c(=O)[nH]2)CSCC3)cc1O]

### Synthesis of methyl 4-cyano-2-methoxybenzoate

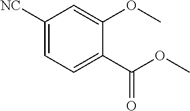

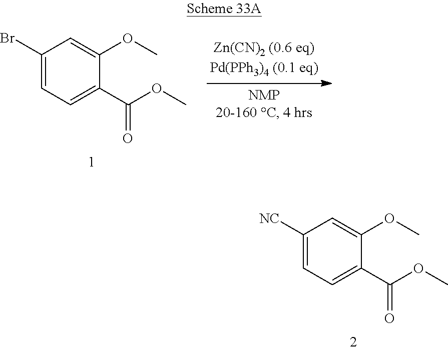

Methyl 4-bromo-2-methoxybenzoate (1) (10 g, 40.80 mmol, 1.0 eq) and Zn(CN)₂ (2.87 g, 24.48 mmol, 1.55 mL, 0.6 eq) were added to NMP (100 mL) in one portion at 20° C. under N₂. Then Pd(PPh₃)₄ (4.72 g, 4.08 mmol, 0.1 eq) was added to the mixture in one portion at 20° C. under N₂, and the mixture stirred at 160° C. for 4 hr and then cooled to 20° C. and filtered. The filter cake was washed with EtOAc (100 mL×2). The organic layer was then washed with aq. sat. Na₂CO₃ (50 mL×2). The organic layer was concentrated under reduced pressure to give a residue that was purified by column chromatography (SiO₂, petroleum ether/ethyl acetate=50/1 to 0/1) to provide methyl 4-cyano-2-methoxybenzoate (2) (4 g, crude) was obtained and used immediately in the next step.

### Synthesis of Methyl 4-carbamimidoyl-2-methoxybenzoate

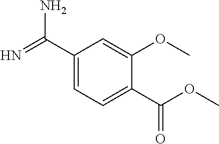

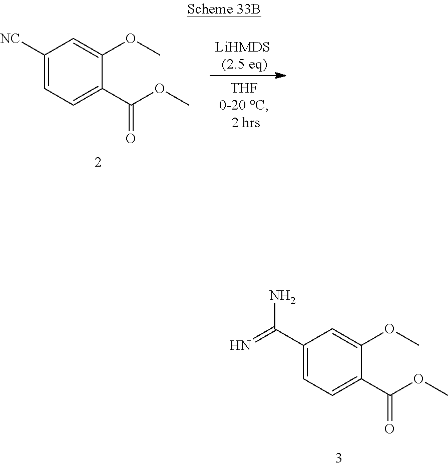

Methyl 4-cyano-2-methoxybenzoate (2) (3.0 g, 15.69 mmol, 1.0 eq) was added to THE (30 mL) at 20° C. under N₂. LiHMDS (1.0 M, 39.23 mL, 2.5 eq) was added dropwise to the mixture at 0° C. under N₂, and the mixture stirred at 20° C. under N₂ for 2 h. LC-MS showed that (2) was consumed completely. The reaction mixture was quenched with HCl/dioxane (30 mL) at 0° C. and then diluted with MeOH (30 mL). The mixture was filtered, and the filtrate concentrated under reduced pressure to give methyl 4-carbamimidoyl-2-methoxybenzoate (3) (4 g, crude, HCl) and used immediately in the next step. LC-MS: 209.2 (M+1).

### Synthesis of methyl 2-methoxy-4-(4-oxo-3,5,7,8-tetrahydro-4H-thiopyrano[4,3-d]pyrimidin-2-yl)benzoate

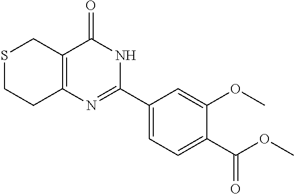 [SMILES: COC(=O)c1ccc(-c2nc3c(c(=O)[nH]2)CSCC3)cc1OC]

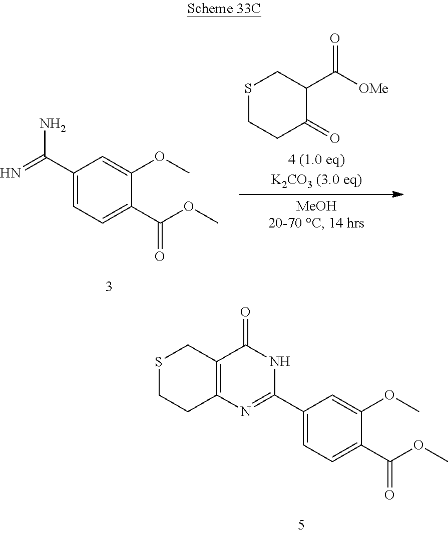 [SMILES: COC(=O)c1ccc(C(=N)N)cc1OC ; COC(=O)C1CSCCC1=O ; COC(=O)c1ccc(-c2nc3c(c(=O)[nH]2)CSCC3)cc1OC]

Methyl 4-carbamimidoyl-2-methoxybenzoate (3) (4.0 g, 19.21 mmol, 1.0 eq) and methyl 4-oxotetrahydro-2H-thiopyran-3-carboxylate (4) (3.35 g, 19.21 mmol, 1.0 eq) were added to MeOH (40 mL) at 20° C. K₂CO₃ was added (7.97 g, 57.63 mmol, 3.0 eq) to the mixture at 20° C. and the mixture stirred at 70° C. for 14 h under N₂. LC-MS showed that (3) was completely consumed. The mixture was filtered and concentrated to provide a residue that was purified by prep-HPLC (column: Agela DuraShell C18 250×70 mm×10 um; mobile phase: [H₂O (10 mM NH₄HCO₃)-ACN]; gradient: 25%-45% B over 17.0 m) to provide Methyl 2-methoxy-4-(4-oxo-3,5,7,8-tetrahydro-4H-thiopyrano[4,3-d]pyrimidin-2-yl)benzoate (5) (0.5 g, 1.50 mmol). LC-MS: 333.3 (M+1).

### Synthesis of methyl 2-hydroxy-4-(4-oxo-3,5,7,8-tetrahydro-4H-thiopyrano[4,3-d]pyrimidin-2-yl)benzoate

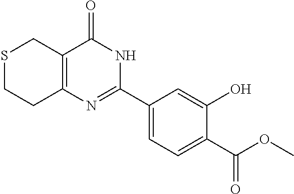 [SMILES: COC(=O)c1ccc(-c2nc3c(c(=O)[nH]2)CSCC3)cc1O]

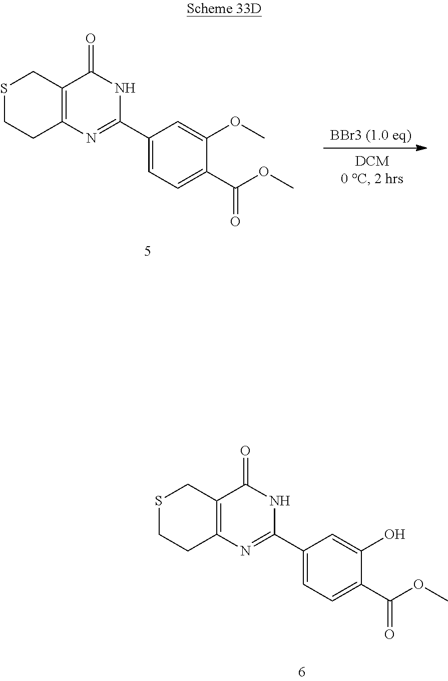 [SMILES: COC(=O)c1ccc(-c2nc3c(c(=O)[nH]2)CSCC3)cc1OC ; COC(=O)c1ccc(-c2nc3c(c(=O)[nH]2)CSCC3)cc1O]

Methyl 2-methoxy-4-(4-oxo-3,5,7,8-tetrahydro-4H-thiopyrano[4,3-d]pyrimidin-2-yl)benzoate (5) (250 mg, 752.17 mol, 1.0 eq) was added to DCM (5.0 mL) at 20° C. and the mixture cooled to 0° C. BBr₃ (188.43 mg, 752.17 mol, 72.47 μL, 1.0 eq) was added dropwise to the mixture at 0° C. and the reaction mixture was stirred at 0° C. for 2 h under N₂. LC-MS indicated that (6) was detected. The reaction mixture was quenched with MeOH (2.0 mL) at 0° C. under N₂. The mixture was filtered, and the filtrate concentrated under reduced pressure to give a residue that was purified by column chromatography (SiO₂, petroleum ether/ethyl acetate=50/1 to 0/1) to provide methyl 2-hydroxy-4-(4-oxo-3,5,7,8-tetrahydro-4H-thiopyrano[4,3-d]pyrimidin-2-yl)benzoate (6) (250 mg) that was used in the next step without further purification. LC-MS: 319.3 (M+1).

### Synthesis of 2-(3-hydroxy-4-(2-hydroxypropan-2-yl)phenyl)-3,5,7,8-tetrahydro-4H-thiopyrano[4,3-d]pyrimidin-4-one

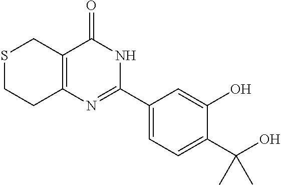 [SMILES: CC(C)(O)c1ccc(-c2nc3c(c(=O)[nH]2)CSCC3)cc1O]

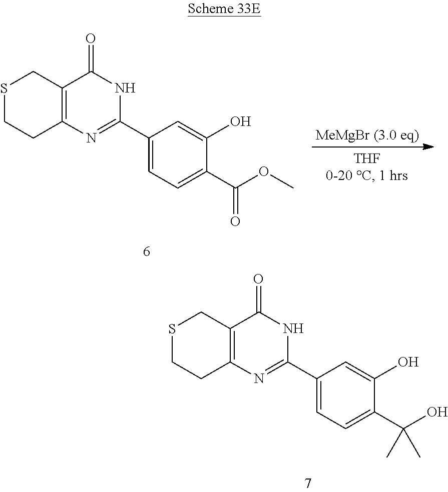 [SMILES: COC(=O)c1ccc(-c2nc3c(c(=O)[nH]2)CSCC3)cc1O ; CC(C)(O)c1ccc(-c2nc3c(c(=O)[nH]2)CSCC3)cc1O]

Methyl 2-hydroxy-4-(4-oxo-3,5,7,8-tetrahydro-4H-thiopyrano[4,3-d]pyrimidin-2-yl)benzoate (6) (80 mg, 251.30 mol, 1.0 eq) was added to THE (0.8 mL) at 20° C. and then the mixture cooled to 0° C. MeMgBr (3.0 M, 251.30 μL, 3.0 eq) was added to the mixture at 0° C. and the reaction mixture stirred at 20° C. under N₂ for 1 h. LC-MS showed that (6) was consumed. The reaction mixture was quenched with NH₄Cl (1.0 mL), and then extracted with EtOAc (1.0 mL×3). The combined organic layers were dried over Na₂SO₄, filtered, and then concentrated under reduced pressure to give a residue. The residue was purified by prep-TLC (petroleum ether/ethyl acetate=0/1) to provide 2-(3-hydroxy-4-(2-hydroxypropan-2-yl)phenyl)-3,5,7,8-tetrahydro-4H-thiopyrano[4,3-d]pyrimidin-4-one (7) (90 mg, crude). LC-MS: 319.3 (M+1).

### Synthesis of methyl (2-hydroxyethyl)carbamate

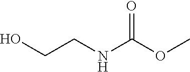

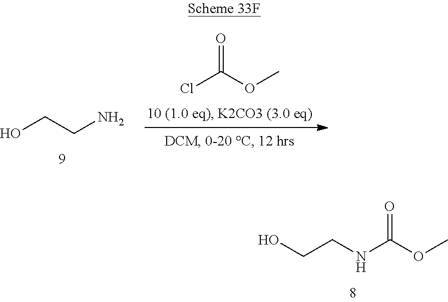

2-aminoethan-1-ol (9) (6.46 g, 105.82 mmol, 6.39 mL, 1.0 eq) and K₂CO₃ (43.88 g, 317.47 mmol, 3.0 eq) were added to DCM (45 mL) at 20° C. under N₂ and then cooled to 0° C. Carbonochloridate (10) (10 g, 105.82 mmol, 8.18 mL, 1.0 eq) was added to the mixture at 0° C. under N₂ and mixture allowed to warm to 20° C. under N₂ for 12 h. The reaction mixture was then quenched with NH₃·H₂O (10%, 100 mL) and extracted with EtOAc (50 mL×3). The organic layers were concentrated under reduced pressure to give crude methyl (2-hydroxyethyl)carbamate (8) (14.2 g, crude). ¹H NMR (400 MHz, CDCl₃-d): δ 5.36-5.11 (m, 1H), 3.78-3.61 (m, 5H), 3.35 (br s, 2H).

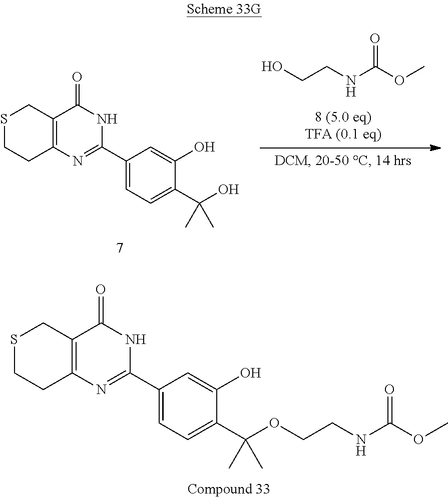

2-(3-hydroxy-4-(2-hydroxypropan-2-yl)phenyl)-3,5,7,8-tetrahydro-4H-thiopyrano[4,3-d]pyrimidin-4-one (7) (90 mg, 282.67 mol, 1.0 eq) and methyl (2-hydroxyethyl)carbamate (8) (168.36 mg, 1.41 mmol, 5.0 eq) were added to DCM (0.9 mL) at 20° C. under N₂. Then TFA (3.22 mg, 28.27 mol, 2.10 μL, 0.1 eq) was at 20° C. and the mixture stirred at 50° C. under N₂ for 14 h. The reaction mixture was concentrated under reduced pressure to give a residue that was subjected to prep-HPLC (column: Agela DuraShell C18 250×70 mm×10 um; mobile phase: [H₂O (10 mM NH₄HCO₃)-ACN]; gradient: 20%-43% B over 17.0 m) to provide methyl (2-((2-(2-hydroxy-4-(4-oxo-3,5,7,8-tetrahydro-4H-thiopyrano[4,3-d]pyrimidin-2-yl)phenyl)propan-2-yl)oxy)ethyl)carbamate (Compound 33). LC-MS: 420.5 (M+1). ¹H NMR (400 MHz, DMSO-d₆): δ 12.82-12.46 (m, 1H), 9.49 (br d, J=1.1 Hz, 1H), 7.58-7.47 (m, 2H), 7.38 (br d, J=8.2 Hz, 1H), 7.21 (br t, J=5.4 Hz, 1H), 3.58-3.47 (m, 6H), 3.18 (q, J=5.5 Hz, 2H), 2.94-2.79 (m, 5H), 1.54 (s, 6H).

### Compound 34

### Synthesis of methyl (2-((2-(2-methoxy-4-(4-oxo-3,5,7,8-tetrahydro-4H-thiopyrano[4,3-d]pyrimidin-2-yl)phenyl)propan-2-yl)oxy)ethyl)carbamate (Compound 34)

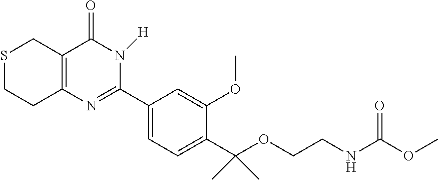 [SMILES: COC(=O)NCCOC(C)(C)c1ccc(-c2nc3c(c(=O)[nH]2)CSCC3)cc1OC]

### Synthesis of 2-(4-bromo-2-methoxyphenyl)propan-2-ol

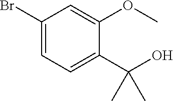

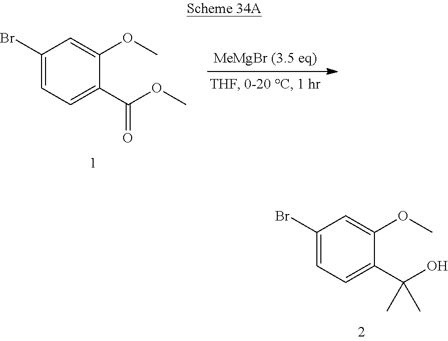

Methyl 4-bromo-2-methoxybenzoate (1) (10 g, 40.80 mmol, 1.0 eq) was added to THE (100 mL) at 20° C. MeMgBr (3 M, 47.61 mL, 3.5 eq) was then added dropwise at 0° C. and stirred at 20° C. under N₂ for 1 h. TLC (petroleum ether/ethyl acetate=5/1) indicated that (1) was consumed completely and many new spots were formed. Saturated NH₄Cl (150 mL) was added dropwise to the reaction mixture at 0° C. and then the mixture extracted with EtOAc (50 mL). The combined organic layers were dried over Na₂SO₄, filtered, and the filtrate was concentrated under reduced pressure to give a residue that was purified by column chromatography (SiO₂, petroleum ether/ethyl acetate=50/1 to 0/1) to obtain 2-(4-bromo-2-methoxyphenyl)propan-2-ol (2) (8 g, 32.64 mmol). LC-MS: 229 (bromo isomers, —OH) [M+1].

### Synthesis of 4-bromo-2-methoxy-1-(2-(2-nitroethoxy)propan-2-yl)benzene

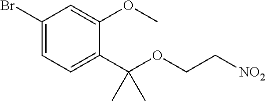 [SMILES: COc1cc(Br)ccc1C(C)(C)OCC[N+](=O)[O-]]

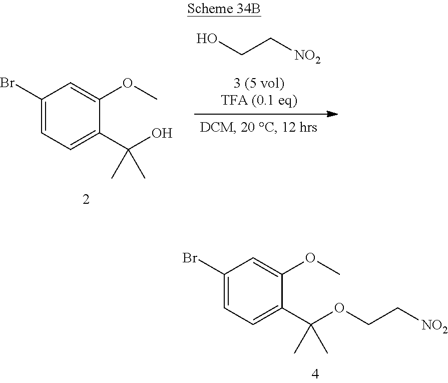 [SMILES: O=[N+]([O-])CCO ; COc1cc(Br)ccc1C(C)(C)O ; COc1cc(Br)ccc1C(C)(C)OCC[N+](=O)[O-]]

2-(4-bromo-2-methoxyphenyl)propan-2-ol (2) (5.0 g, 20.40 mmol, 1.0 eq) in dichloromethane (25 mL) was added (3) (32.30 g, 354.69 mmol, 25 mL, 17.39 eq) at 20 under N₂. TFA (232.59 mg, 2.04 mmol, 151.52 μL, 0.1 eq) was added to the mixture at 20° C. under N₂ and the mixture was stirred at 20° C. under N₂ for 12 h. TLC (petroleum ether/ethyl acetate=5/1) indicated the reaction was consumed completely and a major new spot was detected. The reaction mixture was diluted with H₂O (50.0 mL) and extracted with DCM (30 mL×3). The organic layers were dried over Na₂SO₄, filtered, and the organic layer was concentrated under reduced pressure to give a residue that was purified by column chromatography (SiO₂, petroleum ether/ethyl acetate=100/1 to 0/1) to provide 4-bromo-2-methoxy-1-(2-(2-nitroethoxy)propan-2-yl)benzene (4) (4.0 g, 12.57 mmol). ¹H NMR (400 MHz, CDCl₃): δ=7.21 (br d, J=8.2 Hz, 1H), 7.07 (br d, J=8.3 Hz, 1H), 7.02 (s, 1H), 4.51 (br t, J=5.0 Hz, 2H), 3.86-3.76 (m, 5H), 1.57 (s, 6H).

### Synthesis of 2-((2-(4-bromo-2-methoxyphenyl)propan-2-yl)oxy)ethan-1-amine

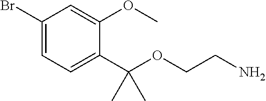 [SMILES: COc1cc(Br)ccc1C(C)(C)OCCN]

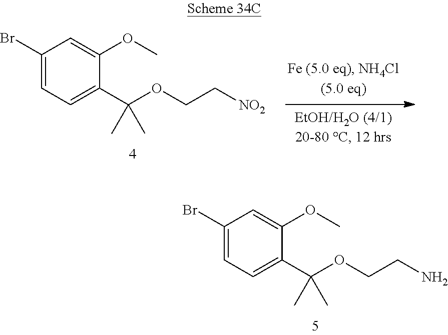 [SMILES: COc1cc(Br)ccc1C(C)(C)OCC[N+](=O)[O-] ; COc1cc(Br)ccc1C(C)(C)OCCN]

4-bromo-2-methoxy-1-(2-(2-nitroethoxy)propan-2-yl)benzene (4) (4.0 g, 12.57 mmol, 1.0 eq) in EtOH (32 mL) and H₂O (8 mL) was added Fe (3.51 g, 62.86 mmol, 5.0 eq) and NH₄Cl (3.36 g, 62.86 mmol, 5.0 eq) at 20° C. and stirred at 80° C. under N₂ for 12 h. TLC (petroleum ether/ethyl acetate=2/1) indicated the reaction was consumed completely and a major new spot was detected. The reaction mixture was filtered and the filtrate concentrated. Then the resulting oil was diluted with H₂O (50 mL) and the mixture extracted with EtOAc (20 mL×3). The combined organic layers were dried over Na₂SO₄, filtered, and concentrated under reduced pressure to give 2-((2-(4-bromo-2-methoxyphenyl)propan-2-yl)oxy)ethan-1-amine (5) (3.0 g, crude). The crude product was used in the next step without purification.

¹H NMR (400 MHz, DMSO-d₆): δ=7.80 (br s, 2H), 7.37 (d, J=8.3 Hz, 1H), 7.19 (d, J=1.7 Hz, 1H), 7.11 (dd, J=1.7, 8.3 Hz, 1H), 3.83 (s, 3H), 3.42 (t, J=5.4 Hz, 2H), 2.97 (t, J=5.4 Hz, 2H), 1.52 (s, 6H).

### Synthesis of methyl (2-((2-(4-bromo-2-methoxyphenyl)propan-2-yl)oxy)ethyl)carbamate

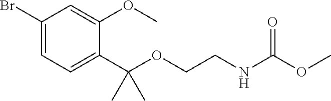 [SMILES: COC(=O)NCCOC(C)(C)c1ccc(Br)cc1OC]

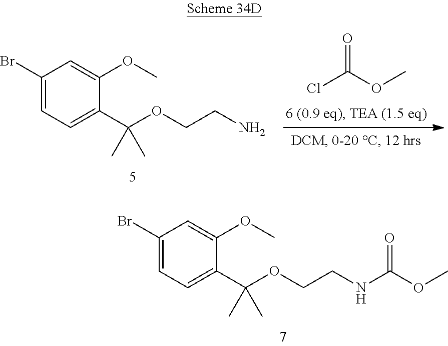

2-((2-(4-bromo-2-methoxyphenyl)propan-2-yl)oxy)ethan-1-amine (5) (3.0 g, 10.41 mmol, 1.0 eq) in DCM (30 mL) was added TEA (1.58 g, 15.62 mmol, 2.17 mL, 1.5 eq) at 20° C. Methyl carbonochloridate (6) (885.35 mg, 9.37 mmol, 723.92 μL, 0.9 eq) was added at 0° C. under N₂ and the mixture was stirred at 20° C. under N₂ for 12 h. LC-MS showed that (5) was consumed. The reaction mixture was quenched with NH₃·H₂O (10%, 20 mL) and extracted with DCM (20 mL×3). The organic layers were concentrated under reduced pressure to give a residue that was purified by column chromatography (SiO₂, petroleum ether/ethyl acetate=100/1 to 0/1) to provide methyl (2-((2-(4-bromo-2-methoxyphenyl)propan-2-yl)oxy)ethyl)carbamate (7) (1.0 g, 2.89 mmol). ¹H NMR (400 MHz, CDCl₃): δ=7.11 (d, J=8.2 Hz, 1H), 7.01-6.95 (m, 2H), 5.40-5.22 (m, 1H), 3.80 (s, 3H), 3.59 (s, 3H), 3.28-3.18 (m, 4H), 1.50 (s, 6H).

### Synthesis of methyl (2-((2-(4-cyano-2-methoxyphenyl)propan-2-yl)oxy)ethyl)carbamate

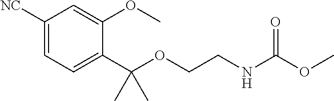 [SMILES: COC(=O)NCCOC(C)(C)c1ccc(C#N)cc1OC]

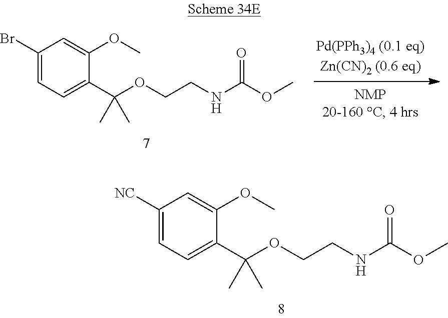 [SMILES: COC(=O)NCCOC(C)(C)c1ccc(Br)cc1OC ; COC(=O)NCCOC(C)(C)c1ccc(C#N)cc1OC]

Methyl (2-((2-(4-bromo-2-methoxyphenyl)propan-2-yl)oxy)ethyl)carbamate (7) (1.0 g, 2.89 mmol, 0.9 eq) was added into NMP (10 mL) at 20° C. under N₂. Zn(CN)₂ (226.10 mg, 1.93 mmol, 122.22 μL, 0.6 eq) and Pd(PPh₃)₄ (370.85 mg, 320.93 mol, 0.1 eq) were added to the mixture at 20° C. under N₂. The reaction mixture was stirred at 160° C. and microwaved under N₂ for 4 h. TLC (petroleum ether/ethyl acetate=2/1) indicated some (7) remained, and some major new spots were detected. The reaction mixture was cooled to 20° C. and saturated Na₂CO₃ (50 mL) was added to the mixture at 0° C. The mixture was extracted with EtOAc (20 mL×3) and the combined organic layers were concentrated under reduced pressure to give a residue that was purified by column chromatography (SiO₂, petroleum ether/ethyl acetate=50/1 to 0/1) to provide methyl (2-((2-(4-cyano-2-methoxyphenyl)propan-2-yl)oxy)ethyl)carbamate (8) (0.34 g, 1.16 mmol). ¹H NMR (400 MHz, CDCl₃): δ=7.48 (d, J=7.9 Hz, 1H), 7.25 (d, J=1.5 Hz, 1H), 7.14 (d, J=1.0 Hz, 1H), 5.28 (br s, 1H), 3.91 (s, 3H), 3.68 (s, 3H), 3.35 (br s, 4H), 1.59 (s, 6H).

### Synthesis of methyl (2-((2-(4-carbamimidoyl-2-methoxyphenyl)propan-2-yl)oxy)ethyl)carbamate

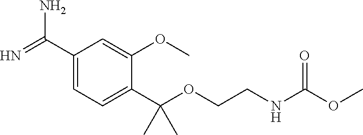 [SMILES: COC(=O)NCCOC(C)(C)c1ccc(C(=N)N)cc1OC]

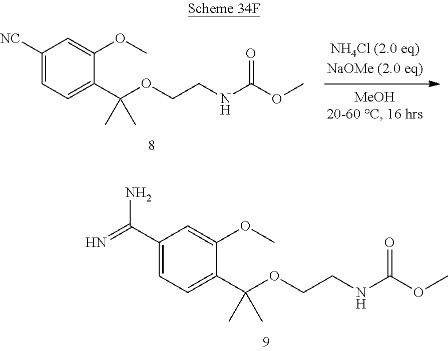 [SMILES: COC(=O)NCCOC(C)(C)c1ccc(C#N)cc1OC ; COC(=O)NCCOC(C)(C)c1ccc(C(=N)N)cc1OC]

Methyl (2-((2-(4-cyano-2-methoxyphenyl)propan-2-yl)oxy)ethyl)carbamate (8) (0.34 g, 1.16 mmol, 1.0 eq) was added to MeOH (3.4 mL) at 20° C. under N₂. NaOMe (125.66 mg, 2.33 mmol, 2.0 eq) was then added to the mixture at 20° C. under N₂ and stirred at 40° C. under N₂ for 12 h. NH₄Cl (124.43 mg, 2.33 mmol, 2.0 eq) was added to the mixture at 40° C. and the mixture was stirred at 60° C. under N₂ for 4 h. LC-MS showed the desired compound was detected. The mixture was then filtered and the filtrate was concentrated under reduced pressure to give methyl (2-((2-(4-carbamimidoyl-2-methoxyphenyl)propan-2-yl)oxy)ethyl)carbamate (9) (1.0 g, crude). LC-MS: 310.3 (M+1).

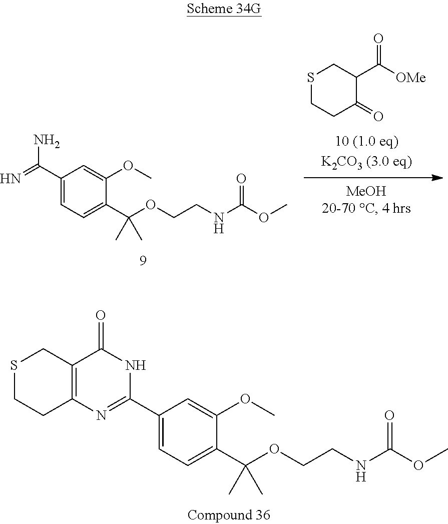 [SMILES: COC(=O)C1CSCCC1=O ; COC(=O)NCCOC(C)(C)c1ccc(C(=N)N)cc1OC ; COC(=O)NCCOC(C)(C)c1ccc(-c2nc3c(c(=O)[nH]2)CSCC3)cc1OC]

Methyl (2-((2-(4-carbamimidoyl-2-methoxyphenyl)propan-2-yl)oxy)ethyl)carbamate (9) (1.0 g, 3.23 mmol, 1.0 eq) and methyl 4-oxotetrahydro-2H-thiopyran-3-carboxylate (10) (563.15 mg, 3.23 mmol, 1.0 eq) were added to MeOH (10 mL) at 20° C. under N₂. K₂CO₃ (1.34 g, 9.70 mmol, 3.0 eq) was added to the mixture at 20° C. and the mixture stirred at 70° C. and microwaved under N₂ for 4 h. LC-MS showed that (9) was consumed. The reaction mixture was filtered and the filtrate concentrated under reduced pressure to give a residue that was purified by prep-HPLC (column: Agela DuraShell C18 250×70 mm×10 um; mobile phase: [H₂O (10 mM NH₄HCO₃)-ACN]; gradient: 30%-55% B over 20.0 min) to provide methyl (2-((2-(2-methoxy-4-(4-oxo-3,5,7,8-tetrahydro-4H-thiopyrano[4,3-d]pyrimidin-2-yl)phenyl)propan-2-yl)oxy)ethyl)carbamate (Compound 34). LC-MS: 435.1 (M+1). ¹H NMR (400 MHz, DMSO-d₆): δ=7.79 (s, 1H), 7.73 (d, J=8.1 Hz, 1H), 7.39 (br d, J=8.1 Hz, 1H), 7.16-7.07 (m, 1H), 3.86 (s, 3H), 3.52 (s, 3H), 3.45 (s, 2H), 3.30-3.24 (m, 2H), 3.16 (q, J=5.9 Hz, 2H), 2.79 (br dd, J=5.3, 18.5 Hz, 4H), 1.52 (s, 6H).

### Compound 35

### Synthesis of methyl (2-((2-(2-methyl-4-(4-oxo-3,5,7,8-tetrahydro-4H-thiopyrano[4,3-d]pyrimidin-2-yl)phenyl)propan-2-yl)oxy)ethyl)carbamate (Compound 35)

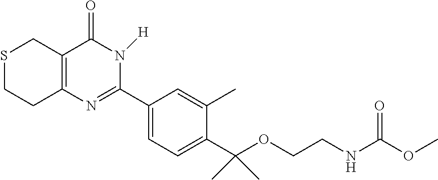 [SMILES: COC(=O)NCCOC(C)(C)c1ccc(-c2nc3c(c(=O)[nH]2)CSCC3)cc1C]

### Synthesis of 2-(4-bromo-2-methylphenyl)propan-2-ol

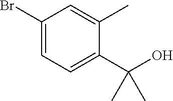

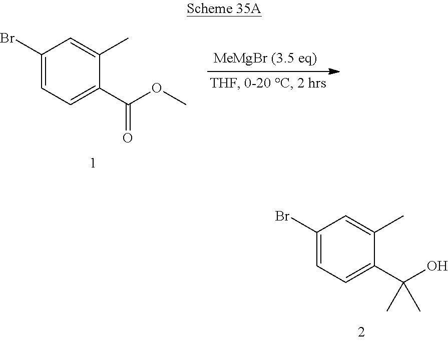

To a solution of methyl 4-bromo-2-methylbenzoate (1) (10 g, 43.65 mmol, 10.00 mL, 1 eq) in THF (100 mL), was added MeMgBr (3 M, 50.93 mL, 3.5 eq) dropwise at 0° C. The mixture was stirred at 20° C. for 2 h. TLC (petroleum ether/ethyl acetate=8/1, Rf(Cpd 1)=0.60) indicated that (1) was consumed, and three new spots were detected. The reaction mixture was poured into sat. NH₄Cl (150 mL) at 0° C. and extracted with EtOAc (50 mL×3). The combined organic layers were dried over Na₂SO₄, filtered, and concentrated under reduced pressure to give a residue that was purified by column chromatography (SiO₂, petroleum ether/ethyl acetate=50/1 to 0/1) to provide 2-(4-bromo-2-methylphenyl)propan-2-ol (2) (6 g, 26.19 mmol). LCMS: 214, 215 (—OH, bromo isomers) [M+1].

### Synthesis of 4-bromo-2-methyl-1-(2-(2-nitroethoxy)propan-2-yl)benzene

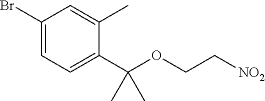 [SMILES: Cc1cc(Br)ccc1C(C)(C)OCC[N+](=O)[O-]]

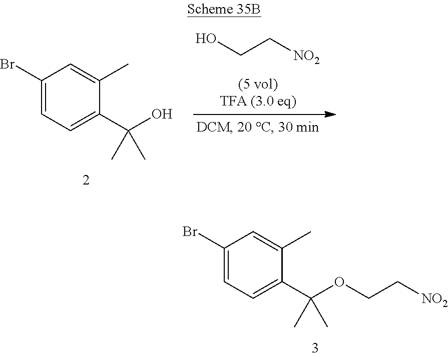 [SMILES: O=[N+]([O-])CCO ; Cc1cc(Br)ccc1C(C)(C)O ; Cc1cc(Br)ccc1C(C)(C)OCC[N+](=O)[O-]]

To a solution of 2-(4-bromo-2-methylphenyl)propan-2-ol (2) (4.59 g, 20.03 mmol, 1 eq) in DCM (50 mL) was added TFA (6.85 g, 60.10 mmol, 4.46 mL, 3 eq) and 2-nitroethanol (32.30 g, 354.69 mmol, 25 mL, 17.70 eq) at 20° C. and stirred for 30 min. TLC (petroleum ether/ethyl acetate=5/1) indicated that (2) was consumed completely and one major new spot with lower polarity was detected. The reaction was quenched with water (60 mL) and extracted with EtOAc (20×10 mL). The organic phase was dried over Na₂SO₄, filtered, and concentrated under reduced pressure to give a residue that was purified by column chromatography (SiO₂, petroleum ether/ethyl acetate=50/1 to 0/1) to provide 4-bromo-2-methyl-1-(2-(2-nitroethoxy)propan-2-yl)benzene (3) (2.68 g, 8.87 mmol). LCMS: 303.5+bromo isomers (M+1).

### Synthesis of 2-((2-(4-bromo-2-methylphenyl)propan-2-yl)oxy)ethan-1-amine

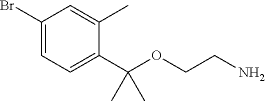

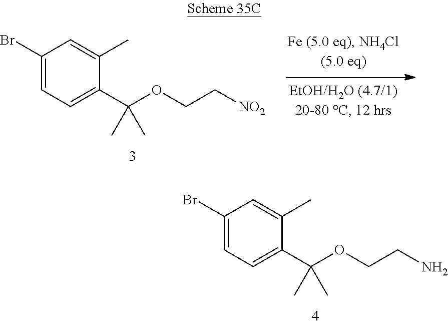 [SMILES: Cc1cc(Br)ccc1C(C)(C)OCC[N+](=O)[O-] ; Cc1cc(Br)ccc1C(C)(C)OCCN]

To a solution of 4-bromo-2-methyl-1-(2-(2-nitroethoxy)propan-2-yl)benzene (3) (1.7 g, 5.63 mmol, 1 eq) in EtOH (14 mL) and H₂O (3 mL), was added Fe (1.57 g, 28.13 mmol, 5 eq) and NH₄Cl (1.50 g, 28.13 mmol, 5 eq) at 20° C. The mixture was stirred at 80° C. for 12 h. The reaction mixture was diluted with H₂O (30 mL), extracted with EtOAc (15 mL×2) and the organic layers combined and concentrated under reduced pressure to give 2-((2-(4-bromo-2-methylphenyl)propan-2-yl)oxy)ethan-1-amine (4) (1.4 g, 5.14 mmol). The crude product was used in the next step without purification. ¹H NMR (400 MHz, CDCl3): δ=7.30 (d, J=1.8 Hz, 1H), 7.25 (d, J=1.9 Hz, 1H), 7.12 (d, J=8.3 Hz, 1H), 3.72 (br s, 2H), 3.22 (t, J=5.3 Hz, 2H), 2.94 (t, J=5.3 Hz, 2H), 2.53 (s, 3H), 1.62 (s, 6H).

### Synthesis of methyl (2-((2-(4-bromo-2-methylphenyl)propan-2-yl)oxy)ethyl)carbamate

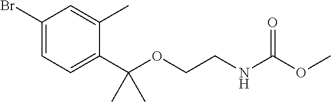 [SMILES: COC(=O)NCCOC(C)(C)c1ccc(Br)cc1C]

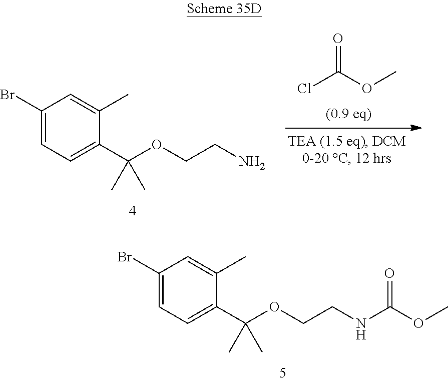

To a solution of 2-((2-(4-bromo-2-methylphenyl)propan-2-yl)oxy)ethan-1-amine (4) (1.4 g, 5.14 mmol, 1 eq) in DCM (14 mL), was added TEA (780.73 mg, 7.72 mmol, 1.07 mL, 1.5 eq) at 20° C. The mixture was cooled to 0° C. and methyl carbonochloridate (437.47 mg, 4.63 mmol, 357.70 μL, 0.9 eq) was added dropwise at 0° C. The mixture was stirred at 20° C. for 12 h. The reaction was quenched with NH₃·H₂O (10%, 20 mL) and extracted with DCM (10 mL×3). The combined organic layers were concentrated under reduced pressure to give a residue that was purified by column chromatography (SiO₂, petroleum ether/ethyl acetate=100/1 to 0/1) to provide methyl (2-((2-(4-bromo-2-methylphenyl)propan-2-yl)oxy)ethyl)carbamate (5) (1.3 g, 3.94 mmol). ¹H NMR (400 MHz, CHLOROFORM-d): δ=7.30 (d, J=2.0 Hz, 1H), 7.27-7.22 (m, 1H), 7.11 (d, J=8.5 Hz, 1H), 5.01 (br s, 1H), 3.67 (s, 3H), 3.36-3.24 (m, 2H), 3.16 (br t, J=5.1 Hz, 2H), 2.53 (s, 3H), 1.58 (s, 6H).

### Synthesis of methyl (2-((2-(4-cyano-2-methylphenyl)propan-2-yl)oxy)ethyl)carbamate

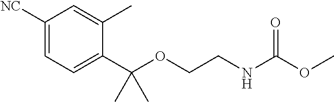 [SMILES: COC(=O)NCCOC(C)(C)c1ccc(C#N)cc1C]

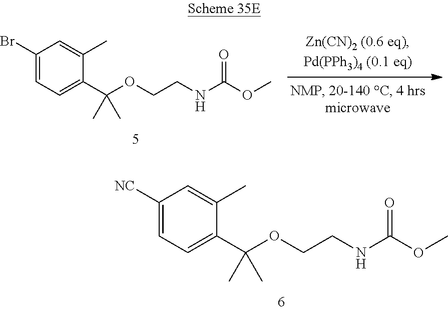 [SMILES: COC(=O)NCCOC(C)(C)c1ccc(Br)cc1C ; COC(=O)NCCOC(C)(C)c1ccc(C#N)cc1C]

To a solution of methyl (2-((2-(4-bromo-2-methylphenyl)propan-2-yl)oxy)ethyl)carbamate (5) (0.6 g, 1.82 mmol, 1 eq) in NMP (6 mL) was added Zn(CN)₂ (128.01 mg, 1.09 mmol, 69.19 μL, 0.6 eq) and Pd(PPh₃)₄ (209.96 mg, 181.70 mol, 0.1 eq) at 20° C. under N₂. The mixture was stirred at 140° C. for 4 h and microwaved under an N₂ atmosphere. The resulting mixture was cooled to 20° C. and diluted with saturated Na₂CO₃ (30 mL). The mixture was extracted with EtOAc (2×20 mL). The combined organic layers were washed with brine (20 mL), dried over Na₂SO₄, filtered, and concentrated under reduced pressure to give a residue that was purified by column chromatography (SiO₂, petroleum ether/ethyl acetate=50/1 to 0/1) to provide methyl (2-((2-(4-cyano-2-methylphenyl)propan-2-yl)oxy)ethyl)carbamate (6) (0.3 g, 1.09 mmol). ¹H NMR (400 MHz, CDCl3): δ=7.46-7.41 (m, 2H), 7.38-7.34 (m, 1H), 5.12-4.91 (m, 1H), 3.67 (s, 3H), 3.37-3.28 (m, 2H), 3.17 (t, J=5.1 Hz, 2H), 2.59 (s, 3H), 1.61 (s, 6H).

### Synthesis of methyl (2-((2-(4-carbamimidoyl-2-methylphenyl)propan-2-yl)oxy)ethyl)carbamate

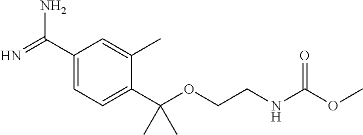 [SMILES: COC(=O)NCCOC(C)(C)c1ccc(C(=N)N)cc1C]

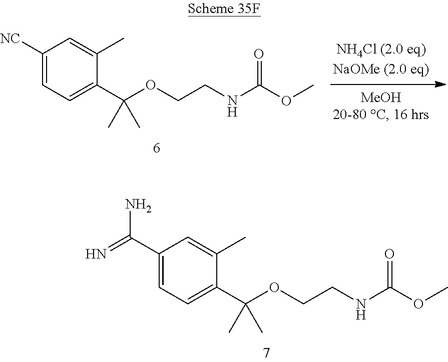 [SMILES: COC(=O)NCCOC(C)(C)c1ccc(C#N)cc1C ; COC(=O)NCCOC(C)(C)c1ccc(C(=N)N)cc1C]

To a solution of methyl (2-((2-(4-cyano-2-methylphenyl)propan-2-yl)oxy)ethyl)carbamate (6) (0.3 g, 1.09 mmol, 1 eq) in MeOH (3 mL) was added NaOMe (117.30 mg, 2.17 mmol, 2 eq) at 20° C. and then stirred at 70° C. for 4 h. NH₄Cl (116.15 mg, 2.17 mmol, 2 eq) was added the mixture stirred at 80° C. for 12 h. The reaction mixture was filtered, and the filtrate concentrated under reduced pressure to give methyl (2-((2-(4-carbamimidoyl-2-methylphenyl)propan-2-yl)oxy)ethyl)carbamate (7) (0.32 g, crude). The crude product was used in the next step without purification. LCMS: 294.7 (M+1).

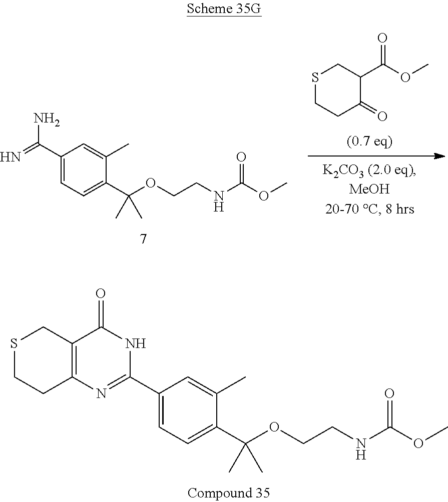 [SMILES: COC(=O)C1CSCCC1=O ; COC(=O)NCCOC(C)(C)c1ccc(C(=N)N)cc1C ; COC(=O)NCCOC(C)(C)c1ccc(-c2nc3c(c(=O)[nH]2)CSCC3)cc1C]

To a solution of methyl (2-((2-(4-carbamimidoyl-2-methylphenyl)propan-2-yl)oxy)ethyl)carbamate (7) (0.32 g, 1.09 mmol, 1 eq) in MeOH (3.2 mL) was added K₂CO₃ (301.52 mg, 2.18 mmol, 2 eq) and methyl 4-oxotetrahydrothiopyran-3-carboxylate (133.03 mg, 763.57 mol, 0.7 eq) at 20° C. under N₂. The mixture was stirred at 70° C. for 8 h. LC-MS showed that (7) was consumed. The reaction mixture was filtered and concentrated under reduced pressure to give a residue that was purified by prep-HPLC (neutral condition: column: Waters Xbridge BEH C18 250×50 mm×10 um; mobile phase: [H₂O (10 mM NH₄HCO₃)-ACN]; gradient: 20%-50% B over 10.0 min) to provide methyl (2-((2-(2-methyl-4-(4-oxo-3,5,7,8-tetrahydro-4H-thiopyrano[4,3-d]pyrimidin-2-yl)phenyl)propan-2-yl)oxy)ethyl)carbamate (Compound 35). LC-MS: 418.1 (M+1). ¹H NMR (400 MHz, CD3OD): δ=7.82 (s, 1H), 7.77 (br d, J=7.9 Hz, 1H), 7.50 (d, J=8.3 Hz, 1H), 3.63 (s, 3H), 3.62 (s, 2H), 3.28-3.22 (m, 2H), 3.21-3.16 (m, 2H), 2.98 (br dd, J=5.2, 14.1 Hz, 4H), 2.68 (s, 3H), 1.66 (s, 6H).

### Compound 36

### Synthesis of (3′-cyano-4′-(4-oxo-3,5,7,8-tetrahydro-4H-thiopyrano[4,3-d]pyrimidin-2-yl)-[1,1′-biphenyl]-4-yl)boronic acid (Compound 36)

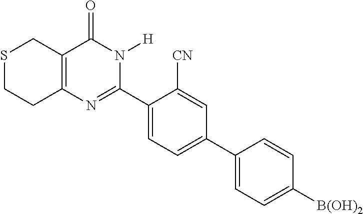 [SMILES: N#Cc1cc(-c2ccc(B(O)O)cc2)ccc1-c1nc2c(c(=O)[nH]1)CSCC2]

### Synthesis of 4-bromo-2-iodobenzimidamide

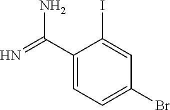 [SMILES: N=C(N)c1ccc(Br)cc1I]

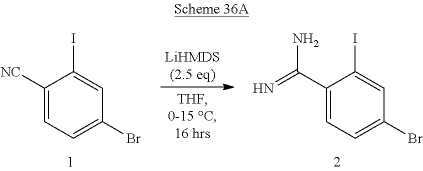 [SMILES: N#Cc1ccc(Br)cc1I ; N=C(N)c1ccc(Br)cc1I]

To a solution of 4-bromo-2-iodobenzonitrile (1) (5.00 g, 16.24 mmol, 1.0 eq) in THF (50 mL), LiHMDS (1 M, 40.60 mL, 2.5 eq) was added dropwise at 0° C. The mixture was stirred at 15° C. for 16 h. TLC (petroleum ether/ethyl acetate=3/1, R_{f} (Cpd.1)=0.72) indicated that (1) was consumed completely, and one new spot formed. The reaction mixture was diluted with 4 N HCl/dioxane (10 mL). The combined organic layers were concentrated under reduced pressure to give a residue. The residue was triturated with MeOH (10 mL), then filtered. The filter cake was dried in vacuo to give a residue. 4-bromo-2-iodobenzimidamide (2) (6 g, crude, HCl) was obtained and used immediately in the next step.

### Synthesis of 2-(4-bromo-2-iodophenyl)-3,5,7,8-tetrahydro-4H-thiopyrano[4,3-d]pyrimidin-4-one

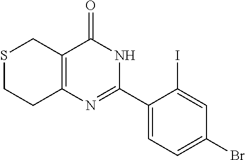 [SMILES: O=c1[nH]c(-c2ccc(Br)cc2I)nc2c1CSCC2]

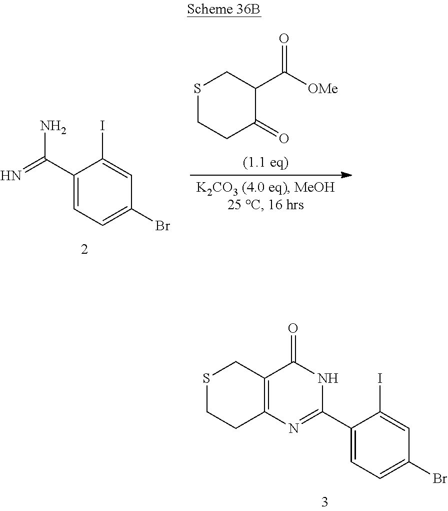 [SMILES: N=C(N)c1ccc(Br)cc1I ; COC(=O)C1CSCCC1=O ; O=c1[nH]c(-c2ccc(Br)cc2I)nc2c1CSCC2]

To a solution of 4-bromo-2-iodobenzimidamide (2) (5.87 g, 16.24 mmol, 1.0 eq, HCl) and methyl 4-oxotetrahydro-2H-thiopyran-3-carboxylate (3.11 g, 17.8 mmol, 1.1 eq) in MeOH (60 mL) was added K₂CO₃ (8.98 g, 64.97 mmol, 4.0 eq) at 25° C. The mixture was stirred at 25° C. for 16 h under N₂ atmosphere. LC-MS indicated most of (2) was consumed, and one main peak with the desired MS was detected. The resulting mixture was filtered. The filter cake was triturated with H₂O (50 mL) at 25° C. for 4 h. Then the mixture was filtered, and the filter cake was dried under reduced pressure to give 2-(4-bromo-2-iodophenyl)-3,5,7,8-tetrahydro-4H-thiopyrano[4,3-d]pyrimidin-4-one (3) (3.78 g, 8.42 mmol, crude), which was used directly in the next step without further purification.

### Synthesis of 5-bromo-2-(4-oxo-3,5,7,8-tetrahydro-4H-thiopyrano[4,3-d]pyrimidin-2-yl)benzonitrile

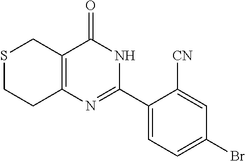 [SMILES: N#Cc1cc(Br)ccc1-c1nc2c(c(=O)[nH]1)CSCC2]

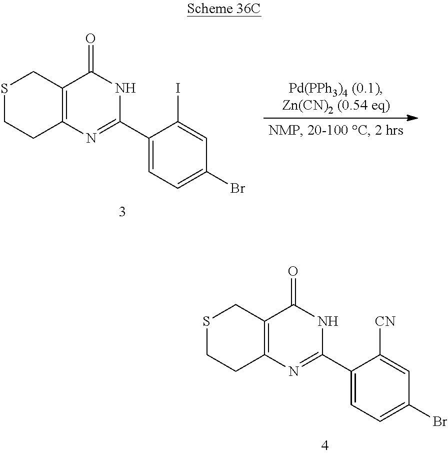 [SMILES: O=c1[nH]c(-c2ccc(Br)cc2I)nc2c1CSCC2 ; N#Cc1cc(Br)ccc1-c1nc2c(c(=O)[nH]1)CSCC2]

To a solution of 2-(4-bromo-2-iodophenyl)-3,5,7,8-tetrahydro-4H-thiopyrano[4,3-d]pyrimidin-4-one (3) (3.28 g, 7.30 mmol, 1.0 eq) in NMP (33 mL) was added Zn(CN)₂ (463 mg, 3.94 mmol, 250.33 μL, 0.54 eq) in one portion and Pd(PPh₃)₄ (844 mg, 730.34 mol, 0.1 eq) at 20° C. under N₂. The mixture was stirred at 100° C. for 2 h under N₂. LC-MS showed 3 was consumed completely. The resulting mixture was cooled to 20° C., and diluted with saturated Na₂CO₃ (60 mL) at 0° C.; then the mixture was extracted with 60 mL of EtOAc (20 mL×3). The organic layers were combined, washed with brine (120 mL), dried over Na₂SO₄, filtered, and concentrated under reduced pressure to give a residue. The residue was purified by column chromatography (SiO₂, petroleum ether/ethyl acetate=50/1 to 0/1) to provide 5-bromo-2-(4-oxo-3,5,7,8-tetrahydro-4H-thiopyrano[4,3-d]pyrimidin-2-yl)benzonitrile (4) (1.56 g, 4.48 mmol). ¹H NMR (400 MHz, DMSO-d₆): δ=8.59 (s, 1H), 8.33 (br d, J=8.3 Hz, 1H), 7.99-7.82 (m, 1H), 3.84-3.60 (m, 2H), 2.88 (br d, J=3.5 Hz, 4H).

### Synthesis of 4′-bromo-4-(4-oxo-3,5,7,8-tetrahydro-4H-thiopyrano[4,3-d]pyrimidin-2-yl)-[1,1′-biphenyl]-3-carbonitrile

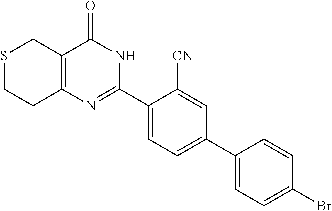 [SMILES: N#Cc1cc(-c2ccc(Br)cc2)ccc1-c1nc2c(c(=O)[nH]1)CSCC2]

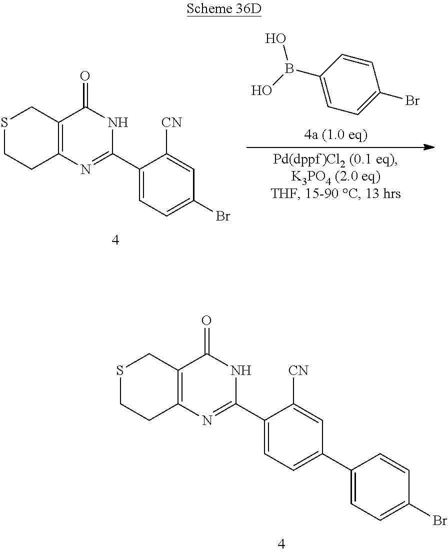 [SMILES: N#Cc1cc(Br)ccc1-c1nc2c(c(=O)[nH]1)CSCC2 ; OB(O)c1ccc(Br)cc1 ; N#Cc1cc(-c2ccc(Br)cc2)ccc1-c1nc2c(c(=O)[nH]1)CSCC2]

To a solution of 5-bromo-2-(4-oxo-3,5,7,8-tetrahydro-4H-thiopyrano[4,3-d]pyrimidin-2-yl)benzonitrile (4) (300 mg, 861.53 mol, 1.0 eq) in THF (10 mL) was added (4-bromophenyl)boronic acid (4a) (173.02 mg, 861.53 mol, 1.0 eq), K₃PO₄ (1 M, 1.72 mL, 2.0 eq), and Pd(dppf)Cl₂ (63.04 mg, 86.15 mol, 28.72 μL, 0.1 eq) at 15° C. The mixture was stirred at 90° C. for 13 h under N₂. TLC (petroleum ether/ethyl acetate=2/1, R_{f} (Cpd.4)=0.50) indicated that (4) was consumed completely, and one major new spot formed. After cooling to 15° C., the mixture was filtered, and the filtrate was concentrated under reduced pressure to give a residue. The residue was purified by prep-TLC (SiO₂, petroleum ether/ethyl acetate=2/1) to provide 4′-bromo-4-(4-oxo-3,5,7,8-tetrahydro-4H-thiopyrano[4,3-d]pyrimidin-2-yl)-[1,1′-biphenyl]-3-carbonitrile (5) (400 mg, crude). The compound was used crude in next step.

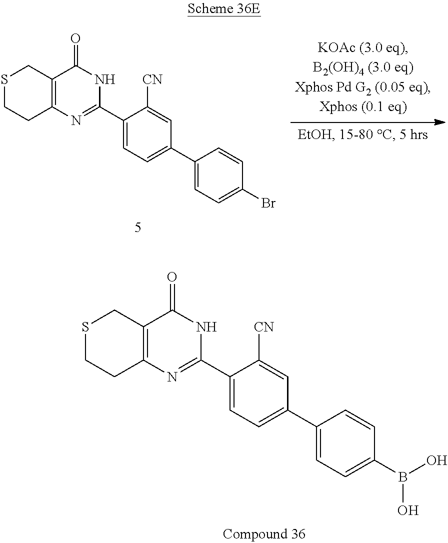 [SMILES: N#Cc1cc(-c2ccc(Br)cc2)ccc1-c1nc2c(c(=O)[nH]1)CSCC2 ; N#Cc1cc(-c2ccc(B(O)O)cc2)ccc1-c1nc2c(c(=O)[nH]1)CSCC2]

To a solution of B₂(OH)₄ (41.6 mg, 463.81 mol, 3.0 eq) in EtOH (1 mL) was added 4′-bromo-4-(4-oxo-3,5,7,8-tetrahydro-4H-thiopyrano[4,3-d]pyrimidin-2-yl)-[1,1′-biphenyl]-3-carbonitrile (5) (65.6 mg, 154.60 mol, 1.0 eq), KOAc (45.5 mg, 463.81 mol, 3.0 eq), XPHOS-PD-G2 (6.08 mg, 7.73 mol, 0.05 eq), and XPhos (7.37 mg, 15.46 mol, 0.1 eq) at 15° C. The mixture was stirred at 80° C. for 5 h under N₂. LC-MS showed that (5) was consumed completely, and one main peak with the desired mass was detected. After cooling to 15° C., the reaction mixture was filtered, and the filtrate was concentrated under reduced pressure to give a residue. The residue was purified by prep-HPLC (neutral condition; column: Waters Xbridge Prep OBD C18 150×40 mm×10 um; mobile phase: [H₂O (10 mM NH₄HCO₃)-ACN]; gradient: 20%-50% B over 8.0 min) to provide (3′-cyano-4′-(4-oxo-3,5,7,8-tetrahydro-4H-thiopyrano[4,3-d]pyrimidin-2-yl)-[1,1′-biphenyl]-4-yl)boronic acid (Compound 36). LC-MS: 369.1 (M+1). ¹H NMR (400 MHz, DMSO-d₆): δ=8.70 (d, J=1.5 Hz, 1H), 8.52 (br d, J=8.0 Hz, 1H), 8.30 (s, 2H), 8.02 (d, J=8.0 Hz, 2H), 7.87 (d, J=8.3 Hz, 1H), 7.69 (d, J=8.1 Hz, 2H), 3.62 (s, 2H), 2.99 (s, 4H).

### Compound 37

### Synthesis of (3-cyano-4-(4-oxo-3,5,7,8-tetrahydro-4H-thiopyrano[4,3-d]pyrimidin-2-yl)phenyl)boronic acid (Compound 37)

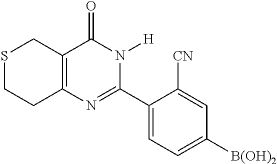 [SMILES: N#Cc1cc(B(O)O)ccc1-c1nc2c(c(=O)[nH]1)CSCC2]

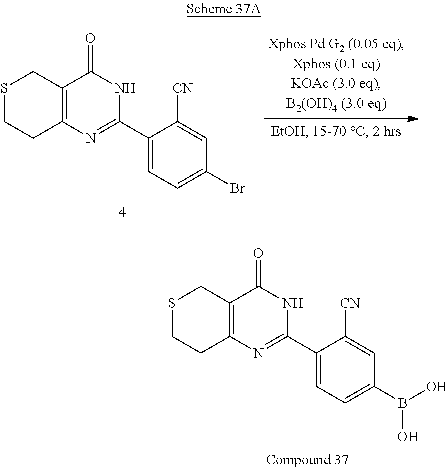 [SMILES: N#Cc1cc(Br)ccc1-c1nc2c(c(=O)[nH]1)CSCC2 ; N#Cc1cc(B(O)O)ccc1-c1nc2c(c(=O)[nH]1)CSCC2]

The synthesis of 5-bromo-2-(4-oxo-3,5,7,8-tetrahydro-4H-thiopyrano[4,3-d]pyrimidin-2-yl)benzonitrile (4) is shown in Schemes 36A-C.

To a solution of B₂(OH)₄ (232 mg, 2.58 mmol, 3.0 eq) in EtOH (12 mL) was added 5-bromo-2-(4-oxo-3,5,7,8-tetrahydro-4H-thiopyrano[4,3-d]pyrimidin-2-yl)benzonitrile (4) (300 mg, 861.53 mol, 1.0 eq), KOAc (254 mg, 2.58 mmol, 3.0 eq), XPHOS-PD-G2 (33.9 mg, 43.08 mol, 0.05 eq), and XPhos (41.1 mg, 86.15 mol, 0.1 eq) at 15° C. under N₂. The mixture was stirred at 70° C. under N₂ for 2 h. LC-MS showed that (4) was consumed completely, and one main peak with the desired mass was detected. After cooling to 15° C., the mixture was filtered, and the filtrate was concentrated under reduced pressure to give a residue. The residue was purified by prep-HPLC (neutral condition; column: Waters Xbridge BEH C18 100×30 mm×10 um; mobile phase: [H₂O (10 mM NH₄HCO₃)-ACN]; gradient: 1%-30% B over 8.0 mins) to provide (3-cyano-4-(4-oxo-3,5,7,8-tetrahydro-4H-thiopyrano[4,3-d]pyrimidin-2-yl)phenyl)boronic acid (Compound 37). LC-MS: 314.1 (M+1). ¹H NMR (400 MHz, DMSO-d₆): δ=12.87-12.47 (m, 1H), 8.53-8.39 (m, 1H), 8.21-7.81 (m, 2H), 7.75-7.62 (m, 1H), 7.57-7.50 (m, 1H), 3.55 (br s, 2H), 2.91 (br s, 4H).

### Compound 38 Synthesis of methyl (2-((2-(3-cyano-4-(4-oxo-3,5,7,8-tetrahydro-4H-thiopyrano[4,3-d]pyrimidin-2-yl)phenyl)propan-2-yl)oxy)ethyl)carbamate (Compound 38)

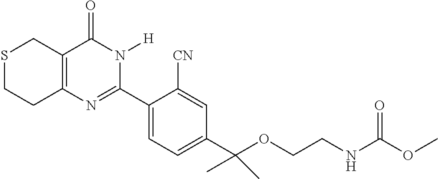 [SMILES: COC(=O)NCCOC(C)(C)c1ccc(-c2nc3c(c(=O)[nH]2)CSCC3)c(C#N)c1]

### Synthesis of methyl 3-bromo-4-cyanobenzoate

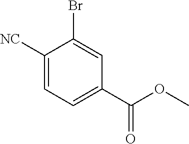

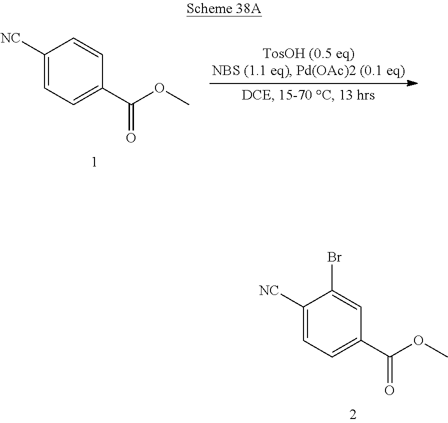

To a solution of methyl 4-cyanobenzoate (1) (10.0 g, 62.1 mmol, 1.00 eq) in DCE (100 mL) was added NBS (12.2 g, 68.3 mmol, 1.10 eq), TosOH (5.34 g, 31.0 mmol, 0.5 eq) and Pd(OAc)₂ (1.39 g, 6.21 mmol, 0.1 eq) at 15° C. The mixture was stirred at 70° C. for 13 h under N₂. TLC (Petroleum ether/Ethyl acetate=8/1, R_{f} (1)=0.60) showed some of (1) remained. After cooling to 15° C., the mixture was filtered through celatom and the filtrate concentrated under reduced pressure to give a residue that was purified by column chromatography (SiO₂, Petroleum ether/Ethyl acetate=100/1 to 0/1) to provide methyl 3-bromo-4-cyanobenzoate (2) (4.50 g, 18.8 mmol). ¹H NMR (400 MHz, CDCl₃-d): δ 8.27 (d, J=1.3 Hz, 1H), 8.00 (dd, J=1.3, 8.1 Hz, 1H), 7.68 (d, J=8.1 Hz, 1H), 3.90 (s, 3H).

### Synthesis of methyl 3-bromo-4-carbamimidoylbenzoate

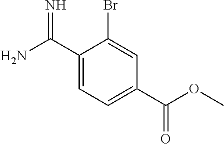

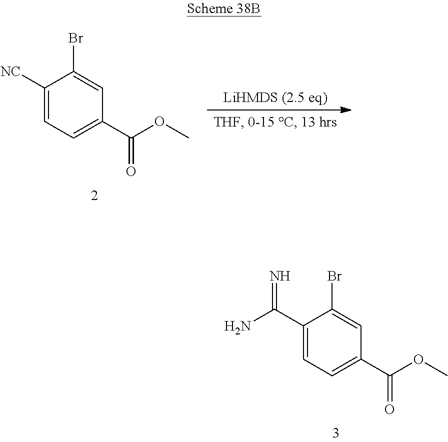

To a solution of methyl 3-bromo-4-cyanobenzoate (2) (4.00 g, 16.7 mmol, 1 eq) in THF (40 mL) was added dropwise LiHMDS (1.00 M, 41.7 mL, 2.50 eq) at 0° C. The mixture was warmed to 15° C. and stirred for 13 hrs. LC-MS showed that (2) was consumed completely and one main peak with desired mass was detected. The reaction mixture was quenched by addition of HCl/dioxane (4M, 20 mL) at 0° C. and then concentrated under reduced pressure to give a residue that was suspended in MeOH (80.0 ml), and stirred at 20° C. for 1 hr. The mixture was filtered and the filtrate concentrated under reduced pressure to give methyl 3-bromo-4-carbamimidoylbenzoate (3) (4.90 g, crude, HCl), which was used in the next step without further purification. LC-MS (product: Rₜ=0.219 min).

### Synthesis of methyl 3-bromo-4-(4-oxo-3,5,7,8-tetrahydro-4H-thiopyrano[4,3-d]pyrimidin-2-yl)benzoate

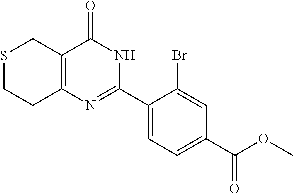 [SMILES: COC(=O)c1ccc(-c2nc3c(c(=O)[nH]2)CSCC3)c(Br)c1]

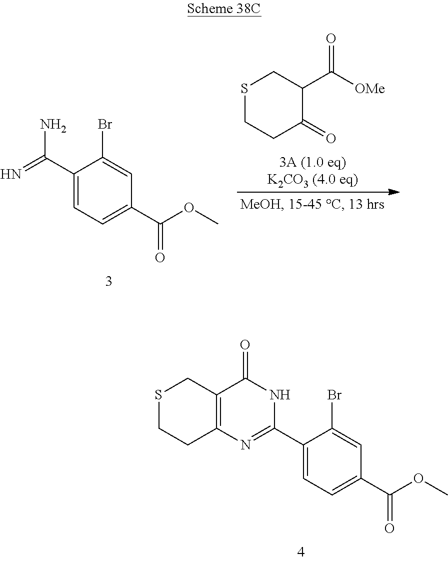 [SMILES: COC(=O)c1ccc(C(=N)N)c(Br)c1 ; COC(=O)C1CSCCC1=O ; COC(=O)c1ccc(-c2nc3c(c(=O)[nH]2)CSCC3)c(Br)c1]

To a solution of methyl 3-bromo-4-carbamimidoylbenzoate (3) (4.90 g, 16.7 mmol, 1 eq, HCl) in MeOH (50 mL) was added methyl 4-oxotetrahydro-2H-thiopyran-3-carboxylate (3A) (2.91 g, 16.7 mmol, 1 eq) and K₂CO₃ (9.23 g, 66.8 mmol, 4 eq) at 15° C. The mixture heated to 45° C. and stirred for 13 hrs. LC-MS showed that (3) was consumed completely and one main peak with desired mass was detected. After cooling to 15° C., the reaction mixture was filtered, and the filtrate was concentrated under reduced pressure to give a residue. The residue was suspended in water (50.0 mL), and stirred at 15° C. for 4 hrs. The mixture was filtered, and the filter cake dried under vacuum to give methyl 3-bromo-4-(4-oxo-3,5,7,8-tetrahydro-4H-thiopyrano[4,3-d]pyrimidin-2-yl)benzoate (4) (3.80 g, crude). ¹H NMR: (400 MHz, DMSO-d₆) δ 13.04-12.82 (m, 1H), 8.21 (d, J=1.2 Hz, 1H), 8.05 (dd, J=1.3, 7.9 Hz, 1H), 7.72 (d, J=8.1 Hz, 1H), 3.91 (s, 3H), 3.54 (s, 2H), 2.92-2.84 (m, 4H). LC-MS (product: Rₜ=0.395 min).

### Synthesis of 2-(2-bromo-4-(2-hydroxypropan-2-yl)phenyl)-3,5,7,8-tetrahydro-4H-thiopyrano[4,3-d]pyrimidin-4-one

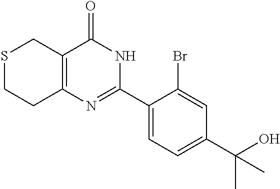 [SMILES: CC(C)(O)c1ccc(-c2nc3c(c(=O)[nH]2)CSCC3)c(Br)c1]

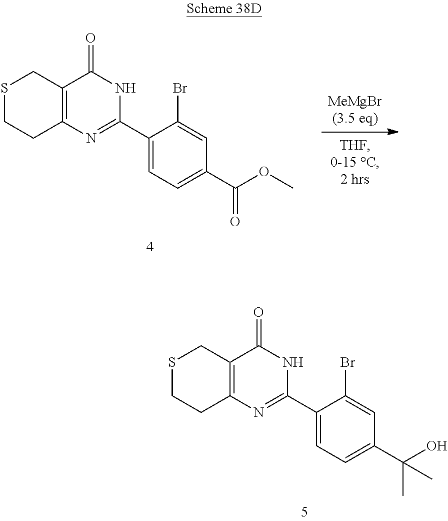 [SMILES: COC(=O)c1ccc(-c2nc3c(c(=O)[nH]2)CSCC3)c(Br)c1 ; CC(C)(O)c1ccc(-c2nc3c(c(=O)[nH]2)CSCC3)c(Br)c1]

To a solution of methyl 3-bromo-4-(4-oxo-3,5,7,8-tetrahydro-4H-thiopyrano[4,3-d]pyrimidin-2-yl)benzoate (4) (3.80 g, 9.97 mmol, 1 eq) in THF (27.0 mL) was added dropwise, MeMgBr (3M, 11.6 mL, 3.50 eq) at 0° C. The mixture was warmed to 15° C. and stirred for 2 hrs. TLC (Petroleum ether/Ethyl acetate=1/1, R_{f} (4)=0.46) indicated that (4) was consumed completely and many new spots formed. The reaction mixture was poured into saturated NH₄Cl (60 mL) at 0° C. and extracted with EtOAc. The combined organic layers were dried over Na₂SO₄, filtered, and concentrated under reduced pressure to give a residue that was purified by prep-HPLC (neutral condition; column: Waters Xbridge BEH C18 250 * 50 mm*10 um; mobile phase: [H₂O (10 mM NH₄HCO₃)-ACN]; gradient: 20%-50% B over 10.0 mins). 2-(2-bromo-4-(2-hydroxypropan-2-yl)phenyl)-3,5,7,8-tetrahydro-4H-thiopyrano[4,3-d]pyrimidin-4-one (5) (2.00 g, 5.19 mmol) was obtained. LC-MS (product: Rₜ=0.358 mins).

### Synthesis of methyl (2-((2-(3-bromo-4-(4-oxo-3,5,7,8-tetrahydro-4H-thiopyrano[4,3-d]pyrimidin-2-yl)phenyl)propan-2-yl)oxy)ethyl)carbamate

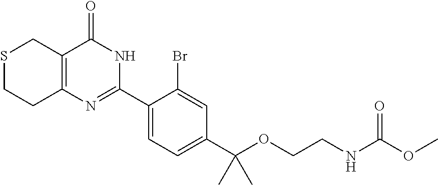 [SMILES: COC(=O)NCCOC(C)(C)c1ccc(-c2nc3c(c(=O)[nH]2)CSCC3)c(Br)c1]

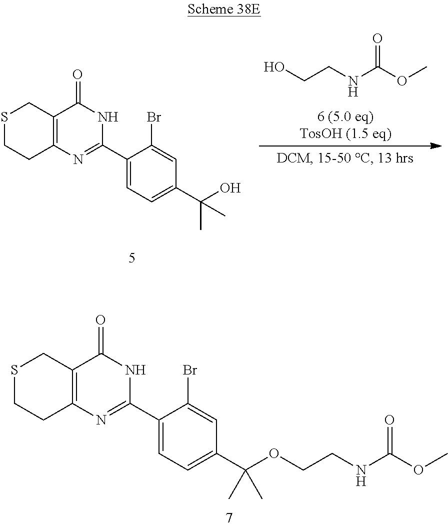

To a solution of 2-(2-bromo-4-(2-hydroxypropan-2-yl)phenyl)-3,5,7,8-tetrahydro-4H-thiopyrano[4,3-d]pyrimidin-4-one (5) (600 mg, 1.57 mmol, 1 eq) and methyl (2-hydroxyethyl)carbamate (6) (937 mg, 7.87 mmol, 5 eq) in DCM (5 mL) was added TosOH (406 mg, 2.36 mmol, 1.50 eq) at 15° C. The mixture was heated to 50° C. and stirred for 13 hrs. LC-MS showed some of (5) remained. After cooling to 15° C., the reaction mixture was concentrated under reduced pressure to give a residue. The residue was purified by prep-HPLC (neutral condition; column: Waters Xbridge Prep OBD C18 150*40 mm*10 um; mobile phase: [H₂O (10 mM NH₄HCO₃)-ACN]; gradient: 20%-50% B over 8.0 mins) to provide methyl (2-((2-(3-bromo-4-(4-oxo-3,5,7,8-tetrahydro-4H-thiopyrano[4,3-d]pyrimidin-2-yl)phenyl)propan-2-yl)oxy)ethyl)carbamate (7) (100 mg, crude). ¹H NMR (400 MHz, CDCl₃-d): δ 7.62 (d, J=1.3 Hz, 1H), 7.50 (s, 1H), 7.46 (d, J=1.5 Hz, 1H), 5.07-4.93 (m, 1H), 3.61 (s, 3H), 3.55 (s, 2H), 3.29-3.19 (m, 4H), 2.96 (s, 2H), 2.86 (s, 2H), 1.46 (s, 6H). LC-MS (product: Rₜ=1.291 min).

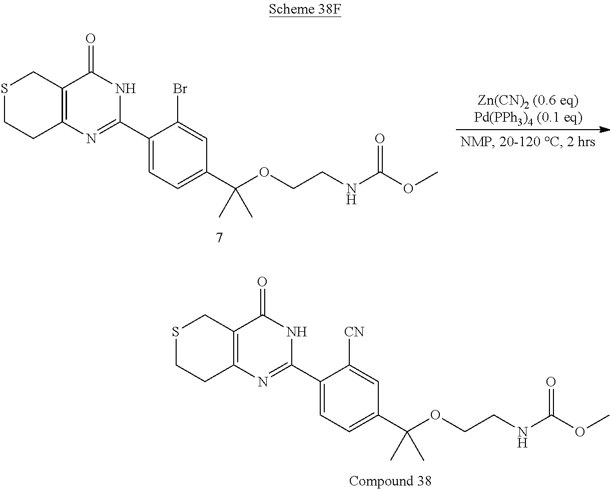 [SMILES: COC(=O)NCCOC(C)(C)c1ccc(-c2nc3c(c(=O)[nH]2)CSCC3)c(Br)c1 ; COC(=O)NCCOC(C)(C)c1ccc(-c2nc3c(c(=O)[nH]2)CSCC3)c(C#N)c1]

To a solution of methyl (2-((2-(3-bromo-4-(4-oxo-3,5,7,8-tetrahydro-4H-thiopyrano[4,3-d]pyrimidin-2-yl)phenyl)propan-2-yl)oxy)ethyl)carbamate (7) (75 mg, 155.48 mol, 1.0 eq) in NMP (1.5 mL) was added in portions, both Zn(CN)₂ (10.95 mg, 93.29 mol, 5.92 μL, 0.6 eq) and Pd(PPh₃)₄ (17.97 mg, 15.55 mol, 0.1 eq) at 20° C. The mixture was heated to 120° C. and stirred for 2 hrs. LC-MS showed that (7) was consumed completely. The resulting mixture was cooled to 20° C., diluted with saturated Na₂CO₃ (3.00 mL) at 0° C., and extracted with EtOAc. The combined organic layers were washed with brine, dried over Na₂SO₄, filtered, and concentrated under reduced pressure to give a residue that was purified by prep-HPLC (neutral condition; column: Waters xbridge 150 * 25 mm 10 um; mobile phase: [water (NH₄HCO₃)-ACN]; gradient: 25%-55% B over 10 mins) to provide methyl (2-((2-(3-cyano-4-(4-oxo-3,5,7,8-tetrahydro-4H-thiopyrano[4,3-d]pyrimidin-2-yl)phenyl)propan-2-yl)oxy)ethyl)carbamate (Compound 38). ¹H NMR (400 MHz, DMSO-d₆): δ 8.03-7.93 (m, 1H), 7.91-7.82 (m, 1H), 7.27-7.15 (m, 1H), 3.65-3.53 (m, 2H), 3.53-3.49 (m, 3H), 3.21-3.12 (m, 4H), 2.98-2.83 (m, 4H), 1.56-1.45 (m, 6H). LC-MS (EC16472-22-P1J2, product: Rₜ=1.600/2.078 min).

### Compound 39

### Synthesis of methyl (2-((2-(2-cyano-4-(4-oxo-3,5,7,8-tetrahydro-4H-thiopyrano[4,3-d]pyrimidin-2-yl)phenyl)propan-2-yl)oxy)ethyl)carbamate (Compound 39)

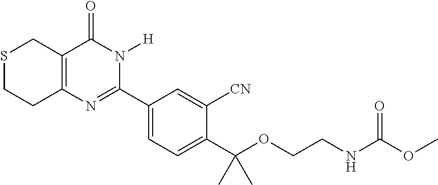 [SMILES: COC(=O)NCCOC(C)(C)c1ccc(-c2nc3c(c(=O)[nH]2)CSCC3)cc1C#N]

### Synthesis of methyl 2-bromo-4-carbamimidoylbenzoate

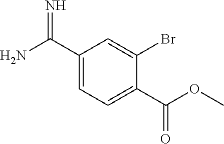

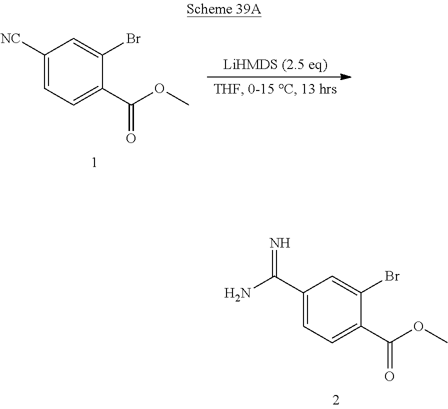

To a solution of Methyl 2-bromo-4-cyanobenzoate (1) (2.5 g, 10.41 mmol, 1 eq) in THF (25 mL) was added drop-wise LiHMDS (1 M, 26.04 mL, 2.5 eq) at 0° C. The mixture was stirred at 15° C. for 13 hrs. LC-MS showed that (1) was consumed completely and one main peak with desired mass was detected. The reaction mixture was quenched by addition of HCl/dioxane (4M, 13 mL) at 0° C. and concentrated under reduced pressure to give a residue that was suspended in MeOH (50 ml) and stirred at 15° C. for 1 hr. The mixture was filtered and the filtrate was concentrated under reduced pressure to give a methyl 2-bromo-4-carbamimidoylbenzoate (2) (3.1 g, crude), which was used into next step without further purification. LC-MS (product: Rₜ=0.231 mins).

### Synthesis of methyl 2-bromo-4-(4-oxo-3,5,7,8-tetrahydro-4H-thiopyrano[4,3-d]pyrimidin-2-yl) benzoate

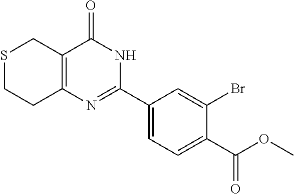 [SMILES: COC(=O)c1ccc(-c2nc3c(c(=O)[nH]2)CSCC3)cc1Br]

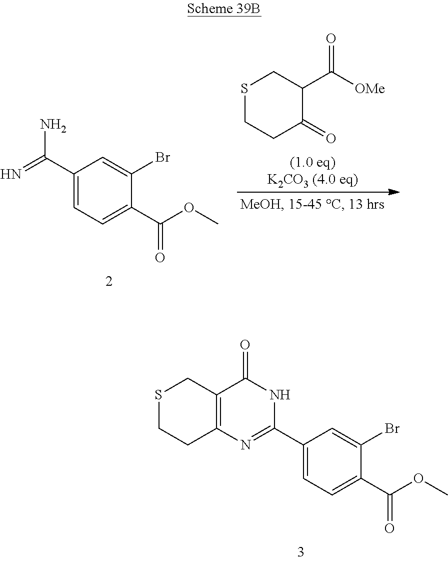 [SMILES: COC(=O)c1ccc(C(=N)N)cc1Br ; COC(=O)C1CSCCC1=O ; COC(=O)c1ccc(-c2nc3c(c(=O)[nH]2)CSCC3)cc1Br]

To a solution of methyl 2-bromo-4-carbamimidoylbenzoate (2) (3.1 g, 10.56 mmol, 1 eq, HCl) in MeOH (30 mL) was added methyl 4-oxotetrahydrothiopyran-3-carboxylate (1.84 g, 10.56 mmol, 1 eq) and K₂CO₃ (5.84 g, 42.24 mmol, 4 eq) at 15° C. The mixture was stirred at 45° C. for 13 hrs. LC-MS showed that (2) was consumed completely and one main peak with desired mass was detected. After cooling to 15° C., the reaction mixture was filtered and the filtrate concentrated under reduced pressure to give a residue that was suspended in water (40 mL) and stirred at 15° C. for 4 hrs. The mixture was then filtered and the filter cake dried under reduced pressure to give methyl 2-bromo-4-(4-oxo-3,5,7,8-tetrahydro-4H-thiopyrano[4,3-d]pyrimidin-2-yl)benzoate (3) (2.89 g, crude). ¹H NMR:(400 MHz, DMSO-d₆) δ 8.45 (s, 1H), 8.19 (br d, J=7.9 Hz, 1H), 7.88 (d, J=8.2 Hz, 1H), 3.92-3.86 (m, 3H), 3.58-3.51 (m, 2H), 2.90 (s, 4H). LC-MS (product: Rₜ=1.559 mins).

### Synthesis of 2-(3-bromo-4-(2-hydroxypropan-2-yl)phenyl)-3,5,7,8-tetrahydro-4H-thiopyrano[4,3-d]pyrimidin-4-one

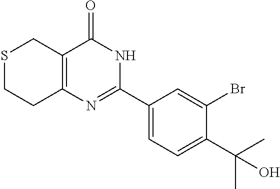 [SMILES: CC(C)(O)c1ccc(-c2nc3c(c(=O)[nH]2)CSCC3)cc1Br]

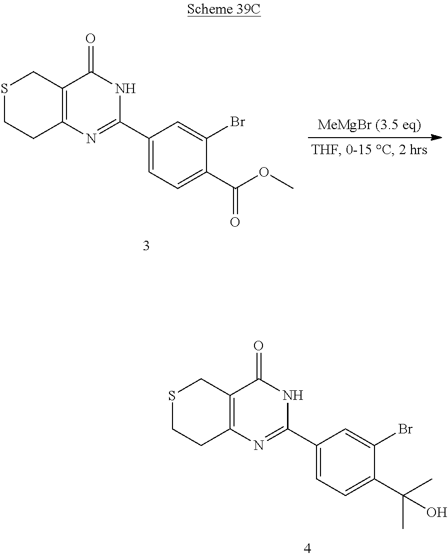 [SMILES: COC(=O)c1ccc(-c2nc3c(c(=O)[nH]2)CSCC3)cc1Br ; CC(C)(O)c1ccc(-c2nc3c(c(=O)[nH]2)CSCC3)cc1Br]

To a solution of methyl 2-bromo-4-(4-oxo-3,5,7,8-tetrahydro-4H-thiopyrano[4,3-d]pyrimidin-2-yl)benzoate (3) (890 mg, 2.33 mmol, 1.0 eq) in THF (8.9 mL) was added dropwise MeMgBr (3 M, 2.72 mL, 3.5 eq) at 0° C. The mixture was stirred at 15° C. for 2 hrs. LC-MS showed that (3) was consumed completely and desired mass was detected. The mixture was poured into saturated NH₄Cl (20 ml) at 0° C. and extracted with EtOAc. The combined organic layers were dried over Na₂SO₄, filtered and concentrated under reduced pressure to give 2-(3-bromo-4-(2-hydroxypropan-2-yl)phenyl)-3,5,7,8-tetrahydro-4H-thiopyrano[4,3-d]pyrimidin-4-one (4) (800 mg, crude). LC-MS (product: Rₜ=0.418 mins).

### Synthesis of methyl (2-((2-(2-bromo-4-(4-oxo-3,5,7,8-tetrahydro-4H-thiopyrano[4,3-d]pyrimidin-2-yl)phenyl)propan-2-yl)oxy)ethyl)carbamate

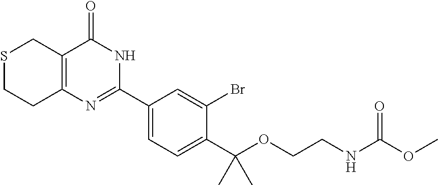 [SMILES: COC(=O)NCCOC(C)(C)c1ccc(-c2nc3c(c(=O)[nH]2)CSCC3)cc1Br]

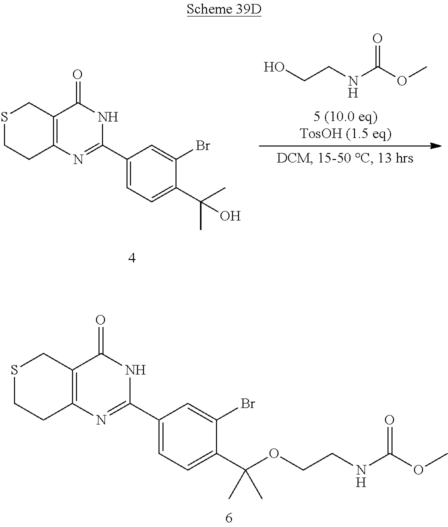

To a solution of compound 2-(3-bromo-4-(2-hydroxypropan-2-yl)phenyl)-3,5,7,8-tetrahydro-4H-thiopyrano[4,3-d]pyrimidin-4-one (4) (500 mg, 1.31 mmol, 1.0 eq) and methyl (2-hydroxyethyl)carbamate (5) (1.56 g, 13.1 mmol, 10.0 eq) in DCM (5.0 mL) was added TosOH (338.72 mg, 1.97 mmol, 1.5 eq) at 15° C. The mixture was stirred at 50° C. for 13 hrs under N₂. LC-MS showed some of (4) remained. After cooling to 15° C., the mixture was concentrated under reduced pressure to give a residue. The residue was purified by prep-HPLC (neutral condition; column: Waters X bridge BEH C18100*30 mm*10 um; mobile phase: [H₂O (10 mM NH₄HCO₃)-ACN]; gradient: 20%-50% B over 8.0 min). Compound methyl (2-((2-(2-bromo-4-(4-oxo-3,5,7,8-tetrahydro-4H-thiopyrano[4,3-d]pyrimidin-2-yl)phenyl)propan-2-yl)oxy)ethyl)carbamate (6) (60 mg, 124.38 mol) was obtained. LC-MS (product: Rₜ=1.478 mins).

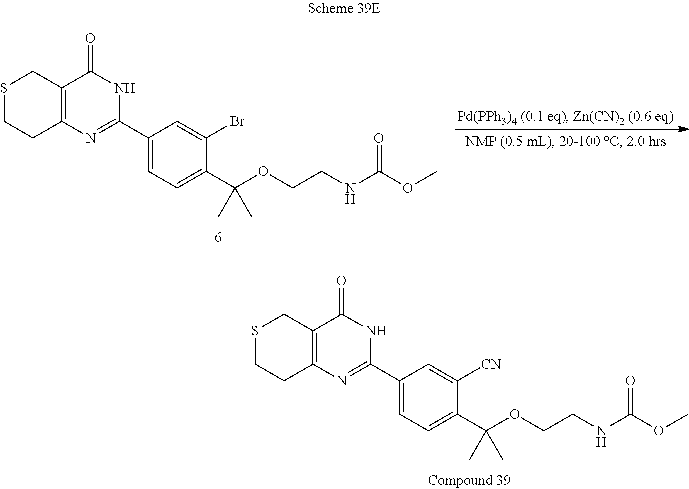 [SMILES: COC(=O)NCCOC(C)(C)c1ccc(-c2nc3c(c(=O)[nH]2)CSCC3)cc1Br ; COC(=O)NCCOC(C)(C)c1ccc(-c2nc3c(c(=O)[nH]2)CSCC3)cc1C#N]

To a solution of methyl (2-((2-(2-bromo-4-(4-oxo-3,5,7,8-tetrahydro-4H-thiopyrano[4,3-d]pyrimidin-2-yl)phenyl)propan-2-yl)oxy)ethyl)carbamate (6) (30 mg, 62.19 mol, 1 eq) in NMP (0.5 mL) was added Zn(CN)₂ (4.38 mg, 37.31 mol, 2.37 μL, 0.6 eq) and Pd(PPh₃)₄ (7.19 mg, 6.22 mol, 0.1 eq) at 20° C. under N₂. The mixture was stirred at 100° C. for 2 hrs. The resulting mixture was cooled to 0° C. and diluted with saturated Na₂CO₃ (5 mL) at 0° C. The mixture was extracted with EtOAc and washed with brine, dried over Na₂SO₄, filtered and concentrated under reduced pressure to give a residue that was purified by prep-HPLC (neutral condition; column: Waters X bridge BEH C18 100*30 mm*10 um; mobile phase: [H₂O (10 mM NH₄HCO₃)-ACN]; gradient: 15%-50% B over 8.0 min) to provide Methyl (2-((2-(2-cyano-4-(4-oxo-3,5,7,8-tetrahydro-4H-thiopyrano[4,3-d]pyrimidin-2-yl)phenyl)propan-2-yl)oxy)ethyl)carbamate (Compound 39). ¹H NMR (400 MHz, DMSO-d₆): δ 13.12-12.68 (m, 1H), 8.53 (s, 1H), 8.33 (br d, J=8.1 Hz, 1H), 7.73 (d, J=8.3 Hz, 1H), 7.08-7.00 (m, 1H), 3.55 (br s, 2H), 3.50 (s, 3H), 3.25-3.17 (m, 4H), 2.97-2.86 (m, 4H), 1.62 (s, 5H). LC-MS (product: Rₜ=2.412 mins).

### Compound 40

### Synthesis of (3-chloro-4-(4-oxo-3,5,7,8-tetrahydro-4H-thiopyrano[4,3-d]pyrimidin-2-yl)phenyl)boronic acid (Compound 40)

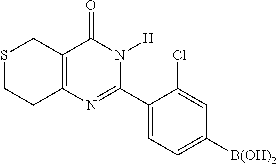 [SMILES: O=c1[nH]c(-c2ccc(B(O)O)cc2Cl)nc2c1CSCC2]

### Synthesis of 2-chloro-4-iodobenzimidamide

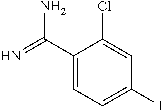 [SMILES: N=C(N)c1ccc(I)cc1Cl]

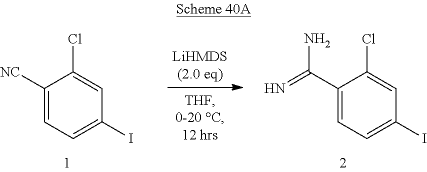 [SMILES: N#Cc1ccc(I)cc1Cl ; N=C(N)c1ccc(I)cc1Cl]

To a solution of 2-chloro-4-iodobenzonitrile (1) (500 mg, 1.90 mmol, 1 eq) in THF (10 mL) was added LiHMDS (1 M, 3.80 mL, 2 eq) dropwise at 0° C. The mixture was stirred at 20° C. for 12 h. LCMS showed that (1) was consumed. The reaction mixture was quenched by addition HCl/dioxane (4 mL) at 0° C., then concentrated under reduced pressure to give a residue that was dissolved in MeOH (5 mL) and stirred at 20° C. for 0.5 hrs. The mixture was filtered and the filtrate concentrated under reduced pressure to give 2-chloro-4-iodobenzimidamide (2) (3.2 g, crude). LCMS: 280.9 (M+1).

### Synthesis of 2-(2-chloro-4-iodophenyl)-3,5,7,8-tetrahydro-4H-thiopyrano[4,3-d]pyrimidin-4-one

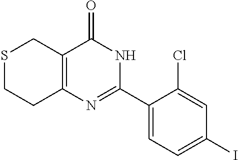 [SMILES: O=c1[nH]c(-c2ccc(I)cc2Cl)nc2c1CSCC2]

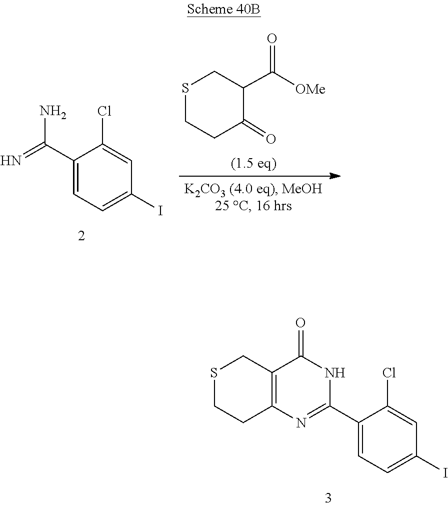 [SMILES: N=C(N)c1ccc(I)cc1Cl ; COC(=O)C1CSCCC1=O ; O=c1[nH]c(-c2ccc(I)cc2Cl)nc2c1CSCC2]

To a solution of 2-chloro-4-iodobenzimidamide (2) (0.5 g, 1.78 mmol, 1.0 eq) and methyl 4-oxotetrahydrothiopyran-3-carboxylate (465.83 mg, 2.67 mmol, 1.5 eq) in MeOH (10 mL) was added K₂CO₃ (985.48 mg, 7.13 mmol, 4 eq) at 25° C. and stirred for 16 h. LCMS showed that (2) was consumed. The reaction mixture was filtered and the filtrate was concentrated under reduced pressure to give a residue that was suspended in water (5 mL) and stirred at 25° C. for 1 h and then filtered. The filtrate was dried in vacuo to give 2-(2-chloro-4-iodophenyl)-3,5,7,8-tetrahydro-4H-thiopyrano[4,3-d]pyrimidin-4-one (3) (1.5 g, crude). The crude product was used in the next step without purification. LCMS: 404.9 (M+1).

### Synthesis of 2-(2-chloro-4-(4,4,5,5-tetramethyl-1,3,2-dioxaborolan-2-yl)phenyl)-3,5,7,8-tetrahydro-4H-thiopyrano[4,3-d]pyrimidin-4-one

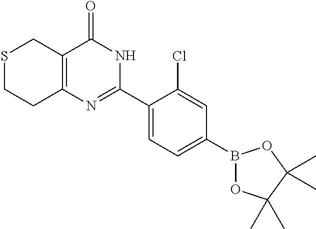 [SMILES: CC1(C)OB(c2ccc(-c3nc4c(c(=O)[nH]3)CSCC4)c(Cl)c2)OC1(C)C]

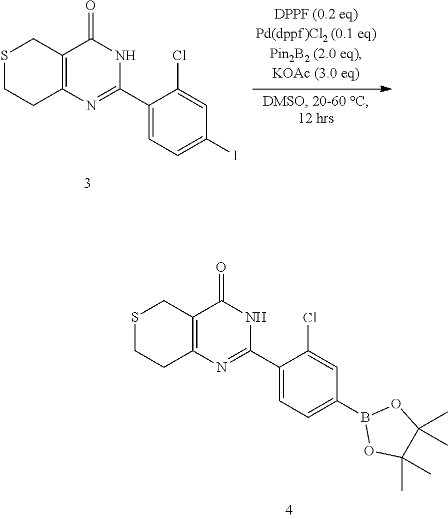 [SMILES: O=c1[nH]c(-c2ccc(I)cc2Cl)nc2c1CSCC2 ; CC1(C)OB(c2ccc(-c3nc4c(c(=O)[nH]3)CSCC4)c(Cl)c2)OC1(C)C]

To a solution of 2-(2-chloro-4-iodophenyl)-3,5,7,8-tetrahydro-4H-thiopyrano[4,3-d]pyrimidin-4-one (3) (500 mg, 1.24 mmol, 1 eq) in DMSO (5.0 mL) was added Pin₂B2 (470.66 mg, 1.85 mmol, 1.5 eq) and KOAc (485.06 mg, 4.94 mmol, 4 eq) at 20° C. The mixture was degassed and purged with N₂ 3 times. DPPF (137.00 mg, 247.13 mol, 0.2 eq) and Pd(dppf)Cl₂ (90.41 mg, 123.56 mol, 0.1 eq) were added in one portion at 20° C. and stirred at 60° C. for 12 h under N₂. The mixture was cooled to 20° C. and diluted with EtOAc (10 mL). The mixture was filtered and the filtrate was washed brine. The organic layer was separated, dried over anhydrous Na₂SO₄, filtered and concentrated under reduced pressure to give 2-(2-chloro-4-(4,4,5,5-tetramethyl-1,3,2-dioxaborolan-2-yl)phenyl)-3,5,7,8-tetrahydro-4H-thiopyrano[4,3-d]pyrimidin-4-one (4) (900 mg, crude) which was used in next step without further purification. LCMS: 405.6 (M+1).

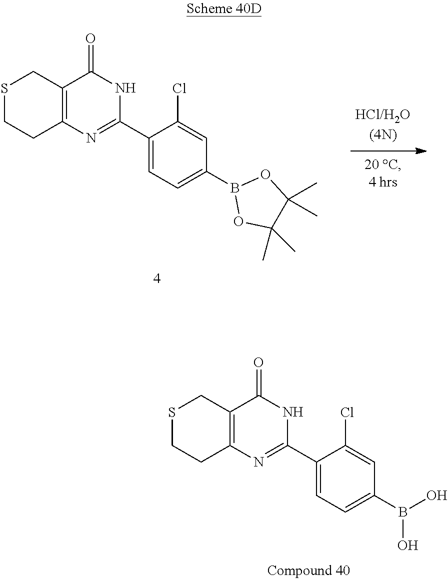 [SMILES: CC1(C)OB(c2ccc(-c3nc4c(c(=O)[nH]3)CSCC4)c(Cl)c2)OC1(C)C ; O=c1[nH]c(-c2ccc(B(O)O)cc2Cl)nc2c1CSCC2]

2-(2-chloro-4-(4,4,5,5-tetramethyl-1,3,2-dioxaborolan-2-yl)phenyl)-3,5,7,8-tetrahydro-4H-thiopyrano[4,3-d]pyrimidin-4-one (4) (300 mg, 741.26 mol, 1 eq) in HCl/H₂O (4 M, 1.5 mL) was stirred at 20° C. for 4 h. The reaction mixture was filtered and the filter cake was dried in vacuo to give a residue. The residue was purified by prep-HPLC (column: Phenomenex luna C18 100 * 40 mm*3 um; mobile phase: [H₂O (0.04% HCl)-ACN]; gradient: 10%-35% B over 18.0 min) to provide (3-chloro-4-(4-oxo-3,5,7,8-tetrahydro-4H-thiopyrano[4,3-d]pyrimidin-2-yl)phenyl)boronic acid (compound 40). LCMS: 323.0 (M+1). ¹H NMR: (400 MHz, DMSO-d₆)=7.91 (s, 1H), 7.82 (d, J=7.6 Hz, 1H), 7.54 (d, J=7.5 Hz, 1H), 3.53 (s, 2H), 2.94-2.81 (m, 4H).

### Compound 41

### Synthesis of (4-(4-(4-oxo-3,5,7,8-tetrahydro-4H-thiopyrano[4,3-d]pyrimidin-2-yl)piperazin-1-yl)phenyl)boronic acid (Compound 41)

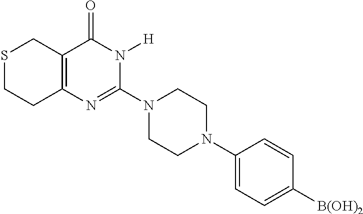 [SMILES: O=c1[nH]c(N2CCN(c3ccc(B(O)O)cc3)CC2)nc2c1CSCC2]

### Synthesis of 2-chloro-7,8-dihydro-5H-thiopyrano[4,3-d]pyrimidin-4-ol

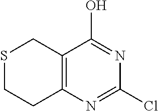 [SMILES: Oc1nc(Cl)nc2c1CSCC2]

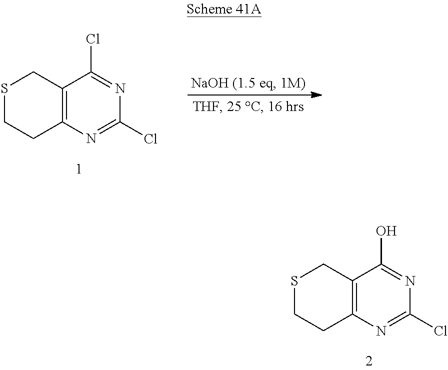 [SMILES: Clc1nc(Cl)c2c(n1)CCSC2 ; "NaOH (1.5 eq, 1M)" ; Oc1nc(Cl)nc2c1CSCC2]

To a solution of 2,4-dichloro-7,8-dihydro-5H-thiopyrano[4,3-d]pyrimidine (1) (0.5 g, 2.26 mmol, 1 eq) in THF (1 mL) was added NaOH (1 M, 3.39 mL, 1.5 eq) at 25° C. The mixture was stirred at 25° C. for 16 h and followed by TLC. The mixture was quenched with H₂O (10 mL) and extracted with EtOAc (10 mL). The aqueous phase was acidified with 0.1 N HCl to pH=3-4 and stirred at 25° C. for 12 h. Then the mixture was extracted with 60 mL of EtOAc (20 mL×3). The combined organic layers were dried over Na₂SO₄, filtered, and concentrated under reduced pressure to give 2-chloro-7,8-dihydro-5H-thiopyrano[4,3-d]pyrimidin-4-ol (2) (0.28 g, crude), which was used as a crude in next step. H NMR (400 MHz, DMSO-d₆): δ 3.49 (br s, 2H), 2.85 (br dd, J=4.5, 14.6 Hz, 4H).

### Synthesis of 2-(4-(4-bromophenyl)piperazin-1-yl)-7,8-dihydro-5H-thiopyrano[4,3-d]pyrimidin-4-ol

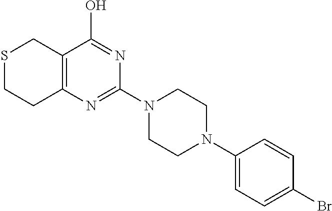 [SMILES: Oc1nc(N2CCN(c3ccc(Br)cc3)CC2)nc2c1CSCC2]

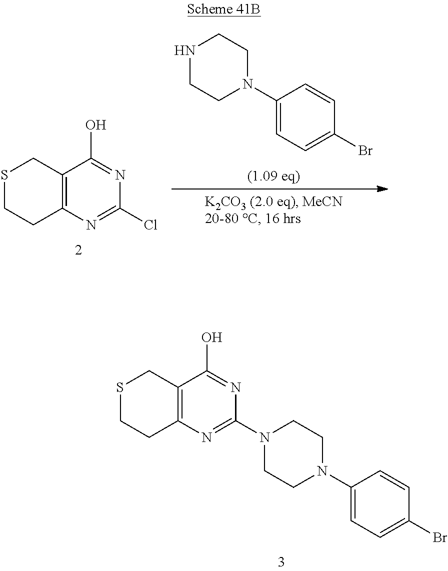 [SMILES: Oc1nc(Cl)nc2c1CSCC2 ; Brc1ccc(N2CCNCC2)cc1 ; Oc1nc(N2CCN(c3ccc(Br)cc3)CC2)nc2c1CSCC2]

To a solution of 2-chloro-7,8-dihydro-5H-thiopyrano[4,3-d]pyrimidin-4-ol (2) (0.2 g, 986.87 mol, 1 eq) in MeCN (2 mL) was added 1-(4-bromophenyl)piperazine (0.26 g, 1.08 mmol, 1.09 eq) and K₂CO₃ (272.78 mg, 1.97 mmol, 2 eq) at 20° C. The mixture was stirred at 80° C. for 16 h. LC-MS showed that (2) was consumed completely, and the desired mass was detected. The mixture was diluted with water (5 mL) and stirred at 20° C. for 4 h. The mixture was filtered, and the filter cake was dried under reduced pressure to give 2-(4-(4-bromophenyl)piperazin-1-yl)-7,8-dihydro-5H-thiopyrano[4,3-d]pyrimidin-4-ol (3) (0.2 g, crude) and used immediately in the next step.

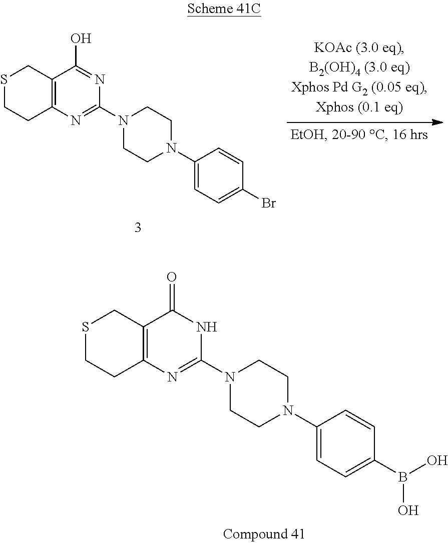 [SMILES: Oc1nc(N2CCN(c3ccc(Br)cc3)CC2)nc2c1CSCC2 ; O=c1[nH]c(N2CCN(c3ccc(B(O)O)cc3)CC2)nc2c1CSCC2]

To a solution of 2-(4-(4-bromophenyl)piperazin-1-yl)-7,8-dihydro-5H-thiopyrano[4,3-d]pyrimidin-4-ol (3) (380 mg, 932.91 mol, 1 eq) and B₂(OH)₄ (250.91 mg, 2.80 mmol, 3 eq) in EtOH (19 mL) was added KOAc (274.67 mg, 2.80 mmol, 3.0 eq), XPHOS-PD-G2 (36.70 mg, 46.65 mol, 0.05 eq), and XPhos (44.47 mg, 93.29 mol, 0.1 eq) at 20° C. The mixture was stirred at 90° C. for 16 h. LC-MS showed that (3) was consumed partly, and one main peak with the desired mass was detected. The reaction mixture was filtered, then the filtrate was concentrated under reduced pressure to give a residue. The residue was purified by prep-HPLC (column: Waters Xbridge Prep OBD C18 150×40 mm×10 um; mobile phase: [H₂O (10 mm NH₄HCO₃)-ACN]; gradient: 15%-55% B over 8.0 min) to provide (4-(4-(4-oxo-3,5,7,8-tetrahydro-4H-thiopyrano[4,3-d]pyrimidin-2-yl)piperazin-1-yl)phenyl)boronic acid (Compound 41). LC-MS: 373.1 (M+1). ¹H NMR (400 MHz, DMSO-d₆): δ 11.29 (br s, 1H), 7.72 (s, 2H), 7.70-7.64 (m, J=8.5 Hz, 2H), 6.98-6.85 (m, J=8.6 Hz, 2H), 3.77-3.62 (m, 4H), 3.42-3.35 (m, 2H), 3.29-3.19 (m, 4H), 2.82 (t, J=5.9 Hz, 2H), 2.66 (br t, J=5.4 Hz, 2H).

### Compound 42

### Synthesis of 2-(4-(2,6-difluoro-4-(2-hydroxyethoxy)phenyl)piperazin-1-yl)-3,5,7,8-tetrahydro-4H-thiopyrano[4,3-d]pyrimidin-4-one (Compound 42)

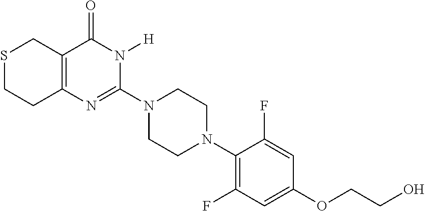 [SMILES: O=c1[nH]c(N2CCN(c3c(F)cc(OCCO)cc3F)CC2)nc2c1CSCC2]

### Synthesis of 5-(2-(benzyloxy)ethoxy)-2-bromo-1,3-difluorobenzene

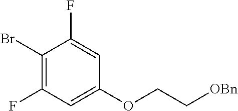 [SMILES: Fc1cc(OCCOCc2ccccc2)cc(F)c1Br]

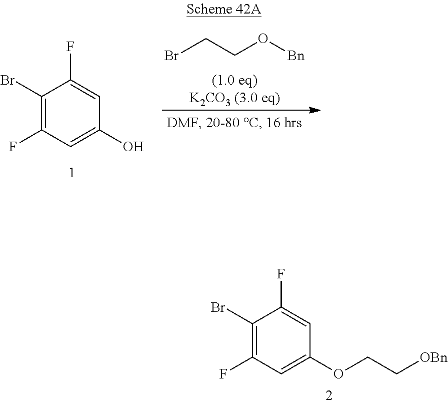 [SMILES: Oc1cc(F)c(Br)c(F)c1 ; BrCCOCc1ccccc1 ; Fc1cc(OCCOCc2ccccc2)cc(F)c1Br]

To a solution of 4-bromo-3,5-difluorophenol (1) (2.00 g, 9.57 mmol, 1.00 eq) in DMF (12.0 mL) was added 2-bromoethoxymethylbenzene (2.06 g, 9.57 mmol, 1.51 mL, 1.00 eq) and K₂CO₃ (3.97 g, 28.7 mmol, 3.00 eq) at 20° C. The mixture was heated to 80° C. and stirred for 16 h. The mixture was cooled to 20° C., then diluted with EtOAc (10.0 mL) and washed with NaCl (10.0 mL×3). The organic layer was dried over Na₂SO₄, filtered, and concentrated under reduced pressure to give 5-(2-(benzyloxy)ethoxy)-2-bromo-1,3-difluorobenzene (2) (3.00 g, crude). The compound was used without further purification in the next step. ¹H NMR (400 MHz, CDCl₃-d) 6 7.34 (m, 5H), 6.60 (m, 2H), 4.64 (m, 2H), 4.13 (m, 2H), 3.83 (m, 2H). ¹⁹F NMR (400 MHz, CDCl₃-d) 6-104.84 (s).

### Synthesis of tert-butyl 4-(4-(2-(benzyloxy)ethoxy)-2,6-difluorophenyl)piperazine-1-carboxylate

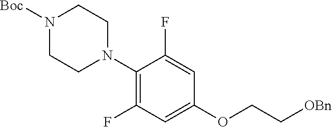 [SMILES: CC(C)(C)OC(=O)N1CCN(c2c(F)cc(OCCOCc3ccccc3)cc2F)CC1]

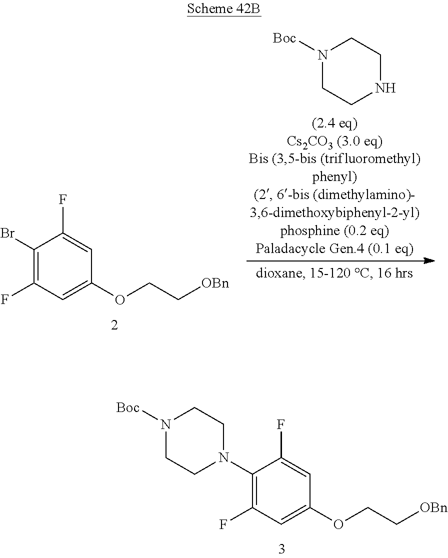 [SMILES: CC(C)(C)OC(=O)N1CCNCC1 ; Fc1cc(OCCOCc2ccccc2)cc(F)c1Br ; CC(C)(C)OC(=O)N1CCN(c2c(F)cc(OCCOCc3ccccc3)cc2F)CC1]

To a solution of 5-(2-(benzyloxy)ethoxy)-2-bromo-1,3-difluorobenzene (2) (1.00 g, 2.91 mmol, 1.00 eq) in dioxane (20.0 mL) was added tert-butyl piperazine-1-carboxylate (1.30 g, 6.99 mmol, 2.40 eq), Cs₂CO₃ (2.85 g, 8.74 mmol, 3.00 eq), Bis (3,5-bis(trifluoromethyl)phenyl) (2′,6′-bis(dimethylamino)-3,6-dimethoxybiphenyl-2-yl)phosphine (440.93 mg, 582.81 mol, 0.20 eq), and Paladacycle Gen.4 (332.5 mg, 291.41 mol, 0.10 eq) at 15° C. under N₂. The mixture was heated to 120° C. and stirred for 16 h under N₂. LC-MS showed that (2) was consumed. After cooling to 15° C., the mixture was filtered, and the filtrate was concentrated under reduced pressure to give a residue that was purified by column chromatography (SiO₂, petroleum ether/ethyl acetate=100/1 to 0/1) to provide tert-butyl 4-(4-(2-(benzyloxy)ethoxy)-2,6-difluorophenyl)piperazine-1-carboxylate (3) (350 mg, 780.38 mol). ¹H NMR (400 MHz, CDCl₃-d) 6:7.29-7.21 (m, 5H), 6.38 (m, 2H), 4.55 (m, 2H), 3.94 (m, 2H), 3.71 (m, 2H), 3.46 (m, 4H), 2.96 (m, 4H), 1.41 (M, 9H). ¹⁹F NMR (400 MHz, CDCl₃-d) 6: -118.34 (s).

### Synthesis of 1-(4-(2-(benzyloxy)ethoxy)-2,6-difluorophenyl)piperazine

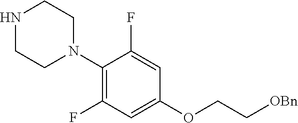 [SMILES: Fc1cc(OCCOCc2ccccc2)cc(F)c1N1CCNCC1]

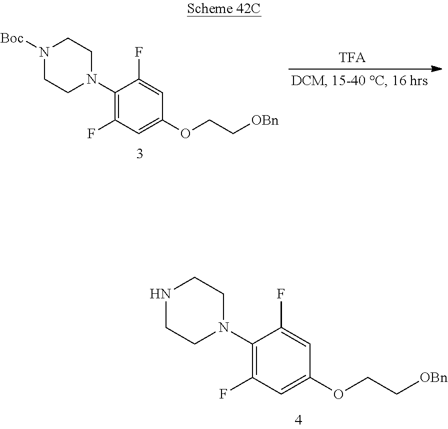 [SMILES: CC(C)(C)OC(=O)N1CCN(c2c(F)cc(OCCOCc3ccccc3)cc2F)CC1 ; Fc1cc(OCCOCc2ccccc2)cc(F)c1N1CCNCC1]

To a solution of tert-butyl 4-(4-(2-(benzyloxy)ethoxy)-2,6-difluorophenyl)piperazine-1-carboxylate (3) (350 mg, 780.38 mol, 1.00 eq) in DCM (3.50 mL) was added TFA (2.69 g, 23.56 mmol, 1.75 mL) at 15° C. The mixture was heated to 40° C. and stirred for 16 h. After cooling to 15° C., the reaction mixture was concentrated under reduced pressure to give 1-(4-(2-(benzyloxy)ethoxy)-2,6-difluorophenyl)piperazine (4) (350 mg, crude, TFA). LC-MS: 349.2 (M+1).

### Synthesis of 2-chloro-7,8-dihydro-5H-thiopyrano[4,3-d]pyrimidin-4-ol

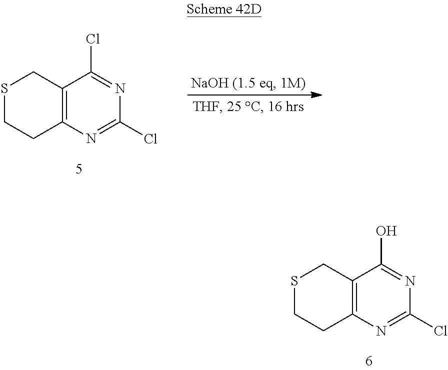 [SMILES: Clc1nc(Cl)c2c(n1)CCSC2 ; "NaOH (1.5 eq, 1M)" ; Oc1nc(Cl)nc2c1CSCC2]

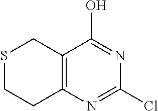 [SMILES: Oc1nc(Cl)nc2c1CSCC2]

To a solution of 2,4-dichloro-7,8-dihydro-5H-thiopyrano[4,3-d]pyrimidine (5) (500 mg, 2.26 mmol, 1.00 eq) in THF (1.00 mL) was added NaOH (1 M, 3.39 mL, 1.50 eq) at 25° C. and stirred for 16 h. The mixture was quenched with H₂O (10.0 mL) and extracted with EtOAc (10.0 mL). The aqueous phase was acidified with 1N HCl (20.0 mL) to adjust the pH to 3-4, and then stirred at 25° C. for 12 h. The mixture was extracted with EtOAc (10.0 mL×3). The combined organic layers were dried over Na₂SO₄, filtered, and concentrated under reduced pressure to give 2-chloro-7,8-dihydro-5H-thiopyrano[4,3-d]pyrimidin-4-ol (6) (0.8 g, 2.84 mmol). ¹H NMR (400 MHz, CDCl₃-d) δ: 3.54 (s, 2H), 2.87 (m, 2H), 2.82 (m, 2H). LC-MS: 203.2 (M+1).

### Synthesis of 2-(4-(4-(2-(benzyloxy)ethoxy)-2,6-difluorophenyl)piperazin-1-yl)-7,8-dihydro-5H-thiopyrano[4,3-d]pyrimidin-4-ol

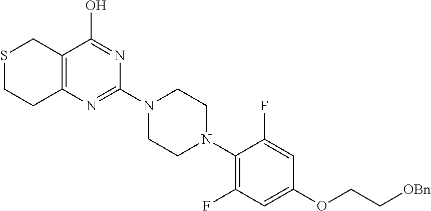 [SMILES: Oc1nc(N2CCN(c3c(F)cc(OCCOCc4ccccc4)cc3F)CC2)nc2c1CSCC2]

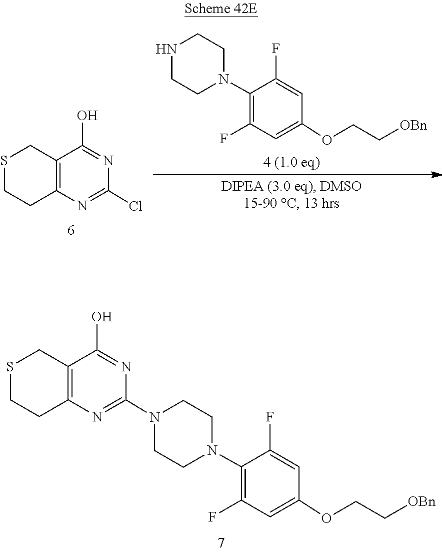 [SMILES: Oc1nc(Cl)nc2c1CSCC2 ; Fc1cc(OCCOCc2ccccc2)cc(F)c1N1CCNCC1 ; Oc1nc(N2CCN(c3c(F)cc(OCCOCc4ccccc4)cc3F)CC2)nc2c1CSCC2]

To a solution of 1-(4-(2-(benzyloxy)ethoxy)-2,6-difluorophenyl)piperazine (4) (300 mg, 779.53 mol, 1.00 eq, TFA) and 2-chloro-7,8-dihydro-5H-thiopyrano[4,3-d]pyrimidin-4-ol (6) (157.98 mg, 779.53 mol, 1.00 eq) in DMSO (3.00 mL) was added DIEA (302.25 mg, 2.34 mmol, 407.34 μL, 3.00 eq) at 15° C. The mixture was stirred at 90° C. for 13 hrs and then cooled to 15° C. The mixture was diluted with water (10.0 mL) and extracted with EtOAc. The combined organic layers were washed with brine, dried over Na₂SO₄, filtered, and concentrated under reduced pressure to give a residue that was purified by prep-HPLC (neutral condition; Waters Xbridge Prep OBD C18 150×40 mm×10 um; mobile phase: [H₂O (0.05% NH₃H₂O⁺; 10 mM NH₄HCO₃)-ACN]; gradient: 40%-70% B over 8.0 m) to provide 2-(4-(4-(2-(benzyloxy)ethoxy)-2,6-difluorophenyl)piperazin-1-yl)-7,8-dihydro-5H-thiopyrano[4,3-d]pyrimidin-4-ol (7) (200 mg, 376.15 mol). LC-MS: 516.2 (M+1).

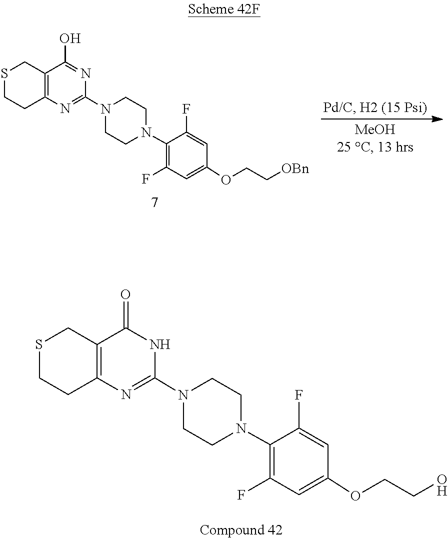 [SMILES: Oc1nc(N2CCN(c3c(F)cc(OCCOCc4ccccc4)cc3F)CC2)nc2c1CSCC2 ; O=c1[nH]c(N2CCN(c3c(F)cc(OCCO)cc3F)CC2)nc2c1CSCC2]

To a solution of 2-(4-(4-(2-(benzyloxy)ethoxy)-2,6-difluorophenyl)piperazin-1-yl)-7,8-dihydro-5H-thiopyrano[4,3-d]pyrimidin-4-ol (7) (100 mg, 194.33 mol, 1.00 eq) in MeOH (5 mL) was added Pd/C (100 mg, 194.33 mol, 1.00 eq) at 25° C. under N₂ atmosphere. The suspension was degassed and purged three times with H₂. The mixture was stirred under H₂ (15 psi) at 25° C. for 13 h. After cooling to 15° C., the reaction mixture was filtered through diatomite and the filtrate was concentrated under reduced pressure to give a residue that was purified by prep-HPLC (neutral conditions; column: Waters X bridge Prep OBD C18 150×40 mm×10 um; mobile phase: [H₂O (10 mM NH₄HCO₃)-ACN]; gradient: 20%-50% B over 8.0 m) to provide 2-(4-(2,6-difluoro-4-(2-hydroxyethoxy)phenyl)piperazin-1-yl)-3,5,7,8-tetrahydro-4H-thiopyrano[4,3-d]pyrimidin-4-one (Compound 42). LC-MS: 425.0 (M+1). ¹H NMR (400 MHz, DMSO-d₆): δ 6.74-6.69 (m, 2H), 4.88 (t, J=5.5 Hz, 1H), 3.97 (t, J=4.9 Hz, 2H), 3.71-3.65 (m, 6H), 3.41-3.36 (m, 2H), 3.03 (br s, 4H), 2.84-2.78 (m, 2H), 2.70-2.61 (m, 2H). ¹⁹F NMR (400 MHz, DMSO-d₆): δ −118.26.

### Compound 44

### Synthesis of 2-(1-hydroxy-3,3-dimethyl-1,3-dihydrobenzo[c][1,2]oxaborol-5-yl)-3,5,7,8-tetrahydro-4H-thiopyrano[4,3-d]pyrimidin-4-one (Compound 44)

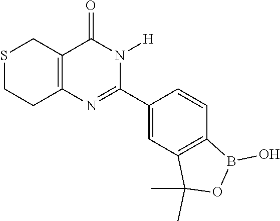 [SMILES: CC1(C)OB(O)c2ccc(-c3nc4c(c(=O)[nH]3)CSCC4)cc21]

### Synthesis of methyl 2-bromo-5-carbamimidoylbenzoate hydrochloride

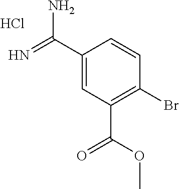

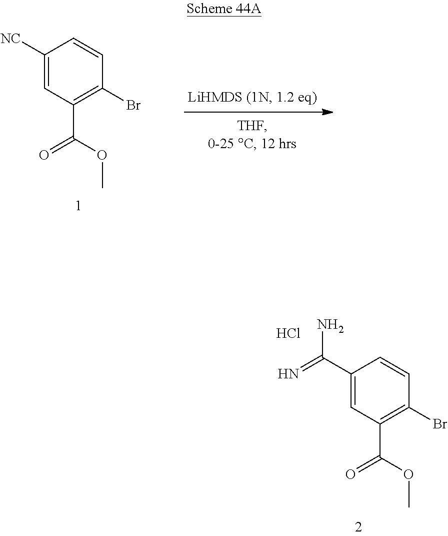

To a solution of methyl 2-bromo-5-cyanobenzoate (1) (3 g, 12.50 mmol, 1 eq) in THF (30 mL) was added LiHMDS (1 M, 15 mL, 1.2 eq) dropwise at 0° C. The mixture was warmed to 25° C. and stirred for 12 h. LCMS indicated that (1) was consumed completely and one main peak formed. The mixture was quenched by addition of HCl/dioxane (4M, 15 mL) at 0° C. and warmed to 15° C. while stirring for 4 h. The mixture was concentrated under reduced pressure to give methyl 2-bromo-5-carbamimidoylbenzoate hydrochloride (2) (5.5 g, crude, HCl). The crude product was used into the next step without further purification. LCMS: (EC16609-21-P1A1, product: Rₜ=0.211 mins).

### Synthesis of methyl 2-bromo-5-(4-oxo-3,5,7,8-tetrahydro-4H-thiopyrano[4,3-d]pyrimidin-2-yl)benzoate

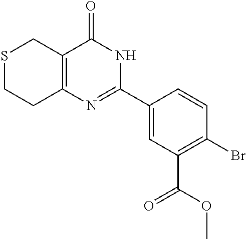 [SMILES: COC(=O)c1cc(-c2nc3c(c(=O)[nH]2)CSCC3)ccc1Br]

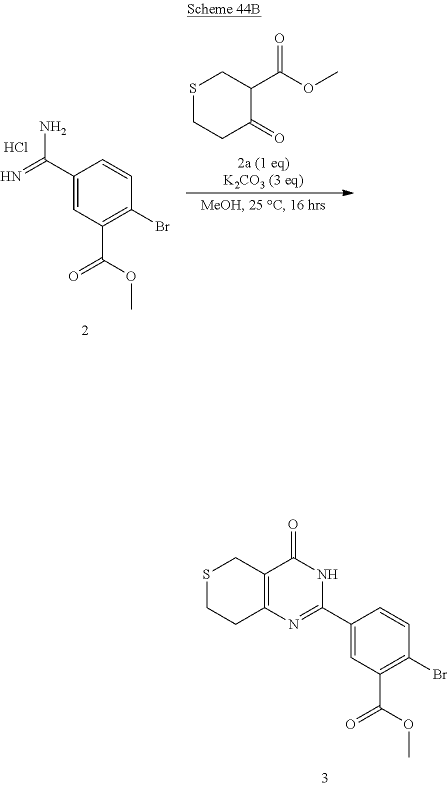 [SMILES: Cl ; COC(=O)c1cc(C(=N)N)ccc1Br ; COC(=O)C1CSCCC1=O ; COC(=O)c1cc(-c2nc3c(c(=O)[nH]2)CSCC3)ccc1Br]

To a solution of methyl 2-bromo-5-carbamimidoylbenzoate hydrochloride (2) (5.6 g, 19.08 mmol, 1 eq, HCl) in MeOH (56 mL) was added methyl 4-oxotetrahydro-2H-thiopyran-3-carboxylate (2a) (3.32 g, 19.08 mmol, 1 eq) and K₂CO₃ (7.91 g, 57.23 mmol, 3 eq) at 25° C. and then stirred for 16 h. LCMS indicated that (2) was consumed completely and one main peak formed. The reaction mixture was filtered and the filtrate was concentrated in vacuum to give a residue that was added to H₂O (50 mL) at 25° C. and stirred for 2 h. The mixture was filtered and methyl 2-bromo-5-(4-oxo-3,5,7,8-tetrahydro-4H-thiopyrano[4,3-d]pyrimidin-2-yl)benzoate (3) (3.8 g, 9.97 mmol, crude) was obtained, which was used in the next step without further purification. LCMS: (EC16609-21-P1A1, product: Rₜ=0.430 mins).

### Synthesis of 2-(4-bromo-3-(2-hydroxypropan-2-yl)phenyl)-3,5,7,8-tetrahydro-4H-thiopyrano[4,3-d]pyrimidin-4-one

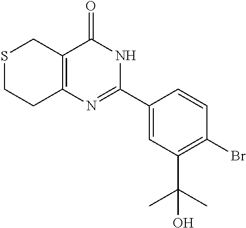 [SMILES: CC(C)(O)c1cc(-c2nc3c(c(=O)[nH]2)CSCC3)ccc1Br]

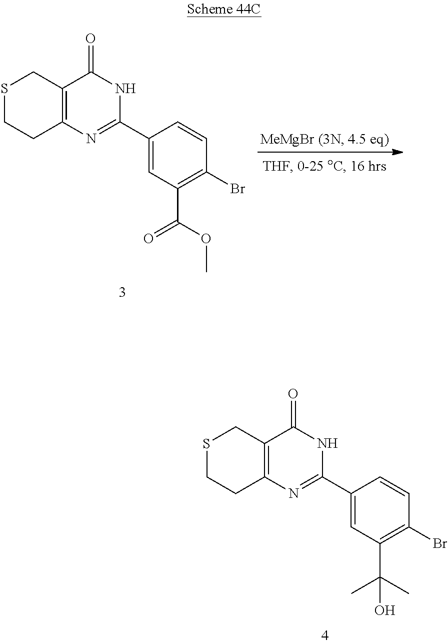 [SMILES: COC(=O)c1cc(-c2nc3c(c(=O)[nH]2)CSCC3)ccc1Br ; CC(C)(O)c1cc(-c2nc3c(c(=O)[nH]2)CSCC3)ccc1Br]

To a solution of methyl 2-bromo-5-(4-oxo-3,5,7,8-tetrahydro-4H-thiopyrano[4,3-d]pyrimidin-2-yl)benzoate (3) (1.5 g, 3.93 mmol, 1 eq) in THF (15 mL) was added MeMgBr (3 M, 5.90 mL, 4.5 eq) dropwise at 0° C. The mixture was heated to 25° C. and stirred for 16 h. TLC (Petroleum ether/Ethyl acetate=2:1) indicated some of (3) remained. The reaction mixture was quenched by addition saturated NH₄Cl at 0° C., and then extracted with ethyl acetate. The organic layers were combined and concentrated under reduced pressure to give 2-(4-bromo-3-(2-hydroxypropan-2-yl)phenyl)-3,5,7,8-tetrahydro-4H-thiopyrano[4,3-d]pyrimidin-4-one (4) (1.2 g, 3.15 mmol, crude), which was used in the next step without further purification. LCMS: (EC16609-14-P1A, product: Rₜ=0.415 mins).

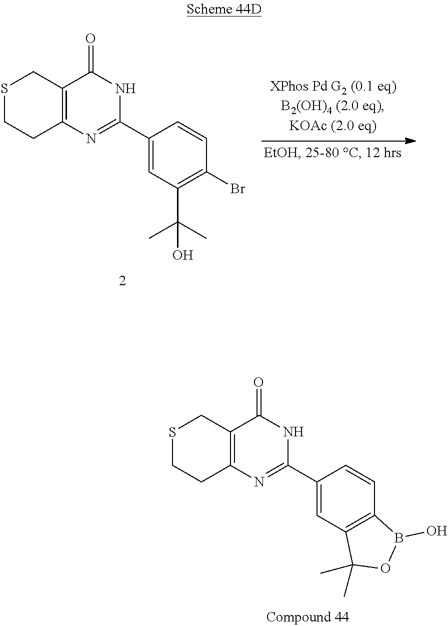 [SMILES: CC(C)(O)c1cc(-c2nc3c(c(=O)[nH]2)CSCC3)ccc1Br ; CC1(C)OB(O)c2ccc(-c3nc4c(c(=O)[nH]3)CSCC4)cc21]

To a solution of 2-(4-bromo-3-(2-hydroxypropan-2-yl)phenyl)-3,5,7,8-tetrahydro-4H-thiopyrano[4,3-d]pyrimidin-4-one (4) (1.2 g, 3.15 mmol, 1 eq), B₂(OH)₄ (564.31 mg, 6.29 mmol, 2 eq) and KOAc (617.75 mg, 6.29 mmol, 2 eq) in EtOH (24 mL) was added XPhos Pd G₂ (247.63 mg, 314.72 mol, 0.1 eq) at 25° C. The mixture was heated to 80° C. and stirred for 12 hr. LCMS indicated that (4) was consumed completely. The mixture was filtered and the filtrate concentrated under reduced pressure to give a residue. The residue was purified by prep-HPLC (column: Waters Xbridge BEH C18 250 * 50 mm*10 um; mobile phase: [water (NH₃H₂O)-ACN]; gradient: 1%-30% B over 10 min) to provide 2-(1-hydroxy-3,3-dimethyl-1,3-dihydrobenzo[c][1,2]oxaborol-5-yl)-3,5,7,8-tetrahydro-4H-thiopyrano[4,3-d]pyrimidin-4-one (Compound 44). ¹H NMR (400 MHz, DMSO-d₆): δ ppm 9.16 (br s, 1H) 8.10 (s, 1H) 8.05 (br d, J=7.38 Hz, 1H) 7.74 (d, J=7.50 Hz, 1H) 3.52 (s, 2H) 2.87 (br s, 4H) 1.49 (s, 6H). LCMS: (EC16609-26-P1A3, product: Rₜ=0.372 mins).

### Compound A1

### Synthesis of 2-((1r,3r)-3-aminocyclobutyl)-3,5,7,8-tetrahydro-4H-thiopyrano[4,3-d]pyrimidin-4-one (A1)

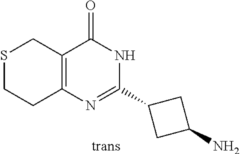

### Synthesis of (Z)-tert-butyl-3-hydroxycyclobutyl)carbamate

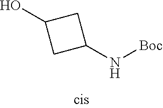 [SMILES: CC(C)(C)OC(=O)NC1CC(O)C1]

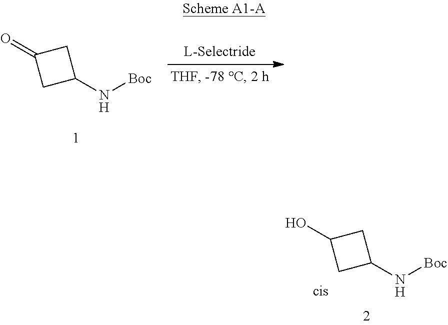 [SMILES: CC(C)(C)OC(=O)NC1CC(=O)C1 ; CC(C)(C)OC(=O)NC1CC(O)C1]

A solution of tert-butyl (3-oxocyclobutyl)carbamate (1) (2.5 g, 13.5 mmol, 1 eq) in 80 mL THF was cooled to −78° C. and treated with a 1 N solution of L-Selectride (16.2 mL, 16.2 mmol, 1.2 eq) in THF. After stirring for 1 h, the reaction was quenched with 5 mL water and warmed to room temperature (rt). The reaction mixture was concentrated and purified by silica gel chromatography (EA:PE=0-50%) to provide (Z)-tert-butyl-3-hydroxycyclobutyl)carbamate (2). ¹H NMR (400 MHz, CDCl₃): δ=4.68 (brs, 1H), 4.05-3.98 (m, 1H), 3.67-3.65 (m, 1H), 2.78-2.75 (m, 2H), 2.08 (brs, 1H), 1.81-1.78 (m, 2H), 1.44 (s, 9H).

### Synthesis of (Z)-3-((tert-butoxycarbonyl)amino)cyclobutyl methanesulfonate

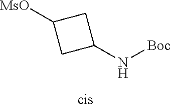 [SMILES: CC(C)(C)OC(=O)NC1CC(OS(C)(=O)=O)C1]

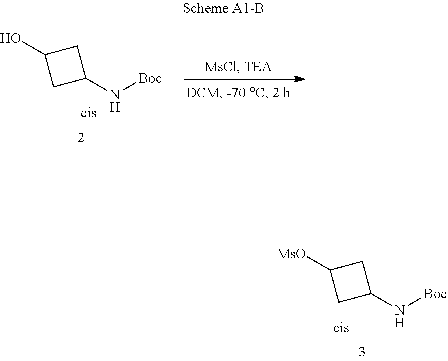 [SMILES: CC(C)(C)OC(=O)NC1CC(O)C1 ; CC(C)(C)OC(=O)NC1CC(OS(C)(=O)=O)C1]

Methanesulfonyl chloride (1.3 g, 11.6 mmol, 1.2 eq) was added dropwise to a solution consisting of (Z)-tert-butyl-3-hydroxycyclobutyl)carbamate (2) (1.8 g, 9.6 mmol, 1 eq), TEA (1.5 g, 14.8 mmol, 1.5 eq) and dichloromethane (60 mL) at −70° C. The resulting solution was stirred for 2 h at −70° C., and then diluted with water. The resulting solution was extracted with dichloromethane and the organic layers combined, washed with brine, dried over anhydrous sodium sulfate, and concentrated under reduced pressure to give (Z)-3-((tert-butoxycarbonyl)amino)cyclobutyl methanesulfonate (3). ¹H NMR (400 MHz, CDCl₃): δ (ppm): 4.75-4.67 (m, 2H), 3.85-3.82 (m, 1H), 2.98 (s, 3H), 2.93-2.88 (m, 2H), 2.22-2.14 (m, 2H), 1.44 (s, 9H).

### Synthesis of (E)-tert-butyl (3-cyanocyclobutyl)carbamate

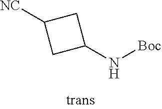 [SMILES: CC(C)(C)OC(=O)NC1CC(C#N)C1]

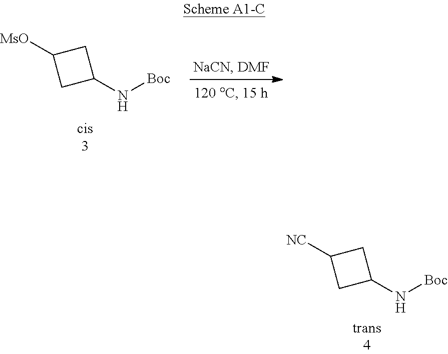 [SMILES: CC(C)(C)OC(=O)NC1CC(OS(C)(=O)=O)C1 ; CC(C)(C)OC(=O)NC1CC(C#N)C1]

(Z)-3-((tert-butoxycarbonyl)amino)cyclobutyl methanesulfonate (3) (1.2 g, 4.5 mmol, 1 eq) in DMF (30 mL) was treated with NaCN (665.7 mg, 13.6 mmol, 3 eq) and the reaction was heated to 120° C. for 15 h. The mixture was then cooled to 0° C. and diluted with water. The resulting solution was extracted with ethyl acetate and the organic layers were combined. The organic layer was washed with brine, dried over anhydrous sodium sulfate, and concentrated under reduced pressure to give (E)-tert-butyl (3-cyanocyclobutyl)carbamate (4).

¹H NMR (400 MHz, CDCl₃): δ=4.77 (br s, 1H), 4.41-4.39 (m, 1H), 3.06-3.01 (m, 1H), 2.74-2.41 (m, 2H), 2.27-2.24 (m, 2H), 1.45 (s, 9H).

### Synthesis of (E)-tert-butyl (3-(N-hydroxycarbamimidoyl)cyclobutyl)carbamate

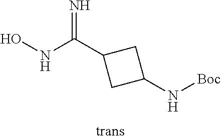 [SMILES: CC(C)(C)OC(=O)NC1CC(C(=N)NO)C1]

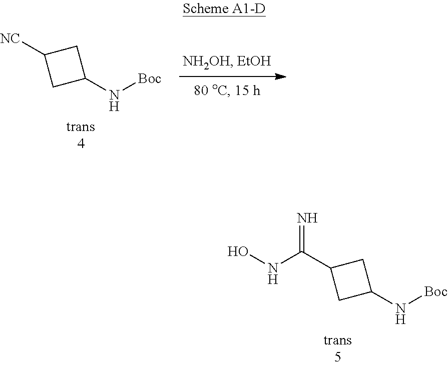 [SMILES: CC(C)(C)OC(=O)NC1CC(C#N)C1 ; CC(C)(C)OC(=O)NC1CC(C(=N)NO)C1]

To a solution of (E)-tert-butyl (3-cyanocyclobutyl)carbamate (4) (730.0 mg, 3.7 mmol, 1 eq) in EtOH (25 mL) was added hydroxylamine in ethanol (2.2 g/2.0 mL of ethanol, 33.3 mmol, 9 eq). The mixture was heated to 80° C. and stirred for 15 h. The reaction mixture was then cooled to RT and concentrated under reduced pressure followed by prep-HPLC purification to give (E)-tert-butyl (3-(N-hydroxycarbamimidoyl)cyclobutyl)carbamate (5). LCMS (ESI): m/z 230.2 [M+H]⁺.

### Synthesis of (E)-tert-butyl (3-carbamimidoylcyclobutyl)carbamate

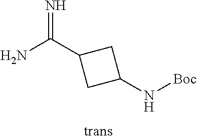 [SMILES: CC(C)(C)OC(=O)NC1CC(C(=N)N)C1]

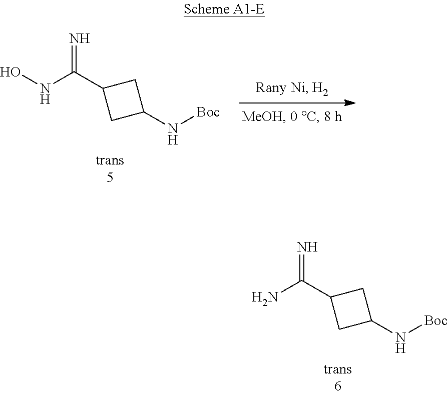 [SMILES: CC(C)(C)OC(=O)NC1CC(C(=N)NO)C1 ; CC(C)(C)OC(=O)NC1CC(C(=N)N)C1]

To a solution of (E)-tert-butyl (3-(N-hydroxycarbamimidoyl)cyclobutyl)carbamate (5) (520.0 mg, 2.3 mmol, 1 eq) in MeOH (100 mL) was added Raney-Ni (200 mg, 2.3 mmol, 1 eq). The suspension was degassed under vacuum and purged with H₂ several times. The mixture was stirred under an H₂ atmosphere at 0° C. for 8 h. The reaction mixture was then filtered, and the filtrate was concentrated under reduced pressure to give (E)-tert-butyl (3-carbamimidoylcyclobutyl)carbamate (6) (480.0 mg). LCMS (ESI): m/z 214.2 [M+H]⁺.

### Synthesis of (E)-tert-butyl ((1r,3r)-3-(4-oxo-3,5,7,8-tetrahydro-4H-thiopyrano[4,3-d]pyrimidin-2-yl)cyclobutyl)carbamate

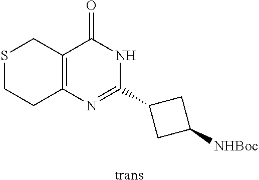 [SMILES: CC(C)(C)OC(=O)N[C@H]1C[C@H](c2nc3c(c(=O)[nH]2)CSCC3)C1 ; "trans"]

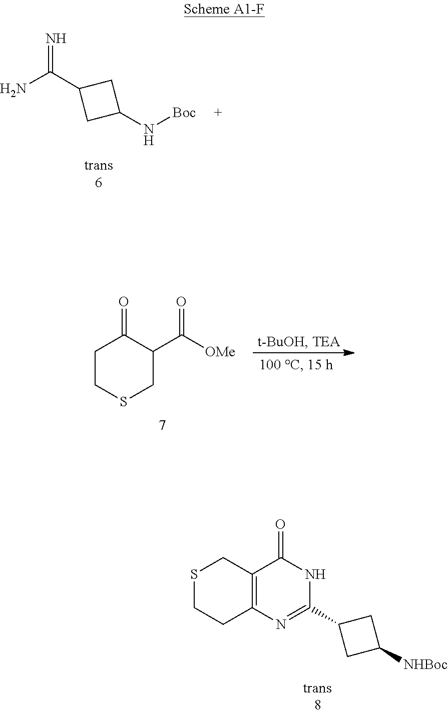 [SMILES: CC(C)(C)OC(=O)NC1CC(C(=N)N)C1 ; COC(=O)C1CSCCC1=O ; CC(C)(C)OC(=O)N[C@H]1C[C@H](c2nc3c(c(=O)[nH]2)CSCC3)C1]

To a solution of E)-tert-butyl (3-carbamimidoylcyclobutyl)carbamate (6) (480.0 mg, 2.3 mmol, 1 eq) and (7) (431.3 mg, 2.5 mmol, 1.1 eq) in t-BuOH (30 mL) was added TEA (1.3 g, 12.8 mmol, 5.6 eq) in one portion at 15° C. The mixture was heated to 100° C. and stirred for 15 h. The reaction was concentrated in vacuo. The resultant residue was purified by silica gel column chromatography (DCM/MeOH=100% to 95% DCM/MeOH) to give (E)-tert-butyl ((1r,3r)-3-(4-oxo-3,5,7,8-tetrahydro-4H-thiopyrano[4,3-d]pyrimidin-2-yl)cyclobutyl)carbamate (8) (630.1 mg). LCMS (ESI): m/z 338.1 [M+H]⁺.

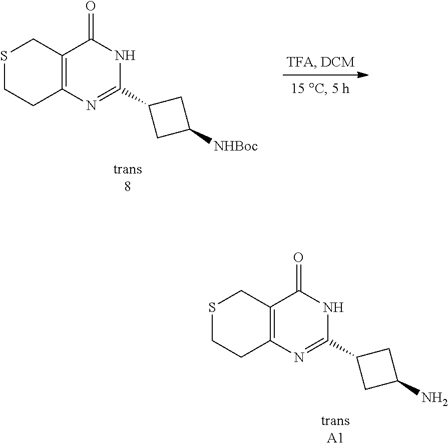 [SMILES: CC(C)(C)OC(=O)N[C@H]1C[C@H](c2nc3c(c(=O)[nH]2)CSCC3)C1 ; N[C@H]1C[C@H](c2nc3c(c(=O)[nH]2)CSCC3)C1]

To a stirred solution of (E)-tert-butyl ((1r,3r)-3-(4-oxo-3,5,7,8-tetrahydro-4H-thiopyrano[4,3-d]pyrimidin-2-yl)cyclobutyl)carbamate (8) (630 mg, 1.9 mmol, 1 eq) in DCM (30 mL) was added TFA (3 mL, 39.5 mmol, 20.8 eq) at 25° C. The reaction mixture was stirred for 5 h. The reaction mixture was concentrated in vacuum to give (E)-2-((1r,3r)-3-aminocyclobutyl)-3,5,7,8-tetrahydro-4H-thiopyrano[4,3-d]pyrimidin-4-one (A1). LCMS (ESI): m/z 238.0 [M+H]⁺. ¹H-NMR (400 MHz, DMSO-d₆): δ=8.41 (brs, 3H), 3.75-3.71 (m, 1H), 3.47 (s, 2H), 3.43-3.80 (m, 1H), 2.90-2.87 (m, 4H), 2.66-2.53 (m, 4H).

### Compound A4

### Synthesis of 2-(2-(6-bromopyridin-3-yl)pyrimidin-5-yl)-3,5,7,8-tetrahydro-4H-thiopyrano[4,3-d]pyrimidin-4-one (A4)

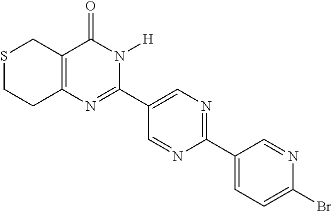 [SMILES: O=c1[nH]c(-c2cnc(-c3ccc(Br)nc3)nc2)nc2c1CSCC2]

### Synthesis of methyl 2-bromopyrimidine-5-carboxylate

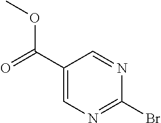

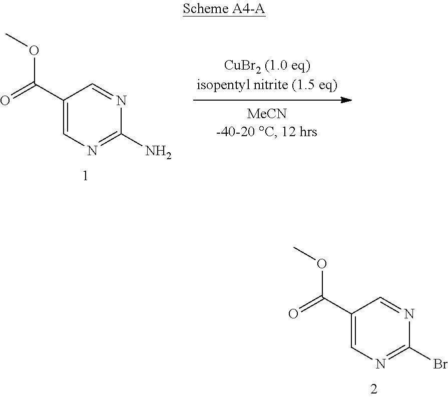

To a solution of methyl 2-aminopyrimidine-5-carboxylate (1) (5.00 g, 32.6 mmol, 1 eq) in MeCN (50 mL) was added CuBr₂ (7.29 g, 32.6 mmol, 1.53 mL, 1 eq) at 20° C. under N₂. The mixture was cooled to −40° C. and then isopentyl nitrite (5.74 g, 48.9 mmol, 6.59 mL, 1.50 eq) added dropwise. The mixture was warmed to 20° C. and stirred for 12 hrs. The mixture was diluted with EtOAc, filtered, and concentrated under reduced pressure to give a residue that was purified by column chromatography (SiO₂, Petroleum ether/Ethyl acetate=100/1 to 5/1) to provide methyl 2-(6-bromopyridin-3-yl)pyrimidine-5-carboxylate (2) (1.60 g, 7.37 mmol).

### Synthesis of methyl 2-(6-bromopyridin-3-yl)pyrimidine-5-carboxylate

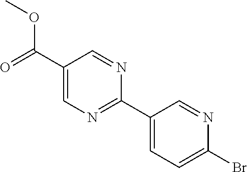 [SMILES: COC(=O)c1cnc(-c2ccc(Br)nc2)nc1]

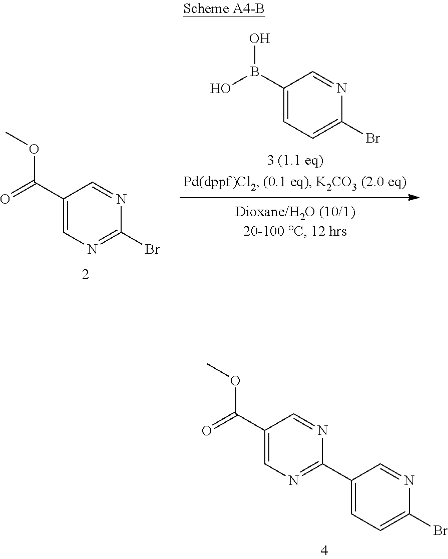 [SMILES: COC(=O)c1cnc(Br)nc1 ; OB(O)c1ccc(Br)nc1 ; COC(=O)c1cnc(-c2ccc(Br)nc2)nc1]

To a solution of methyl 2-bromopyrimidine-5-carboxylate (2) (1.60 g, 7.36 mmol, 1 eq) in dioxane (16 mL) and H₂O (1.60 mL) was added K₂CO₃ (2.03 g, 14.7 mmol, 2 eq) and (6-bromopyridin-3-yl)boronic acid (3) (1.63 g, 8.09 mmol, 1.10 eq) at 20° C. followed by Pd(dppf)Cl₂ (538 mg, 735 umol, 0.10 eq). The mixture was heated to 100° C. and stirred for 12 hrs. The reaction mixture was filtered, and the filtrate concentrated under reduced pressure to give a residue that was purified by prep-HPLC (column: Phenomenex luna C18 (250*70 mm, 15 um); mobile phase: [water (HCl) -ACN]; B %: 35%-65%, 18 mins) to provide methyl 2-(6-bromopyridin-3-yl)pyrimidine-5-carboxylate (4) (600 mg, 2.04 mmol). ¹H NMR (400 MHz, chloroform-d) δ=9.49 (d, J=2.4 Hz, 1H), 9.36 (s, 2H), 8.65 (dd, J=2.4, 8.4 Hz, 1H), 7.67 (d, J=8.3 Hz, 1H), 4.04 (s, 3H).

### Synthesis of 2-(6-bromopyridin-3-yl)pyrimidine-5-carboximidamide

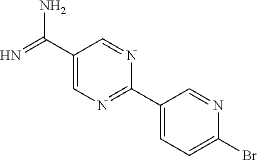 [SMILES: N=C(N)c1cnc(-c2ccc(Br)nc2)nc1]

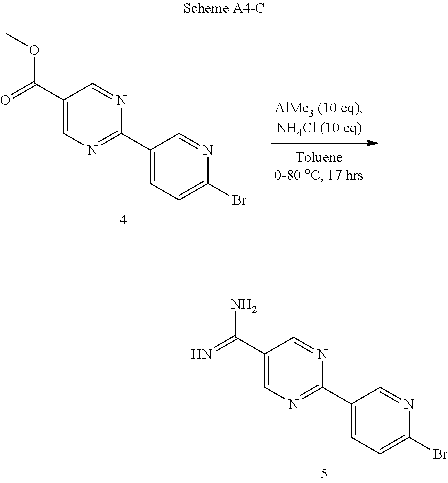 [SMILES: COC(=O)c1cnc(-c2ccc(Br)nc2)nc1 ; N=C(N)c1cnc(-c2ccc(Br)nc2)nc1]

NH₄Cl (672 mg, 12.5 mmol, 10 eq) was added to toluene (5.55 mL) at 25° C. AlMe₃ (1.00 M, 6.29 mL, 10 eq) was added dropwise at 0° C. The mixture was stirred at 0° C. for 1 hr. Then, methyl 2-(6-bromopyridin-3-yl)pyrimidine-5-carboxylate (4) (370 mg, 1.26 mmol, 1 eq) in toluene (1.85 mL) was added dropwise at 0° C. The mixture was warmed to 80° C. and stirred for 16 hrs under N₂. The reaction mixture was cooled to 0° C. and MeOH added dropwise at 0° C. The mixture was then filtered, and the filtrate concentrated under reduced pressure to give a residue that was purified by prep-HPLC (0.1% NH₄HCO₃) to provide 2-(6-bromopyridin-3-yl)pyrimidine-5-carboximidamide (5) (50.0 mg, 179 μmol).

### Synthesis of 2-(2-(6-bromopyridin-3-yl)pyrimidin-5-yl)-3,5,7,8-tetrahydro-4H-thiopyrano[4,3-d]pyrimidin-4-one

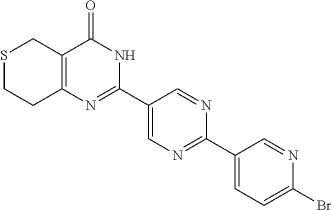 [SMILES: O=c1[nH]c(-c2cnc(-c3ccc(Br)nc3)nc2)nc2c1CSCC2]

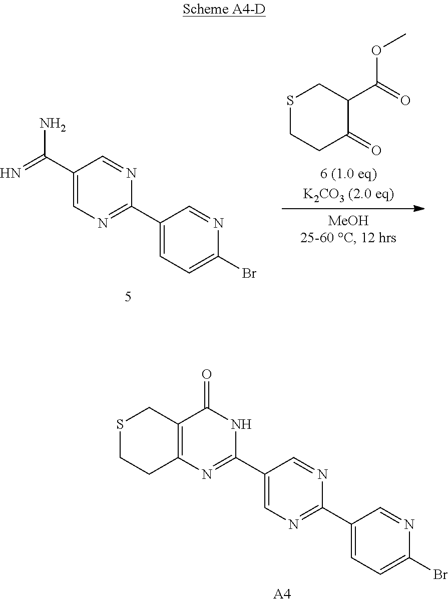 [SMILES: N=C(N)c1cnc(-c2ccc(Br)nc2)nc1 ; COC(=O)C1CSCCC1=O ; O=c1[nH]c(-c2cnc(-c3ccc(Br)nc3)nc2)nc2c1CSCC2 ; "A4"]

To a mixture of 2-(6-bromopyridin-3-yl)pyrimidine-5-carboximidamide (5) (50.0 mg, 179 umol, 1 eq) in MeOH (1 mL) was added methyl 4-oxotetrahydro-2H-thiopyran-3-carboxylate (6) (31.3 mg, 179 umol, 1 eq) and K₂CO₃ (49.7 mg, 359 umol, 2 eq) at 25° C. The mixture was warmed to 60° C. and stirred for 12 hrs. The reaction mixture was filtered, and the filtrate concentrated under reduced pressure to give a residue that was purified by prep-HPLC (column: Phenomenex luna C18 80*40 mm*3 um; mobile phase: [water (HCl)-ACN]; B %: 20%-50%, 8 mins) to provide 2-(2-(6-bromopyridin-3-yl)pyrimidin-5-yl)-3,5,7,8-tetrahydro-4H-thiopyrano[4,3-d]pyrimidin-4-one (A4). ¹H NMR (400 MHz, DMSO-d6): δ=13.51-12.57 (m, 1H), 9.50 (s, 2H), 9.35 (d, J=1.9 Hz, 1H), 8.66 (dd, J=2.5, 8.4 Hz, 1H), 7.88 (d, J=8.4 Hz, 1H), 3.58 (s, 2H), 2.93 (s, 4H).

### Compound A5

### Synthesis of 2-(4-(6-bromopyridin-3-yl)-2-fluorophenyl)-3,5,7,8-tetrahydro-4H-thiopyrano[4,3-d]pyrimidin-4-one (A5)

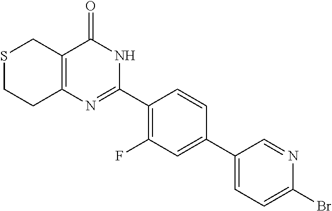 [SMILES: O=c1[nH]c(-c2ccc(-c3ccc(Br)nc3)cc2F)nc2c1CSCC2]

### Synthesis of 4-(6-bromopyridin-3-yl)-2-fluorobenzonitrile

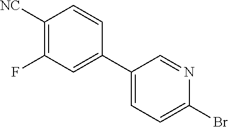 [SMILES: N#Cc1ccc(-c2ccc(Br)nc2)cc1F]

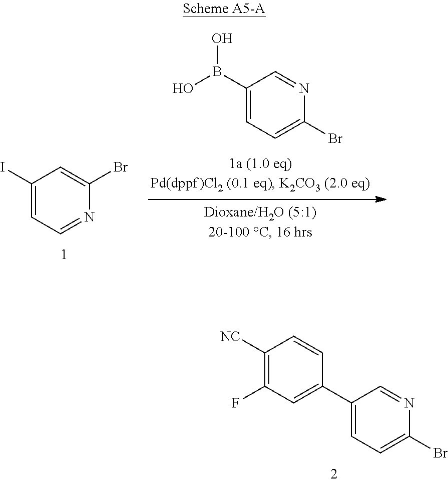 [SMILES: OB(O)c1ccc(Br)nc1 ; Brc1cc(I)ccn1 ; N#Cc1ccc(-c2ccc(Br)nc2)cc1F]

To a solution of 2-fluoro-4-iodobenzonitrile (1) (0.5 g, 2.02 mmol, 1.0 eq) in dioxane (5 mL)/H₂O (1 mL) was added (6-bromopyridin-3-yl)boronic acid (1a) (408.52 mg, 2.02 mmol, 1.0 eq), K₂CO₃ (559.52 mg, 4.05 mmol, 2.0 eq), and Pd(dppf)Cl₂ (148.11 mg, 202.42 μmol, 0.1 eq) at 20° C. under N₂. The mixture was stirred at 100° C. for 16 h under N₂. TLC (petroleum ether/ethyl acetate=5/1, R_{f}(cpd. 1)=0.77) indicated that (1) was consumed, and one new spot with larger polarity was detected. After cooling to 20° C., the mixture was filtered, and then the filtrate was concentrated under reduced pressure to give a residue. 4-(6-bromopyridin-3-yl)-2-fluorobenzonitrile (2) (650 mg, crude) was obtained. ¹H NMR: (400 MHz, CDCl₃): δ=8.53 (d, J=2.4 Hz, 1H), 7.71-7.65 (m, 2H), 7.56 (d, J=8.3 Hz, 1H), 7.41-7.33 (m, 2H).

### Synthesis of 4-(6-bromopyridin-3-yl)-2-fluorobenzimidamide

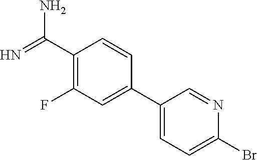 [SMILES: N=C(N)c1ccc(-c2ccc(Br)nc2)cc1F]

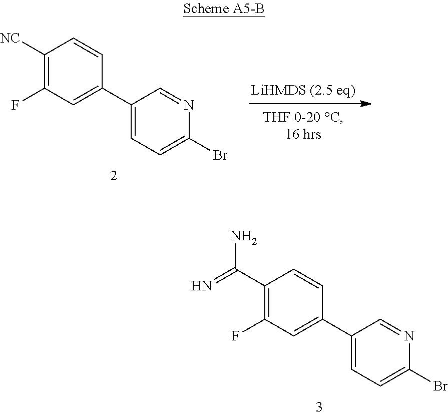 [SMILES: N#Cc1ccc(-c2ccc(Br)nc2)cc1F ; N=C(N)c1ccc(-c2ccc(Br)nc2)cc1F]

To a solution of 4-(6-bromopyridin-3-yl)-2-fluorobenzonitrile (2) (500 mg, 1.80 mmol, 1.0 eq) in THF (5 mL) was added LHMDS (1 M, 4.51 mL, 2.5 eq) at 0° C. under N₂. The mixture was warmed to 20° C. and stirred at 20° C. for 16 h. The reaction mixture was quenched by addition of HCl/dioxane (4 mol/L, 2.5 mL) at 0° C., and then concentrated under reduced pressure to give a residue. The residue was suspended in MeOH (15 ml), and the mixture was stirred at 20° C. for 1 hr. The mixture was then filtered, and the filtrate was concentrated under reduced pressure to give a residue. 4-(6-bromopyridin-3-yl)-2-fluorobenzimidamide (3) (540 mg, crude, HCl) was obtained and used immediately in the next step.

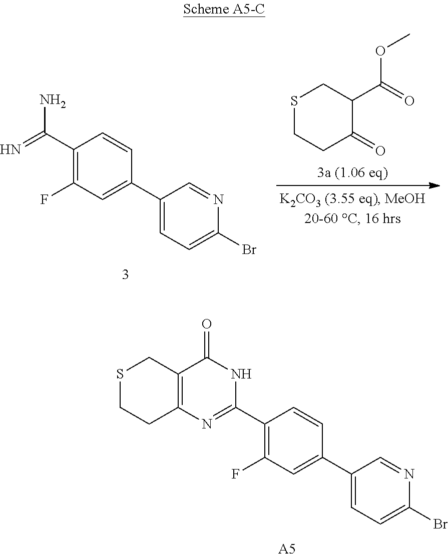 [SMILES: N=C(N)c1ccc(-c2ccc(Br)nc2)cc1F ; COC(=O)C1CSCCC1=O ; O=c1[nH]c(-c2ccc(-c3ccc(Br)nc3)cc2F)nc2c1CSCC2 ; "A5"]

To a solution of 4-(6-bromopyridin-3-yl)-2-fluorobenzimidamide (3) (220 mg, 747.99 mol, 1.0 eq) in MeOH (2.2 mL) was added methyl 4-oxotetrahydro-2H-thiopyran-3-carboxylate (3a) (137.50 mg, 789.25 mol, 1.06 eq) and K₂CO₃ (366.68 mg, 2.65 mmol, 3.55 eq) at 20° C.; the mixture was stirred at 60° C. for 16 h. The mixture was filtered, and the filtrate was concentrated under reduced pressure to give a residue. The residue was purified by prep-HPLC (FA condition; column: Phenomenex luna C18 80*40 mm*3 m; mobile phase: [water (FA)-ACN]; B %: 30%-70%, 8 mins). 2-(4-(6-bromopyridin-3-yl)-2-fluorophenyl)-3,5,7,8-tetrahydro-4H-thiopyrano[4,3-d]pyrimidin-4-one (A5) (30 mg) was obtained. ¹H NMR: (400 MHz, DMSO-d₆): δ=8.85 (d, J=2.4 Hz, 1H), 8.19 (dd, J=2.5, 8.4 Hz, 1H), 7.88-7.84 (m, 1H), 7.83-7.79 (m, 2H), 7.77-7.73 (m, 1H), 3.54 (s, 2H), 2.89 (br dd, J=4.5, 11.8 Hz, 4H). LC-MS: 420+bromo isomers (M+1).

### Compound A6

### Synthesis of 2-(4-(6-bromopyridin-3-yl)-2,5-difluorophenyl)-3,5,7,8-tetrahydro-4H-thiopyrano[4,3-d]pyrimidin-4-one (A6)

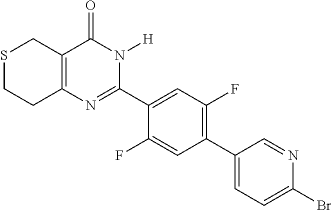 [SMILES: O=c1[nH]c(-c2cc(F)c(-c3ccc(Br)nc3)cc2F)nc2c1CSCC2]

### Synthesis of 4-amino-2,5-difluorobenzonitrile

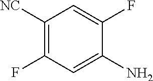 [SMILES: N#Cc1cc(F)c(N)cc1F]

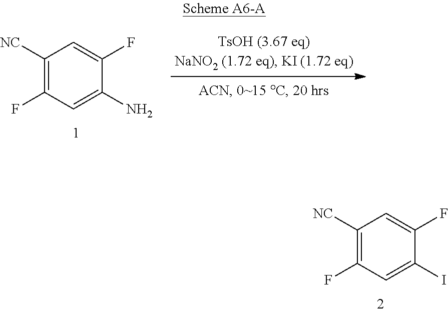 [SMILES: N#Cc1cc(F)c(N)cc1F ; N#Cc1cc(F)c(I)cc1F]

A mixture of 4-amino-2,5-difluorobenzonitrile (1) (3.00 g, 19.5 mmol, 1.00 eq), TsOH (12.3 g, 71.4 mmol, 3.67 eq) in ACN (60.0 mL) was stirred for 4 hrs at 15° C. Then NaNO₂ (2.31 g, 33.5 mmol, 1.72 eq) and KI (5.56 g, 33.5 mmol, 1.72 eq) were added into the reaction mixture at 0° C. Then the mixture was stirred at 15° C. for 16 hrs. LCMS showed that (1) was consumed completely. The reaction mixture was diluted with Na₂SO₃ (20 mL) and extracted with EtOAc 120 mL (40.0 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) and obtained 2.8 g crude. 1.3 g of crude product was purified by prep-HPLC (column: Phenomenex luna C18 80*40 mm*3 um; mobile phase: [water (HCl)-ACN]; B %: 35%-65%, 7 min) to give 0.5 g desired product. 2,5-Difluoro-4-iodobenzonitrile (2) (1.5 g, crude) was obtained, verified by NMR, and used into the next step.

### Synthesis of 4-(6-bromopyridin-3-yl)-2,5-difluorobenzonitrile

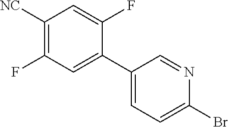 [SMILES: N#Cc1cc(F)c(-c2ccc(Br)nc2)cc1F]

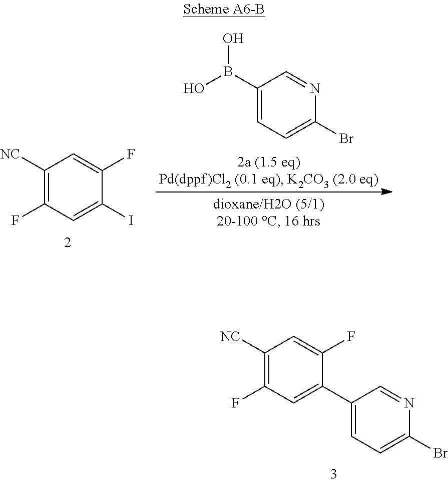 [SMILES: OB(O)c1ccc(Br)nc1 ; N#Cc1cc(F)c(I)cc1F ; N#Cc1cc(F)c(-c2ccc(Br)nc2)cc1F]

To a solution of 2,5-difluoro-4-iodobenzonitrile (2) (400 mg, 1.51 mmol, 1.00 eq) and (6-bromopyridin-3-yl)boronic acid (2a) (457 mg, 2.26 mmol, 1.50 eq) in dioxane (4.00 mL) and H₂O (0.80 mL) was added K₂CO₃ (417 mg, 3.02 mmol, 2.00 eq) at 20° C. The suspension was degassed and purged with N₂ for 3 times. To the mixture was added Pd(dppf)Cl₂ (110 mg, 151 umol, 0.10 eq) at 20° C. under N₂. The suspension was degassed and purged with N₂ for 3 times. The mixture was stirred at 100° C. for 16 hrs under N₂. LCMS showed that (2) was consumed completely and one main peak with desired mass was detected. The reaction mixture was diluted with H₂O 5.00 mL and extracted with EtOAc 30.0 mL (15.0 mL*3). The combined organic layers were dried over Na₂SO₄, filtered and concentrated under reduced pressure to give a residue. The residue was purified by prep-TLC (SiO₂, Petroleum ether/Ethyl acetate=2/1). 4-(6-bromopyridin-3-yl)-2,5-difluorobenzonitrile (3) (80.0 mg, 244.00 umol) was obtained.

### Synthesis of 4-(6-bromopyridin-3-yl)-2,5-difluorobenzimidamide

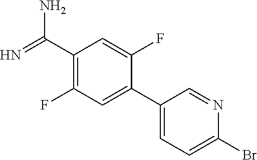 [SMILES: N=C(N)c1cc(F)c(-c2ccc(Br)nc2)cc1F]

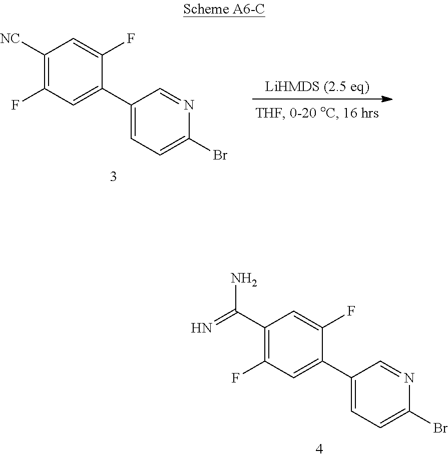 [SMILES: N#Cc1cc(F)c(-c2ccc(Br)nc2)cc1F ; N=C(N)c1cc(F)c(-c2ccc(Br)nc2)cc1F]

To a solution of 4-(6-bromopyridin-3-yl)-2,5-difluorobenzonitrile (3) (360 mg, 1.22 mmol, 1.00 eq) in THE (3.60 mL) was added LiHMDS (1 M, 3.05 mL, 2.50 eq) at 0° C. The mixture was stirred at 20° C. for 16 hrs. LCMS indicated that (3) was consumed completely. The reaction mixture was quenched by addition of HCl/Dioxane (4 M, 1.50 mL) at 0° C. and then concentrated under reduced pressure to give a residue. The residue was suspended in MeOH (5.00 ml) and the mixture was stirred at 25° C. for 1 hr. Then the mixture was filtered and the filtrate was concentrated under reduced pressure to give a residue. 4-(6-bromopyridin-3-yl)-2,5-difluorobenzimidamide (4) (390 mg, crude) was obtained.

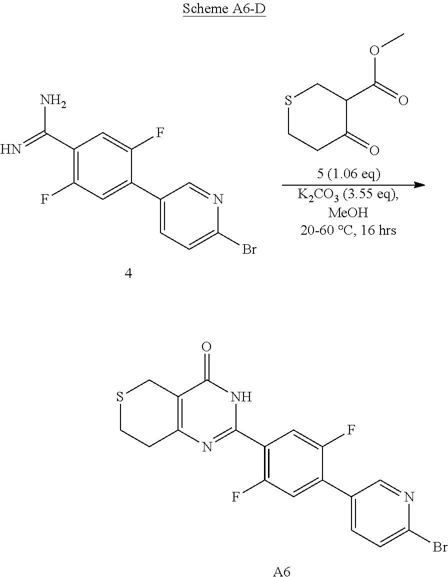 [SMILES: N=C(N)c1cc(F)c(-c2ccc(Br)nc2)cc1F ; COC(=O)C1CSCCC1=O ; O=c1[nH]c(-c2cc(F)c(-c3ccc(Br)nc3)cc2F)nc2c1CSCC2 ; "A6"]

To a solution of 4-(6-bromopyridin-3-yl)-2,5-difluorobenzimidamide (4) (160 mg, 513 umol, 1.00 eq) in MeOH (1.60 mL) was added methyl 4-oxotetrahydro-2H-thiopyran-3-carboxylate (5) (94.7 mg, 544 umol, 1.06 eq) and K₂CO₃ (252 mg, 1.82 mmol, 3.55 eq) at 25° C. The mixture was stirred at 25° C. for 12 hrs. LCMS indicated that (4) was consumed completely. The mixture was filtered, and the filtrate was concentrated under reduced pressure to give a residue. The residue was purified by prep-HPLC (FA condition; column: Phenomenex Luna C18 200*40 mm*10 um; mobile phase: [water (FA)-ACN]; B %: 40%-70%, 8 min). (A6) (28.0 mg, 64.2 umol) was obtained. Confirmed by HNMR and FNMR. ¹H NMR: (400 MHz, DMSO-d6). δ=8.68 (s, 1H), 8.39 (s, 1H), 8.07-8.03 (m, 1H), 7.83 (d, J=8.3 Hz, 1H), 7.76-7.70 (m, 2H), 3.53 (s, 2H), 2.86 (br dd, J=4.9, 15.0 Hz, 4H). LC-MS (product: Rₜ=2.500 mins).

### Pharmaceutical Composition Example 1: Preparation of Wound-Treating Composition (Compound 1)

The procedure of preparing the composition is disclosed in US20210000959. In brief, graphene oxide (1.25 g, 250 mL of 5 mg/mL GO dispersion in de-ionized water; Goographene Inc.) was added to 250 mL of ultra-pure de-ionized water and stirred for 5 minutes. Sodium hydroxide pellets (3 g, 0.075 moles; Sigma-Aldrich) were added in small solid portions to the mixture over 30 minutes. Once addition was complete, it was stirred for 1 hour at room temperature. Next the solution was ultrasonicated for 30 minutes, and then chloroacetic acid (3.54 g, 0.0375 moles; Alfa Aesar) added in small, solid portions over 20 minutes. The reaction mixture was then stirred for 18 hours at room temperature. The reaction mixture was acidified with hydrochloric acid (7 mL, 12N). The solution was then transferred to centrifuge tubes and centrifuged for 15 minutes at 5,000 rpm. The water layer was decanted and more ultra-pure deionized water (~30 mL) added to the tubes before re-centrifugation. This process was repeated 3 times. Methanol (~30 mL) was added to the precipitated, modified graphene oxide remaining in the centrifuged tubes and centrifuged at 5,000 rpm for 15 minutes. This process was repeated 3 times. Once the methanol was decanted, the tubes were put under vacuum for 48 hours at room temperature for drying. The product was then derivatized by addition of 1-(3-dimethylaminopropyl)-3-ethyl carbodiimide hydrochloride (52 mg, 0.00027 moles, Alfa Aesar) while maintaining pH with 0.1 N hydrochloric acid, and then added dropwise to a separate mixture containing adipic dihydrazide (17 mg, 0.00027 moles; Alfa Aesar) in 5 mL of ultra-pure deionized water at room temperature. Once the addition was complete, it was stirred at room temperature for 18 hours. The solution was then subjected to dialysis (MWCO=3500) for 24 hours and lyophilized, resulting in the GO-HA referenced further herein.

GO-HA (11 mg) was added to ultra-pure water (11 mL), to create an effective concentration of 1 mg/mL. The solution was ultrasonicated for 10 minutes. Compound 1 (11 mg), a compound of Formula (I), was added to PEG-400 (0.5 mL) and subjected to ultrasonication for 30 minutes. Compound 1 (a compound of Formula (I)) solution was added dropwise to the GO-HA and vigorously stirred for 5 minutes. The combined solution was then subjected to ultrasonication for 1 hour and then stirred at room temperature for 18 hours. Hydroxypropyl cellulose (0.2 g; supplier: Sigma-Aldrich) was added in small portions at room temperature with vigorous stirring. Once addition was complete, the solution was stirred at room temperature for 24 hours to form a viscous solution of the GO-HA+Compound 1. The GO-HA+Compound 1 was used with a final concentration of 1.0 mg/mL Compound 1 in 1.0 mg/mL GO-HA, and 0.182 g/mL hydroxypropyl cellulose (GO-HA+Compound 1).

### Pharmaceutical Composition Example 2: Preparation of Wound-Treating Composition (Compound 7)

The procedure of preparing the composition is disclosed in US20210000959. In brief, graphene oxide (1.25 g, 250 mL of 5 mg/mL GO dispersion in de-ionized water; Goographene Inc.) was added to 250 mL of ultra-pure de-ionized water and stirred for 5 minutes. Sodium hydroxide pellets (3 g, 0.075 moles; Sigma-Aldrich) were added in small solid portions to the mixture over 30 minutes. Once addition was complete, it was stirred for 1 hour at room temperature. Next the solution was ultrasonicated for 30 minutes, and then chloroacetic acid (3.54 g, 0.0375 moles; Alfa Aesar) added in small, solid portions over 20 minutes. The reaction mixture was then stirred for 18 hours at room temperature. The reaction mixture was acidified with hydrochloric acid (7 mL, 12 N). The solution was then transferred to centrifuge tubes and centrifuged for 15 minutes at 5,000 rpm. The water layer was decanted and more ultra-pure deionized water (~30 mL) added to the tubes before re-centrifugation. This process was repeated 3 times. Methanol (~30 mL) was added to the precipitated, modified graphene oxide remaining in the centrifuged tubes and centrifuged at 5,000 rpm for 15 minutes. This process was repeated 3 times. Once the methanol was decanted, the tubes were put under vacuum for 48 hours at room temperature for drying. The product was then derivatized by addition of 1-(3-dimethylaminopropyl)-3-ethyl carbodiimide hydrochloride (52 mg, 0.00027 moles, Alfa Aesar) while maintaining pH with 0.1 N hydrochloric acid, and then added dropwise to a separate mixture containing adipic dihydrazide (17 mg, 0.00027 moles; Alfa Aesar) in 5 mL of ultra-pure deionized water at room temperature. Once the addition was complete, it was stirred at room temperature for 18 hours. The solution was then subjected to dialysis (MWCO=3500) for 24 hours and lyophilized, resulting in the GO-HA referenced further herein.

GO-HA (11 mg) was added to ultra-pure water (11 mL), to create an effective concentration of 1 mg/mL. The solution was ultrasonicated for 10 minutes. Compound 7 (11 mg), a compound of Formula (I), was added to PEG-400 (0.5 mL) and subjected to ultrasonication for 30 minutes. Compound 7 solution was added dropwise to the GO-HA and vigorously stirred for 5 minutes. The combined solution was then subjected to ultrasonication for 1 hour and then stirred at room temperature for 18 hours. Hydroxypropyl cellulose (0.2 g; supplier: Sigma-Aldrich) was added in small portions at room temperature with vigorous stirring. Once addition was complete, the solution was stirred at room temperature for 24 hours to form a viscous solution of the GO-HA+Compound 7. The GO-HA+Compound 7 was used with a final concentration of 1.0 mg/mL Compound 7 in 1.0 mg/mL GO-HA, and 0.182 g/mL hydroxypropyl cellulose (GO-HA+Compound 7).

### Pharmaceutical Composition Example 3: Preparation of Wound-Treating Composition (Compound 8)

The procedure of preparing the composition is disclosed in US20210000959. In brief, graphene oxide (1.25 g, 250 mL of 5 mg/mL GO dispersion in de-ionized water; Goographene Inc.) was added to 250 mL of ultra-pure de-ionized water and stirred for 5 minutes. Sodium hydroxide pellets (3 g, 0.075 moles; Sigma-Aldrich) were added in small solid portions to the mixture over 30 minutes. Once addition was complete, it was stirred for 1 hour at room temperature. Next the solution was ultrasonicated for 30 minutes, and then chloroacetic acid (3.54 g, 0.0375 moles; Alfa Aesar) added in small, solid portions over 20 minutes. The reaction mixture was then stirred for 18 hours at room temperature. The reaction mixture was acidified with hydrochloric acid (7 mL, 12N). The solution was then transferred to centrifuge tubes and centrifuged for 15 minutes at 5,000 rpm. The water layer was decanted and more ultra-pure deionized water (~30 mL) added to the tubes before re-centrifugation. This process was repeated 3 times. Methanol (~30 mL) was added to the precipitated, modified graphene oxide remaining in the centrifuged tubes and centrifuged at 5,000 rpm for 15 minutes. This process was repeated 3 times. Once the methanol was decanted, the tubes were put under vacuum for 48 hours at room temperature for drying. The product was then derivatized by addition of 1-(3-dimethylaminopropyl)-3-ethyl carbodiimide hydrochloride (52 mg, 0.00027 moles, Alfa Aesar) while maintaining pH with 0.1 N hydrochloric acid, and then added dropwise to a separate mixture containing adipic dihydrazide (17 mg, 0.00027 moles; Alfa Aesar) in 5 mL of ultra-pure deionized water at room temperature. Once the addition was complete, it was stirred at room temperature for 18 hours. The solution was then subjected to dialysis (MWCO=3500) for 24 hours and lyophilized, resulting in the GO-HA referenced further herein.

GO-HA (11 mg) the was added to in ultra-pure water (11 mL), to create an effective concentration of 1 mg/mL. The solution was ultrasonicated for 10 minutes. Compound 8 (11 mg) was added to PEG-400 (0.5 mL) and subjected to ultrasonication for 30 minutes. Compound 8 solution was added dropwise to the GO-HA and vigorously stirred for 5 minutes. The combined solution was then subjected to ultrasonication for 1 hour and then stirred at room temperature for 18 hours. Hydroxypropyl cellulose (0.2 g; supplier: Sigma-Aldrich) was added in small portions at room temperature with vigorous stirring. Once addition was complete, the solution was stirred at room temperature for 24 hours to form a viscous solution of the GO-HA+Compound 8. The GO-HA+Compound 8 was used with a final concentration of 1.0 mg/mL Compound 8 in 1.0 mg/mL GO-HA, and 0.182 g/mL hydroxypropyl cellulose (GO-HA+Compound 8).

### Biological Example 1

### HEK CELL Wnt Assay

In order to determine the effect of the compounds of Formula (I) on Wnt inhibition, an in vitro assay using HEK293 SuperTOPFlash reporter cells, with the Wnt promoter driving a luciferase reporter gene, were utilized. 40,000 HEK293 SuperTOPFlash reporter cells were seeded, per well, in a 48 well plate for 24 hours, prior to administration of serial dilutions of the compound of Formula (I) to individual wells. Serial dilutions ranging from 10 μm to 1 nM of the compound of Formula (I) were added with or without 50 ng/mL Wnt₃a (Time Biosciences) and allowed to incubate with the reporter cells for 18 hours. Reporter cells with no treatment were used as negative control. Following the 18-hour incubation, luciferase activity was measured using Steady-Glo® Luciferase Assay System (Promega) and a Luminometer; with luciferase activity correlated to Wnt activation, and 50% inhibition of the Wnt promoter (IC₅₀) calculated. Results are shown in Table 3.

### Biological Example 2

### TNK2 Binding Assay

Tankyrase 2 assay (n=3) was performed with the following steps: step 1, coating a 96-well plate with 50 μL/well of histone solution; step 2, conducting ribosylation reaction; and step 3, detection. Results are shown in Table 3.

Step 1: Coating a 96-Well Plate with 50 μL/Well of Histone Solution.

A 5× histone mixture was diluted 1:5 with PBS to make a 1× histone in PBS mixture. 50 μL of the histone mixture was added to each well and incubated at 4° C. overnight. The plate was washed three times using 200 μL PBST buffer (1×PBS containing 0.05% Tween*20 (polysorbate 20) per well. The plate was tapped onto a clean paper towel to remove the liquid. The wells were blocked by adding 200 μL of blocking buffer 3 to each well. The plate was incubated at room temperature for at least 90 minutes. The plate was washed three times with 200 μL PBST buffer. The plate was tapped onto a clean paper towel to remove the liquid.

### Step 2: Ribosylation Reaction.

A fresh solution of 10 mM DTT in water was prepared.

A master mix was prepared and delivered to each well (25 μL/well). Master mix delivered to each well: (2.5 μL of 10×PARP assay buffer (purchased from BPS Bioscience)+2.5 μL of PARP substrate mixture 2+17.5 μL of water+2.5 μL of 10 mM fresh DTT). The concentration of DTT in the master mix was 1 mM. 25 μL of master mix was added to each well.

1×PARP buffer with DTT was prepared. 10×PARP assay buffer was diluted to 1×PARP assay buffer containing DTT by adding 1 volume of 10×PARP assay buffer+1 volume of 10 mM DTT+8 volumes of water. The concentration of DTT in the 1×PARP assay buffer was 1 mM.

Serial dilutions of each test inhibitor were made (in 1×PARP assay buffer with DTT), starting from 10 μM to 0 μM (10 μM, 1 μM, 0.1 μM, 0.05 μM, 0.025 μM, 0.01 μM, 0.001 μM, 0 μM). 5 μL of a test inhibitor was added to each well labeled as “Test Inhibitor.” For the “Positive Control,” the PARP inhibitor, XAV939, was used. For the negative control, no inhibitor is added to the assay mix.

The Tankyrase 2 (TNKS2) enzyme on ice was thawed. The tube containing the enzyme was spun briefly to recover the full content of the tube. The amount of TNKS2 enzyme required for the assay was calculated and the enzyme was diluted to 0.5 ng/μL with 1×PARP assay buffer with DTT. 16) The final concentration of TNKS2 enzyme in the assay was 2 nM. The reaction was initiated by adding 10 μL of diluted TNKS2 enzyme to each well designated “Positive Control” and “Test Inhibitor.” To each well designated as “Blank,” 10 μL of 1×PARP assay buffer with DTT was added. The plate was incubated at room temperature for 1 hour. The plate was washed three times with 100 μL of PBST buffer and the plate was tapped onto a clean paper towel.

### Step 3: Detection.

1) Streptavidin-HRP was diluted 1:50 in Blocking buffer (purchased from BPS Bioscience) 3.50 μL of diluted Streptavidin-RP was added to each well and incubated for 30 minutes at room temperature. The plate was washed three times with 100 μL of PBST buffer and tapped onto a clean paper towel. Just before use, 1 volume of ELISA ECL Substrate A and 1 volume of ELISA ECL Substrate B were mixed on ice; and 100 μL was added to each well. Immediately, the plate was read in a luminometer or microtiter-plate reader capable of reading chemiluminescence. The “Blank” value was subtracted from all other values. The results are shown in Table 3.

TABLE 3
Assay Results

| | | | Binding Assay |
|---|---|---|---|
| Cmpd | | HEK CELL Wnt | (TNK2) IC₅₀ |
| No. | Structure | IC₅₀ (nM) | (nM) |
| XAV939 | 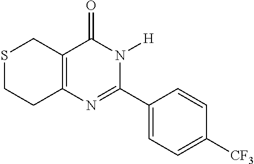 [SMILES: O=c1[nH]c(-c2ccc(C(F)(F)F)cc2)nc2c1CSCC2] | 50 nM | 6 nM |
| 1 | 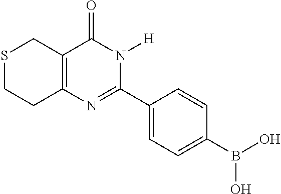 [SMILES: O=c1[nH]c(-c2ccc(B(O)O)cc2)nc2c1CSCC2] | 2,000 nM | 26 nM |
| 2 | 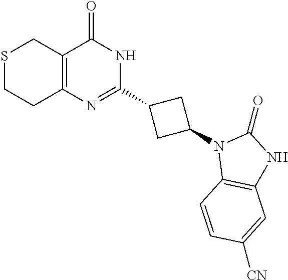 [SMILES: N#Cc1ccc2c(c1)[nH]c(=O)n2[C@H]1C[C@H](c2nc3c(c(=O)[nH]2)CSCC3)C1] | 60% inhibition @ >10,000 nM | NT |
| 3 | 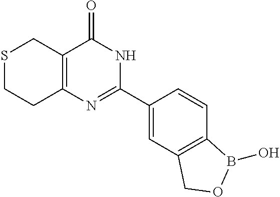 [SMILES: O=c1[nH]c(-c2ccc3c(c2)COB3O)nc2c1CSCC2] | 7,500 nM | 28 nM |
| 4 | 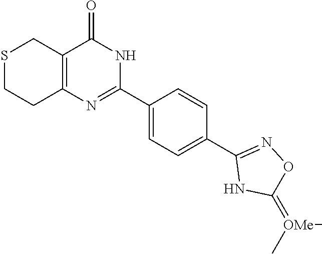 | >10,000 nM | 13 nM |
| 5 | 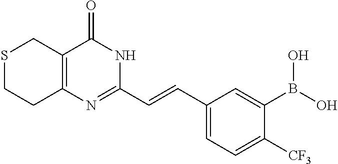 [SMILES: O=c1[nH]c(/C=C/c2ccc(C(F)(F)F)c(B(O)O)c2)nc2c1CSCC2] | 5,000 nM | NT |
| 6 | 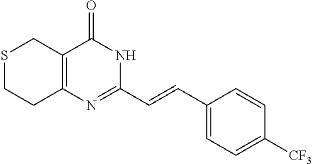 [SMILES: O=c1[nH]c(/C=C/c2ccc(C(F)(F)F)cc2)nc2c1CSCC2] | 2,000 nM | 2500 nM |
| 7 | 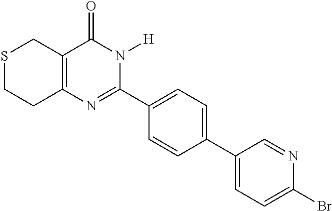 [SMILES: O=c1[nH]c(-c2ccc(-c3ccc(Br)nc3)cc2)nc2c1CSCC2] | 17 nM | 5 nM |
| 8 | 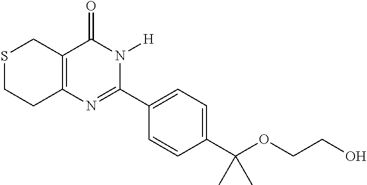 [SMILES: CC(C)(OCCO)c1ccc(-c2nc3c(c(=O)[nH]2)CSCC3)cc1] | 4.4 nM | 3.16 nM |
| | 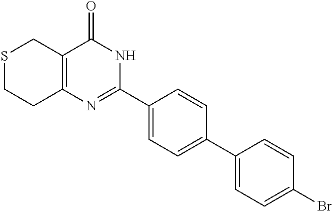 [SMILES: O=c1[nH]c(-c2ccc(-c3ccc(Br)cc3)cc2)nc2c1CSCC2] | 100 nM | NT |
| 10 | 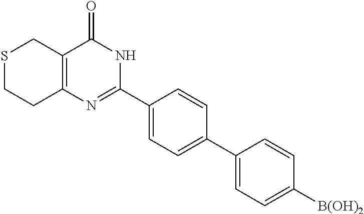 [SMILES: O=c1[nH]c(-c2ccc(-c3ccc(B(O)O)cc3)cc2)nc2c1CSCC2] | 5 nM | 3.29 nM |
| 11 | 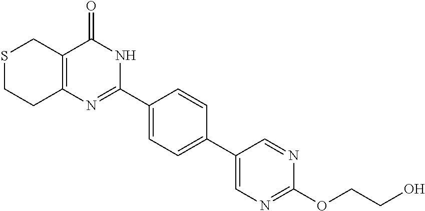 [SMILES: O=c1[nH]c(-c2ccc(-c3cnc(OCCO)nc3)cc2)nc2c1CSCC2] | 100 nM (solubility issue) | 21 nM |
| 12 | 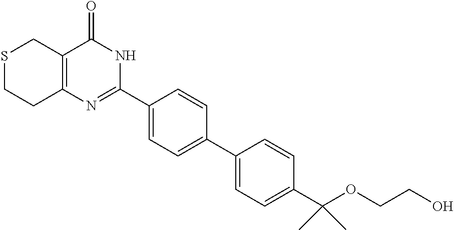 [SMILES: CC(C)(OCCO)c1ccc(-c2ccc(-c3nc4c(c(=O)[nH]3)CSCC4)cc2)cc1] | 3 nM | 6.5 nM |
| 13 | 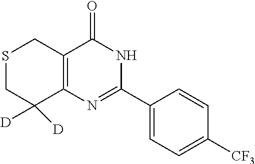 [SMILES: [2H]C1([2H])CSCc2c1nc(-c1ccc(C(F)(F)F)cc1)[nH]c2=O] | 4 nM | 3 nM |
| 14 | 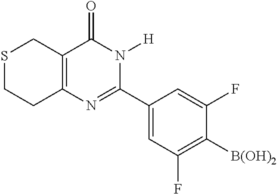 [SMILES: O=c1[nH]c(-c2cc(F)c(B(O)O)c(F)c2)nc2c1CSCC2] | 8,000 nM | 11 nM |
| 15 | 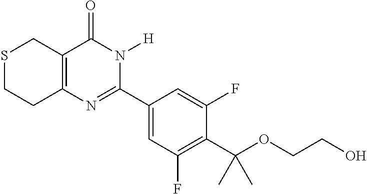 [SMILES: CC(C)(OCCO)c1c(F)cc(-c2nc3c(c(=O)[nH]2)CSCC3)cc1F] | NT | 90 nM |
| 16 | 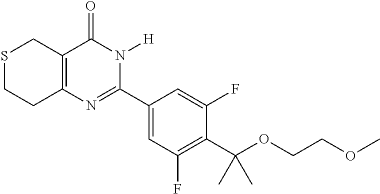 [SMILES: COCCOC(C)(C)c1c(F)cc(-c2nc3c(c(=O)[nH]2)CSCC3)cc1F] | 400 nM | 50 nM |
| 17 | 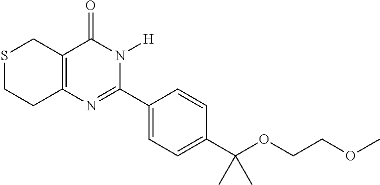 [SMILES: COCCOC(C)(C)c1ccc(-c2nc3c(c(=O)[nH]2)CSCC3)cc1] | 60 nM | 3 nM |
| 18 | 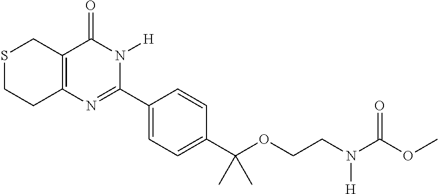 [SMILES: COC(=O)NCCOC(C)(C)c1ccc(-c2nc3c(c(=O)[nH]2)CSCC3)cc1] | 6 nM | 4 nM |
| 19 | 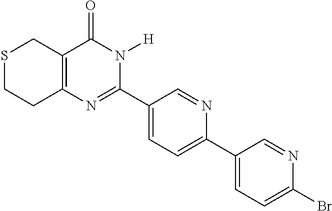 [SMILES: O=c1[nH]c(-c2ccc(-c3ccc(Br)nc3)nc2)nc2c1CSCC2] | 2,500 nM | 40 nM |
| 20 | 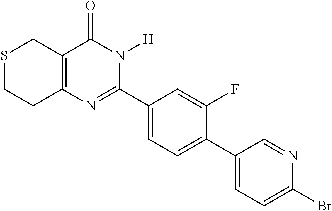 [SMILES: O=c1[nH]c(-c2ccc(-c3ccc(Br)nc3)c(F)c2)nc2c1CSCC2] | 400 nM | 4 nM |
| 21 | 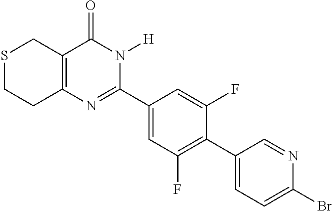 [SMILES: O=c1[nH]c(-c2cc(F)c(-c3ccc(Br)nc3)c(F)c2)nc2c1CSCC2] | 2,000 nM | 12 nM |
| 22 | 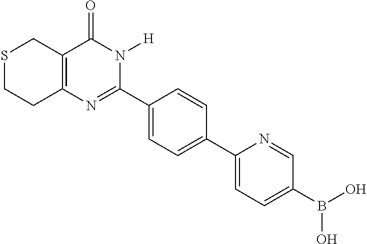 [SMILES: O=c1[nH]c(-c2ccc(-c3ccc(B(O)O)cn3)cc2)nc2c1CSCC2] | 250 nM | 4 nM |
| 23 | 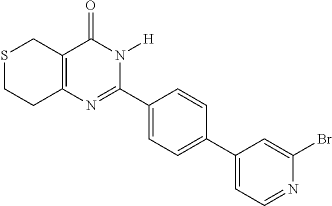 [SMILES: O=c1[nH]c(-c2ccc(-c3ccnc(Br)c3)cc2)nc2c1CSCC2] | 80 nM | 14 nM |
| 24 | 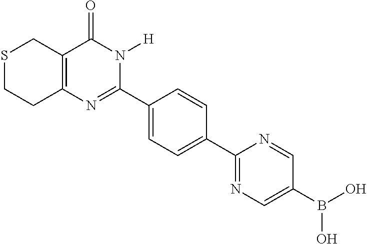 [SMILES: O=c1[nH]c(-c2ccc(-c3ncc(B(O)O)cn3)cc2)nc2c1CSCC2] | 500 nM | 3 nM |
| 25 | 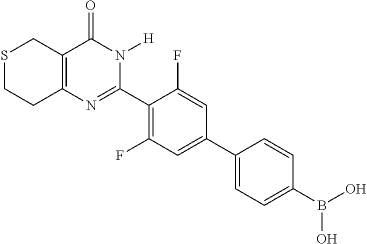 [SMILES: O=c1[nH]c(-c2c(F)cc(-c3ccc(B(O)O)cc3)cc2F)nc2c1CSCC2] | 2,500 nM | 63 nM |
| 26 | 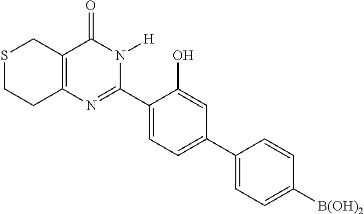 [SMILES: O=c1[nH]c(-c2ccc(-c3ccc(B(O)O)cc3)cc2O)nc2c1CSCC2] | 652 nM | 24 nM |
| 27 | 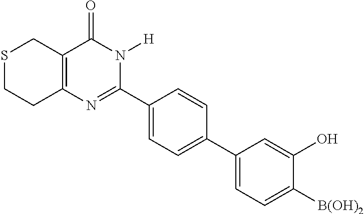 [SMILES: O=c1[nH]c(-c2ccc(-c3ccc(B(O)O)c(O)c3)cc2)nc2c1CSCC2] | 36 nM | 2 nM |
| 28 | 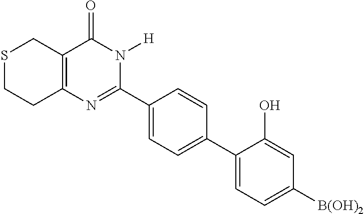 [SMILES: O=c1[nH]c(-c2ccc(-c3ccc(B(O)O)cc3O)cc2)nc2c1CSCC2] | 2 nM | 2 nM |
| 29 | 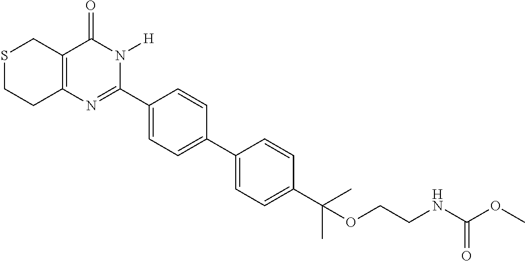 [SMILES: COC(=O)NCCOC(C)(C)c1ccc(-c2ccc(-c3nc4c(c(=O)[nH]3)CSCC4)cc2)cc1] | 100 nM | 1 nM |
| 30 | 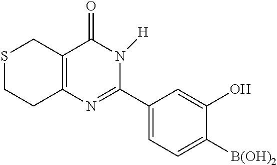 [SMILES: O=c1[nH]c(-c2ccc(B(O)O)c(O)c2)nc2c1CSCC2] | NT | 20 nM |
| 31 | 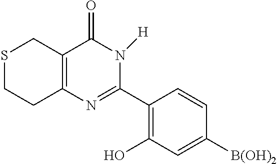 [SMILES: O=c1[nH]c(-c2ccc(B(O)O)cc2O)nc2c1CSCC2] | NT | 3534 nM |
| 32 | 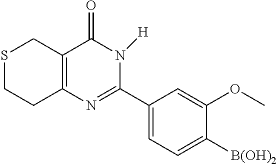 [SMILES: COc1cc(-c2nc3c(c(=O)[nH]2)CSCC3)ccc1B(O)O] | NT | 111 nM |
| 33 | 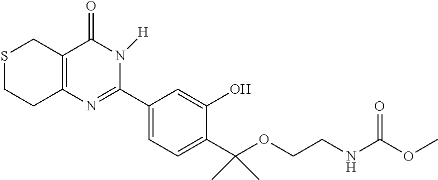 [SMILES: COC(=O)NCCOC(C)(C)c1ccc(-c2nc3c(c(=O)[nH]2)CSCC3)cc1O] | NT | 12 nM |
| 34 | 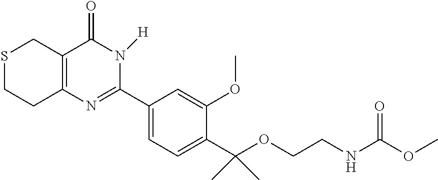 [SMILES: COC(=O)NCCOC(C)(C)c1ccc(-c2nc3c(c(=O)[nH]2)CSCC3)cc1OC] | NT | 164 nM |
| 35 | 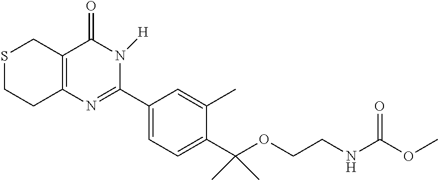 [SMILES: COC(=O)NCCOC(C)(C)c1ccc(-c2nc3c(c(=O)[nH]2)CSCC3)cc1C] | NT | 13 nM |
| 36 | 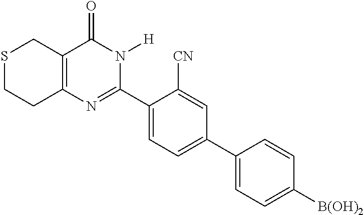 [SMILES: N#Cc1cc(-c2ccc(B(O)O)cc2)ccc1-c1nc2c(c(=O)[nH]1)CSCC2] | NT | 29 nM |
| 37 | 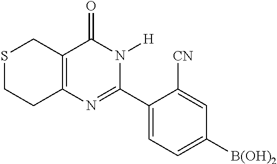 [SMILES: N#Cc1cc(B(O)O)ccc1-c1nc2c(c(=O)[nH]1)CSCC2] | NT | 16 nM |
| 38 | 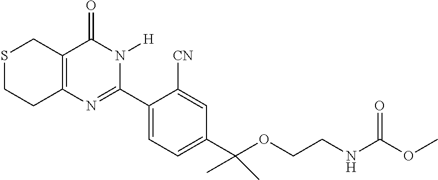 [SMILES: COC(=O)NCCOC(C)(C)c1ccc(-c2nc3c(c(=O)[nH]2)CSCC3)c(C#N)c1] | NT | 72 nM |
| 39 | 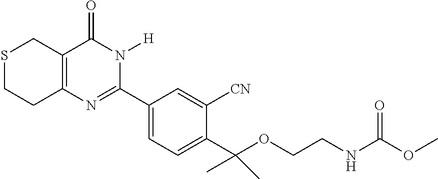 [SMILES: COC(=O)NCCOC(C)(C)c1ccc(-c2nc3c(c(=O)[nH]2)CSCC3)cc1C#N] | NT | 650 nM |
| 40 | 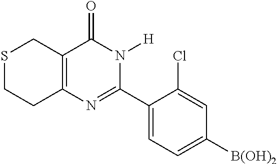 [SMILES: O=c1[nH]c(-c2ccc(B(O)O)cc2Cl)nc2c1CSCC2] | NT | 443 nM |
| 41 | 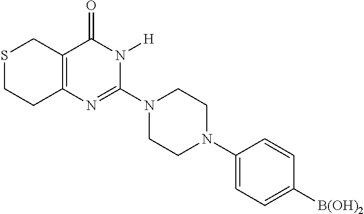 [SMILES: O=c1[nH]c(N2CCN(c3ccc(B(O)O)cc3)CC2)nc2c1CSCC2] | NT | 8.7 nM |
| 42 | 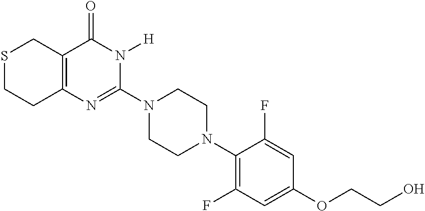 [SMILES: O=c1[nH]c(N2CCN(c3c(F)cc(OCCO)cc3F)CC2)nc2c1CSCC2] | NT | 9.7 nM |
| 43 | 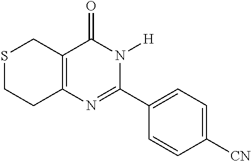 [SMILES: N#Cc1ccc(-c2nc3c(c(=O)[nH]2)CSCC3)cc1] | 1,000 nM | NT |
| 44 | 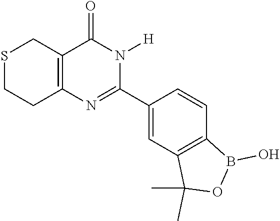 [SMILES: CC1(C)OB(O)c2ccc(-c3nc4c(c(=O)[nH]3)CSCC4)cc21] | NT | 630 nM |

### Biological Example 3: Demonstration of Wnt Inhibition by a Composition of Formula (I)

In order to determine the effect of the compounds of Formula (I) on Wnt inhibition, an invitro assay using HEK293 SuperTOPFlash reporter cells, with the Wnt promoter driving a luciferase reporter gene, were utilized. 40,000 HEK293 SuperTOPFlash reporter cells were seeded, per well, in a 48 well plate for 24 hours, prior to administration of serial dilutions of the compounds of Formula (I) to individual wells. Serial dilutions ranging from 10 m to 1 nM of compounds of of Formula (I) were added with or without 50 ng/mL Wnt₃α (Time Biosciences) and allowed to incubate with the reporter cells for 18 hours. Reporter cells with no treatment were used as negative control. Following the 18 hour incubation, luciferase activity was measured using Steady-Glo® Luciferase Assay System (Promega) and a Luminometer; with luciferase activity correlated to Wnt activation, and 5000 inhibition of the Wnt promoter (IC₅₀) calculated.

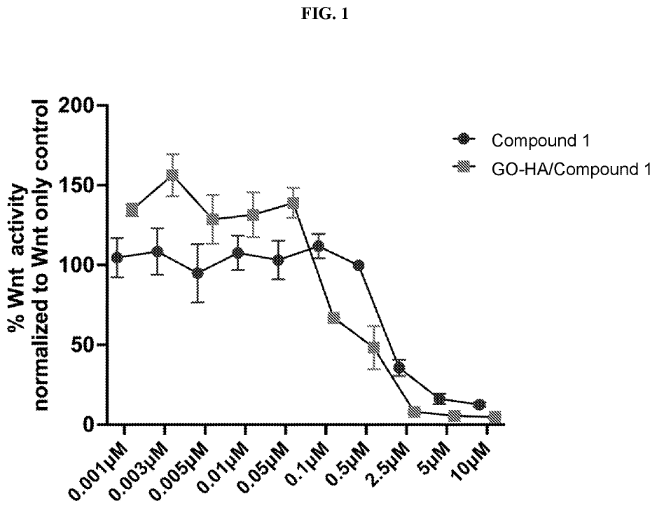

FIG. 1 presents the results of IC₅₀ assessment of Compound 1 (a compound of Formula (I)) in DMSO and of GO-HA+Compound 1 in an aqueous formulation as further described in Pharmaceutical Composition Example 1. IC₅₀ of Wnt activity was calculated as 2 μM for Compound 1 and 500 nM for GO-HA+Compound 1, demonstrating the suspension of Compound 1 in aqueous GO-HA and Compound 1 with DMSO as the solvent both demonstrate inhibition of Wnt activity.

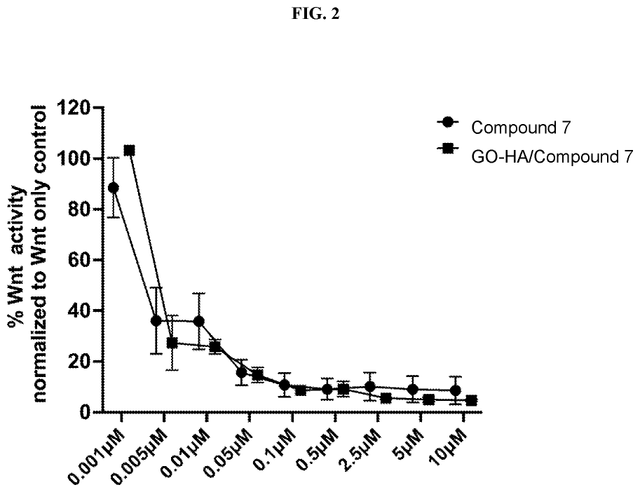

FIG. 2 presents the results of IC₅₀ assessment of a compound of Formula (I), Compound 7, in DMSO, and of GO-HA+Compound 7 in an aqueous formulation as further described in Pharmaceutical Composition Example 2. IC₅₀ of Wnt activity was calculated as 5-10 nM for Compound 7 and 4 nM for GO-HA+Compound 7, demonstrating the suspension of Compound 7 in aqueous GO-HA and Compound 7 with DMSO as the solvent both demonstrate inhibition of Wnt activity.

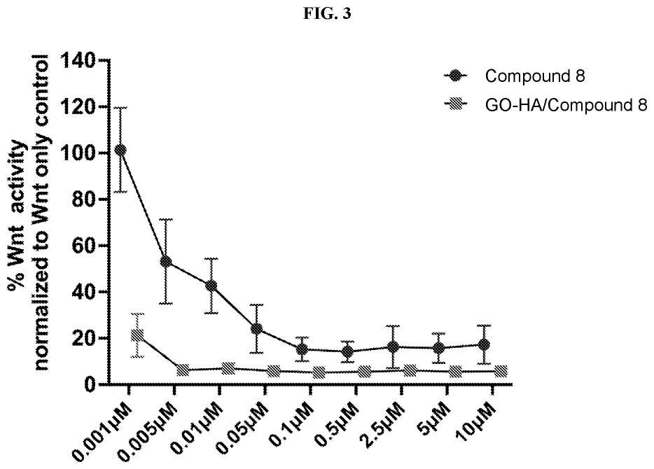

FIG. 3 presents the results of IC₅₀ assessment of Compound 8 (a compound of Formula (I)) in DMSO, and of GO-HA+Compound 8 in an aqueous formulation as further described in Pharmaceutical Composition Example 3. IC₅₀ of Wnt activity was calculated as 5-10 nM for Compound 8 and less than 1 nM for GO-HA+Compound 8, demonstrating the suspension of Compound 8 (a compound of Formula (I)) in aqueous GO-HA and Compound 8 (a compound of Formula (I)) with DMSO as the solvent both demonstrate inhibition of Wnt activity.

### Biological Example 4: Bacteriostasis

In order to assess the log reduction capabilities of compounds of Formula (1), testing was performed on organisms that are found in acute and chronic wounds. Triplicate tubes were prepared for each bacteria using 50 μL of inoculum (about 1×10⁶ CFU/mL) which was added to 450 μL of compound solution (compound of Formula (I)) (or 100 μL of inoculum in 900 μL of compound solution), resulting in a final inoculum of about 1×10⁵ CFU/mL, as shown in Table 4.

Tubes were mixed with a vortex and then incubated in a shaking incubator at 175 RPM and 35° C. to 37° C.

Positive control in Tables 5a and 5b was 1 mL of 0.89% saline with 1 mL of the 1×10⁶ CFU/mL of the organism.

Negative control in Table 6 was a neat sample in Meuller Hinton Broth with no organism spike (one for the sample and one for the vehicle).

For Compounds 1, 7, 8, 10, and 18 in Tables 6, 7-A, 7-B, 8, 9, 10-A, 10-B, and 11: The compound solutions were prepared with compound (30 mg), polysorbate 20 (1.6 mL), butylene glycol (1.6 mL), phosphatidyl choline (0.66 g), ethanol (1.6 mL), hydroxypropyl cellulose (0.26 g), and ultra pure water (24.9 mL).

XAV939 Control in Table X1 was XAV939 (30 mg), polysorbate 20 (1.6 mL), butylene glycol (1.6 mL), phosphatidyl choline (0.66 g), ethanol (1.6 mL), ultra pure water (24.9 mL), and hydroxypropyl cellulose (0.26 g).

GO-HA Control in Table X2 was GO-HA (30 mg), polysorbate 20 (1.6 mL), butylene glycol (1.6 mL), phosphatidyl choline (0.66 g), ethanol (1.6 mL), ultra pure water (24.9 mL), and hydroxypropyl cellulose (0.26 g).

For Compound 18+GO-HA in Tables 6 and X4: compound 18 (45 mg), GO-HA (45 mg), polysorbate 20 (2 mL), butylene glycol (2 mL), phosphotidyl choline (0.5 g), ethanol (2 mL), hydroxypropyl cellulose (0.40 g), and ultra pure water (40 mL).

For XAV939+GO-HA in Tables 6 and X3-A, and X3-B: XAV939 (45 mg), GO-HA (45 mg), butylene glycol (2 mL), polysorbate 20 (2 mL), phosphotidyl choline (0.5 g), ethanol (2 mL), hydroxypropyl cellulose (0.40 g), and ultra pure water (40 mL).

Testing was performed at two time points: 18 to 24 hours, and 724 hours. Test results are shown in Tables 4-11 and X1-X4.

TABLE 4
Inoculum Results

| | Positive | Inoculum | Spiked | Spike |
|---|---|---|---|---|
| | Control 24 | Count | Inoculum | Inoculum |
| Organism | hours | (CFU/mL) | (CFU/mL) | Log Value |
| A. baumannii (derived from | Growth | 2.2 × 10⁶ | 2.2 × 10⁵ | 5.3 |
| ATCC ® 19606 ™) | | | | |
| C. acnes (derived from ATCC ® | Growth | 7.8 × 10⁵ | 7.8 × 10⁴ | 4.9 |
| 11827 ™) | | | | |
| E. coli (derived from ATCC ® | Growth | 1.8 × 10⁶ | 1.8 × 10⁵ | 5.3 |
| 8739 ™) (2 tests) | Growth | 2.8 × 10⁵ | 2.8 × 10⁴ | 4.5 |
| E. coli (derived from ATCC ® | Growth | 2.6 × 10⁶ | 2.6 × 10⁵ | 5.4 |
| 25922 ™) (2 tests) | Growth | 3.8 × 10⁵ | 3.8 × 10⁴ | 4.6 |
| E. coli 0157:H7 (derived from | Growth | 1.4 × 10⁵ | 1.4 × 10⁴ | 4.2 |
| ATCC ® 12900 ™) | | | | |
| E. faecalis (derived from | Growth | 6.2 × 10⁵ | 6.2 × 10⁴ | 4.8 |
| ATCC ® 19433 ™) (2 tests) | Growth | 1.2 × 10⁶ | 1.2 × 10⁵ | 5.1 |
| K. pneumoniae (derived from | Growth | 5.8 × 10⁵ | 5.8 × 10⁴ | 4.8 |
| ATCC ® 10031 ™) | | | | |
| P. vulgaris (derived from | Growth | 8.0 × 10⁶ | 8.0 × 10⁵ | 5.9 |
| ATCC ® 8427 ™) | | | | |
| P. aeruginosa (derived from | Growth | 6.0 × 10⁵ | 6.0 × 10⁴ | 4.8 |
| ATCC ® 9027 ™) (2 tests) | Growth | 1.8 × 10⁶ | 1.8 × 10⁵ | 5.3 |
| P. aeruginosa (derived from | Growth | 2.6 × 10⁶ | 2.6 × 10⁵ | 5.4 |
| ATCC ® 15442 ™) (2 tests) | Growth | 1.4 × 10⁶ | 1.4 × 10⁵ | 5.2 |
| S. aureus (MRSA) (derived from | Growth | 4.4 × 10⁶ | 4.4 × 10⁵ | 4.6 |
| ATCC ® 33593 ™) (2 tests) | Growth | 6.4 × 10⁵ | 6.4 × 10⁴ | 4.8 |
| S. aureus (derived from ATCC ® | Growth | 2.2 × 10⁶ | 2.2 × 10⁵ | 5.3 |
| 6538 ™) (2 tests) | Growth | 1.4 × 10⁶ | 1.4 × 10⁵ | 5.2 |
| S. epidermidis (derived from | Growth | 6.8 × 10⁵ | 6.8 × 10⁴ | 5.3 |
| ATCC ® 14990 ™) (2 tests) | Growth | 8.0 × 10⁵ | 8.0 × 10⁴ | 4.9 |
| S. maltophilia (derived from | Growth | 1.2 × 10⁶ | 1.2 × 10⁵ | 5.1 |
| ATCC ® 13637 ™) | | | | |
| S. pyogenes (derived from | Growth | 3.0 × 10⁶ | 3.0 × 10⁵ | 5.5 |
| ATCC ® 19615 ™) | | | | |

TABLE 5a
Positive control comparison

| | Positive Control | | | |
|---|---|---|---|---|
| | C. acnes | E. coli | E. coli | E. faecalis |
| | (derived | (derived | (derived | (derived |
| Time | from ATCC ® | from ATCC ® | from ATCC ® | from ATCC ® |
| Point | 11827 ™) | 8739 ™) | 25922 ™) | 19433 ™) |
| 24 | Growth | Growth | Growth | Growth |
| hours | | | | |
| 72 | Growth | Growth | Growth | Growth |
| hours | | | | |

TABLE 5b
Positive control comparison

| | Positive Control | | | |
|---|---|---|---|---|
| | S. aureus | | | |
| | (MRSA) | P. aeruginosa | S. aureus | S. epidermidis |
| | (derived | (derived | (derived | (derived |
| Time | from ATCC ® | from ATCC ® | from ATCC ® | from ATCC ® |
| Point | 33593 ™) | 15442 ™) | 6538 ™) | 14990 ™) |
| 24 | Growth | Growth | Growth | Growth |
| hours | | | | |
| 72 | Growth | Growth | Growth | Growth |
| hours | | | | |

TABLE 6
Negative control comparison

| | Negative Control | | | | |
|---|---|---|---|---|---|
| Time | | | XAV939 + | | Compound 18 + |
| Point | Compound 1 | Compound 10 | GO-HA | Compound 18 | GO-HA |
| 24 hours | No Growth | No Growth | No Growth | No Growth | No Growth |
| 72 hours | No Growth | No Growth | No Growth | No Growth | No Growth |

TABLE 7-A
Compound 1 results, test 1
Compound 1

| | | | | | S. aureus | P. | | S. |
|---|---|---|---|---|---|---|---|---|
| | | E. coli | E. coli | E. faecalis | (MRSA) | aeruginosa | S. aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 | Average | 1.3 × 10⁴ | <200 | 1.1 × 10³ | 9.3 × 10³ | <200 | 8.7 × 10⁴ | 3.1 × 10⁵ |
| h | result | | | | | | | |
| | Log | 4.1 | <2.3 | 3.0 | 4.0 | <2.3 | 4.9 | 5.5 |
| | Value | | | | | | | |
| | Log | 1.2 | >3.1 | 1.8 | 0.6 | >3.1 | 0.4 | No |
| | Reduction | | | | | | | reduction |
| 72 | Average | <200 | <200 | 1.1 × 10⁴ | 4.0 × 10⁴ | <200 | <800 | <200 |
| h | result | | | | | | | |
| | Log | <2.3 | <2.3 | 4.1 | 4.6 | <2.3 | <2.9 | <2.3 |
| | Value | | | | | | | |
| | Log | >3.0 | >3.1 | 0.7 | No | >3.1 | >2.4 | >2.5 |
| | Reduction | | | | reduction | | | |

TABLE 7-B
Compound 1 results, test 2
Compound 1

| | | A. | E. coli | K. | P. | P. | S. | S. |
|---|---|---|---|---|---|---|---|---|
| | | baumannii | 0157:H7 | pneumoniae | aeruginosa | vulgaris | maltophilia | pyogenes |
| | | (ATCC | (ATCC ® | (ATCC ® | (ATCC ® | (ATCC ® | (ATCC ® | (ATCC ® |
| | | 19606 ® ™) | 12900 ™) | 10031 ™) | 9027 ™) | 8427 ™) | 13637 ™) | 19615 ™) |
| 24 h | Average | <200 | 5.5 × 10³ | 1.1 × 10³ | <200 | <200 | <200 | <200 |
| | result | | | | | | | |
| | Log Value | <2.3 | 3.7 | 3.0 | <2.3 | <2.3 | <2.3 | <2.3 |
| | Log | >3.0 | 0.5 | 1.8 | >3.0 | >3.6 | >2.8 | >3.2 |
| | Reduction | | | | | | | |
| 72 h | Average | <200 | <200 | <200 | <200 | <200 | <200 | <200 |
| | result | | | | | | | |
| | Log Value | <2.3 | <2.3 | <2.3 | <2.3 | <2.3 | <2.3 | <2.3 |
| | Log | >3.0 | >1.9 | >2.5 | >3.0 | >3.6 | >2.8 | >3.2 |
| | Reduction | | | | | | | |

TABLE 8
Compound 7 results
Compound 7

| | | | | E. | S. aureus | P. | S. | S. |
|---|---|---|---|---|---|---|---|---|
| | | E. coli | E. coli | faecalis | (MRSA) | aeruginosa | aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | 7.9 × 10⁴ | 3.2 × 10⁵ | 9.3 × 10⁵ | 2.7 × 10³ | <200 | 7.3 × 10² | 2.8 × 10⁴ |
| | result | | | | | | | |
| | Log Value | 4.9 | 5.5 | 6.0 | 3.4 | <2.3 | 2.9 | 4.4 |
| | Log | 0.4 | No | No | 1.2 | >3.1 | 2.4 | 0.4 |
| | Reduction | | reduction | reduction | | | | |
| 72 h | Average | >3.6 × 10⁶ | 2.7 × 10⁴ | 6.9 × 10⁵ | 1.4 × 10⁵ | <200 | <200 | <200 |
| | result | | | | | | | |
| | Log Value | >6.6 | 4.4 | 5.8 | 5.1 | <2.3 | <2.3 | <2.3 |
| | Log | No | 1.0 | No | No | >3.1 | >3.0 | >2.5 |
| | Reduction | reduction | | reduction | reduction | | | |

TABLE 9
Compound 8 results
Compound 8

| | | | | E. | S. aureus | P. | S. | S. |
|---|---|---|---|---|---|---|---|---|
| | | E. coli | E. coli | faecalis | (MRSA) | aeruginosa | aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | 3.1 × 10³ | 200 | 3.4 × 10⁵ | 3.3 × 10⁶ | 7.3 × 10² | 5.4 × 10⁴ | 6.0 × 10² |
| | result | | | | | | | |
| | Log Value | 3.5 | 2.3 | 5.5 | 6.5 | 2.9 | 4.7 | 2.8 |
| | Log | 1.8 | 3.1 | No | No | 2.5 | 0.6 | 2.0 |
| | Reduction | | | reduction | reduction | | | |
| 72 h | Average | 6.8 × 10⁴ | <200 | 6.5 × 10⁴ | 1.0 × 10⁶ | <200 | <7.4 × 10³ | <200 |
| | result | | | | | | | |
| | Log Value | 4.8 | <2.3 | 4.8 | 6.0 | <2.3 | <3.9 | <2.3 |
| | Log | 0.5 | >3.1 | No | No | >3.1 | >1.4 | >2.5 |
| | Reduction | | | reduction | reduction | | | |

TABLE 10-A
Compound 10 results, test 1
Compound 10

| | | | | E. | S. aureus | P. | S. | S. |
|---|---|---|---|---|---|---|---|---|
| | | E. coli | E. coli | faecalis | (MRSA) | aeruginosa | aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | <200 | <200 | 2.9 × 10⁴ | 3.1 × 10⁴ | <200 | 1.5 × 10⁵ | 1.1 × 10⁴ |
| | result | | | | | | | |
| | Log Value | <2.1 | <2.3 | 4.5 | 4.5 | <2.3 | 5.2 | 4.0 |
| | Log | >3.2 | >3.1 | 0.3 | 0.1 | >3.1 | 0.1 | 0.8 |
| | Reduction | | | | | | | |
| 72 h | Average | 6.0 × 10² | <200 | <200 | 2.7 × 10³ | <200 | 6.7 × 10³ | 3.3 × 10² |
| | result | | | | | | | |
| | Log Value | 2.8 | <2.3 | <2.3 | 3.4 | <2.3 | 3.8 | 2.5 |
| | Log | 2.5 | >3.1 | >2.5 | 1.2 | >3.1 | 1.5 | 2.3 |
| | Reduction | | | | | | | |

TABLE 10-B
Compound 10 results, test 2
Compound 10

| | | A. | E. coli | K. | P. | P. | S. | S. |
|---|---|---|---|---|---|---|---|---|
| | | baumannii | 0157:H7 | pneumoniae | aeruginosa | vulgaris | maltophilia | pyogenes |
| | | (ATCC ® | (ATCC ® | (ATCC ® | (ATCC ® | (ATCC ® | (ATCC ® | (ATCC ® |
| | | 19606 ™) | 12900 ™) | 10031 ™) | 9027 ™) | 8427 ™) | 13637 ™) | 19615 ™) |
| 24 h | Average | <200 | <200 | <200 | <200 | <200 | <200 | <200 |
| | result | | | | | | | |
| | Log Value | <2.3 | <2.3 | <2.3 | <2.3 | <2.3 | <2.3 | <2.3 |
| | Log | >3.0 | >1.9 | >2.5 | >3.0 | >3.6 | >2.8 | >3.2 |
| | Reduction | | | | | | | |
| 72 h | Average | <200 | <200 | <200 | <200 | <200 | <200 | <200 |
| | result | | | | | | | |
| | Log Value | <2.3 | <2.3 | <2.3 | <2.3 | <2.3 | <2.3 | <2.3 |
| | Log | >3.0 | >1.9 | >2.5 | >3.0 | >3.6 | >2.8 | >3.2 |
| | Reduction | | | | | | | |

TABLE 11
Compound 18 results
Compound 18

| | | A. | | | | E. coli | E. | K |
|---|---|---|---|---|---|---|---|---|
| | | baumannii | C. acnes | E. coli | E. coli | 0157:H7 | faecalis | pneumonia |
| | | (ATCC ® | (ATCC ® | (ATCC ® | (ATCC ® | (ATCC ® | (ATCC ® | (ATCC ® |
| | | 19606 ™) | 8739 ™) | 8739 ™) | 25922 ™) | 12900 ™) | 19433 ™) | 10031 ™) |
| 24 h | Average | <200 | 10.7 × 10⁴ | 5.3 × 10² | <200 | <200 | 2.7 × 10⁵ | <200 |
| | result | | | | | | | |
| | Log | <2.3 | 4.2 | 2.7 | <2.3 | <2.3 | 5.4 | <2.3 |
| | Value | | | | | | | |
| | Log | >3.0 | 0.7 | 1.8 | >2.3 | >1.9 | No | >2.5 |
| | Reduction | | | | | | reduction | |
| 72 h | Average | <200 | 6.5 × 10³ | <200 | <200 | <200 | 1.9 × 10⁵ | <200 |
| | result | | | | | | | |
| | Log | <2.3 | 3.8 | <2.3 | <2.3 | <2.3 | 5.3 | <2.3 |
| | Value | | | | | | | |
| | Log | >3.0 | 1.1 | >2.2 | >2.3 | >1.9 | No | >2.5 |
| | Reduction | | | | | | reduction | |

| | | P. | P. | P | | S. aureus | S. | S. | S. |
|---|---|---|---|---|---|---|---|---|---|
| | | aeruginosa | aeruginosa | vulgaris | S. aureus | (MRSA) | epidermidis | maltophilia | pyogenes |
| | | (ATCC ® | (ATCC ® | (ATCC ® | (ATCC ® | (ATCC ® | (ATCC ® | (ATCC ® | (ATCC ® |
| | | 9027) | 15442) | 8427) | 6538) | 33593) | 14990) | 13637) | 19615) |
| 24 h | Avg | <200 | <200 | <200 | 4.7 × 10⁴ | 1.5 × 10³ | 8.0 × 10² | <200 | <200 |
| | result | | | | | | | | |
| | Log | <2.3 | <2.3 | <2.3 | 4.7 | 3.2 | 2.9 | <2.3 | <2.3 |
| | Value | | | | | | | | |
| | Log | >2.5 | >2.9 | >3.6 | 0.5 | 1.6 | 2.0 | >2.8 | >3.2 |
| | Redn | | | | | | | | |
| 72 h | Avg | <200 | <200 | <200 | 9.3 × 10² | <200 | <200 | <200 | <200 |
| | result | | | | | | | | |
| | Log | <2.3 | <2.3 | <2.3 | 3.0 | <2.3 | <2.3 | <2.3 | <2.3 |
| | Value | | | | | | | | |
| | Log | >2.5 | >2.9 | >3.6 | 2.2 | >2.5 | >2.6 | >2.8 | >3.2 |
| | Redn | | | | | | | | |

TABLE X1
XAV939 results (control)
XAV939 (control)

| | | | | E. | S. aureus | P. | S. | S. |
|---|---|---|---|---|---|---|---|---|
| | | E. coli | E. coli | faecalis | (MRSA) | aeruginosa | aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | 2.7 × 10³ | 2.8 × 10⁴ | 2.6 × 10⁵ | 8.7 × 10⁴ | <200 | 2.5 × 10³ | 5.5 × 10⁴ |
| | result | | | | | | | |
| | Log Value | 3.4 | 4.4 | 5.4 | 4.9 | <2.3 | 3.4 | 4.7 |
| | Log | 1.9 | 1.0 | No | No | >3.1 | 1.9 | 0.1 |
| | Reduction | | | reduction | reduction | | | |
| 72 h | Average | <200 | <200 | 1.7 × 10⁵ | <6.8 × 10³ | <200 | 1.7 × 10⁵ | <200 |
| | result | | | | | | | |
| | Log Value | <2.3 | <2.3 | 5.1 | <3.8 | <2.3 | 5.2 | <2.3 |
| | Log | >3.0 | >3.1 | No | >0.8 | >3.1 | 0.1 | >2.5 |
| | Reduction | | | reduction | | | | |

TABLE X2
GO-HA results (control)
GO-HA (control)

| | | | | E. | S. aureus | P. | | S. |
|---|---|---|---|---|---|---|---|---|
| | | E. coli | E. coli | faecalis | (MRSA) | aeruginosa | S. aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | 3.0 × 10³ | 1.9 × 10³ | 1.0 × 10⁵ | 8.4 × 10⁵ | <200 | 3.9 × 10⁵ | 8.2 × 10³ |
| | result | | | | | | | |
| | Log Value | 3.5 | 3.3 | 5.0 | 5.9 | <2.3 | 5.6 | 3.9 |
| | Log | 1.8 | 2.1 | No | No | >3.1 | No | 0.9 |
| | Reduction | | | reduction | reduction | | reduction | |
| 72 h | Average | <7.4 × 10³ | <200 | 1.1 × 10⁵ | 6.9 × 10⁴ | <200 | 4.0 × 10⁵ | <200 |
| | result | | | | | | | |
| | Log Value | <3.9 | <2.3 | 5.0 | 4.8 | <2.3 | 5.6 | <2.3 |
| | Log | >1.4 | >3.1 | No | No | >3.1 | No | >2.5 |
| | Reduction | | | reduction | reduction | | reduction | |

TABLE X3-A
Compound XAV939 + GO-HA results, test 1
XAV939 + GO-HA

| | | | | E. | S. aureus | P. | S. | S. |
|---|---|---|---|---|---|---|---|---|
| | | E. coli | E. coli | faecalis | (MRSA) | aeruginosa | aureus | epidermidis |
| | | (derived | (derived | (derived | (derived | (derived | (derived | (derived |
| | | from | from | from | from | from | from | from |
| | | ATCC ® | ATCC ® | ATCC ® | ATCC ® | ATCC ® | ATCC ® | ATCC ® |
| | | 8739 ™) | 25922 ™) | 19433 ™) | 33593 ™) | 15442 ™) | 6538 ™) | 14990 ™) |
| 24 h | Average | <200 | 2.7 × 10² | 7.9 × 10⁴ | 2.2 × 10⁶ | <200 | 1.3 × 10⁵ | 5.0 × 10⁴ |
| | result | | | | | | | |
| | Log Value | <2.1 | 2.4 | 4.9 | 6.4 | <2.3 | 5.1 | 4.7 |
| | Log | >3.2 | 3.0 | No | No | >3.1 | 0.2 | 0.1 |
| | Reduction | | | reduction | reduction | | | |
| 72 h | Average | 9.3 × 10³ | <200 | 2.9 × 10³ | 6.5 × 10³ | <200 | 8.7 × 10⁴ | <200 |
| | result | | | | | | | |
| | Log Value | 4.0 | <2.3 | 3.5 | 3.8 | <2.3 | 4.9 | <2.3 |
| | Log | 1.3 | >3.1 | 1.3 | 0.8 | >3.1 | 0.4 | >2.5 |
| | Reduction | | | | | | | |

TABLE X3-B
Compound XAV939 + GO-HA results, test 2
XAV939 + GO-HA

| | | A. | E. coli | K. | P. | P. | S. | S. |
|---|---|---|---|---|---|---|---|---|
| | | baumannii | 0157:H7 | pneumoniae | aeruginosa | vulgaris | maltophilia | pyogenes |
| | | (ATCC ® | (ATCC ® | (ATCC ® | (ATCC ® | (ATCC ® | (ATCC ® | (ATCC ® |
| | | 19606 ™) | 12900 ™) | 10031 ™) | 9027 ™) | 8427 ™) | 13637 ™) | 19615 ™) |
| 24 h | Average | <200 | 1.1 × 10⁵ | <200 | <200 | <200 | <200 | <200 |
| | result | | | | | | | |
| | Log | <2.3 | 5.0 | <2.3 | <2.3 | <2.3 | <2.3 | <2.3 |
| | Value | | | | | | | |
| | Log | >3.0 | No | >2.5 | >3.0 | >3.6 | >2.8 | >3.2 |
| | Reduction | | reduction | | | | | |
| 72 h | Average | <200 | 2.7 × 10² | <200 | <200 | <200 | <200 | <200 |
| | result | | | | | | | |
| | Log | <2.3 | 2.4 | <2.3 | <2.3 | <2.3 | <2.3 | <2.3 |
| | Value | | | | | | | |
| | Log | >3.0 | 1.8 | >2.5 | >3.0 | >3.6 | >2.8 | >3.2 |
| | Reduction | | | | | | | |

TABLE X4
Compound 18 + GO-HA
Compound 18 + GO-HA

| | | A. | | | | E. coli | E. | K. |
|---|---|---|---|---|---|---|---|---|
| | | baumannii | C. acnes | E. coli | E. coli | 0157:H7 | faecalis | pneumonia |
| | | (ATCC ® | (ATCC ® | (ATCC ® | (ATCC ® | (ATCC ® | (ATCC ® | (ATCC ® |
| | | 19606 ™) | 8739 ™) | 8739 ™) | 25922 ™) | 12900 ™) | 19433 ™) | 10031 ™) |
| 24 h | Average | <200 | 2.9 × 10⁴ | 3.7 × 10³ | <200 | <200 | 4.9 × 10⁴ | 3.0 × 10² |
| | result | | | | | | | |
| | Log Value | <2.3 | 4.5 | 3.6 | <2.3 | <2.3 | 4.7 | 2.5 |
| | Log | >3.0 | 0.4 | 0.9 | >2.3 | >1.9 | 0.4 | 2.3 |
| | Reduction | | | | | | | |
| 72 h | Average | <200 | 6.5 × 10³ | <200 | <200 | <200 | 1.3 × 10⁴ | <200 |
| | result | | | | | | | |
| | Log Value | <2.3 | 3.8 | <2.3 | <2.3 | <2.3 | 4.1 | <2.3 |
| | Log | >3.0 | 1.1 | >2.2 | >2.3 | >1.9 | 1.0 | >2.5 |
| | Reduction | | | | | | | |

| | | | | | | S. | | | |
|---|---|---|---|---|---|---|---|---|---|
| | | P. | P. | P. | S. | aureus | S. | S. | S. |
| | | aeruginosa | aeruginosa | vulgaris | aureus | (MRSA) | epidermidis | maltophilia | pyogenes |
| | | (ATCC ® | (ATCC ® | (ATCC ® | (ATCC ® | (ATCC ® | (ATCC ® | (ATCC ® | (ATCC ® |
| | | 9027 ™) | 15442 ™) | 8427 ™) | 6538 ™) | 33593 ™) | 14990 ™) | 13637 ™) | 19615 ™) |
| 24 h | Average | <200 | <200 | <200 | 4.9 × 10⁴ | <200 | <200 | <200 | <200 |
| | result | | | | | | | | |
| | Log | <2.3 | <2.3 | <2.3 | 4.7 | <2.3 | <2.3 | <2.3 | <2.3 |
| | Value | | | | | | | | |
| | Log | >2.5 | >2.9 | >3.6 | 0.5 | >2.5 | >2.6 | >2.8 | >3.2 |
| | Reduction | | | | | | | | |
| 72 h | Average | <200 | <200 | <200 | 9.3 × 10² | <200 | <200 | <200 | <200 |
| | result | | | | | | | | |
| | Log | <2.3 | <2.3 | <2.3 | 3.0 | <2.3 | <2.3 | <2.3 | <2.3 |
| | Value | | | | | | | | |
| | Log | >2.5 | >2.9 | >3.6 | 2.2 | >2.5 | >2.6 | >2.8 | >3.2 |
| | Reduction | | | | | | | | |

Compound 1 inhibited the growth of 6 of 7 bacteria in test 1 at 72 hours (Table 7-A), all common skin pathogenic bacteria. Compound 1 did not affect MRSA growth in test 1 at 72 hours. Compound 1 inhibited the growth of 7 of 7 bacteria in test 2 at 72 hours (Table 7-B). Compound 7 inhibited the growth of 4 of 7 bacteria at 72 hours (Table 8). Compound 8 inhibited the growth of 5 of 7 bacteria at 72 hours (Table 9). Compound 10 inhibited the growth of 7 of 7 bacteria in test 1 at 72 hours (Table 10-A). Compound 10 also inhibited the growth of 7 of 7 bacteria in test 2 at 72 hours (Table 10-B). Compound 18 inhibited the growth of 14 of 15 bacteria in test 1 at 72 hours (Table 11). As seen in Biological Example 9, the strong bacteriostatic properties of compounds of Formula (I) enhance the wound healing process by minimizing the complicating effects of bacteria, for example, during surgical wound repair.

### Biological Example 5: Elastic Cartilage Regeneration

Compounds of Formula (I) were tested and compared to saline control in elastic cartilage regeneration tests. Rabbit ears were punched through and through with an 8 mm punch. 4 holes were punched in each ear of the specimen (four positions L1, L2, L3, and L4 in the left ear and four positions R5, R6, R7, and R8 in right ear). Saline was applied via a spray on the control specimen, Compound 8 was applied via a phospholipid spray on another specimen, and Compound 18 was applied via a phospholipid spray on yet another specimen. The phospholipid spray contained compound (45 mg), butylene glycol (2 mL), polysorbate 20 (2 mL), phosphotidyl choline (0.5 g), ethanol (2 mL), and ultra pure water (40 mL).

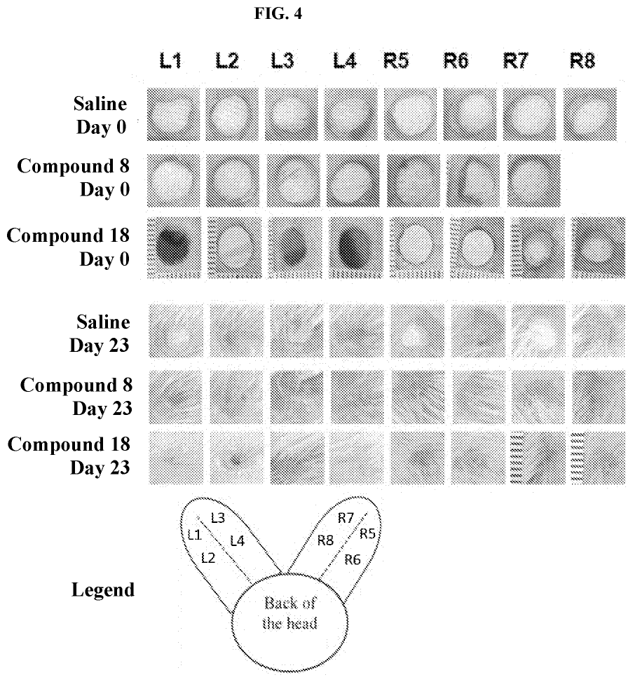

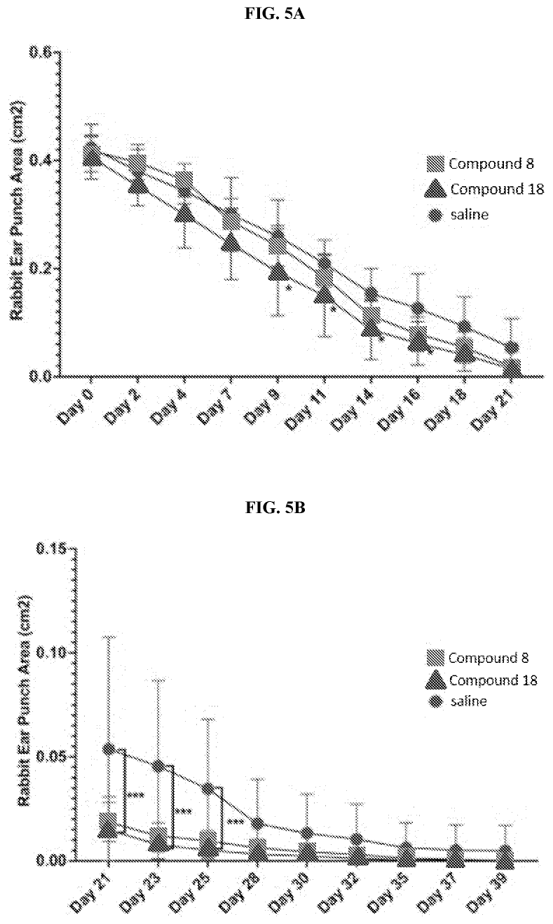

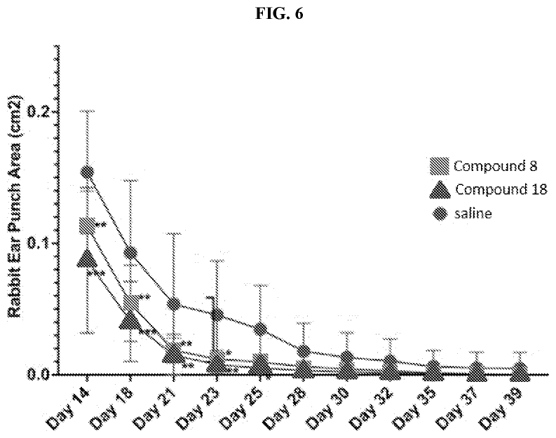

FIG. 4 provides photographs of the wounds after treatment with saline, compound 8, or compound 18 on Days 0 and 23. Ear punch area related to wound closure was measured at 2- to 3-day intervals from day 0 to day 39. Results of the rate of ear closure from day 1-21 is shown in FIG. 5A, and from day 21-39 is shown in FIG. 5B. Additional results from a separate test are shown in FIG. 6 (day 14 to 39).

Results of the rate of ear closure show that compounds of Formula (I) (Compound 8 and Compound 18) improved the rate of ear closure over the control in all tests after day 4. FIG. 5B shows that compounds of Formula (I) provided up to 2-3 times the rate of wound closure (0.01-0.02 cm² area) at day 21 compared to the control (0.055 cm² area).

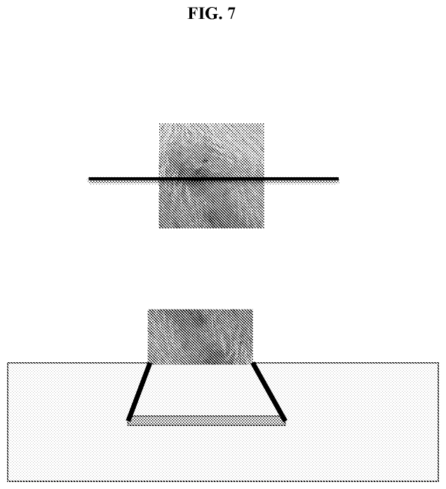

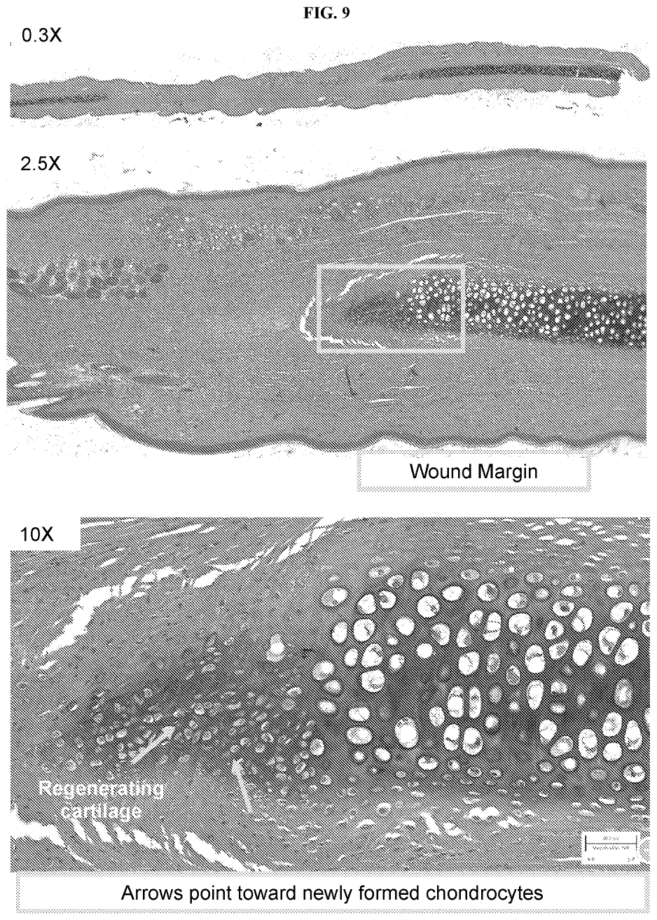

At day 45, the tests were stopped and the tissue was processed. An embed cut was made across the center point of the healed wounds in each site (FIG. 7, top). A slide was used to obtain sectioned tissue sample for staining and imaging (FIG. 7, bottom). Representative examples of cross sectioned and stained tissues are shown in FIGS. 9-11.

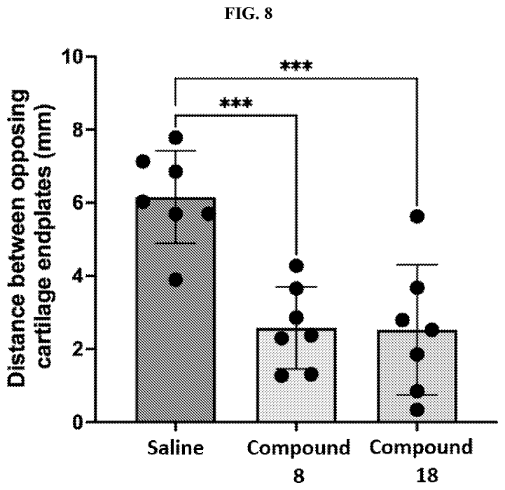

After staining with either Safronin O or Trichrome Blue, the distance between the opposing cartilage endplates was measured. From the original 8 mm wound margin distance, the saline control averaged 6 mm of distance between opposing cartilage endplates (FIG. 8), or 2 mm of average cartilage growth upon closure of the wound (about 25% average cartilage tissue regrowth). Compounds of Formula (I) (Compound 8 and Compound 18) provided about 2-3 mm average distance between opposing cartilage endplates (FIG. 8), or 5.5-6 mm of average cartilage growth upon closure of the wound (about 68-75% average cartilage tissue regrowth).

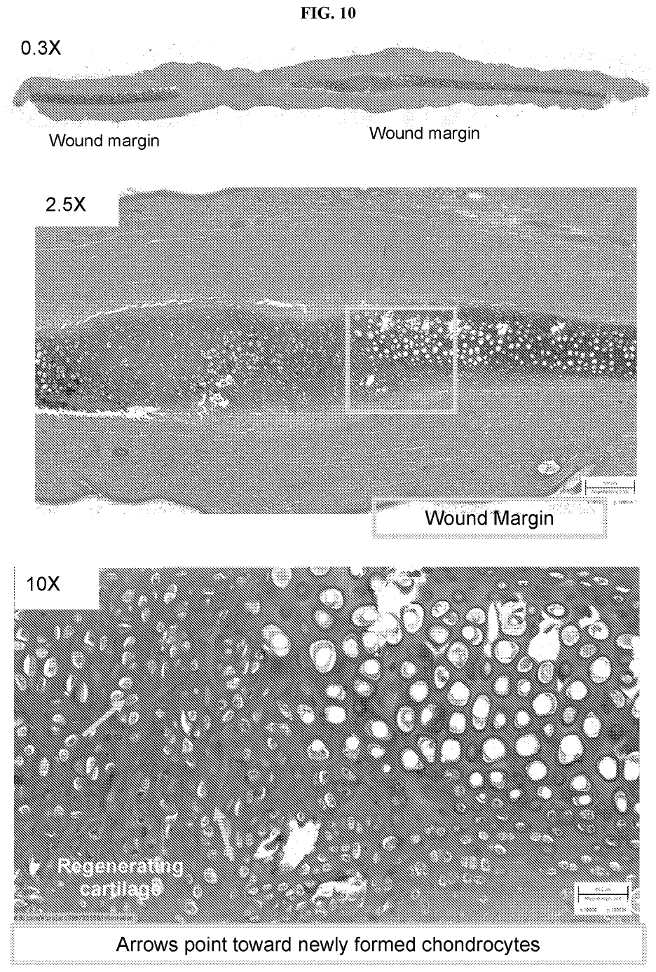

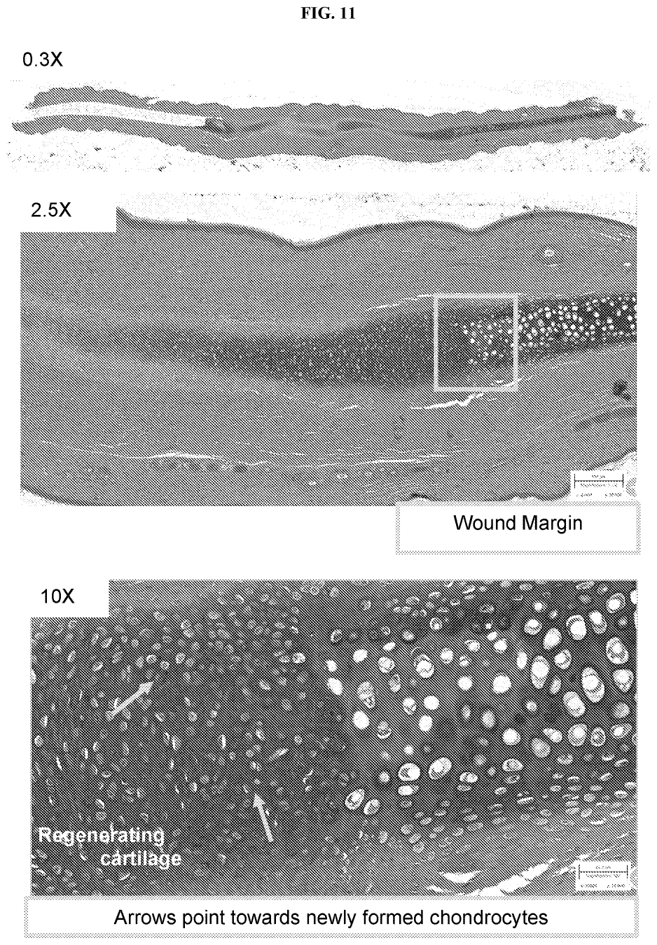

FIGS. 9-11 display the original 8 mm wound margin (top, two vertical gray lines) and in an inset (bottom left, curved gray line). FIGS. 9-11 show expanded views of the cartilage regeneration site where chondrocytes are newly formed within the wound margin. Comparing FIG. 10 (Compound 8) and FIG. 11 (Compound 18) to FIG. 9 (control), cartilage regeneration using compounds of Formula (I) was confirmed. Within the wound margin, newly formed chondrocytes are more numerous and larger than in the control and cartilage regrowth is shown in samples that were treated with compounds of Formula (I) (dark tissue at center of the cross sections in the figures represents cartilage tissue).

As seen in Biological Example 5, compounds of Formula (I) were effective in wound healing and as small molecule elastic cartilage regenerators. Compounds of Formula (I) provided increased rates of wound healing up to 2-3 times the rate compared to a control at 21 days. Compounds of Formula (I) also provided up to 90-95% cartilage regeneration.

### Biological Example 6: Wound Healing—Full Thickness Open Excisional Wounds, Closed Excisional Wounds, and Burn Studies

### Full Thickness Wound Healing Procedure

A 21-day full thickness open excisional wound healing study was conducted on Yucatan™ Miniature Swine (Sinclair BioResources, Auxvasse, Missouri, USA) to evaluate the efficacy of healing and local tissue reaction of compounds of Formula (I). Compounds and controls were topically administered on full thickness open excisional wounds once every other day (total 11 doses).

The negative control for this study was saline (0.9%). The tes compositions for these studies are described as follows.

The test composition in the full thickness open excisional wound studies for compounds without GO-HA (no GO-HA added) was a mixture with an overall concentration of 0.25 to 0.27 mg/mL of the respective compound in serum formulation as follows: compound (10 mg), butylene glycol (1.80 mL), polysorbate 20 (1.80 mL), phosphatidyl choline (0.44 g), ethanol (1.80 mL), ultra pure water (30 mL), and hydroxypropyl cellulose (0.285 g).

The test composition in the full thickness open excisional wounds studies for compounds with GO-HA (Compound+GO-HA) was a mixture with an overall concentration of 1.0 mg/mL of the respective compound and 1.0 mg/mL of GO-HA in serum formulation as follows: compound (35 mg), butylene glycol (1.8 mL), polysorbate 20 (1.8 mL), phosphotidyl choline (0.44 g), ethanol (1.8 mL), GO-HA (35 mg), ultra pure water (30 mL), and hydroxypropyl cellulose (0.285 g).

The test composition in the closed excisional wound and burn studies for compounds without GO-HA (no GO-HA added) was a mixture with an overall concentration of 0.20 to 0.25 mg/mL of the respective compound in serum formulation as follows: compound (4 mg), butylene glycol (0.80 mL), polysorbate 20 (0.40 mL), ultra pure water (18 mL), and hydroxypropyl cellulose (0.12 g).

The test composition in the closed excisional wound and burn studies for compounds with GO-HA (Compound+GO-HA) was a mixture with an overall concentration of 1.0 mg/mL of the respective compound and 0.5 mg/mL of GO-HA in serum formulation as follows: compound (18 mg), PEG 400 (0.8 mL), GO-HA (9 mg), ultra pure water (18 mL), and hydroxypropyl cellulose (0.12 g).

Miniature swine used in this study are a well-accepted non-rodent model for dermal wound healing studies due to the anatomical similarities between humans and swine, including dermal thickness, subcutaneous tissues, and underlying muscular structures. For this reason, Yucatan™ Miniature Swine were chosen as an appropriate species to evaluate dermal materials and wound healing properties of compounds of Formula (I).

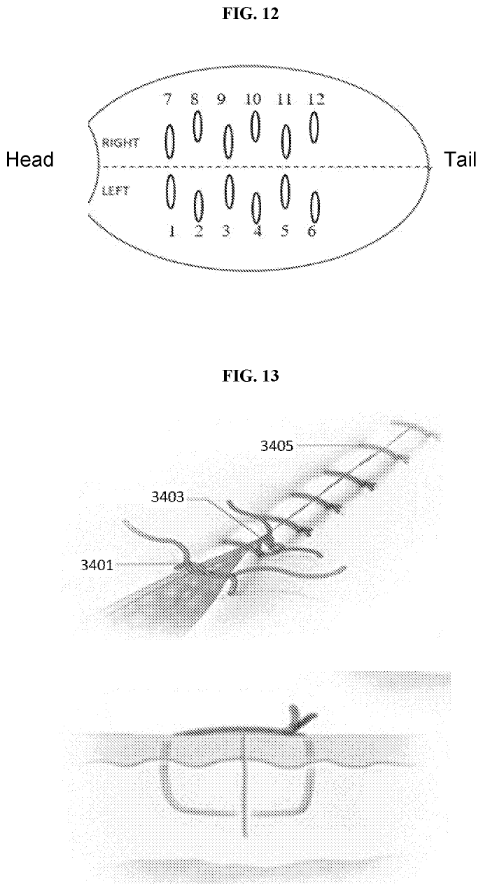

The dermal wounds were generated to mimic a full-thickness skin lesion consistent with worst case clinical scenarios. The number of animals (6) ensured sufficient endpoints from 12 total wounds per animal for the designated test articles, which were collected for evaluation from each treatment group (FIG. 12).

The dose volume related to the full thickness open excisional wounds was selected based on the wound size and intended clinical application to have the wound surface covered and effective. The dose concentration and volume (0.2 mL/site per dose) were chosen for the wounds at the specified size.

Full-thickness 60-day burn wound healing study. Full-thickness, 3^{rd} degree burn wounds of 3×3 cm² size were created on the back of the Yucatan minipig (5 wounds on each side of the spine, 10 total per swine) by using a brass cylinder (circular cross-section, 3 cm diameter) that was preheated in an oven at 100° C. and applied to the dorsal skin with light pressure for 30 seconds. Debridement of the wounds occurred day 7 post-dose. Compounds were dosed topically three times a week (0.24 mL) for thirty days. At 30 day time-point, the wounds were photographed once a week and NO dosing of compounds conducted from day 31-60. On day 60 animals were sacrificed and tissue prepared for analysis.

The animals were fasted overnight (or at least 4 hours) prior to anesthesia. The animals were given analgesic treatments for surgery procedures on dosing phase (day 1), and dressing changes when needed.

The frequency of wound creation was once on dosing phase day 1. The frequency of dose administration was once on dosing phase days 1, 3, 5, 7, 9, 11, 13, 15, 17, 19, and 21. Each animal had twelve 5 cm×0.5 cm full thickness fusiform(spindle shaped) wound sites, spaced at least 3 cm apart to the appropriate depth and in a staggered pattern to reduce central cross-sectional wound tension.

Once homeostasis was obtained, each wound was sutured closed using non-absorbable suture, the simple interrupted closure technique is shown in FIG. 13. Each wound had 5 simple interrupted sutures with a 0.5 to 1 cm interval between sutures.

Compounds of Formula (I) and controls were applied directly to the wound site in a generous layer evenly over the sutured wound using a sterile applicator as needed. After application, each wound site was covered with suitable sterile dressing materials.

Dressings were changed every dosing phase day at day 3, 5, 7, 9, 11, 13, 15, 17, 19, and 21, and sutures were removed on dosing phase day 15. After dose administration on each dosing day, the wounds were covered with dressings as described above.

All animals were scheduled to be terminated on dosing phase day 22. At termination, the entire wound site (dose site) was collected separately for each site and included at least 0.5 cm margin skin and a depth below the wound bed.

### Full Thickness Open Excisional Wound Healing Study Results

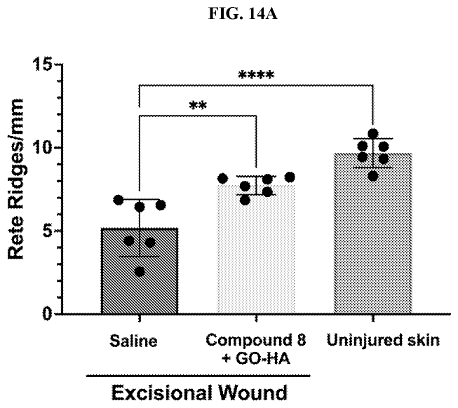

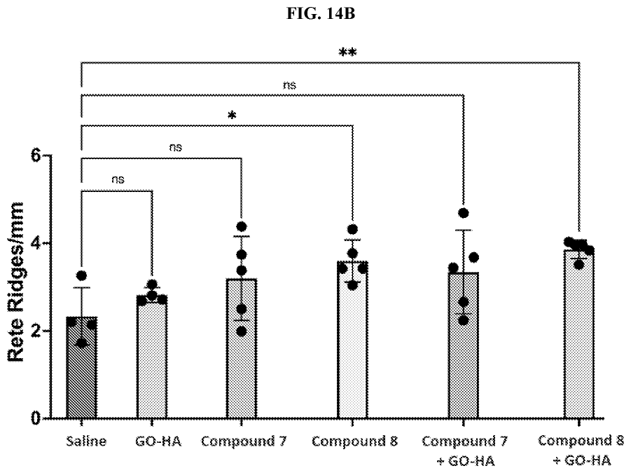

FIGS. 14A-14B show the resulting full thickness excisional wound area (cm²) from this study for compounds of Formula (I) (Compound 1, Compound 7, Compound 8, and Compound 10), compounds of Formula (I) and GO-HA (Compound 1+GO-HA, Compound 7+GO-HA, Compound 8+GO-HA, and Compound 10+GO-HA) compared to the vehicle (previously described), and the control (saline).

In all tests, compounds of Formula (I) and compounds of Formula (I) with GO-HA provided improved wound healing from day 5 to day 15 compared to the vehicle alone and the control. In some tests, compounds of Formula (I) and compounds of Formula (I) with GO-HA provided improved wound healing throughout the entire test (day 3 to day 22), such as Compound 7, Compound 8, and Compound 8+GO-HA.

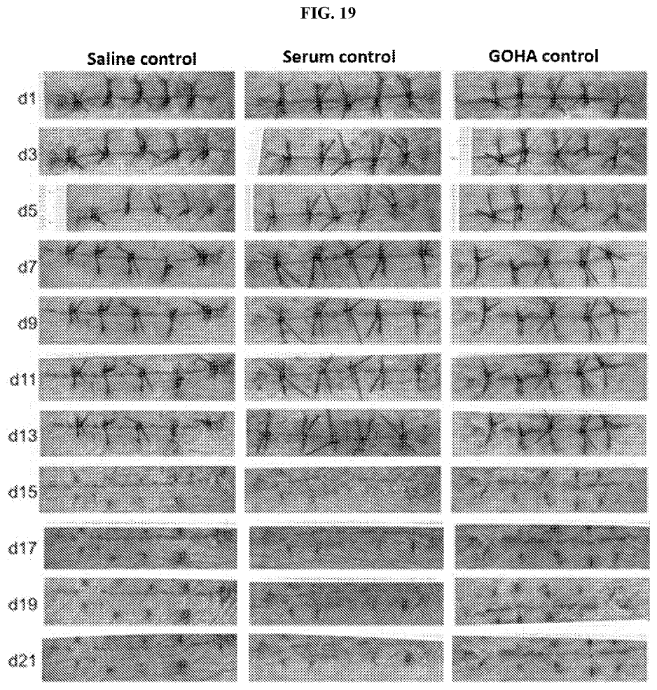

Closed excisional wound healing study results (cosmesis) As shown in FIGS. 19-23, Compound 1 resulted in 7 of 8 wounds with improved cosmesis (appearance such as greater amount of visible wound closure and less scaring or smaller scar size) than the control; Compound 1+GO-HA resulted in 3 of 8 wounds with improved visible appearance than the control; Compound 10 resulted in 3 of 8 wounds with improved visible appearance than the control; and Compound 8 resulted in 2 of 8 wounds with improved visible appearance than the control.

FIG. 19 shows results of the closed excisional wound study for saline control, serum control (aka serum formulation control), and GO-HA control, from day 1 to day 21. The sutures were removed at day 13. Serum formulation control was butylene glycol (0.80 mL), polysorbate 20 (0.40 mL), ultra pure water (18 mL), and hydroxypropyl cellulose (0.12 g). GO-HA control was PEG 400 (0.8 mL), GO-HA (9 mg), ultra pure water (18 mL), and hydroxypropyl cellulose (0.12 g).

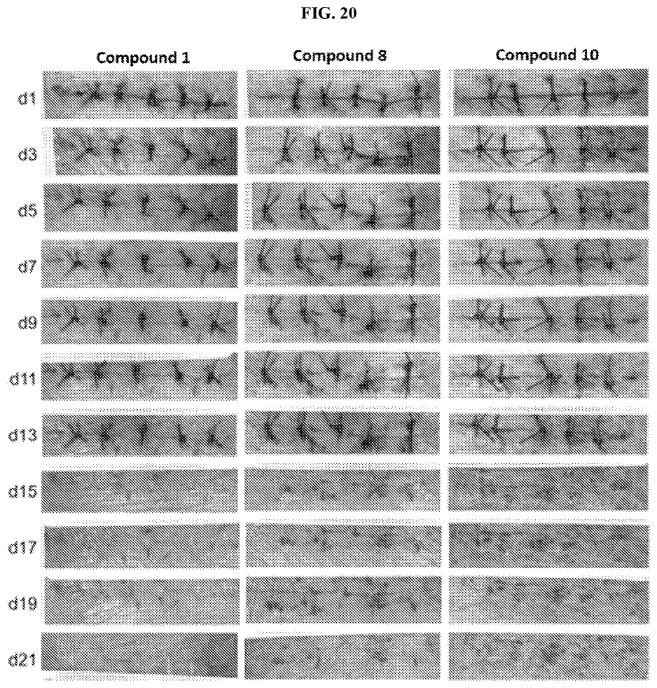

FIG. 20 shows that compounds of Formula (I) provided improved wound closure with less scaring compared to the controls (FIG. 19) at day 21.

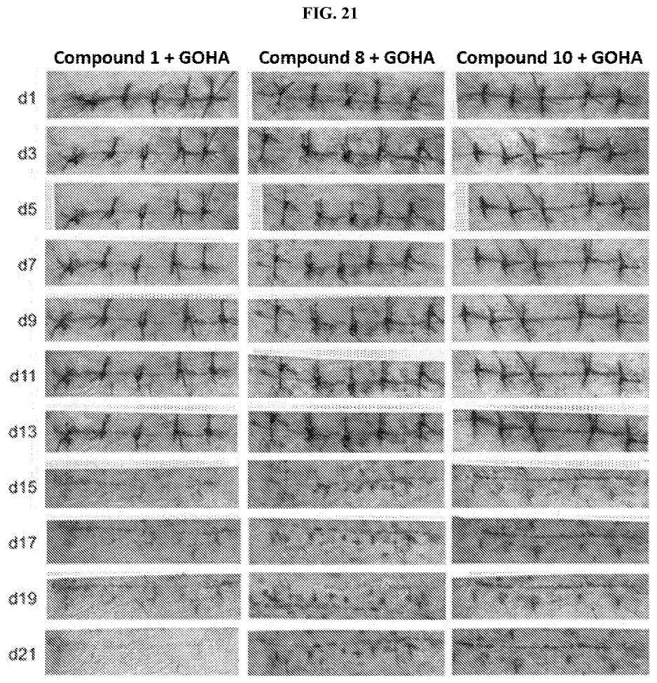

FIG. 21 shows results of the closed excisional wound study: compounds of Formula (I) with GO-HA (Compound 1+GO-HA, Compound 8+GO-HA, and Compound 10+GO-HA), from day 1 to day 21. The sutures were removed at day 13.

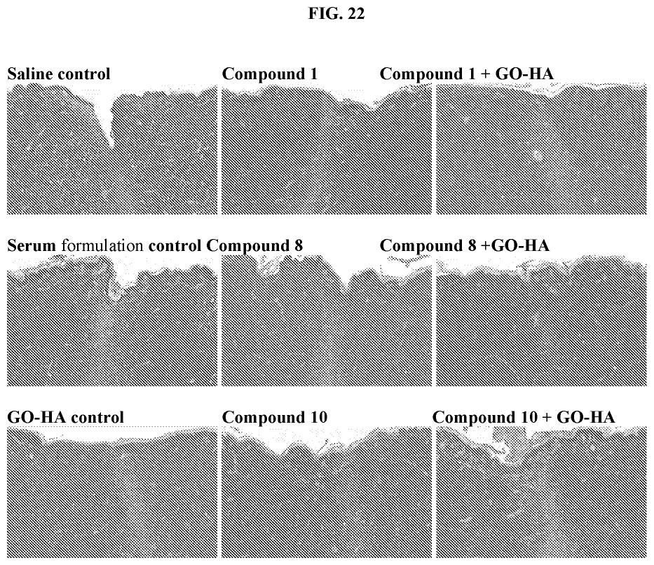

FIG. 22 shows results of the closed excisional wound study: cross sectional tissue samples stained with Trichrome Blue. Wound sites treated with saline control, serum formulation control [butylene glycol (0.80 mL), polysorbate 20 (0.40 mL), ultra pure water (18 mL), and hydroxypropyl cellulose (0.12 g)], GO-HA control [PEG 400 (0.8 mL), GO-HA (9 mg), ultra pure water (18 mL), and hydroxypropyl cellulose (0.12 g)], compounds of Formula (I) (Compound 1, Compound 8, Compound 10), and compounds of Formula (I) with GO-HA (Compound 1+GO-HA, Compound 8+GO-HA, and Compound 10+GO-HA) are compared.

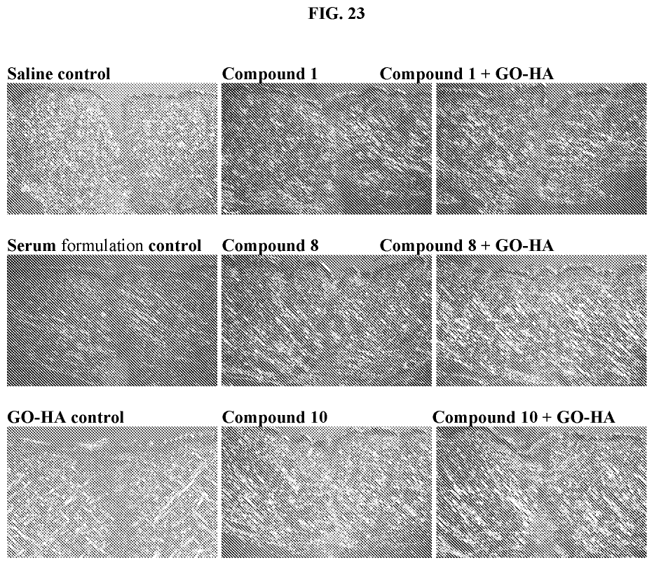

FIG. 23 shows results of the closed excisional wound study: cross sectional tissue samples under polarized microscopy. Wound sites treated with saline control, serum formulation control [butylene glycol (0.80 mL), polysorbate 20 (0.40 mL), ultra pure water (18 mL), and hydroxypropyl cellulose (0.12 g)], GO-HA control [PEG 400 (0.8 mL), GO-HA (9 mg), ultra pure water (18 mL), and hydroxypropyl cellulose (0.12 g)], GO-HA control, compounds of Formula (I) (Compound 1, Compound 8, Compound 10), and compounds of Formula (I) with GO-HA (Compound 1+GO-HA, Compound 8+GO-HA, and Compound 10+GO-HA) are compared.

### Burn Wound Healing Study Results

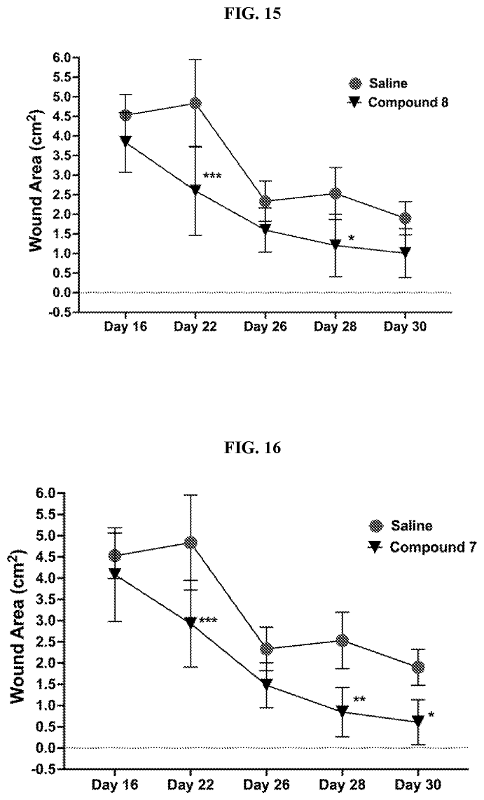

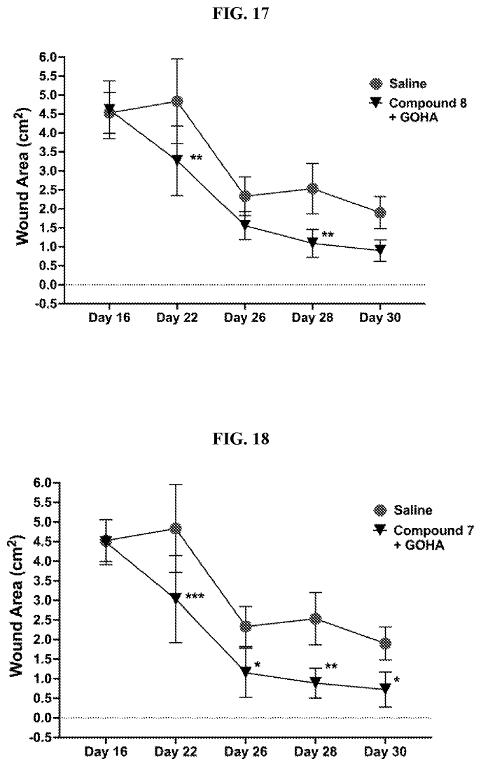

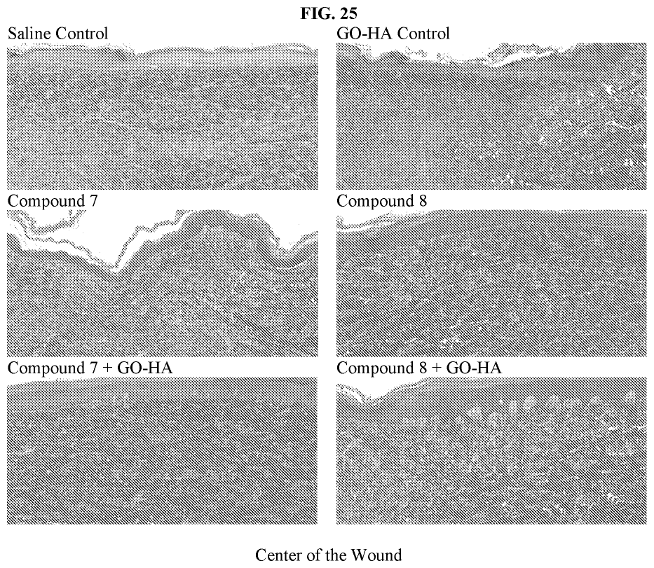

As shown in FIGS. 15-18, Compound 8, Compound 7, Compound 8+GO-HA, and Compound 7+GO-HA resulted in statistically significant improved burn wound healing rates compared to saline control from about day 16 to day 30. Histology (H&E staining: hematoxylin and eosin) demonstrated that the compounds promoted regeneration of tissue (improved organized reticular collagen and rete ridge formation) and reduced scar formation as compared to saline and GOHA. Results of the histological stains are shown in FIG. 25. GO-HA Control is as provided above for FIG. 19.

### Physical Properties of Healed Wounds

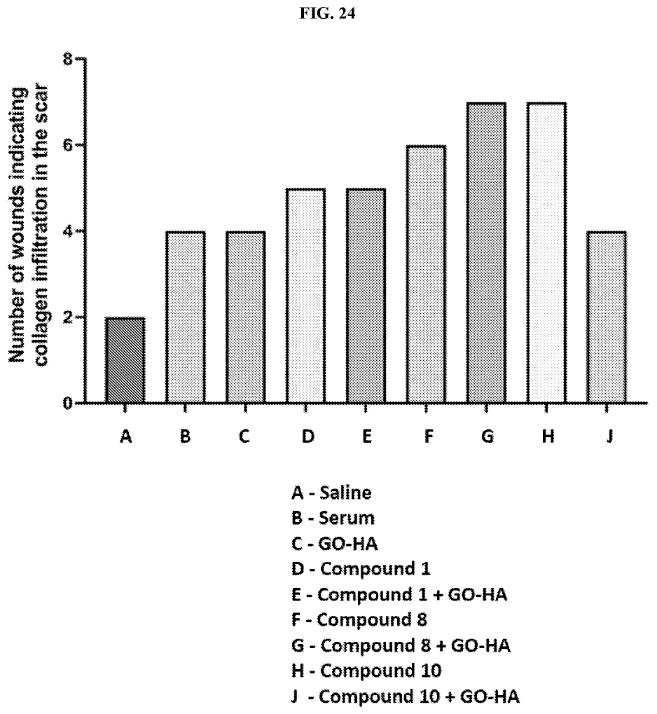

Additional tests were performed including elasticity (Table 12); max force (Table 13); tensile strength (strain) (Table 14); energy peak (Table 15); rete-ridge formation; and collagen organization (plane polarized light) (FIG. 24) of the healed wound sites, described as follows.

TABLE 12
Elasticity of new porcine skin topically treated with compounds
of Formula (I) (mechanical and measured by elastin) (day 21)

| | Elasticity (N/mm) | | | | | | | | |
|---|---|---|---|---|---|---|---|---|---|
| | A | B | C | D | E | F | G | H | J |
| 1 | 13.427 | 22.789 | 37.729 | 27.299 | 37.757 | 55.607 | 59.303 | 52.568 | 23.620 |
| 2 | 37.769 | 38.947 | 19.017 | 33.002 | 67.483 | 46.485 | 58.036 | 74.191 | 39.158 |
| 3 | 32.830 | 29.511 | 48.957 | 50.617 | 45.378 | 28.331 | 54.893 | 44.962 | 55.709 |
| 4 | 28.367 | 33.011 | — | 39.593 | 31.494 | 47.748 | 51.866 | 43.004 | 37.715 |
| 5 | — | — | — | — | — | 22.23 | — | — | — |
| Mean | 28.098 | 31.064 | 35.234 | 37.628 | 45.528 | 40.080 | 56.024 | 53.682 | 39.050 |
| STDEV | 10.508 | 6.753 | 15.125 | 10.011 | 15.699 | 14.121 | 3.335 | 14.282 | 13.133 |

A is saline (control), B is serum formulation (vehicle), C is GO-HA (vehicle), D is Compound 1; E is Compound 1+GO-HA; F is Compound 8; G is Compound 8+GO-HA; H is Compound 10; and J is Compound 10+GO-HA.

Elasticity (N/mm) is the ability of a material to return to its original shape after stress has been removed. All compounds of Formula (I) tested with or without GO-HA ranged from 33% to 99% improvement in elasticity compared to the saline control. Notably, Compound 10 provided a 91% increase and Compound 8+GO-HA provided a 99% increase in elasticity compared to the saline control.

TABLE 13
Max force of new porcine skin topically treated with compounds of Formula (I) (day 21)

| | Max Force (N) | | | | | | | | |
|---|---|---|---|---|---|---|---|---|---|
| | A | B | C | D | E | F | G | H | J |
| 1 | 158.975 | 239.052 | 323.715 | 290.184 | 229.942 | 404.094 | 371.235 | 406.879 | 211.166 |
| 2 | 305.553 | 332.215 | 264.140 | 278.868 | 496.001 | 488.094 | 455.581 | 474.825 | 475.377 |
| 3 | 184.179 | 216.024 | 405.855 | 250.554 | 383.447 | 273.110 | 370.527 | 344.860 | 330.911 |
| 4 | 279.416 | 248.901 | — | 273.195 | 234.632 | 330.413 | 261.925 | 335.864 | 264.003 |
| 5 | — | — | — | — | — | 169.61 | — | — | — |
| Mean | 232.031 | 259.048 | 331.237 | 273.200 | 336.005 | 333.196 | 364.817 | 390.607 | 320.364 |
| STDEV | 71.362 | 50.686 | 71.156 | 16.668 | 128.290 | 121.954 | 79.370 | 64.413 | 114.369 |

A is saline (control), B is serum formulation control (vehicle), C is GO-HA (vehicle), D is Compound 1; E is Compound 1+GO-HA; F is Compound 8; G is Compound 8+GO-HA; H is Compound 10; and J is Compound 10+GO-HA. Descriptions for saline control, serum formulation control, and GO-HA are as provided above for FIG. 19.

Max Force (N) is the average force required to break/rupture a material. All compounds of Formula (I) tested with or without GO-HA ranged from 18% to 68% improvement in max force compared to the saline control. Notably, Compound 8+GO-HA provided a 57% improvement and Compound 10 provided a 68% improvement in max force over the saline control.

TABLE 14
Strain (tensile strength) of new porcine skin topically
treated with compounds of Formula (I) (day 21)

| | Strain (N/mm²) | | | | | | | | |
|---|---|---|---|---|---|---|---|---|---|
| | A | B | C | D | E | F | G | H | J |
| 1 | 1.134 | 2.172 | 4.397 | 2.568 | 6.200 | 7.640 | 9.473 | 6.792 | 2.642 |
| 2 | 4.669 | 4.566 | 1.369 | 3.906 | 9.181 | 4.427 | 7.393 | 11.592 | 3.226 |
| 3 | 5.852 | 4.032 | 5.906 | 10.226 | 5.370 | 2.939 | 8.132 | 5.862 | 9.379 |
| 4 | 2.880 | 4.378 | — | 5.738 | 4.227 | 6.900 | 10.271 | 5.506 | 5.388 |
| 5 | — | — | — | — | — | 2.913 | — | — | — |
| Mean | 3.634 | 3.787 | 3.891 | 5.609 | 6.245 | 4.964 | 8.817 | 7.438 | 5.159 |
| STDEV | 2.066 | 1.099 | 2.310 | 3.341 | 2.118 | 2.208 | 1.296 | 2.822 | 3.051 |

A is saline (control), B is serum formulation (vehicle), C is GO-HA (vehicle), D is Compound 1; E is Compound 1+GO-HA; F is Compound 8; G is Compound 8+GO-HA; H is Compound 10; and J is Compound 10+GO-HA.

Strain (tensile strength) (N/mm²) is the force required to pull/stretch a material to a breaking point. A 20% improvement over saline considered good. All compounds of Formula (I) tested with or without GO-HA ranged from 36% to 142% improvement in tensile strength compared to the saline control. Notably, Compound 10 provided a 105% improvement and Compound 8+GO-HA provided a 142% improvement in tensile strength (strain) over the saline control.

TABLE 15
Energy peak of new porcine skin topically treated with compounds of Formula (I) (day 21)

| | Energy Peak (N*mm) | | | | | | | | |
|---|---|---|---|---|---|---|---|---|---|
| | A | B | C | D | E | F | G | H | J |
| 1 | 1882.263 | 2507.653 | 2777.473 | 3084.657 | 1400.346 | 2946.121 | 2323.932 | 3149.241 | 1887.823 |
| 2 | 2471.921 | 2833.797 | 3668.901 | 2356.435 | 3645.607 | 5124.987 | 3576.311 | 3038.880 | 5771.077 |
| 3 | 1033.244 | 1581.296 | 3364.5383 | 1240.242 | 3240.127 | 2632.780 | 2501.057 | 2645.076 | 1965.611 |
| 4 | 2752.248 | 1876.714 | — | 1885.046 | 1748.008 | 2286.458 | 1322.721 | 2623.098 | 1848.021 |
| 5 | — | — | — | — | — | 1294.14 | — | — | — |
| Mean | 2034.919 | 2199.865 | 3270.304 | 2141.595 | 2508.522 | 2856.897 | 2431.005 | 2864.074 | 2868.133 |
| STDEV | 759.866 | 572.613 | 453.124 | 777.555 | 1100.704 | 1411.562 | 923.109 | 269.510 | 1935.912 |

A is saline (control), B is serum formulation (vehicle), C is GO-HA (vehicle), D is Compound 1; E is Compound 1+GO-HA; F is Compound 8; G is Compound 8+GO-HA; H is Compound 10; and J is Compound 10+GO-HA.

Energy Peak (N*mm) is the maximum force applied to a material over time. All compounds of Formula (I) tested with or without GO-HA ranged from 5% to 41% improvement in energy peak compared to the saline control. Notably, Compound 8 provided a 29% improvement and Compound 10 provided a 41% improvement in energy peak over the saline control. The vehicle, GO-HA, provided a 61% improvement in energy peak over the saline control.

### Rete Ridges

Regarding rete ridge formation, representative example images from the full thickness wound healing study are shown in FIGS. 14A and 15B. FIGS. 14A and 14B show that compounds of Formula (I) (Compound 7, Compound 8) and compounds of Formula (I) with GO-HA (Compound 7+GO-HA, Compound 8+GO-HA) improved wound healing compared to the saline control (rete ridge/mm), for example, approaching the rete ridge/mm value of uninjured skin. Improved rete ridge formation is described as epithelial extensions that project into the underlying connective tissue in both skin and mucous membrances—improved rete ridge formation is a sign of tissue regeneration rather than fibrosis/scarring (reduced rete ridge formation).

### Collagen Organization

Regarding collagen organization, the number of wounds indicating collagen infiltration into the scar were identified by polarized imaging (plane polarized light). As shown in FIG. 24, all compounds of Formula (I) with or without GO-HA provided greater collagen organization compared to the saline control. Notably, Compound 10 and Compound 8+GO-HA showed 7 wounds indicating collagen infiltration into the scar.

In summary, in all tests compounds of Formula (I) with or without GO-HA showed improvements in wound healing (physical properties of the healed wound site) compared to the control. Wounds treated with Compound 8+GO-HA and Compound 10 showed the greatest wound healing integrity for elasticity, strain, and rete ridge formation (Compound 8+GO-HA), max force (Compound 10), and collagen organization compared to the control. The wounds treated with GO-HA vehicle had the greatest energy peak. Wounds treated with 0.9% saline provided the least wound healing integrity for elasticity, max force, strain, and energy peak parameters.

All publications, patents, and patent applications cited in this specification are herein incorporated by reference as if each individual publication, patent, or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

1. A compound of Formula (I): 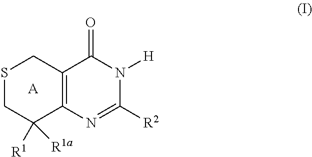
or a single stereoisomer or mixture of stereoisomers thereof; a single tautomer or mixture of tautomers thereof, and/or a pharmaceutically acceptable salt thereof;
wherein
R¹ is hydrogen, deuterium, C₁-C₃alkyl, —OH, —O—C₁-C₃alkyl, —CH₂OH, or —B(OH)₂;
R^{1a} is hydrogen, deuterium, or C₁-C₃alkyl;
R² is
(a) phenyl substituted with R³ and additionally optionally substituted with 1, 2, or 3 R^{3a} groups;
(b) phenyl substituted with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups,
(c) phenyl optionally substituted with 1, 2, or 3 R^{3a} groups and additionally substituted with -phenyl-R³ where the phenyl in -phenyl-R³ is optionally substituted with 1, 2, or 3 R^{3a} groups;
(d) 5- or 6-membered heteroaryl substituted with R³ and additionally optionally substituted with 1, 2, or 3 R^{3a} groups;
(e) 5- or 6-membered heteroaryl optionally substituted with 1, 2, or 3 R^{3a} groups and additionally substituted with -phenyl-R³ where the phenyl is additionally optionally substituted with 1, 2, or 3 R^{3a} groups;
(f) 5- or 6-membered heteroaryl optionally substituted with 1, 2, or 3 R^{3a} groups and additionally substituted with -(5- or 6-membered heteroaryl)-R³ where the 5- or 6-membered heteroaryl in -(5- or 6-membered heteroaryl)-R³ is optionally substituted with 1, 2, or 3 R^{3a} groups;
(g) C₃-C₆cycloalkyl substituted with R³ and additionally optionally substituted with 1 or 2 R^{3a} groups;
(h) C₃-C₆cycloalkyl substituted with 5- or 6-membered heteroaryl where the 5- or 6-membered heteroaryl is substituted with R³ and additionally optionally substituted with 1, 2, or 3 R^{3a} groups, and where the cycloalkyl is optionally substituted with 1 or 2 R^{3a} groups;
(i) C₃-C₆cycloalkyl substituted with phenyl where the phenyl is substituted with R³ and additionally optionally substituted with 1, 2, or 3 R^{3a} groups, and where the cycloalkyl is optionally substituted with 1 or 2 R^{3a} groups;
(j) 3- to 8-membered heterocycloalkyl substituted with phenyl or substituted with 5- or 6-membered heteroaryl, where the phenyl and the 5- to 6-membered heteroaryl are substituted with R³ and additionally optionally substituted with 1, 2, or 3 R^{3a} groups;
(k) —CH═CH—R⁵ where R⁵ is phenyl or 5- or 6-membered heteroaryl, where the phenyl and the 5- or 6-membered heteroaryl are substituted with R³ and additionally optionally substituted with 1, 2, or 3 R^{3a} groups;
R³ is independently selected from —B(OH)₂, cyano, halo, halo-C₁-C₆alkyl, —(C₀-C₆alkylene)-O—R⁴, or 5- to 10-membered heterocyclic wherein the 5- to 10-membered heterocyclic is optionally substituted with cyano; or when R² is (a), then R³ and one R^{3a}, when on adjacent carbons, together with the carbons to which they are attached form 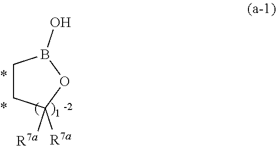
where the * indicate the carbons shared with the phenyl ring and where the remaining optional R^{3a} on the phenyl portion are as defined below, and each R^{7a} is independently hydrogen or C₁-C₆alkyl;
each R^{3a} is independently selected from cyano, halo, —OH, C₁-C₆alkyl, halo-C₁-C₆alkyl, and C₁-C₆alkoxy;
R⁴ is hydroxy-C₁-C₆alkyl, C₁-C₆alkoxy-C₁-C₆alkyl, or C₁-C₆alkoxycarbonyl-NH—C₁-C₆alkyl; and
provided that the compound is not
2-(4-(trifluoromethyl)phenyl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
2-(4-(4-methoxyphenyl)piperazin-1-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
2-(4-(3-methoxyphenyl)piperazin-1-yl)-3,5,7,8-tetrahydro-4H-thiopyrano[4,3-d]pyrimidin-4-one or a single stereoisomer or mixture of stereoisomers thereof;
a single tautomer or mixture of tautomers thereof; and/or a pharmaceutically acceptable salt thereof;
2-(4-(2-methoxyphenyl)piperazin-1-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
2-(4-chlorophenyl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof,
2-(5-(trifluoromethyl)pyridin-2-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof;
2-(3-(trifluoromethyl)phenyl)-3,5,7,8-tetrahydro-4H-thiopyrano[4,3-d]pyrimidin-4-one or a single stereoisomer or mixture of stereoisomers thereof, a single tautomer or mixture of tautomers thereof; and/or a pharmaceutically acceptable salt thereof;
2-(3-(trifluoromethyl)phenyl)-3,5,7,8-tetrahydro-4H-thiopyrano[4,3-d]pyrimidin-4-one or a single stereoisomer or mixture of stereoisomers thereof, a single tautomer or mixture of tautomers thereof; and/or a pharmaceutically acceptable salt thereof; or
2-(5-chlorothiophen-3-yl)-3,5,7,8-tetrahydro-4H-thiopyrano[4,3-d]pyrimidin-4-one or a single stereoisomer or mixture of stereoisomers thereof; a single tautomer or mixture of tautomers thereof; and/or a pharmaceutically acceptable salt thereof.

2. The compound of claim 1, or a single stereoisomer or mixture of stereoisomers thereof, a single tautomer or mixture of tautomers thereof, and/or a pharmaceutically acceptable salt thereof, wherein R¹ and R^{1a} are independently selected from is hydrogen and deuterium.

3. The compound of claim 1 or 2, or a single stereoisomer or mixture of stereoisomers thereof, a single tautomer or mixture of tautomers thereof, and/or a pharmaceutically acceptable salt thereof, wherein ring A is 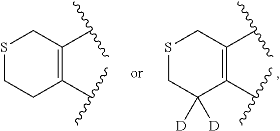 [SMILES: *C1=C(*)CSCC1 ; *C1=C(*)C([2H])([2H])CSC1]
and wherein [symbol] designates attachment to the remainder of the compound of Formula (I).

4. The compound of any one of claims 1-3, or a single stereoisomer or mixture of stereoisomers thereof, a single tautomer or mixture of tautomers thereof, and/or a pharmaceutically acceptable salt thereof, wherein R² is phenyl substituted with R³ and additionally optionally substituted with 1, 2, or 3 R^{3a} groups.

5. The compound of claim 4, or a single stereoisomer or mixture of stereoisomers thereof, a single tautomer or mixture of tautomers thereof, and/or a pharmaceutically acceptable salt thereof, wherein
R³ is attached to the para-position of the phenyl ring; or
R³ and one R^{3a}, when on adjacent carbons, together with the carbons to which they are attached form ring (a-1) and
where the phenyl portion is optionally substituted with the remaining R^{3a} groups.

6. The compound of any one of claims 1-5, or a single stereoisomer or mixture of stereoisomers thereof, a single tautomer or mixture of tautomers thereof, and/or a pharmaceutically acceptable salt thereof, wherein R² is selected from the group consisting of

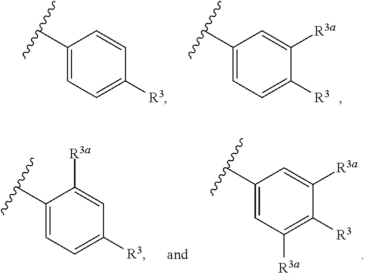

7. The compound of any one of claims 1-6, or a single stereoisomer or mixture of stereoisomers thereof, a single tautomer or mixture of tautomers thereof, and/or a pharmaceutically acceptable salt thereof, wherein R² is selected from the group consisting of

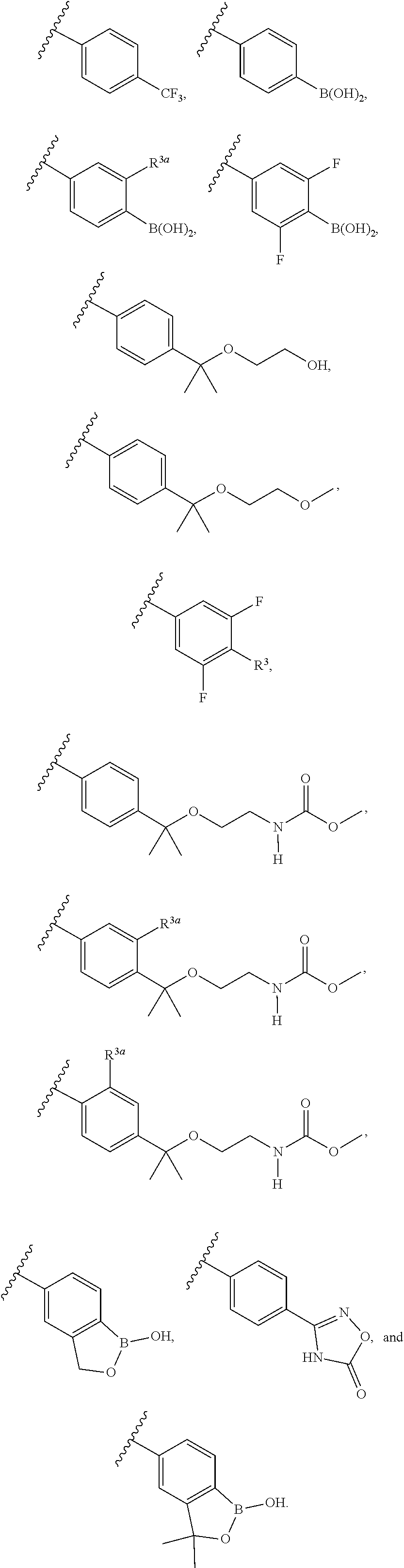

8. The compound of any one of claims 1-7, or a single stereoisomer or mixture of stereoisomers thereof, a single tautomer or mixture of tautomers thereof, and/or a pharmaceutically acceptable salt thereof, wherein R² is selected from the group consisting of

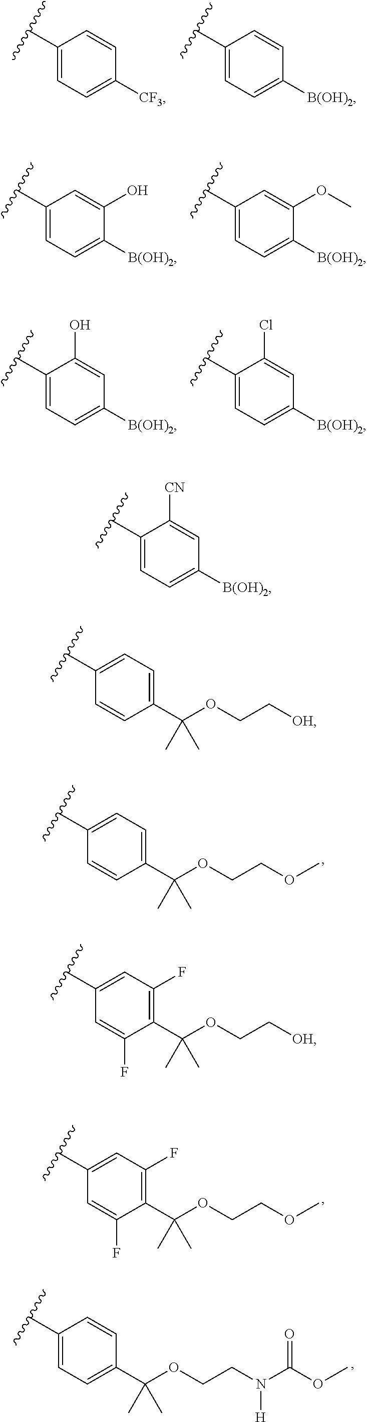

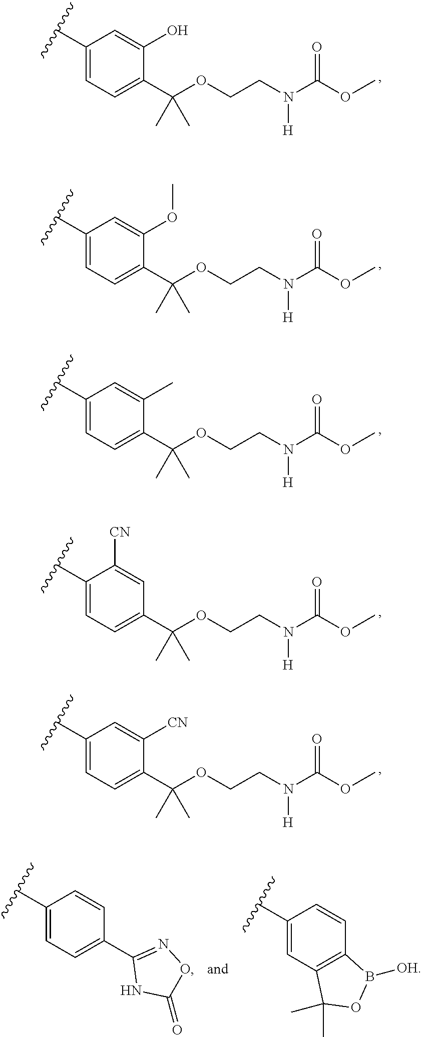

9. The compound of any one of claims 1-3, or a single stereoisomer or mixture of stereoisomers thereof, a single tautomer or mixture of tautomers thereof, and/or a pharmaceutically acceptable salt thereof, wherein R² is phenyl substituted with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups.

10. The compound of claim 9, or a single stereoisomer or mixture of stereoisomers thereof, a single tautomer or mixture of tautomers thereof, and/or a pharmaceutically acceptable salt thereof, wherein R² is phenyl substituted at its para-position with 5- or 6-membered heteroaryl where the 5- or 6-membered heteroaryl is substituted with R³ and additionally optionally substituted with 1, 2, or 3 R^{3a} groups and where the phenyl is additionally optionally substituted with 1, 2, or 3 R^{3a} groups; and when the 5- or 6-membered heteroaryl is a 6-membered heteroaryl then R³ is substituted on the para-position of the 6-membered heteroaryl.

11. The compound of any one of claims 1-3, 9, and 10, or a single stereoisomer or mixture of stereoisomers thereof, and/or a pharmaceutically acceptable salt thereof, wherein R² is selected from the group consisting of

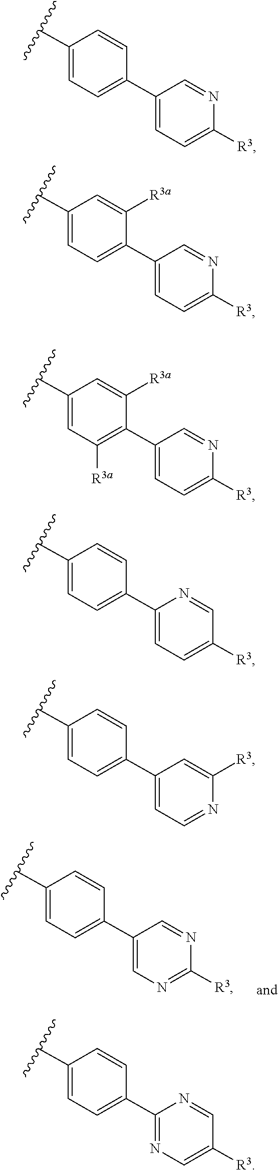

12. The compound of any one of claims 1-3 and 9-11, or a single stereoisomer or mixture of stereoisomers thereof, a single tautomer or mixture of tautomers thereof, and/or a pharmaceutically acceptable salt thereof, wherein R² is selected from the group consisting of

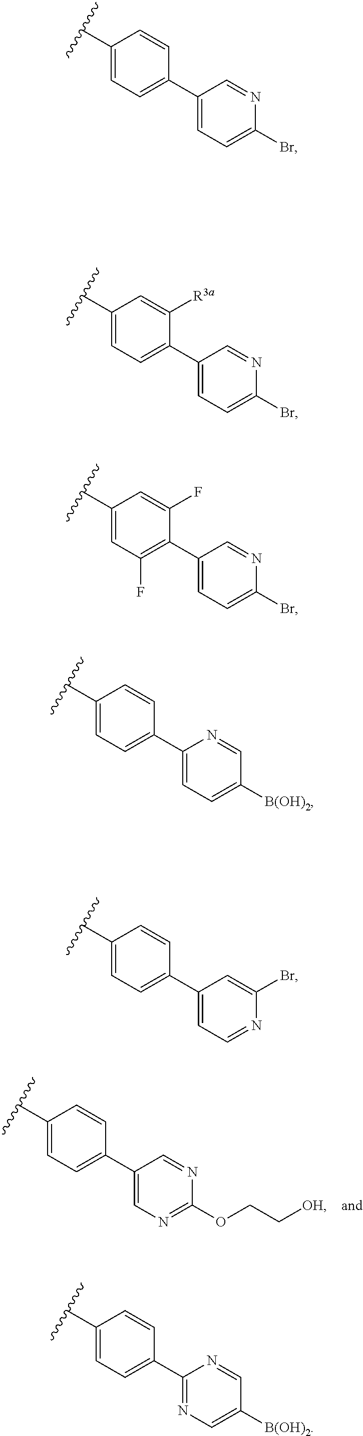

13. The compound of any one of claims 1-3, or a single stereoisomer or mixture of stereoisomers thereof, a single tautomer or mixture of tautomers thereof, and/or a pharmaceutically acceptable salt thereof, wherein R² is phenyl optionally substituted with 1, 2, or 3 R^{3a} groups and additionally substituted with -phenyl-R³ where the phenyl in -phenyl-R³ is optionally substituted with 1, 2, or 3 R^{3a} groups.

14. The compound of claim 13, or a single stereoisomer or mixture of stereoisomers thereof, a single tautomer or mixture of tautomers thereof, and/or a pharmaceutically acceptable salt thereof, wherein R² is phenyl optionally substituted with 1, 2, or 3 R^{3a} groups and additionally substituted at its para-position with -phenyl-R³ where the phenyl in -phenyl-R³ is optionally substituted with 1, 2, or 3 R^{3a} groups and where the R³ is in the para-position of the phenyl in -phenyl-R³.

15. The compound of any one of claims 1-3 and 13-14, or a single stereoisomer or mixture of stereoisomers thereof, a single tautomer or mixture of tautomers thereof, and/or a pharmaceutically acceptable salt thereof, wherein R² is selected from the group consisting of

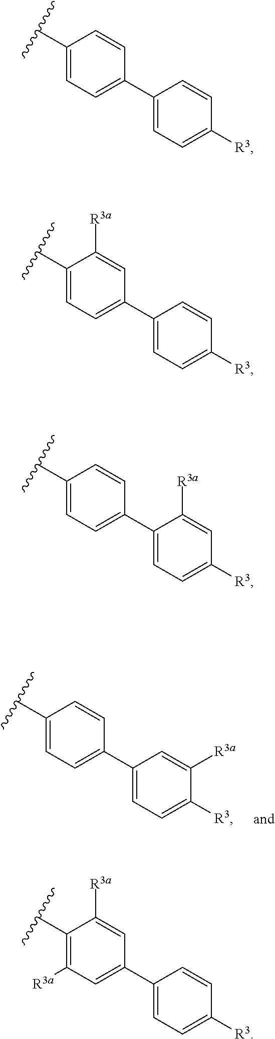

16. The compound of any one of claims 1-3 and 13-15, or a single stereoisomer or mixture of stereoisomers thereof, a single tautomer or mixture of tautomers thereof, and/or a pharmaceutically acceptable salt thereof, wherein R² is selected from the group consisting of

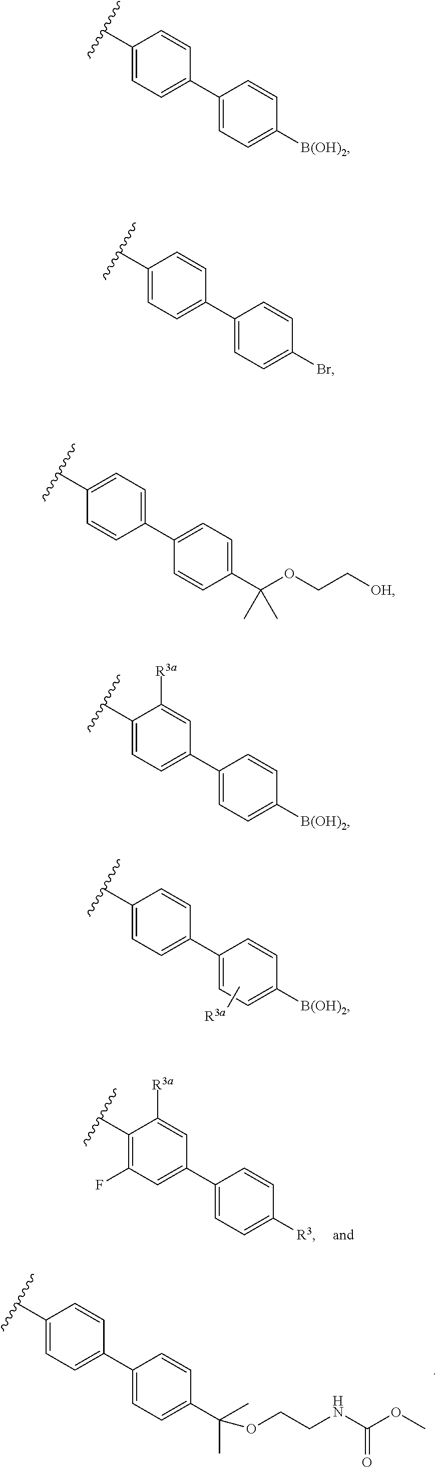

17. The compound of any one of claims 1-3, or a single stereoisomer or mixture of stereoisomers thereof, a single tautomer or mixture of tautomers thereof, and/or a pharmaceutically acceptable salt thereof, wherein R² is 5- to 6-membered heteroaryl substituted with R³ and additionally optionally substituted with 1, 2, or 3 R^{3a} groups; optionally wherein the R³ is at the para-position of the 6-membered heteroaryl.

18. The compound of any one of claims 1-3, or a single stereoisomer or mixture of stereoisomers thereof, a single tautomer or mixture of tautomers thereof, and/or a pharmaceutically acceptable salt thereof, wherein R² is 5- or 6-membered heteroaryl optionally substituted with 1, 2, or 3 R^{3a} groups and additionally substituted with -phenyl-R³ where the phenyl is additionally optionally substituted with 1, 2, or 3 R^{3a} groups; optionally wherein the -phenyl-R³ is at the para-position of the 6-membered heteroaryl; and optionally wherein the R³ is at the para-position of the phenyl.

19. The compound of any one of claims 1-3, or a single stereoisomer or mixture of stereoisomers thereof, a single tautomer or mixture of tautomers thereof, and/or a pharmaceutically acceptable salt thereof, wherein R² is 5- or 6-membered heteroaryl optionally substituted with 1, 2, or 3 R^{3a} groups and additionally substituted with -(5- or 6-membered heteroaryl)-R³ where the 5- or 6-membered heteroaryl in -(5- or 6-membered heteroaryl)-R³ is optionally substituted with 1, 2, or 3 R^{3a} groups; optionally wherein the -(6-membered heteroaryl)-R³ is at the para-position of the first 6-membered heteroaryl; and optionally wherein the R³ is at the para-position of the 6-membered heteroaryl to which it is attached.

20. The compound of any one of claims 1-3 and 19, or a single stereoisomer or mixture of stereoisomers thereof, a single tautomer or mixture of tautomers thereof, and/or a pharmaceutically acceptable salt thereof, wherein R² is

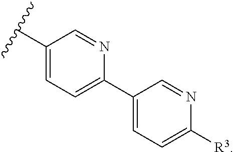

21. The compound of any one of claims 1-3, or a single stereoisomer or mixture of stereoisomers thereof, a single tautomer or mixture of tautomers thereof, and/or a pharmaceutically acceptable salt thereof, wherein R² is C₃-C₆cycloalkyl substituted with R³ and additionally optionally substituted with 1 or 2 R^{3a} groups.

22. The compound of any one of claims 1-3 and 21, or a single stereoisomer or mixture of stereoisomers thereof, a single tautomer or mixture of tautomers thereof, and/or a pharmaceutically acceptable salt thereof, wherein R² is 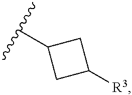
optionally wherein R³ is 5- to 10-membered heterocyclic optionally substituted with cyano.

23. The compound of any one of claims 1-3, or a single stereoisomer or mixture of stereoisomers thereof, a single tautomer or mixture of tautomers thereof, and/or a pharmaceutically acceptable salt thereof, wherein R² is C₃-C₆cycloalkyl substituted with 5- or 6-membered heteroaryl where the 5- or 6-membered heteroaryl is substituted with R³ and additionally optionally substituted with 1, 2, or 3 R^{3a} groups, and where the cycloalkyl is optionally substituted with 1 or 2 R^{3a} groups.

24. The compound of any one of claims 1-3, or a single stereoisomer or mixture of stereoisomers thereof, a single tautomer or mixture of tautomers thereof, and/or a pharmaceutically acceptable salt thereof, wherein R² is C₃-C₆cycloalkyl substituted with phenyl where the phenyl is substituted with R³ and additionally optionally substituted with 1, 2, or 3 R^{3a} groups, and where the cycloalkyl is optionally substituted with 1 or 2 R^{3a} groups.

25. The compound of any one of claims 1-3, or a single stereoisomer or mixture of stereoisomers thereof, a single tautomer or mixture of tautomers thereof, and/or a pharmaceutically acceptable salt thereof, wherein R² is 3- to 8-membered heterocycloalkyl substituted with phenyl or 5- or 6-membered heteroaryl, where the phenyl and the 5- to 6-membered heteroaryl are substituted with R³ and additionally optionally substituted with 1, 2, or 3 R^{3a} groups.

26. The compound of any one of claims 1-3 and 25, or a single stereoisomer or mixture of stereoisomers thereof, a single tautomer or mixture of tautomers thereof, and/or a pharmaceutically acceptable salt thereof, wherein R² is selected from the group consisting of

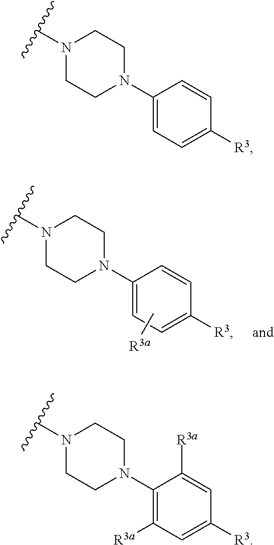

27. The compound of any one of claims 1-3, or a single stereoisomer or mixture of stereoisomers thereof, a single tautomer or mixture of tautomers thereof, and/or a pharmaceutically acceptable salt thereof, wherein R² is —CH═CH—R⁵ where R⁵ is phenyl or 5- or 6-membered heteroaryl, where the phenyl and the 5- or 6-membered heteroaryl are substituted with R³ and additionally optionally substituted with 1, 2, or 3 R^{3a} groups.

28. The compound of any one of claims 1-3 and 27, or a single stereoisomer or mixture of stereoisomers thereof, a single tautomer or mixture of tautomers thereof, and/or a pharmaceutically acceptable salt thereof, wherein R² is selected from the group consisting of

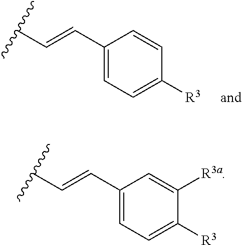

29. The compound of any one of claims 1-28, or a single stereoisomer or mixture of stereoisomers thereof; a single tautomer or mixture of tautomers thereof; and/or a pharmaceutically acceptable salt thereof; wherein R³ is cyano, —B(OH)₂, or —(C₀-C₆alkylene)-O—R⁴.

30. The compound of any one of claims 1-7, 9-11, and 13-29, or a single stereoisomer or mixture of stereoisomers thereof; a single tautomer or mixture of tautomers thereof; and/or a pharmaceutically acceptable salt thereof, wherein R³ is cyano.

31. The compound of any one of claims 1-7, 9-11, and 13-29, or a single stereoisomer or mixture of stereoisomers thereof; a single tautomer or mixture of tautomers thereof; and/or a pharmaceutically acceptable salt thereof, wherein R³ is —B(OH)₂.

32. The compound of any one of claims 1-7, 9-11, and 13-29, or a single stereoisomer or mixture of stereoisomers thereof; a single tautomer or mixture of tautomers thereof; and/or a pharmaceutically acceptable salt thereof; wherein R³ is —(C₀-C₆alkylene)-O—R⁴.

33. The compound of any one of claims 1-7, 9-11, 13-29, and 32, or a single stereoisomer or mixture of stereoisomers thereof; a single tautomer or mixture of tautomers thereof; and/or a pharmaceutically acceptable salt thereof; wherein R³ is —(C₁₋₆alkylene)-O—R⁴.

34. The compound of any one of claims 1-7, 9-11, 13-29, 32, and 33, or a single stereoisomer or mixture of stereoisomers thereof; a single tautomer or mixture of tautomers thereof; and/or a pharmaceutically acceptable salt thereof; wherein R⁴ is hydroxy-C₁-C₆alkyl.

35. The compound of any one of claims 1-7, 9-11, 13-29, 32, and 33, or a single stereoisomer or mixture of stereoisomers thereof; a single tautomer or mixture of tautomers thereof; and/or a pharmaceutically acceptable salt thereof; wherein R⁴ is C₁-C₆alkoxy-C₁-C₆alkyl.

36. The compound of any one of claims 1-7, 9-11, 13-29, 32, and 33, or a single stereoisomer or mixture of stereoisomers thereof; a single tautomer or mixture of tautomers thereof; and/or a pharmaceutically acceptable salt thereof, wherein R⁴ is C₁-C₆alkoxycarbonyl-NH—C₁-C₆alkyl.

37. The compound of any one of claims 1-3, 9-11, 13-16, and 18-20, or a single stereoisomer or mixture of stereoisomers thereof; a single tautomer or mixture of tautomers thereof; and/or a pharmaceutically acceptable salt thereof; wherein R² is (b), (c), (e), or (f) and R³ is halo, cyano, —B(OH)₂, or —(C₀-C₆alkylene)-O—R⁴.

38. The compound of claim 1, or a single stereoisomer or mixture of stereoisomers thereof; a single tautomer or mixture of tautomers thereof, and/or a pharmaceutically acceptable salt thereof, selected from the group consisting of the Compounds 1-44 provided in Table 1.

39. A pharmaceutical composition comprising the compound of any one of claims 1-38, or a single stereoisomer or mixture of stereoisomers thereof, a single tautomer or mixture of tautomers thereof, and/or a pharmaceutically acceptable salt thereof, and comprising a pharmaceutically acceptable carrier.

40. The pharmaceutical composition of claim 39, where the pharmaceutical carrier comprises:
a matrix component comprising a graphene oxide (GO) and hyaluronic acid (HA) conjugate (GO-HA), wherein GO and HA are covalently linked via a linker; polyethylene glycol (PEG), where the PEG is optional;
a thickener, where the thickener is optional; and
water,
optionally wherein the compound optionally constitutes from about 0.001 wt % to about 5 wt % of the total composition.

41. A method of inhibiting Wnt signaling pathway activity in a subject comprising contacting an effective amount of a compound of any one of claims 1-38 or a single stereoisomer or mixture of stereoisomers thereof, a single tautomer or mixture of tautomers thereof, and/or a pharmaceutically acceptable salt thereof, with the subject.

42. A method of treating a disease, disorder, or condition associated with Wnt signaling pathway activity, comprising administering a compound of any one of claims 1-38, or a single stereoisomer or mixture of stereoisomers thereof, a single tautomer or mixture of tautomers thereof, and/or a pharmaceutically acceptable salt thereof; or administering a pharmaceutical composition of claim 39 or 40 to a mammal in need thereof.

43. The method of claim 42, wherein the method is for stimulating regeneration of tissue at a wound in the mammal in need thereof and wherein the wound is contacted with an effective amount of the compound (or a single stereoisomer or mixture of stereoisomers thereof, a single tautomer or mixture of tautomers thereof, and/or a pharmaceutically acceptable salt thereof) or the pharmaceutical composition.

44. The method of claim 42, wherein the disease, disorder, or condition is selected from a chronic wound, an acute wound, an alkali-burned corneal wound, a burn, a lesion (including lesions caused by HPV and/or a virus selected from the Poxviridae family of viruses), an inflammatory dermatitis disease (including acne, psoriasis, rosacea, and scleroderma), a cartilage disease (including osteoarthritis, rheumatoid arthritis, internal derangement of the joints, and degenerative cartilage disease), a bone disease (including osteoporosis), organ fibrosis (including lung fibrosis, heart fibrosis, liver fibrosis, and kidney fibrosis), cancer (including melanoma, breast cancer, and prostate cancer), a de-nerved body part in need of reinnervation, tissue in need of regeneration (including damaged elastic cartilage), bacterial growth in need of inhibition, fungal growth in need of inhibition, tissue in need of neovascularization, osteoclast differentiation in need of inhibition, osteoblast differentiation disorders (where inhibition of osteoblast differentiation is needed), and/or bone destruction associated with breast cancer.

45. A method of inducing bacteriostasis associated with Wnt signaling pathway activity, comprising administering XAV939 or tautomer thereof and/or pharmaceutically acceptable salt thereof, optionally in a pharmaceutically acceptable carrier; administering a compound of any one of claims 1-38, or a single stereoisomer or mixture of stereoisomers thereof, a single tautomer or mixture of tautomers thereof, and/or a pharmaceutically acceptable salt thereof, or administering a pharmaceutical composition of claim 39 or 40 to a mammal in need thereof.

46. A compound, or a salt thereof, and/or a stereoisomer or mixture of stereoisomers according to any one of the following formulas: 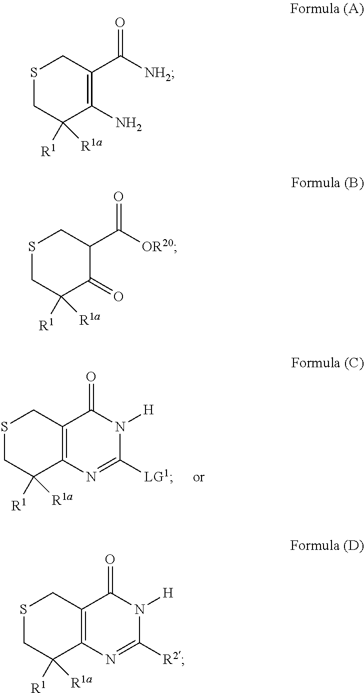
and
wherein
LG¹ is a leaving group, such as fluoro, chloro, bromo, iodo, triflate, mesylate, a triazole, a pyrazole, boronic acid, boronic ester, or aryl trifluoroborate;
R¹ is hydrogen, deuterium, C₁-C₃alkyl, —OH, —O—C₁-C₃alkyl, —CH₂OH, or —B(OH)₂;
R^{1a} is hydrogen, deuterium, or C₁-C₃alkyl;
R²⁰ is alkyl, preferably methyl or ethyl, or CD₃;
R^{2′} is
(b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups,
(c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups;
(e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups;
(f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups;
(h1) C₃-C₆cycloalkyl substituted with NH₂ or OH and additionally optionally substituted with 1 or 2 R^{3a} groups; or
(i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and
provided that the compound is not:
methyl 4-oxotetrahydro-2H-thiopyran-3-carboxylate 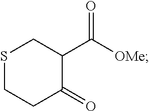 [SMILES: COC(=O)C1CSCCC1=O]
or
a salt thereof and/or a stereoisomer or mixture of stereoisomers thereof.

46. A method of preparing a compound of Formula (I) of any one of claims 1-37, comprising:
a) contacting a compound of Formula (A): 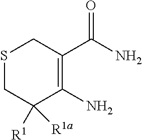
with R^{2′}—C(O)H; or
b) contacting a compound of Formula (B): 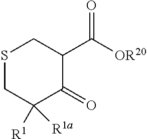
with R^{2′}—C(NH)NH₂, wherein R²⁰ is Me or CD₃; or
c) contacting a compound of Formula (C): 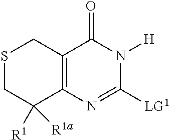
with R^{2′}—H, wherein LG¹ is fluoro, chloro, bromo, iodo, triflate, mesylate, a triazole, a pyrazole, boronic acid, boronic ester, or aryl trifluoroborate;
and optionally isolating the compound of Formula (I);
wherein R^{2′} is
(b1) phenyl substituted with 5- or 6-membered heteroaryl where the 5- or 6-membered heteroaryl is substituted with LG¹ and additionally optionally substituted with 1, 2, or 3 R^{3a} groups and where the phenyl is additionally optionally substituted with 1, 2, or 3 R^{3a} groups,
(c1) phenyl optionally substituted with 1, 2, or 3 R^{3a} groups and additionally substituted with -phenyl-LG¹ where the phenyl in -phenyl-LG¹ is optionally substituted with 1, 2, or 3 R^{3a} groups;
(e1) 5- or 6-membered heteroaryl optionally substituted with 1, 2, or 3 R^{3a} groups and additionally substituted with -phenyl-LG¹ where the phenyl is additionally optionally substituted with 1, 2, or 3 R^{3a} groups;
(f1) 5- or 6-membered heteroaryl optionally substituted with 1, 2, or 3 R^{3a} groups and additionally substituted with -(5- or 6-membered heteroaryl)-LG¹ where the 5- or 6-membered heteroaryl in -(5- or 6-membered heteroaryl)-LG¹ is optionally substituted with 1, 2, or 3 R^{3a} groups;
(h1) C₃-C₆cycloalkyl substituted with NH₂ or OH and additionally optionally substituted with 1 or 2 R^{3a} groups; or
(i1) C₃-C₆cycloalkyl substituted with phenyl where the phenyl is substituted with LG¹ and the phenyl is additionally optionally substituted with 1, 2, or 3 R^{3a} groups, and where the cycloalkyl is optionally substituted with 1 or 2 R^{3a} groups; and
provided that the compound is not:
methyl 4-oxotetrahydro-2H-thiopyran-3-carboxylate 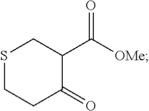 [SMILES: COC(=O)C1CSCCC1=O]
or
a salt thereof and/or a stereoisomer or mixture of stereoisomers thereof.

47. The method of claim 46, wherein the contacting is under basic conditions.
